SEQUENCE LISTING (5 sequences)

SEQ ID NO: 1 - protein, 5 aa, synthetic construct
FYPWM

SEQ ID NO: 2 - protein, 6 aa, synthetic construct
FYPWMK

SEQ ID NO: 3 - protein, 5 aa, synthetic construct
YPWMK

SEQ ID NO: 4 - DNA, 15 bp, synthetic construct
  source 1..15: mol_type = other DNA
tgattgataaggtat

SEQ ID NO: 5 - protein, 4 aa, synthetic construct
YPWM

(19) United States
(12) Patent Application Publication
(10) Pub. No.: US 2026/0317308 A1
(43) Pub. Date: Oct. 15, 2026

(54) A SEQUENCE SELECTIVE DNA BINDING MOLECULE (SYNGRADER)

(71) Applicant: ST. JUDE CHILDREN'S RESEARCH HOSPITAL, INC., Memphis, TN (US)

(72) Inventors: Aseem Z. Ansari, Germantown, TN (US); Ashraf Mohammed, Collierville, TN (US); Erik Bonten, Arlington, TN (US)

(21) Appl. No.: 19/479,686

(22) PCT Filed: May 2, 2024
(86) PCT No.: PCT/US2024/027540

§ 371 Date: Oct. 28, 2025

Related U.S. Application Data
(60) Provisional application No. 63/463,834, filed on May 3, 2023.

(51) Int. Cl. C07D 403/14; A61K 31/4178; A61K 31/427; A61K 31/5517; C07D 401/14; C07D 417/14; C07D 495/14

(52) U.S. Cl. CPC C07D 403/14; A61K 31/4178; A61K 31/427; A61K 31/5517; C07D 401/14; C07D 417/14; C07D 495/14

(57) ABSTRACT

The present disclosure relates to chemical compounds that enable targeted degradation or non-proteolytic inactivation of DNA binding proteins and confer selectivity of action on proteins that are bound to the genome, pharmaceutical compositions containing such compounds, and their use in treatment. This abstract is intended as a scanning tool for purposes of searching in the particular art and is not intended to be limiting of the present invention.

### CROSS-REFERENCE TO RELATED APPLICATIONS

This application claims the benefit of U.S. Application No. 63/463,834, filed on May 3, 2023, the contents of which are incorporated herein by reference in their entirety.

### BACKGROUND

As a traditional treatment method, chemotherapy plays an irreplaceable role in the cancer treatment process. The main disadvantages of traditional anticancer drugs are that most of them have poor selectivity and are easy to develop drug resistance (Mangal et al. (2017) Acta Pharmacol. Sin. 38:782-797; Dong et al. (2020) ChemMedChem 13:1490-1507; Yuan et al. (2020) Mol. Cancer 19:96). As a result, the targeted therapy of cancer has attracted people's attention (Zhou et al. (2020) Drug Discov. Today 25(11): 1988-1997; Qi et al. (2020) Front Cel Dev Biol. 8: 233; Yu et al. (2020) Front. Oncol. 10: 1389). On this basis, the discoveries of new targets and small molecule inhibitors (SMIs) have become powerful treatment strategies (Dong et al. (2018) ChemMedChem 13: 1490-1507). In particular, the development of small molecule kinase inhibitors has become one of the most widely pursued fields in the process of drug discovery and has made great achievements in cancer treatment (Wu et al. (2015) Trends Pharmacol. Sci. 36: 422-439). However, after the success, the treatment strategy also faces the same problem of drug resistance as chemotherapy (Dong et al. (2020) ChemMedChem 13: 1490-1507; Xu et al. (2020) Front. Cel. Dev Biol 8: 621428). Therefore, drug resistance is the main limitation for cancer therapy and needs to be solved urgently.

In recent years, targeted protein degradation (TBD) has gained tremendous attention. Proteolysis-targeting chimeras (PROTACs) or bivalent chemical protein degraders are heterobifunctional molecules that degrade target proteins by hijacking the ubiquitin-proteasome system (Potjewyd et al. (2020) Cel Chem. Biol. 27: 47-56). Unlike small molecules, PROTACs inhibit the whole biological function of the target protein by binding to the target protein and inducing subsequent proteasomal degradation.

Transcription factors (TFs) are DNA-binding proteins that directly or indirectly regulate gene expression. Many diseases, disorders, and cancers can result from abnormalities in the TF-controlled gene regulatory circuits, and, as such, much effort has been devoted to therapeutically target TFs implicated in human diseases (Sharifnia et al. (2019) Nat. Med. 25: 292-300). Unfortunately, because most TFs mediate their regulatory functions through interactions with DNA and/or with other proteins, they frequently lack enzymatic activity and ligandable pockets—necessary features that have been successfully exploited in developing small-molecule inhibitors for more ready druggable proteins. These features (or the lack thereof) has also rendered the development of traditional PROTACs for TF degradation challenging, as a target ligand is still required.

One strategy recently used to bypass the need for target protein ligand development is to exploit the intrinsic TF DNA-binding ability. See, e.g., Samarasinghe et al. (2021) Cell Chemical Biology 28:648-661. Specifically, chimeric oligonucleotides having a TF-specific DNA sequence attached to an E3-ligase-recruiting moiety have been used to induce ubiquitination and proteasomal degradation of the target of interest. Although this strategy has widespread implications for the targeted degradation of DNA-binding proteins such as TFs, the development of such chimeras has remained limited. Thus, there is a need for heterofunctional compounds and compositions to target and inactivate DNA-binding proteins that are involved in a range of genomic functions.

### SUMMARY

In accordance with the purpose(s) of the invention, as embodied and broadly described herein, the invention, in one aspect, relates to sequence selective DNA binding compounds that associate with DNA-bound proteins, thereby promoting their proteolysis/degradation and inactivation. The invention further relates to pharmaceutical compositions comprising the compounds, and methods of using the compounds for treating disorders such as, for example, cancer (e.g., a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma).

Thus, in one aspect, disclosed are sequence selective DNA binding compounds comprising a polyamide moiety configured to bind a deoxyribonucleic acid (DNA) sequence, wherein the polyamide moiety is tethered via a first chemical linker to an Ubiquitin-Proteosome System (UPS) targeting moiety.

Also disclosed are sequence selective DNA binding compounds comprising a polyamide moiety configured to bind a deoxyribonucleic acid (DNA) sequence, wherein the polyamide moiety is tethered via a first chemical linker to a PBX or TCF3-PBX transcription factor binding moiety.

Also disclosed are sequence selective DNA binding compounds comprising: (a) a polyamide moiety configured to bind a deoxyribonucleic acid (DNA) sequence; and (b) two of a Ubiquitin-Proteosome System (UPS) targeting moiety, a bromodomain extraterminal domain (BET) binding ligand, and a PBX or TCF3-PBX transcription factor binding moiety, wherein the polyamide moiety and two of the UPS targeting moiety, the BET binding ligand, and the transcription factor binding moiety are tethered via a first chemical linker and via a second chemical linker.

Also disclosed are pharmaceutical compositions comprising a disclosed compound or a pharmaceutically acceptable salt thereof and a pharmaceutically acceptable carrier.

Also disclosed are methods of treating a disorder comprising administering to a subject having the disorder a therapeutically effective amount of a disclosed compound or a pharmaceutically acceptable salt thereof.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

The accompanying figures, which are incorporated in and constitute a part of this specification, illustrate several aspects and together with the description serve to explain the principles of the invention.

FIG. 1 shows a representative schematic of a bi-functional degrader having an E3 ligase binding ligand tethered to a polyamide via a PEG linker.

FIG. 2 shows a representative schematic of a tri-functional degrader having both an E3 ligase binding ligand and a BET binding ligand, each tethered to a polyamide via a PEG linker.

FIG. 3 shows a representative schematic of a tri-functional degrader having a polyamide tethered to a TCF3-PBX binding peptide that is further linked to an E3 ligase binding ligand.

FIG. 4 shows representative polyamide groups.

FIG. 5 shows representative E3 ligase binding ligands.

FIG. 6 shows representative TCF3-PBX binding peptides.

FIG. 7 shows representative chemical linkers.

FIG. 8 shows representative bi-functional degraders.

FIG. 9 shows a representative synthetic scheme illustrating the preparation of polyamides.

FIG. 10A and FIG. 10B show representative spectral data for PA1. Specifically, a mass spectra (FIG. 10A) and a ¹H NMR spectra (FIG. 10B) are shown.

FIG. 11A and FIG. 11B show representative spectral data for PA1-(PEG)₆-NH₂. Specifically, a mass spectra (FIG. 11A) and a ¹H NMR spectra (FIG. 11B) are shown.

FIG. 12A and FIG. 12B show representative synthetic schemes illustrating the preparation of DGR-Syn-TEFs (FIG. 12A) and N-DGR-Syn-TEFs (FIG. 12B).

FIG. 13A and FIG. 13B show representative mass spectral data corresponding to SynTEF-DRG7 (FIG. 13A) and SynTEF-DRG10 (FIG. 13B).

FIG. 14 shows a representative synthetic scheme illustrating the preparation of N-DGR-Syn-TEFs.

FIG. 15A and FIG. 15B show representative synthetic schemes illustrating the preparation of hairpin polyamides.

FIG. 16A-D show representative mass spectral data corresponding to PBX-PA1 (FIG. 16A), PBX-PA2 (FIG. 16B), PBX-PA1 PEG₃-NH₂ (FIG. 16C), and PBX-PA2 PEG₃-NH₂ (FIG. 16D).

FIG. 17 shows a representative synthetic scheme illustrating the preparation of the DRG-Peptide.

FIG. 18 shows a representative synthetic scheme illustrating the preparation of PBX-SynTEF-DGRs.

FIG. 19A and FIG. 19B show representative mass spectral data of PBX-SynTEF-DRG1 (FIG. 19A) and PBX-SynTEF-DRG2 (FIG. 19B).

FIG. 20 shows a representative synthetic scheme illustrating the preparation of PBX-SynTEF-NDGRs.

FIG. 21A and FIG. 21B show representative mass spectral data of SynTEF-DRG5 (FIG. 21A) and SynTEF-DRG6 (FIG. 21B).

FIG. 22A and FIG. 22B show representative data illustrating WGGWWW-targeting PBX-SynTEF (Synthetic HOX-Mimic) compounds.

FIG. 23A and FIG. 23B show representative data illustrating WGGWWW-targeting PBX-SynTEF (Synthetic HOX-Mimic) degrader compounds.

FIG. 24A and FIG. 24B show representative data from Electrophoretic Mobility Shift Assays (EMSA) of purified PBX1 extended homeodomain (PBX1-HD-Ext) and full length TCF3-PBX1 proteins with PBX-SynTEF2 polyamide and duplex DNAs containing PBX1 and PBX-SynTEF2 binding sites.

FIG. 25 shows representative data from a P2RY10 luciferase reporter assay in HEK293T cells expressing Doxycycline-inducible TCF3-PBX1-HaloTag, transfected with WGGWWW-SynTF compounds PBX-SynTF2, SynTF-PBX-DRG1, and SynTF-PBX-DRG2.

FIG. 26A and FIG. 26B shows representative data from a cell viability assay in NALM6 (N6) Acute Lymphoblastic Leukemia (ALL) B cells expressing Doxycycline-induced TCF3-PBX1-HaloTag, co-transfected with P2RY10 and CMV Luciferase reporter plasmid DNAs and treated with the SynTEF compounds PBX-SynTEF-FL, SynTEF-PBX-DRG1, and SynTEF-PBX-DRG2.

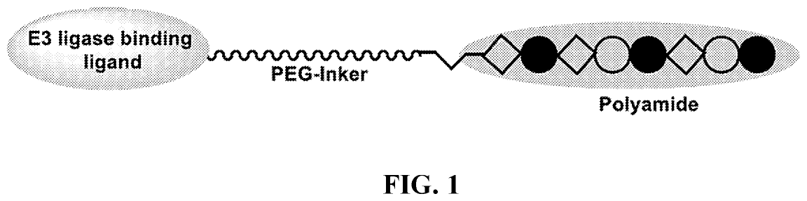

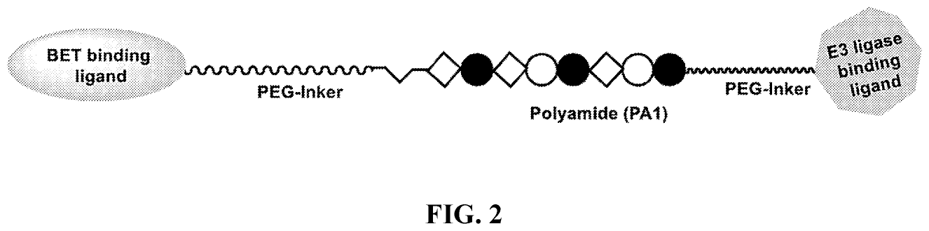

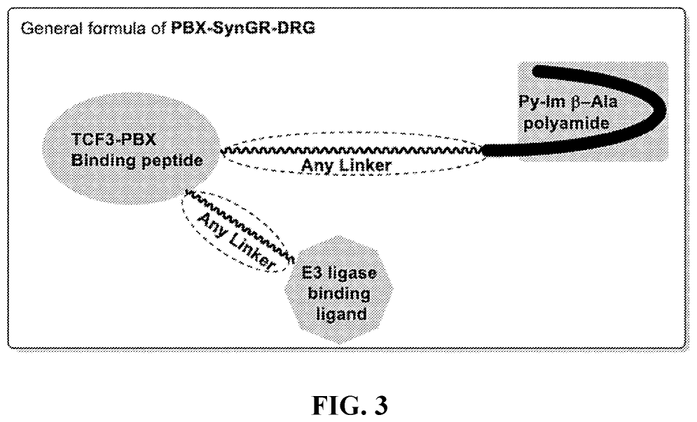

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage form can comprise a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. For example, the cycloalkyl group and heterocycloalkyl group can be substituted with 0, 1, 2, 3, or 4 groups independently selected from C1-C4 alkyl, C3-C7 cycloalkyl, C1-C4 alkoxy, —NH2, (C1-C4) alkylamino, (C1-C4)(C1-C4) dialkylamino, ether, halogen, —OH, C1-C4 hydroxyalkyl, —NO₂, silyl, sulfo-oxo, —SH, and C1-C4 thioalkyl, as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. For example, the cycloalkenyl group and heterocycloalkenyl group can be substituted with 0, 1, 2, 3, or 4 groups independently selected from C1-C4 alkyl, C3-C7 cycloalkyl, C1-C4 alkoxy, C2-C4 alkenyl, C3-C6 cycloalkenyl, C2-C4 alkynyl, aryl, heteroaryl, aldeyhyde, —NH₂, (C1-C4) alkylamino, (C1-C4)(C1-C4) dialkylamino, carboxylic acid, ester, ether, halogen, —OH, C1-C4 hydroxyalkyl, ketone, azide, —NO₂, silyl, sulfo-oxo, —SH, and C1-C4 thioalkyl, as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” or “CO” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide,” as used herein can be used interchangeably and refer to F, Cl, Br, or I.

The terms “pseudohalide,” “pseudohalogen,” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl”, “heteroaryl”, “bicyclic heterocycle” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxy” or “hydroxyl” as used herein is represented by the formula-OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN or —C≡N.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S) R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR∘; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘)}₂; or —(C₁₋₄ straight or branched alkylene)C(O)O—N(R^{∘)}₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•)}₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

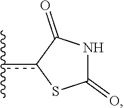

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and(S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or(S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

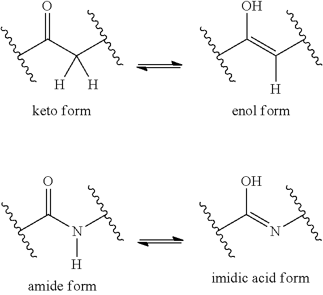 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

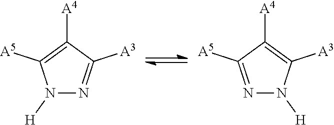

Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

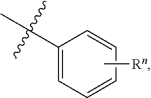

which is understood to be equivalent to a formula:

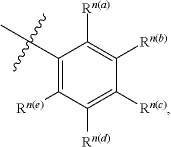

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow: plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. SEQUENCE SELECTIVE DNA BINDING COMPOUNDS

In one aspect, sequence selective DNA binding compounds that associate with DNA-bound proteins, thereby promoting their proteolysis/degradation and inactivation. Such compounds can be useful in treating a variety of disorders including, but not limited to cancers such as a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma).

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In one aspect, disclosed are sequence selective DNA binding compounds comprising a polyamide moiety configured to bind a deoxyribonucleic acid (DNA) sequence, wherein the polyamide moiety is tethered via a first chemical linker to an Ubiquitin-Proteosome System (UPS) targeting moiety.

In one aspect, disclosed are sequence selective DNA binding compounds comprising a polyamide moiety configured to bind a deoxyribonucleic acid (DNA) sequence, wherein the polyamide moiety is tethered via a first chemical linker to a PBX or TCF3-PBX transcription factor binding moiety.

In one aspect, disclosed are sequence selective DNA binding compounds comprising: (a) a polyamide moiety configured to bind a deoxyribonucleic acid (DNA) sequence; and (b) two of a Ubiquitin-Proteosome System (UPS) targeting moiety, a bromodomain extraterminal domain (BET) binding ligand, and a PBX or TCF3-PBX transcription factor binding moiety, wherein the polyamide moiety and two of the UPS targeting moiety, the BET binding ligand, and the transcription factor binding moiety are tethered via a first chemical linker and via a second chemical linker.

In various aspects, the compound is selected from:

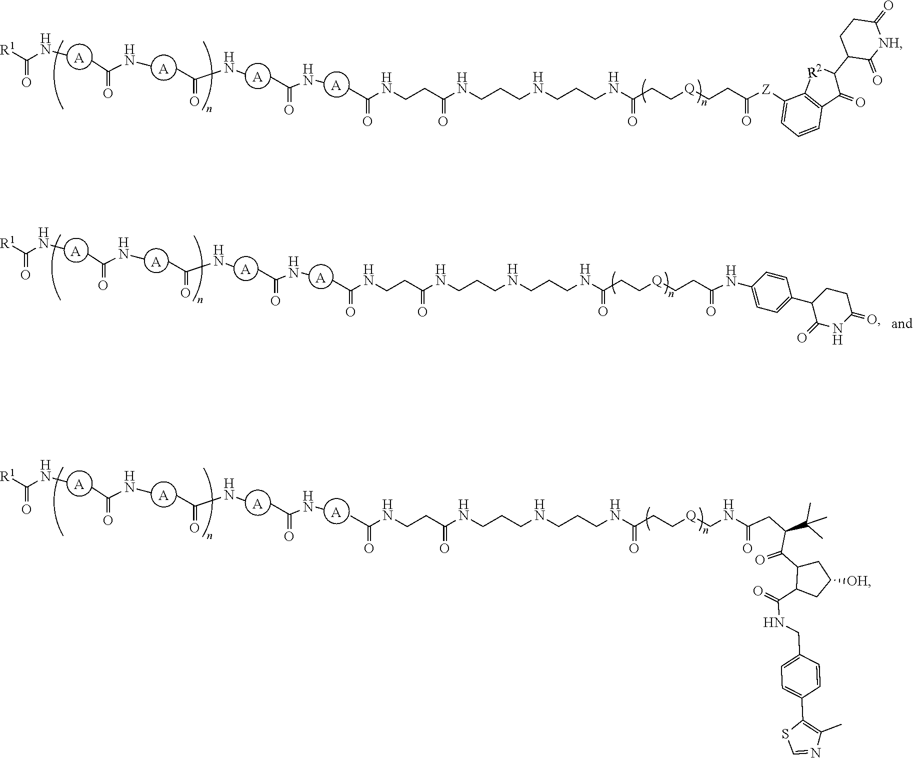

wherein each occurrence of n is independently an integer selected from 1 to 20; wherein Q is selected from CH₂ and O; wherein Z is selected from O and NH; wherein R¹ is selected from:

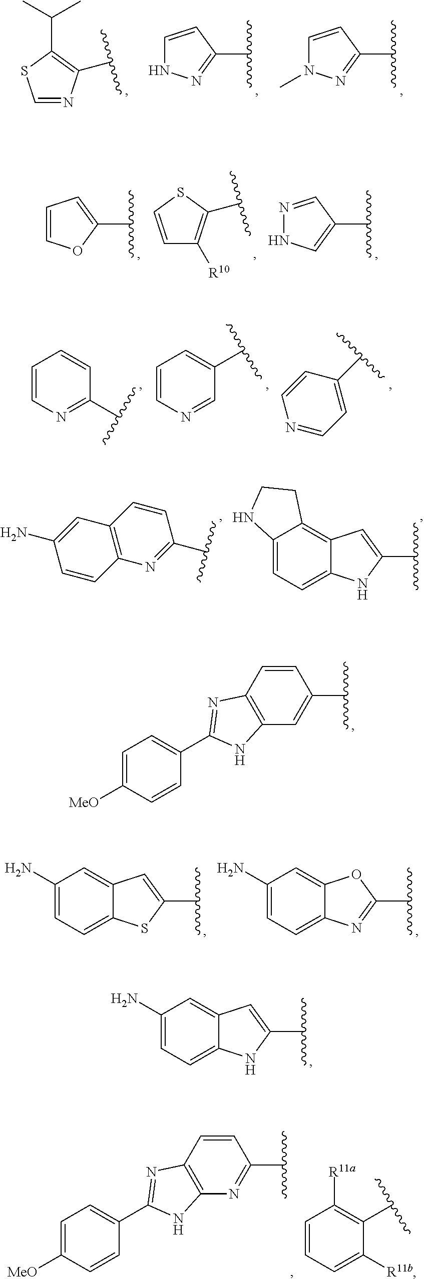

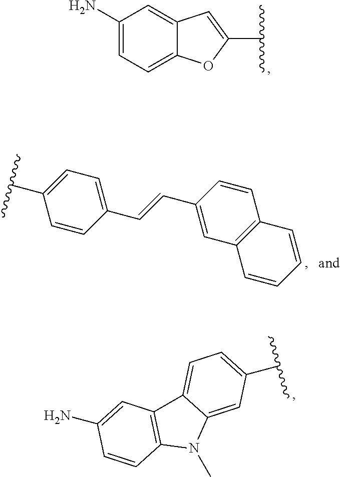

wherein R¹⁰ is selected from hydrogen, halogen, —OH, —NH₂, —NHC(O)CH₃, methyl, and methoxy; and wherein each of R^{11a} and R^{11b} is independently selected from hydrogen, —OH, methyl, and methoxy; and wherein each occurrence of \textcircled{A} is independently selected from:

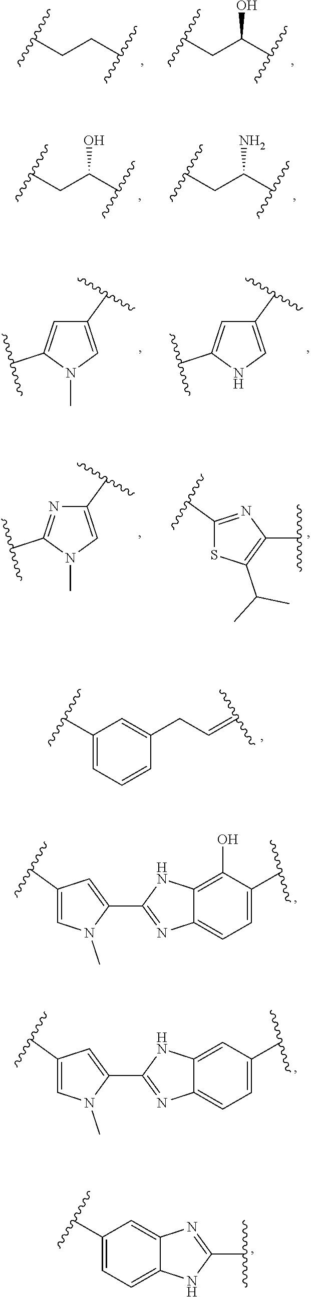

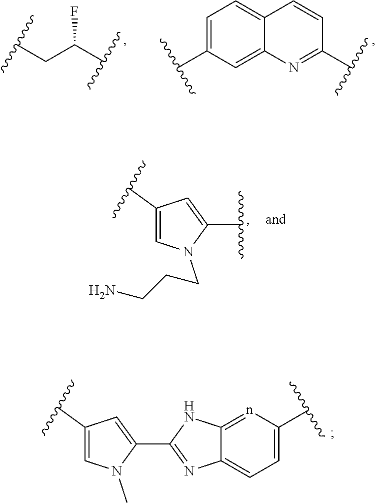

and
wherein R² is selected from CH₂ and C(O).

In various aspects, the compound is selected from:

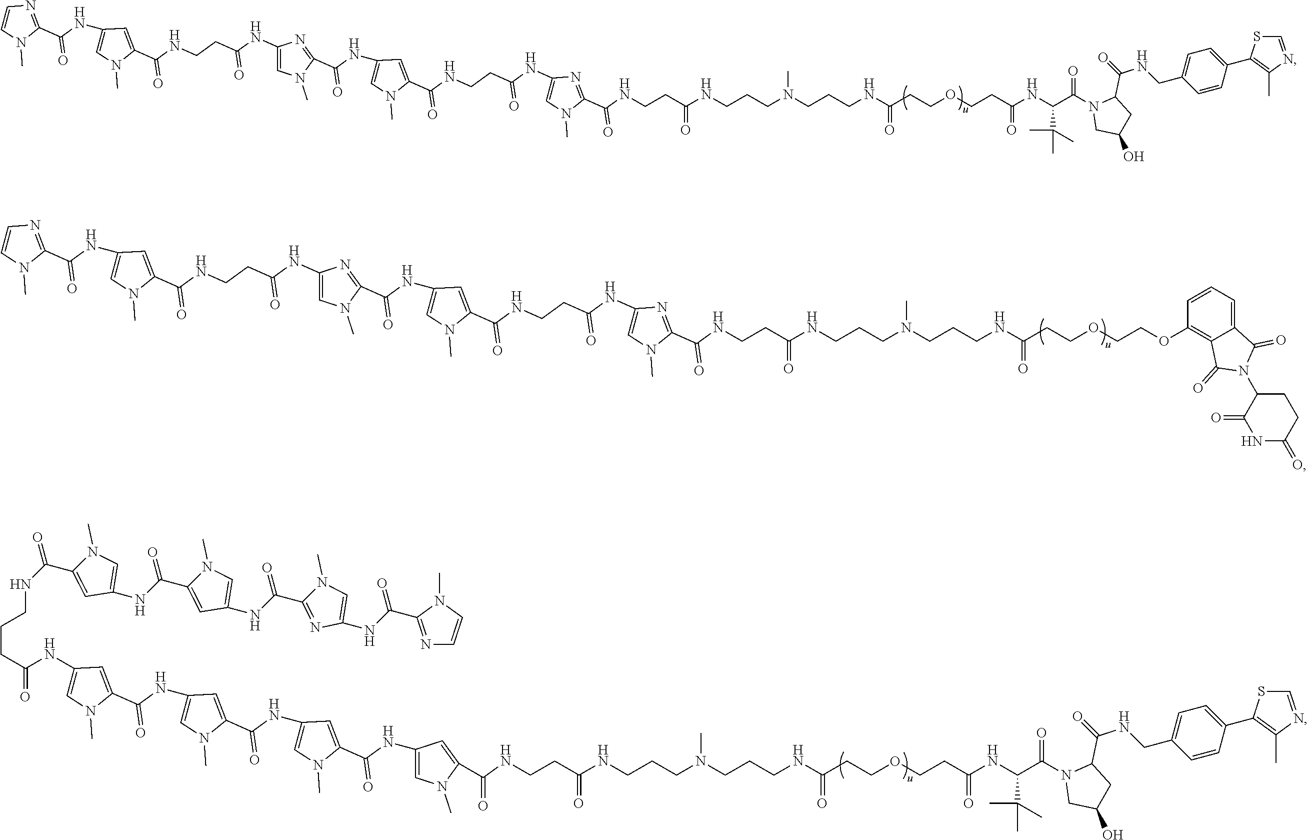

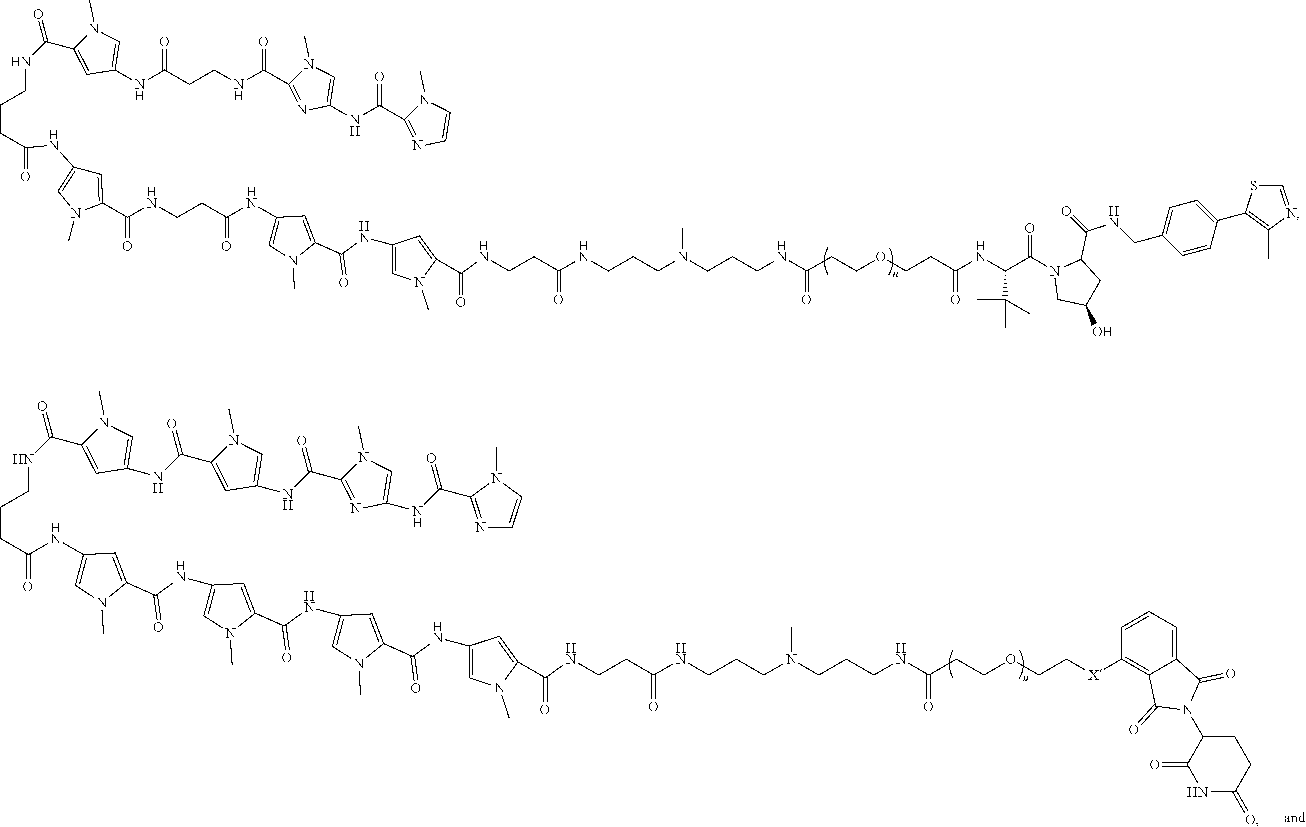

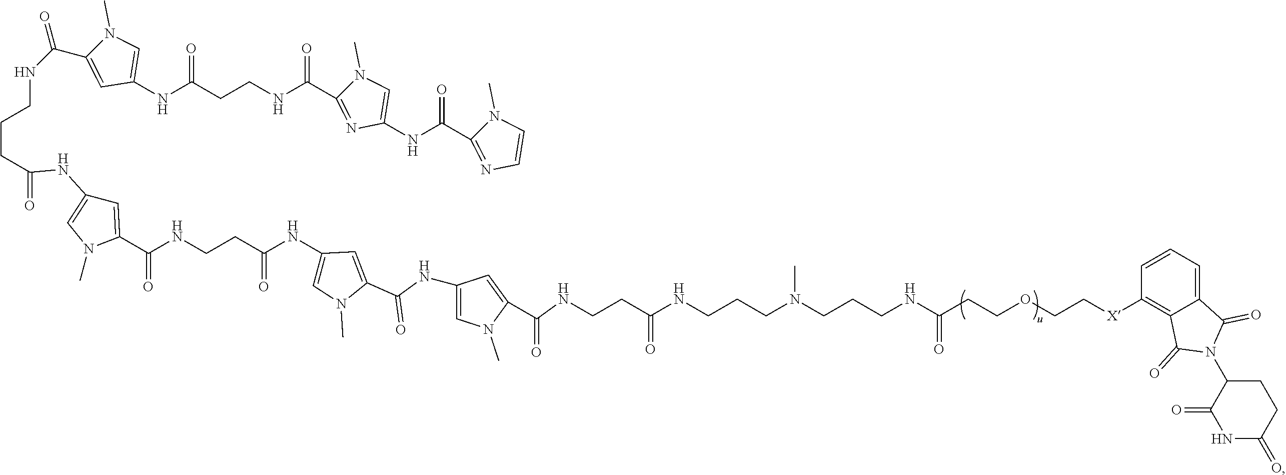

wherein u is an integer selected from 1 to 13; and wherein X′ is selected from NH and O.

In various aspects, the compound is selected from:

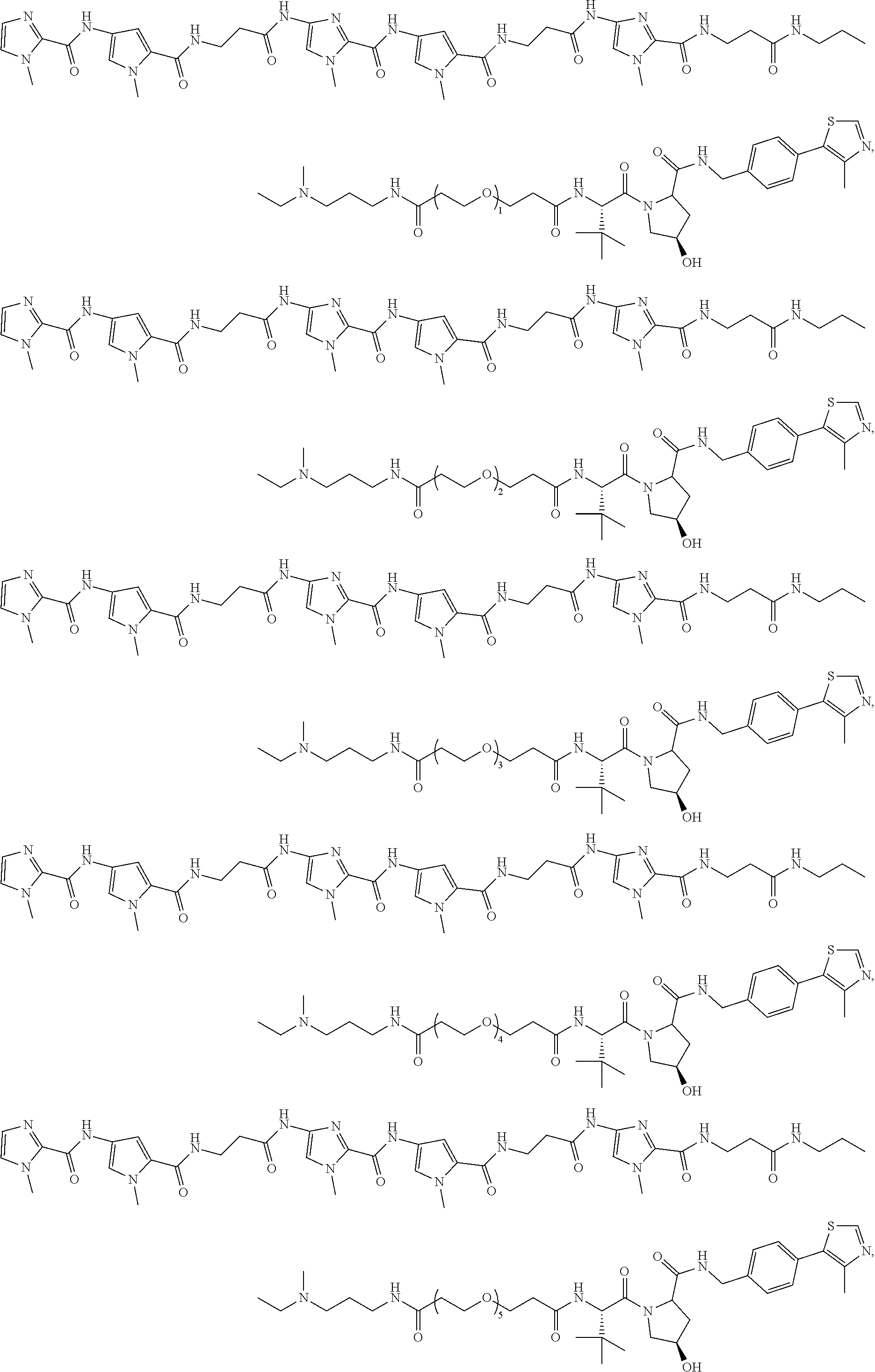

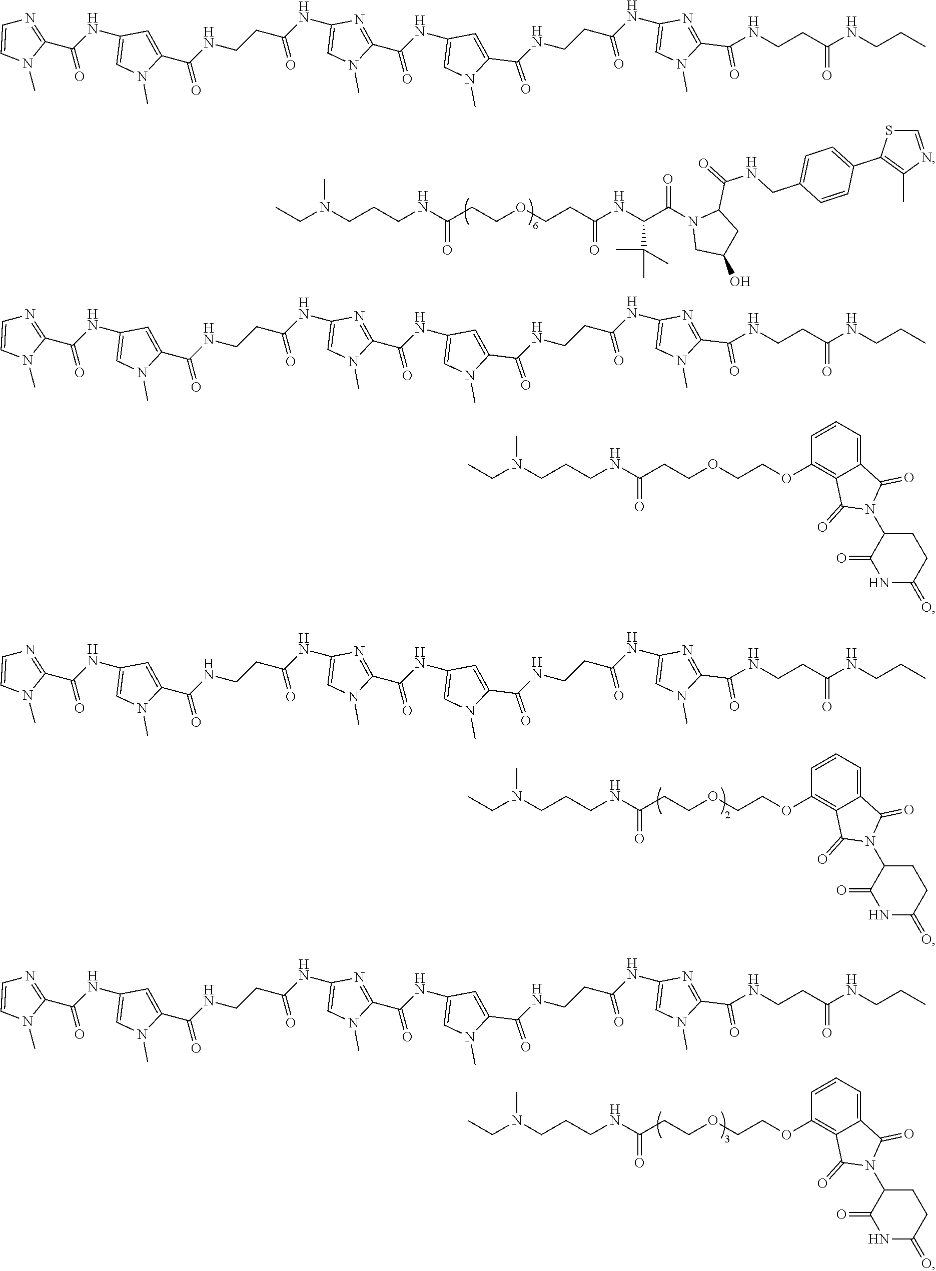

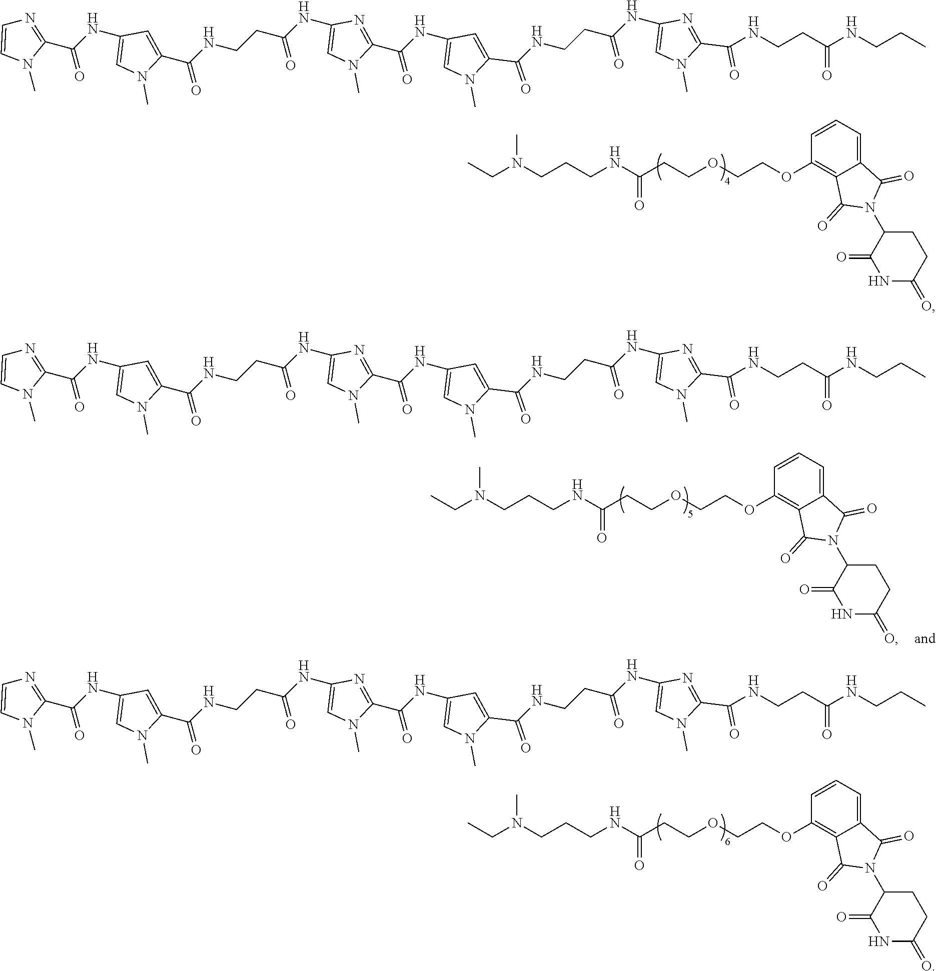

In various aspects, the compound has a structure selected from:

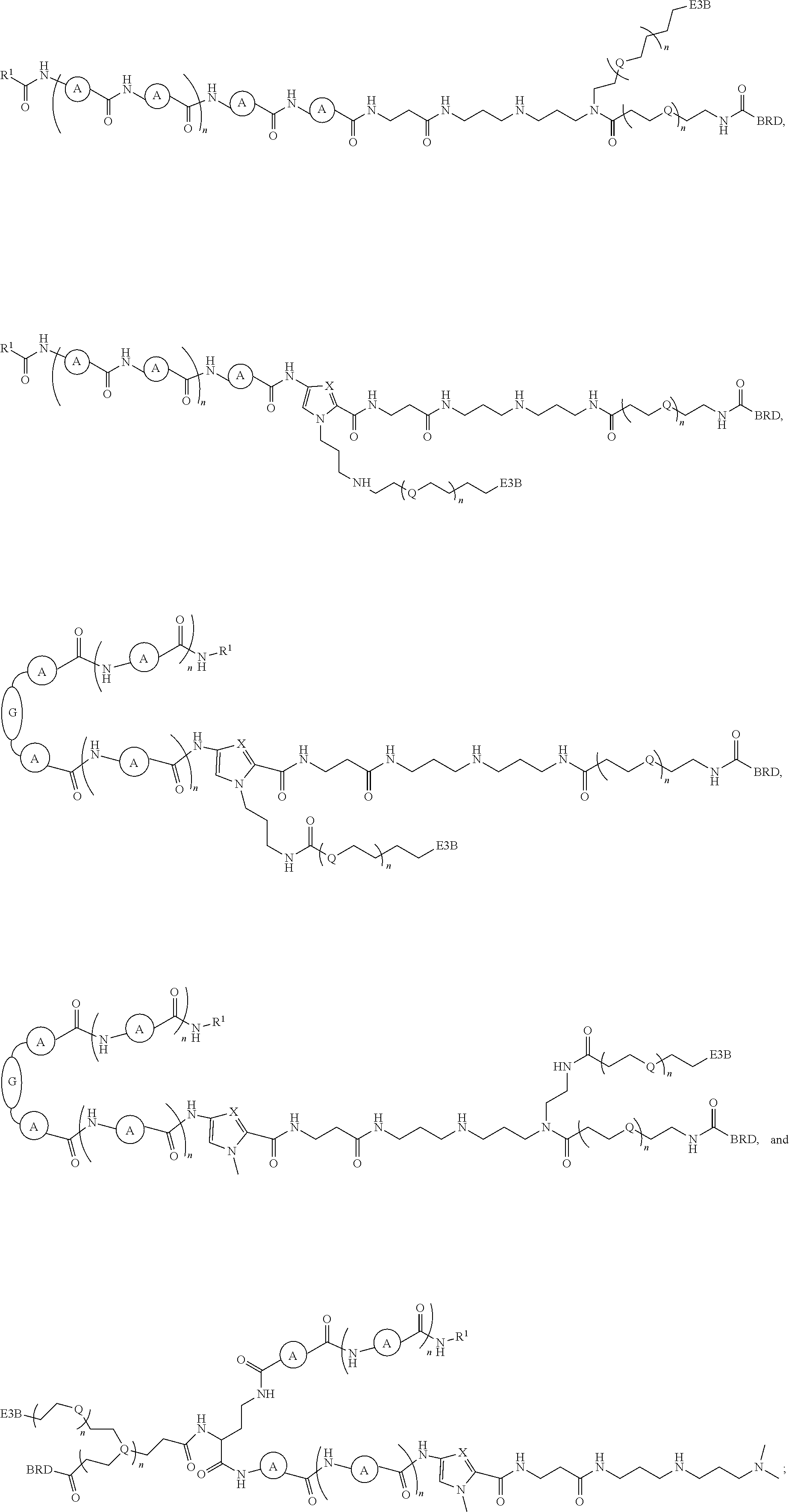

wherein E3B is an E3 ligase binding ligand; wherein BRD is a BET binding ligand; wherein each occurrence of n is independently an integer selected from 1 to 20; wherein G is a polyamide turn linker; wherein each occurrence of Q is independently selected from CH₂ and O; wherein X is selected from N and CH; wherein R¹ is selected from:

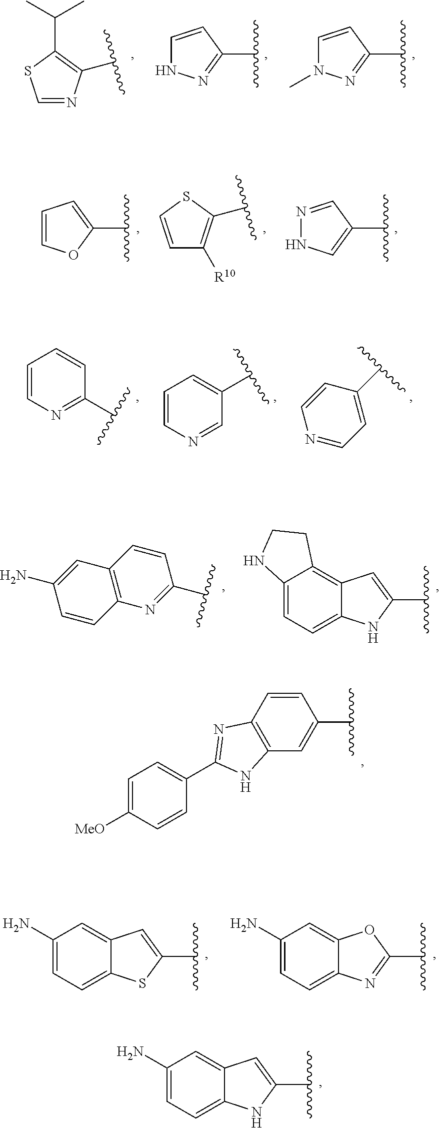

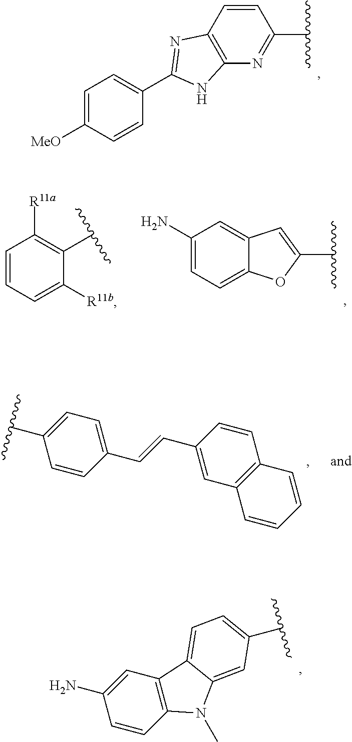

wherein R¹⁰ is selected from hydrogen, halogen, —OH, —NH₂, —NHC(O)CH₃, methyl, and methoxy; and wherein each of R^{11a} and R^{11b} is independently selected from hydrogen, —OH, methyl, and methoxy; and wherein each occurrence of \textcircled{A} is independently selected from:

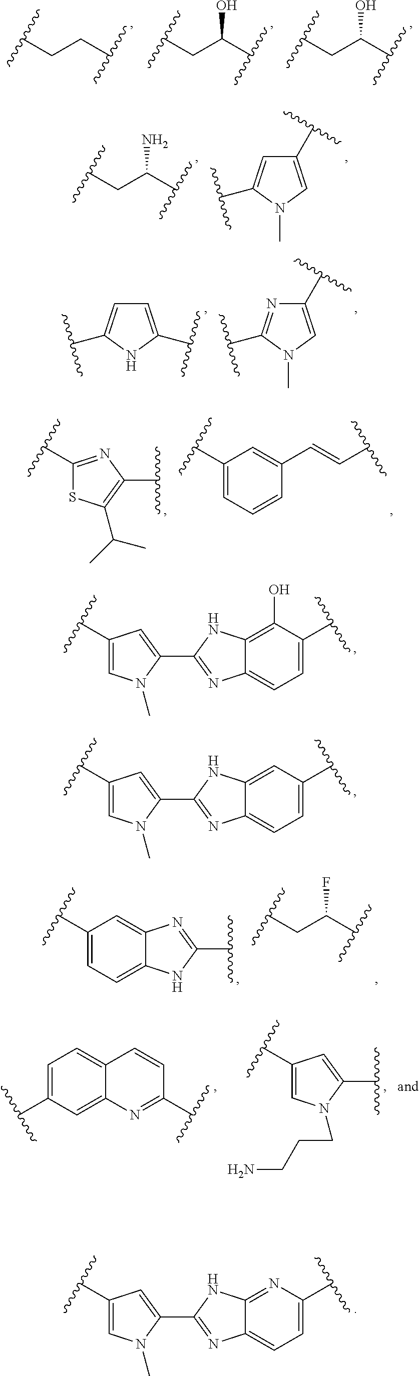

In various aspects, the compound is selected from:

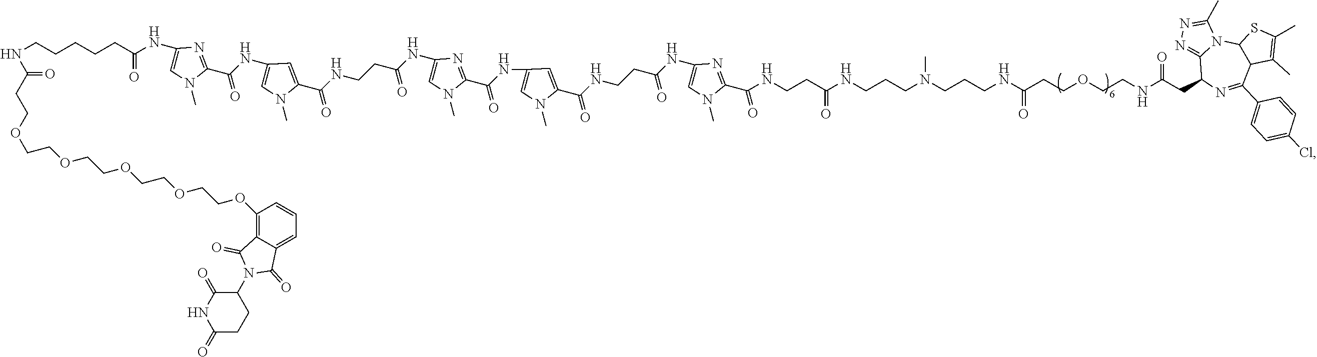

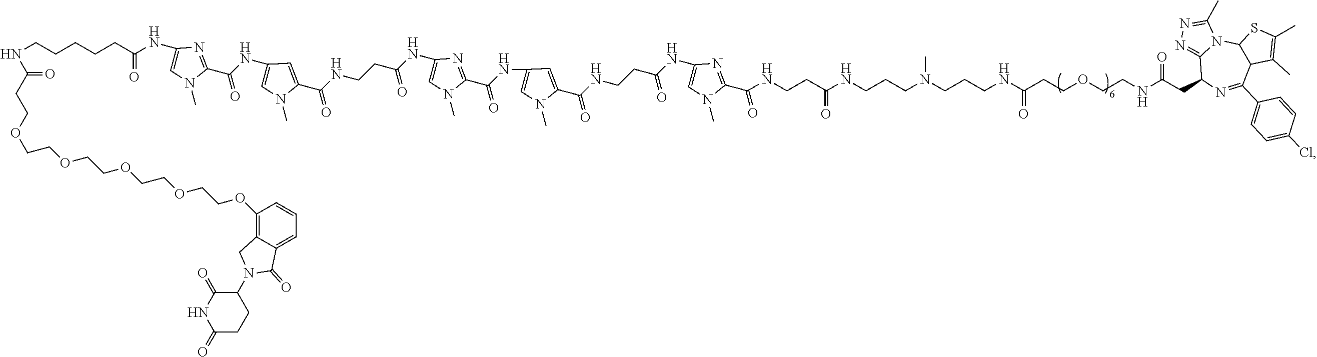

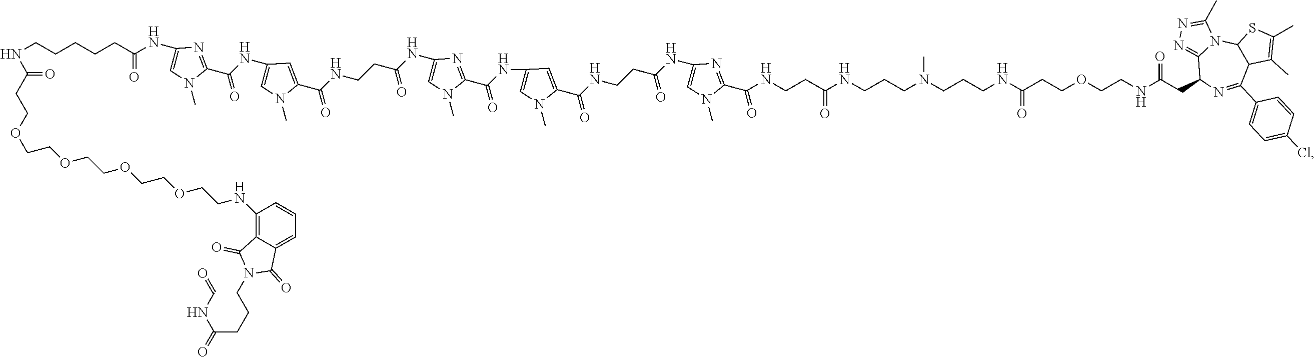 [SMILES: CC1=C(C)C2C(c3ccc(Cl)cc3)=N[C@@H](CC(=O)NCCOCCC(=O)NCCCN(C)CCCNC(=O)CCNC(=O)c3nc(NC(=O)CCNC(=O)c4cc(NC(=O)c5nc(NC(=O)CCNC(=O)c6cc(NC(=O)c7nc(NC(=O)CCCCCNC(=O)CCOCCOCCOCCOCCNc8cccc9c8C(=O)N(CCCC(=O)NC=O)C9=O)cn7C)cn6C)cn5C)cn4C)cn3C)c3nnc(C)n3C2S1]

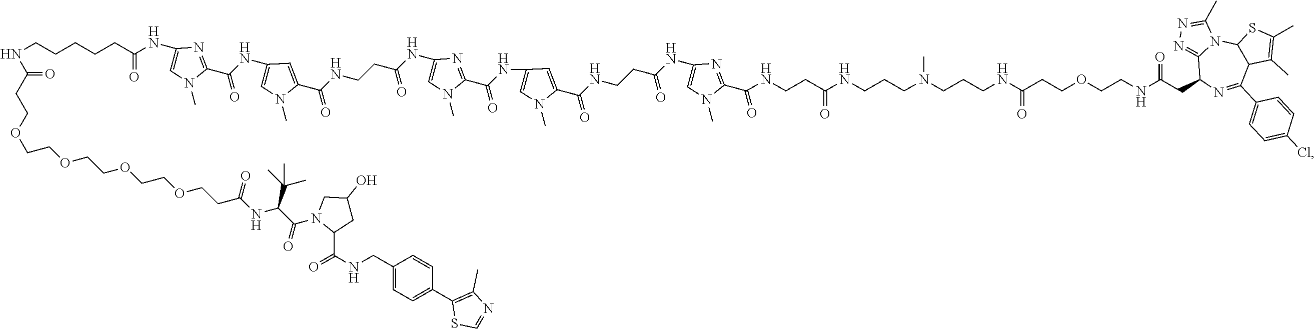 [SMILES: CC1=C(C)C2C(c3ccc(Cl)cc3)=N[C@@H](CC(=O)NCCOCCC(=O)NCCCN(C)CCCNC(=O)CCNC(=O)c3nc(NC(=O)CCNC(=O)c4cc(NC(=O)c5nc(NC(=O)CCNC(=O)c6cc(NC(=O)c7nc(NC(=O)CCCCCNC(=O)CCOCCOCCOCCOCCC(=O)N[C@H](C(=O)N8CC(O)CC8C(=O)NCc8ccc(-c9scnc9C)cc8)C(C)(C)C)cn7C)cn6C)cn5C)cn4C)cn3C)c3nnc(C)n3C2S1]

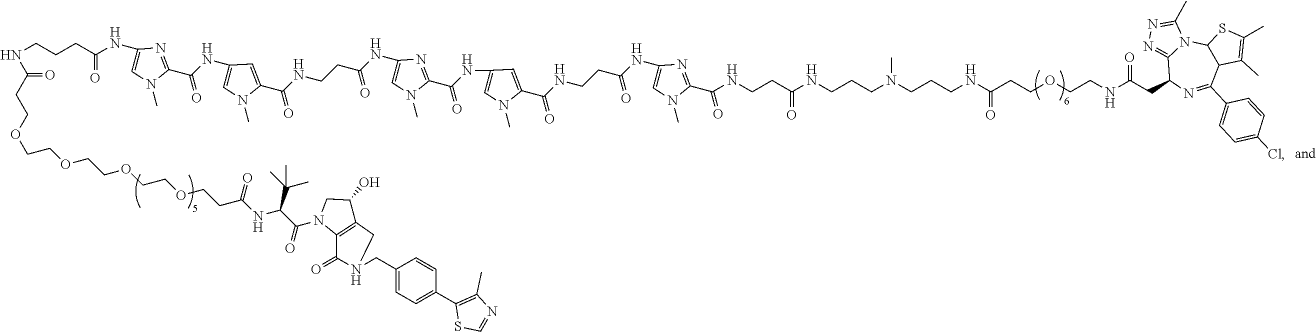

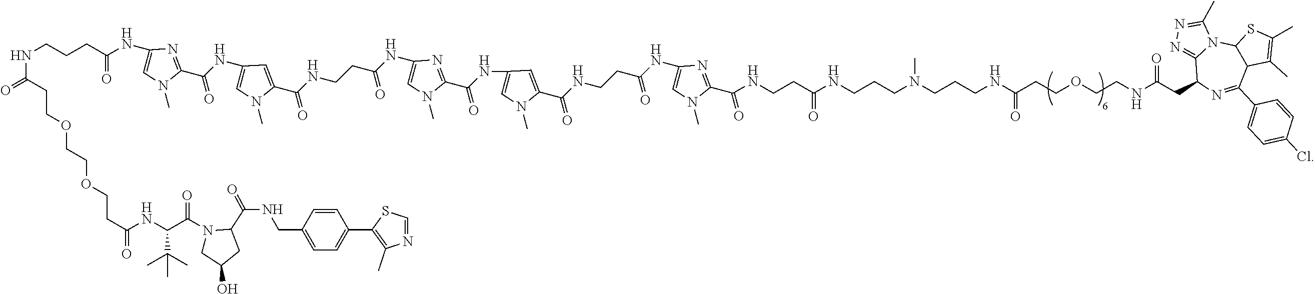

In various aspects, the compound has a structure selected from:

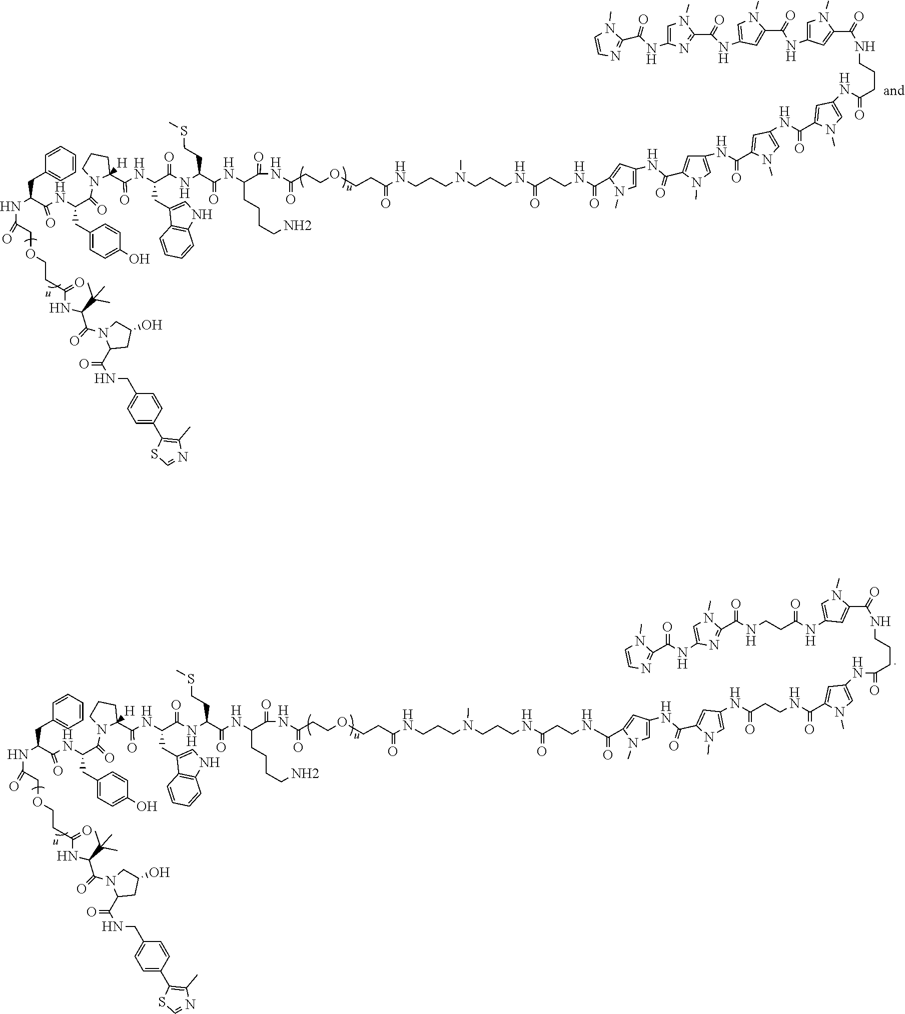

wherein each occurrence of u is independently an integer from 1 to 15.

In various aspects, the compound is selected from:

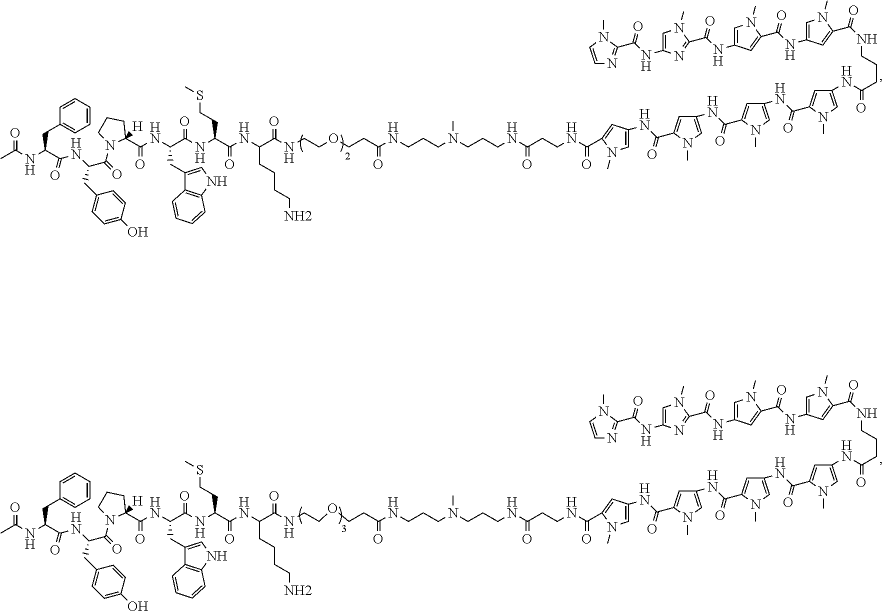

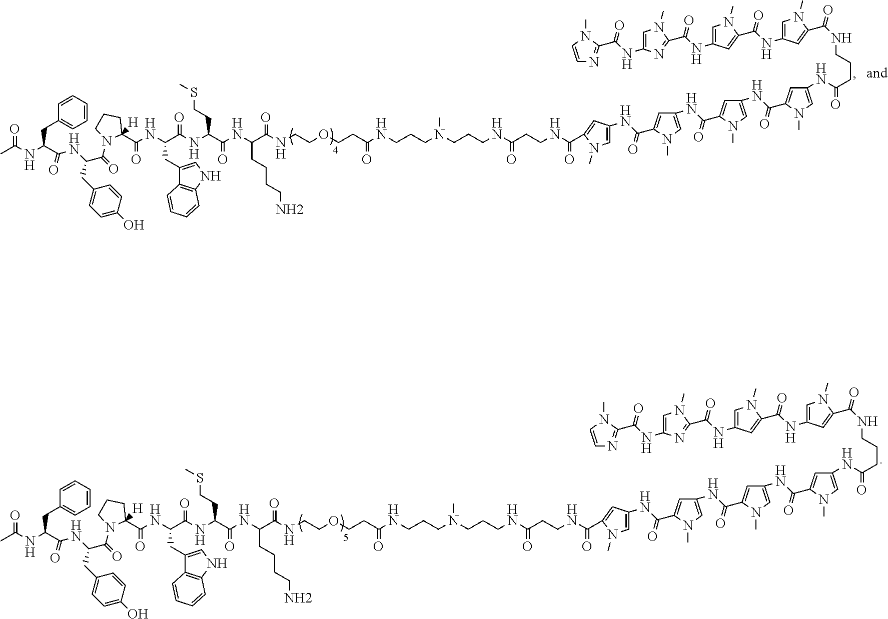

In various aspects, the polyamide moiety and the UPS targeting moiety or the BET binding ligand are tethered via the first chemical linker. In a further aspect, the polyamide moiety and the UPS targeting moiety are tethered via the first chemical linker. In a still further aspect, the polyamide moiety and the BET binding ligand are tethered via the first chemical linker.

In a further aspect, the polyamide moiety is tethered to the UPS targeting moiety via the first chemical linker, and the polyamide moiety is tethered to the BET binding ligand via the second chemical linker.

In various aspects, the polyamide moiety and the transcription factor binding moiety are tethered via the first chemical linker. In a further aspect, the polyamide moiety and the transcription factor binding moiety are tethered via the second chemical linker.

In various aspects, the transcription factor binding moiety and the UPS targeting moiety or the BET binding ligand are tethered via the second chemical linker. In a further aspect, the transcription factor binding moiety and the UPS targeting moiety are tethered via the second chemical linker. In a still further aspect, the transcription factor binding moiety and the BET binding ligand are tethered via the second chemical linker.

In various aspects, the compound comprises the UPS targeting moiety. In a further aspect, the UPS targeting moiety is an E3 ligase binding ligand. In a still further aspect, the E3 ligase binding ligand is selected from:

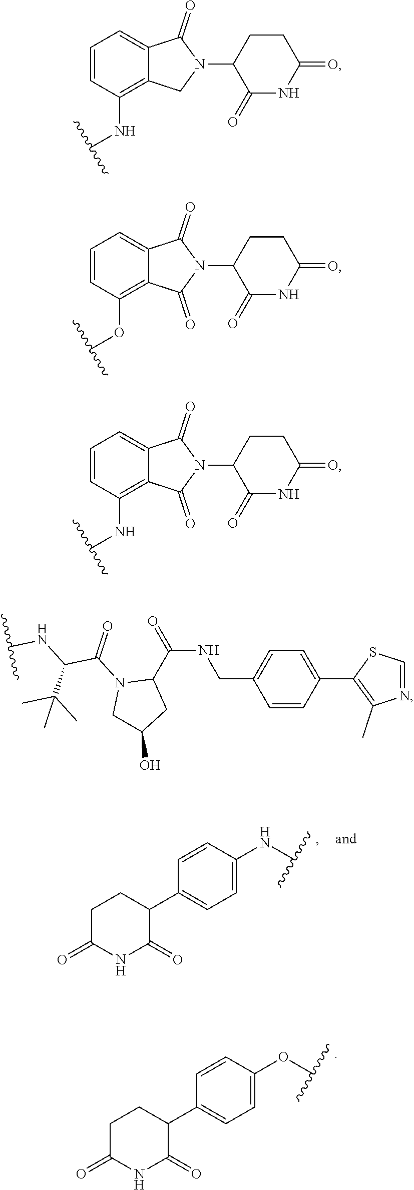 [SMILES: *Nc1cccc2c1CN(C1CCC(=O)NC1=O)C2=O ; *Oc1cccc2c1C(=O)N(C1CCC(=O)NC1=O)C2=O ; *Nc1cccc2c1C(=O)N(C1CCC(=O)NC1=O)C2=O ; *N[C@H](C(=O)N1C[C@H](O)CC1C(=O)NCc1ccc(-c2scnc2C)cc1)C(C)(C)C ; *Nc1ccc(C2CCC(=O)NC2=O)cc1 ; *Oc1ccc(C2CCC(=O)NC2=O)cc1]

In various aspects, the compound comprises the BET binding ligand. In a further aspect, the BET binding ligand is selected from:

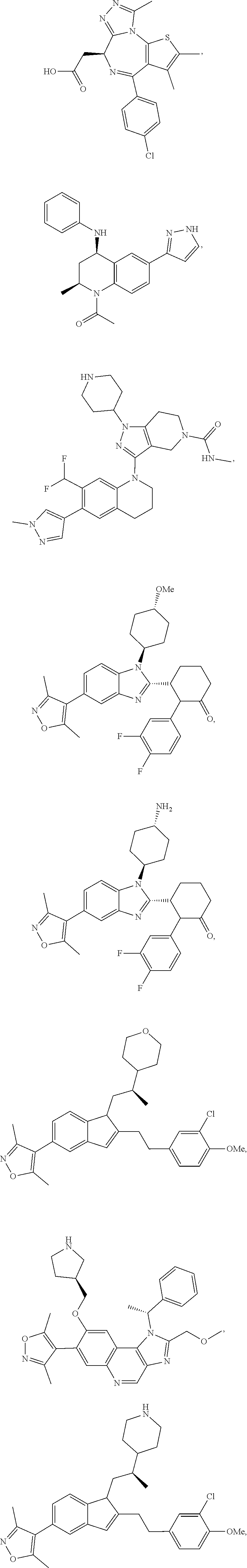

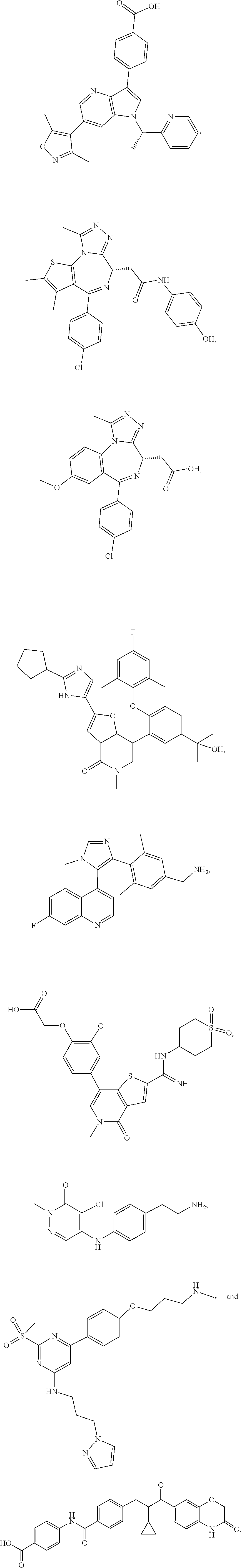

In various aspects, the compound has a structure represented by a formula selected from:

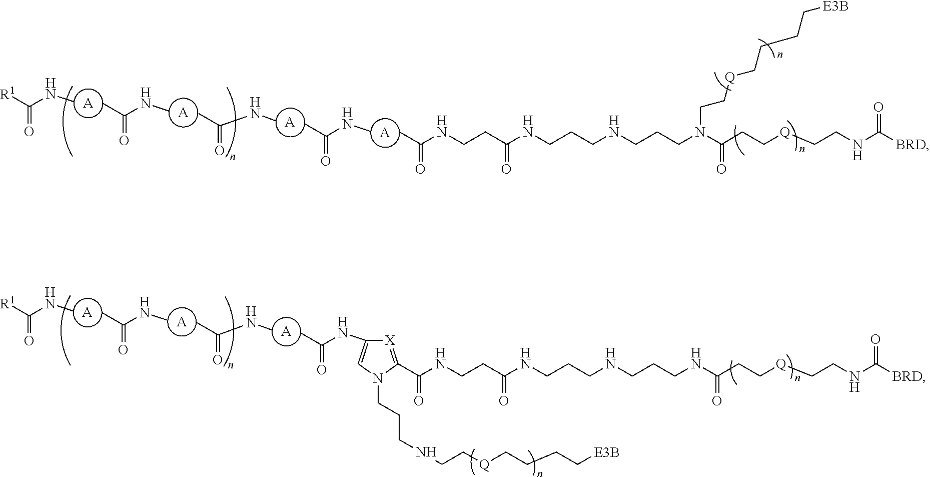

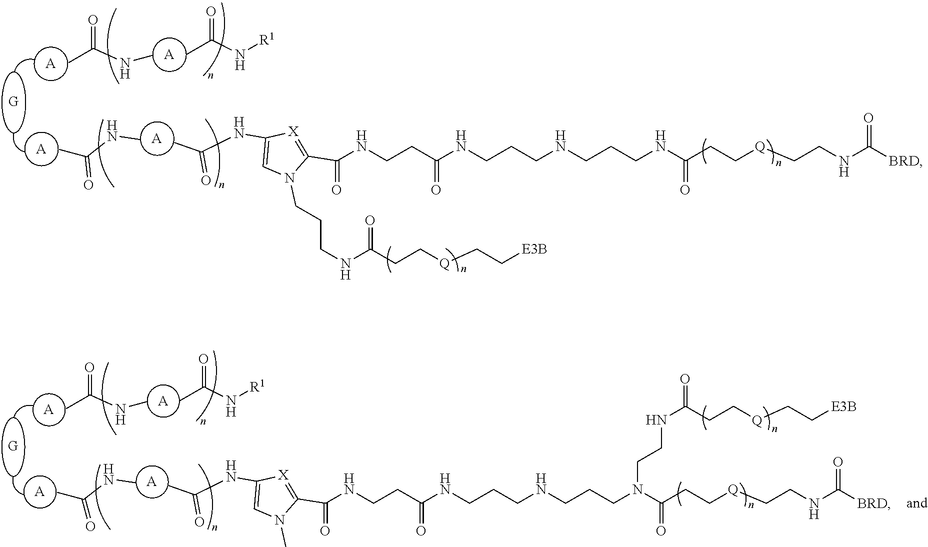

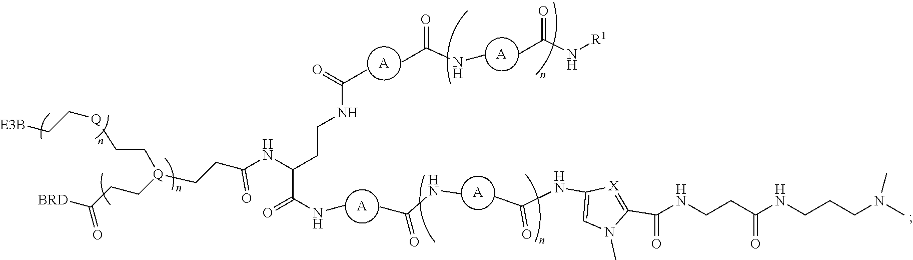

wherein E3B is an E3 ligase binding ligand; wherein BRD is a BET binding ligand; wherein each occurrence of n is independently an integer selected from 1 to 20; wherein G is a polyamide turn linker; wherein each occurrence of Q is independently selected from CH₂ and O; wherein X is selected from N and CH; wherein R¹ is selected from:

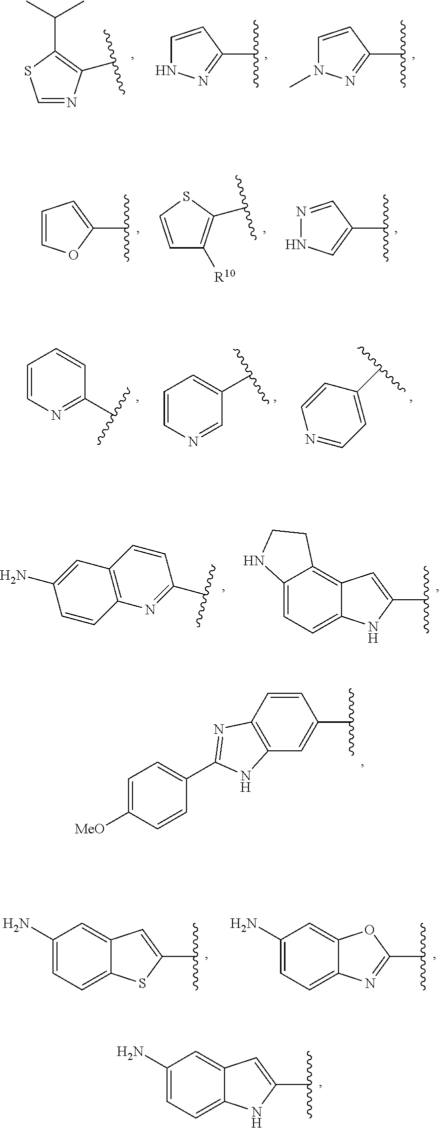

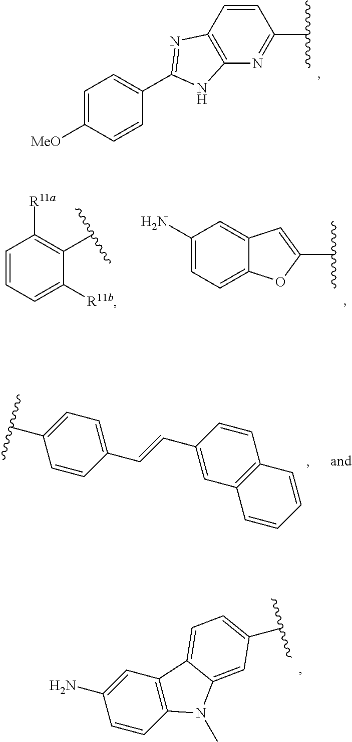

wherein R¹⁰ is selected from hydrogen, halogen, —OH, —NH₂, —NHC(O)CH₃, methyl, and methoxy; and wherein each of R^{11a} and R^{11b} is independently selected from hydrogen, —OH, methyl, and methoxy; and wherein each occurrence of A is independently selected from:

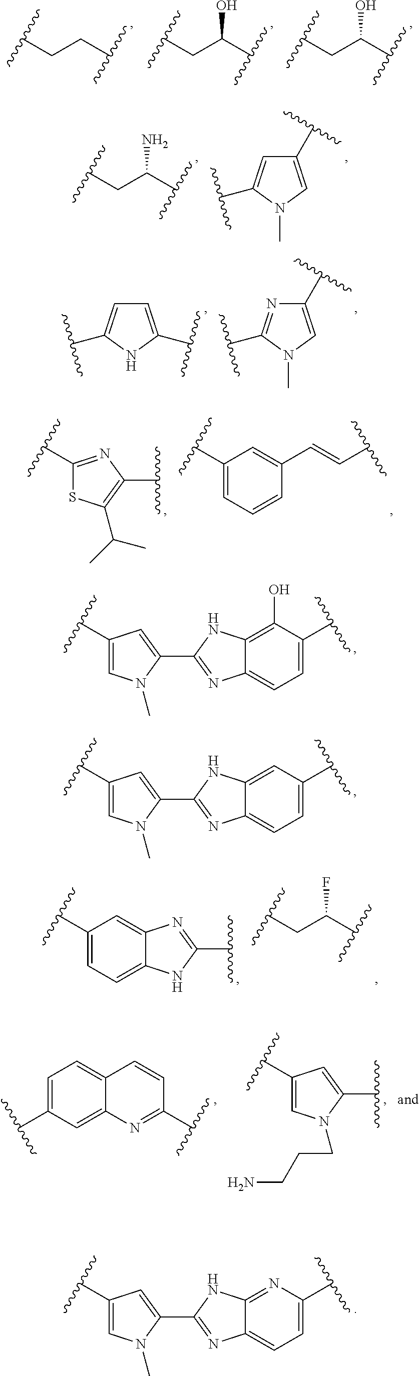

In various aspects, the compound has a structure represented by a formula selected from:

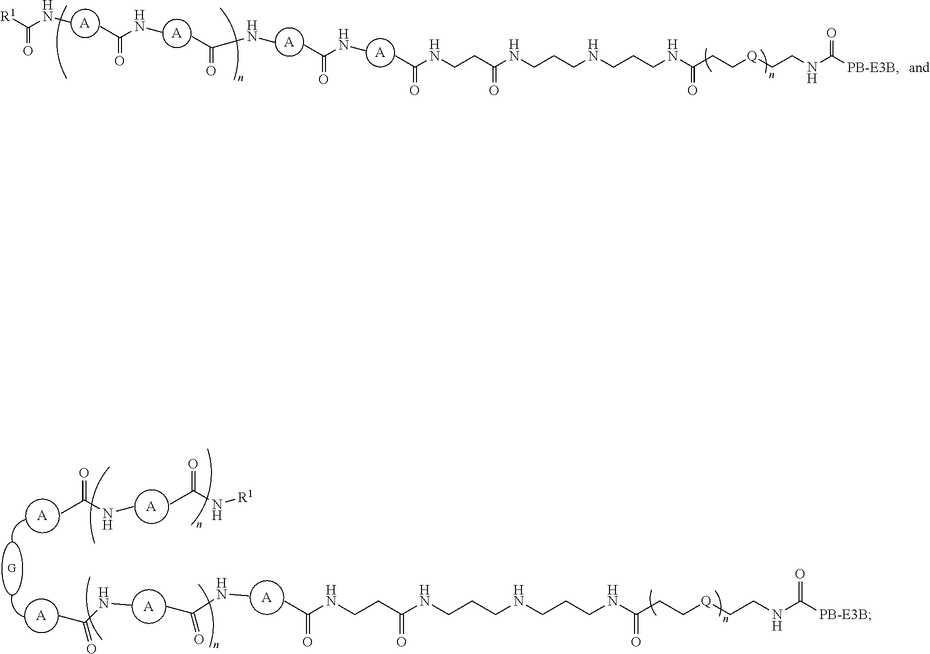

wherein E3B is an E3 ligase binding ligand; wherein PB is a transcription factor binding moiety; wherein each occurrence of n is independently an integer selected from 1 to 20; wherein G is a polyamide turn linker; wherein Q is selected from CH₂ and O; wherein R¹ is selected from:

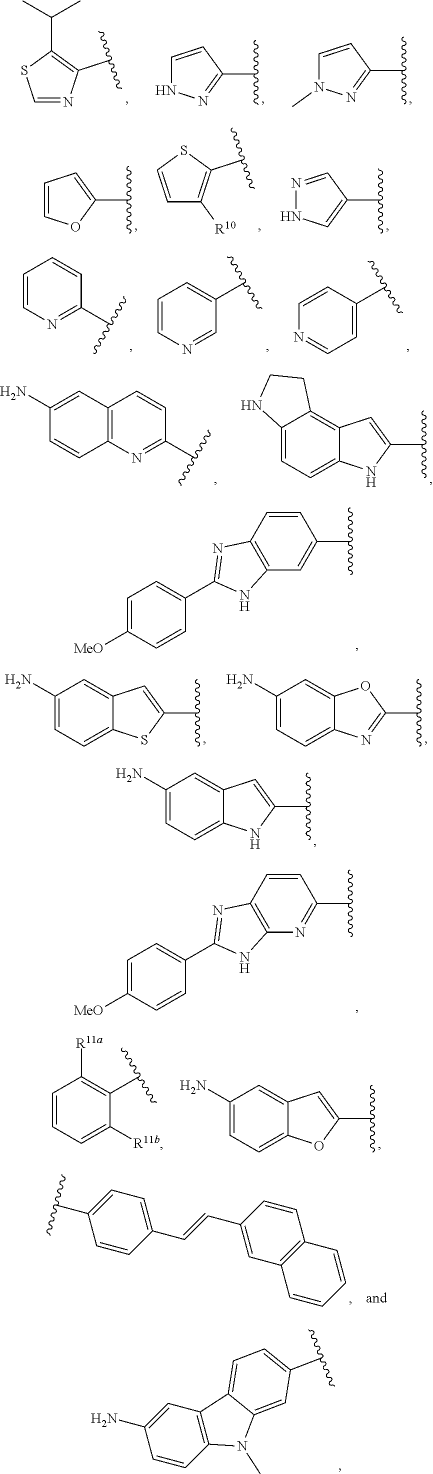

wherein R¹⁰ is selected from hydrogen, halogen, —OH, —NH₂, —NHC(O)CH₃, methyl, and methoxy; and wherein each of R^{11a} and R^{11b} is independently selected from hydrogen, —OH, methyl, and methoxy; and wherein each occurrence of \textcircled{A} is independently selected from:

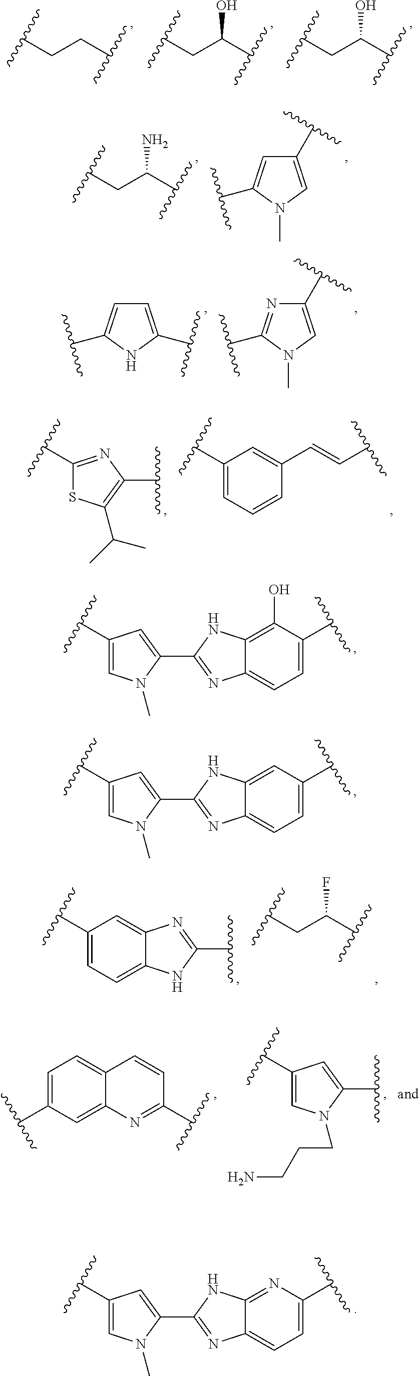

In various aspects, the compound has a structure represented by a formula selected from:

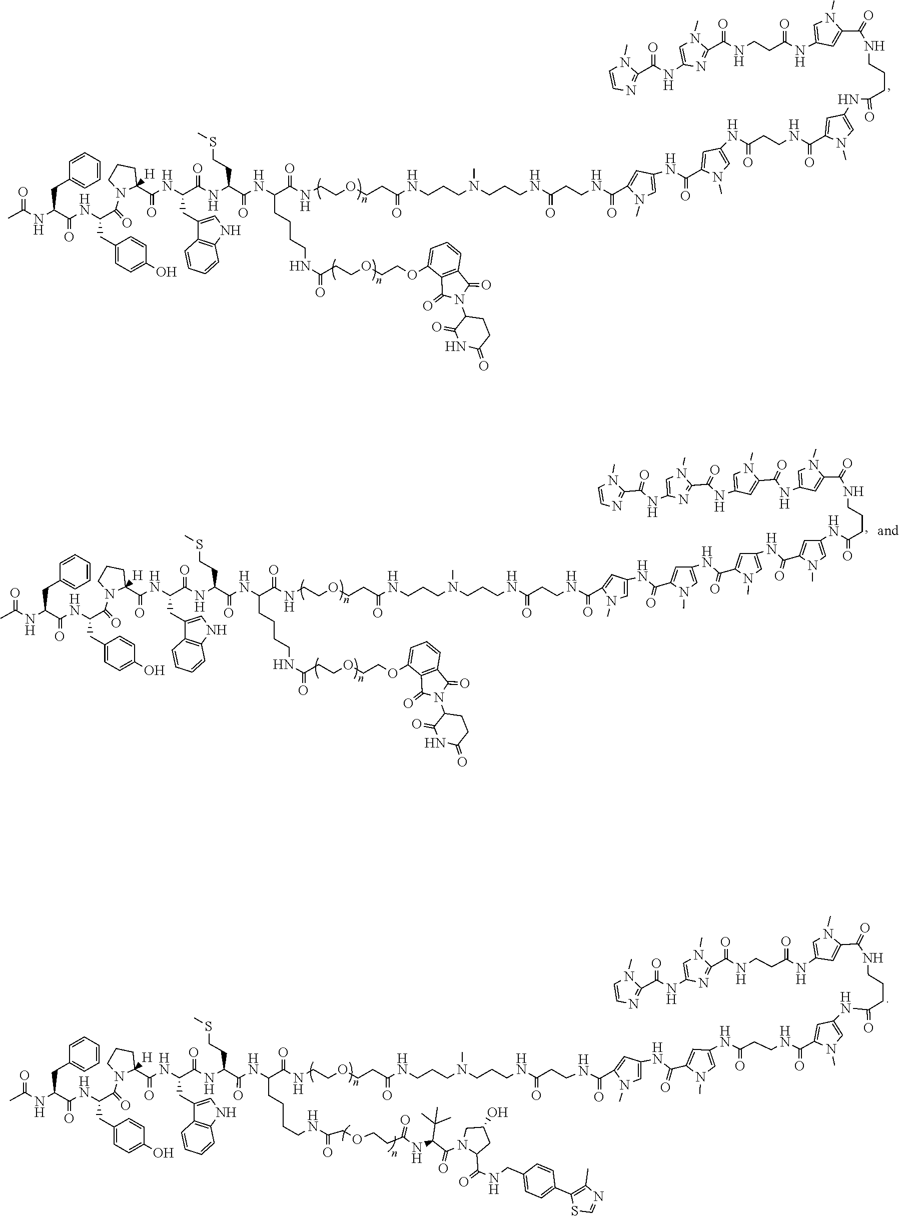

wherein each of u and u′ is independently an integer selected from 1 to 10.

In various aspects, the compound has a structure represented by a formula selected from:

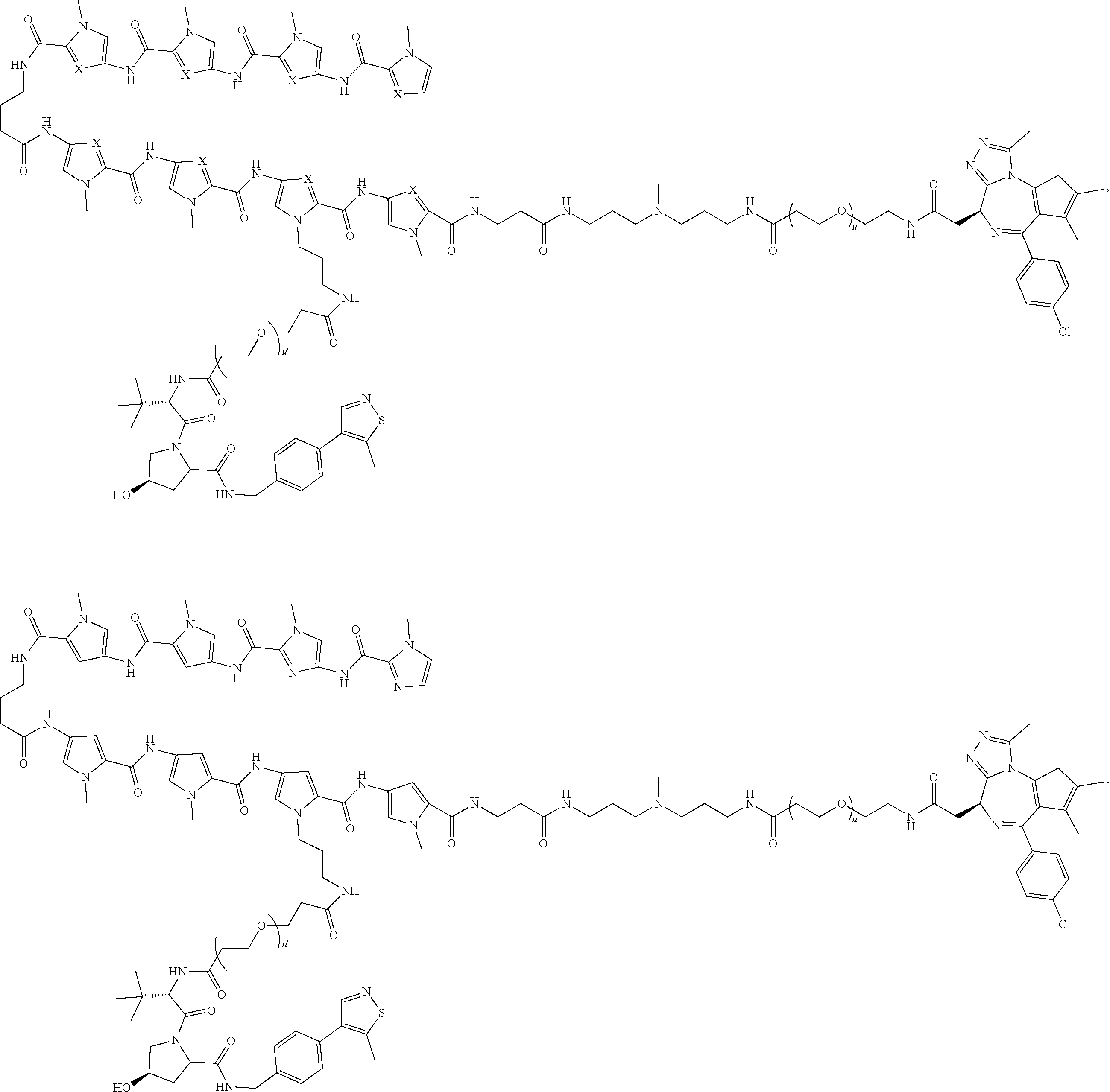

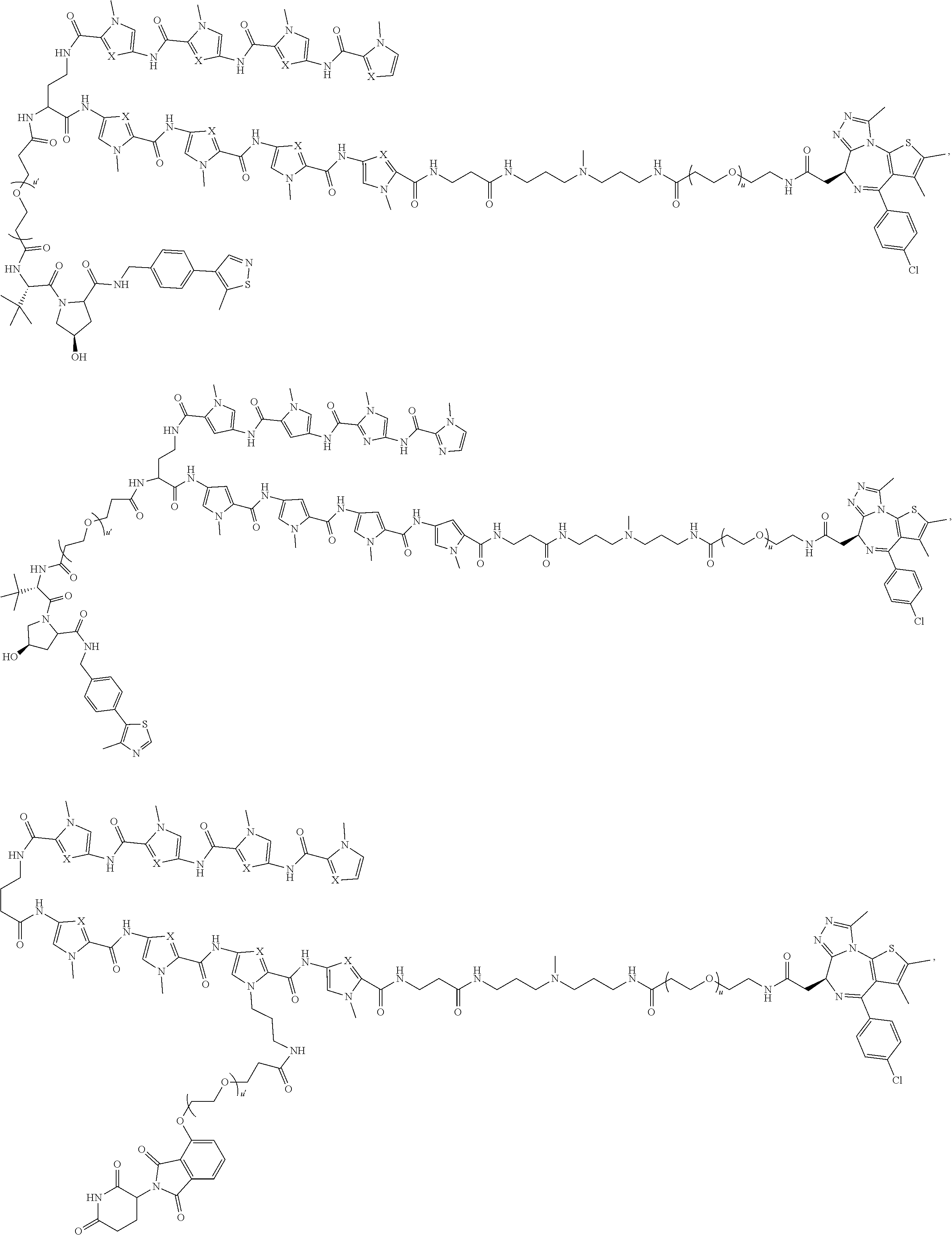

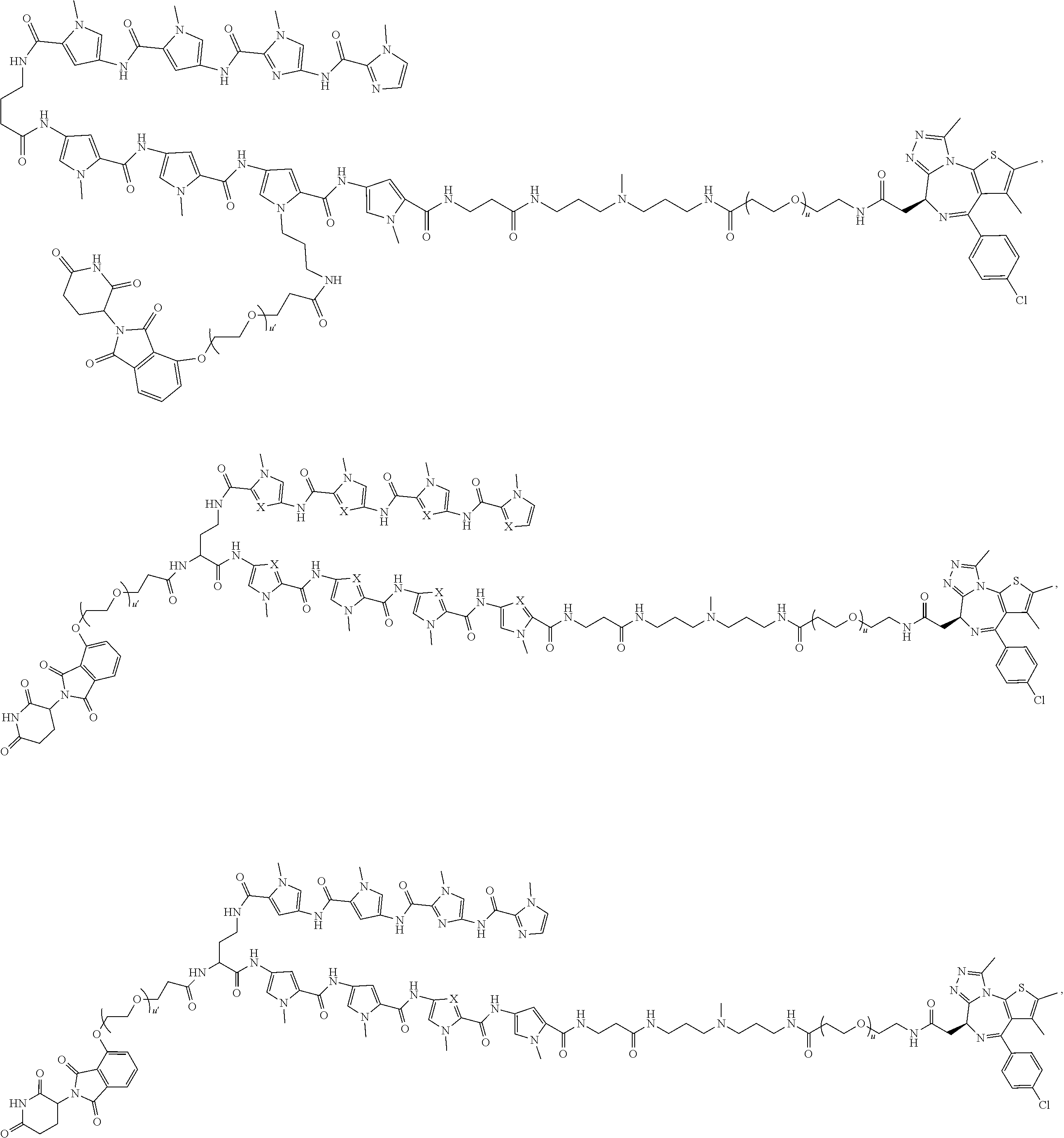

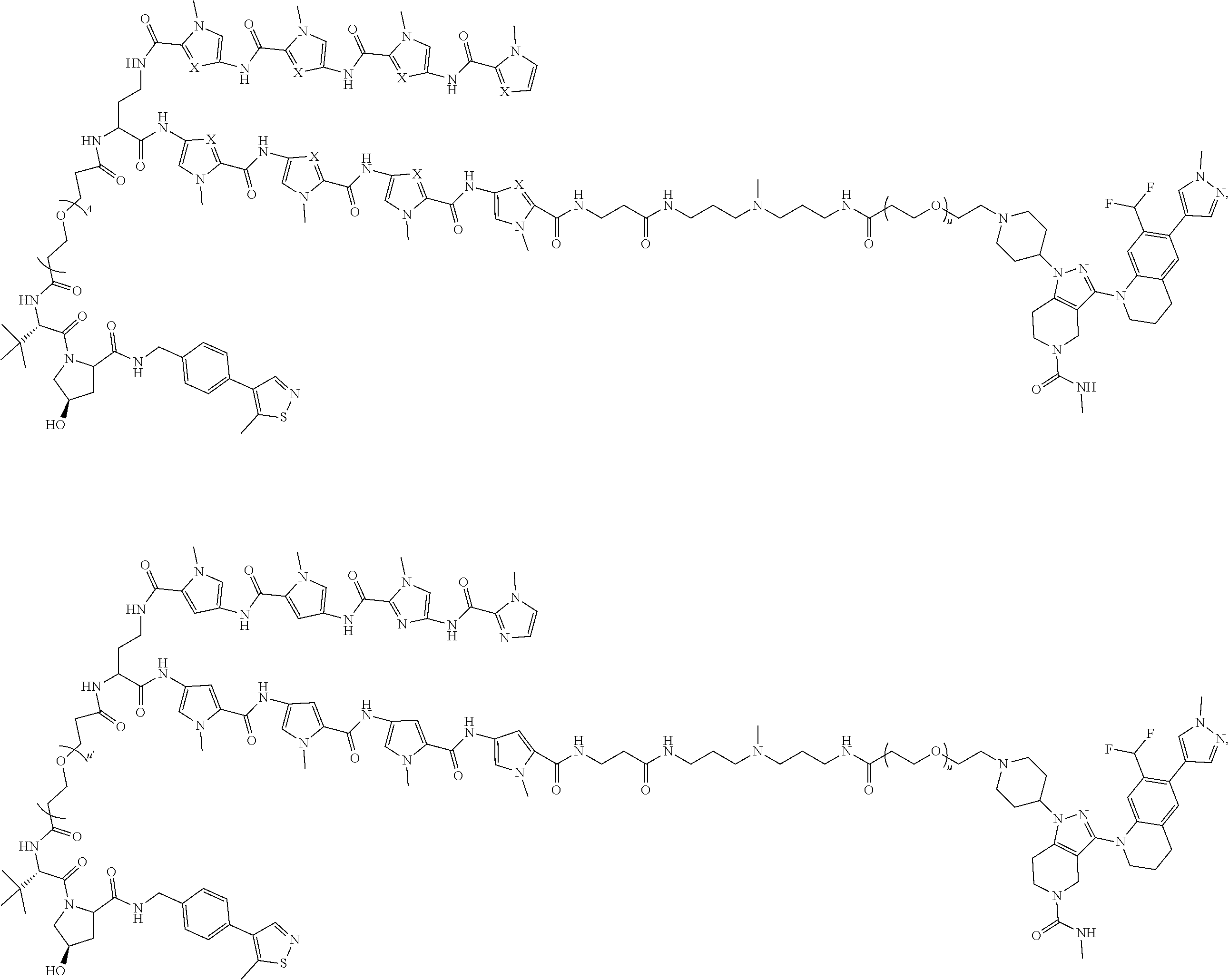

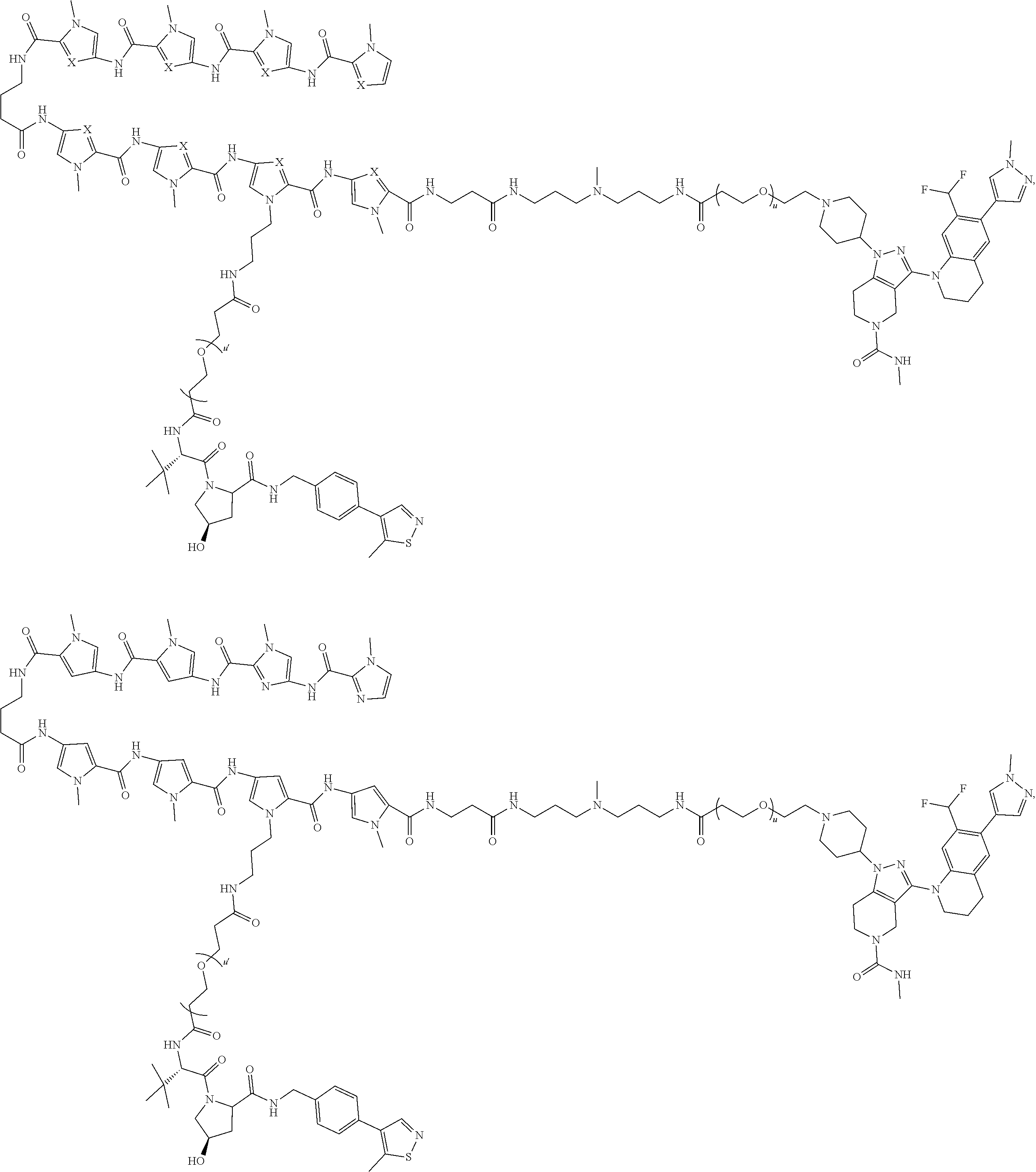

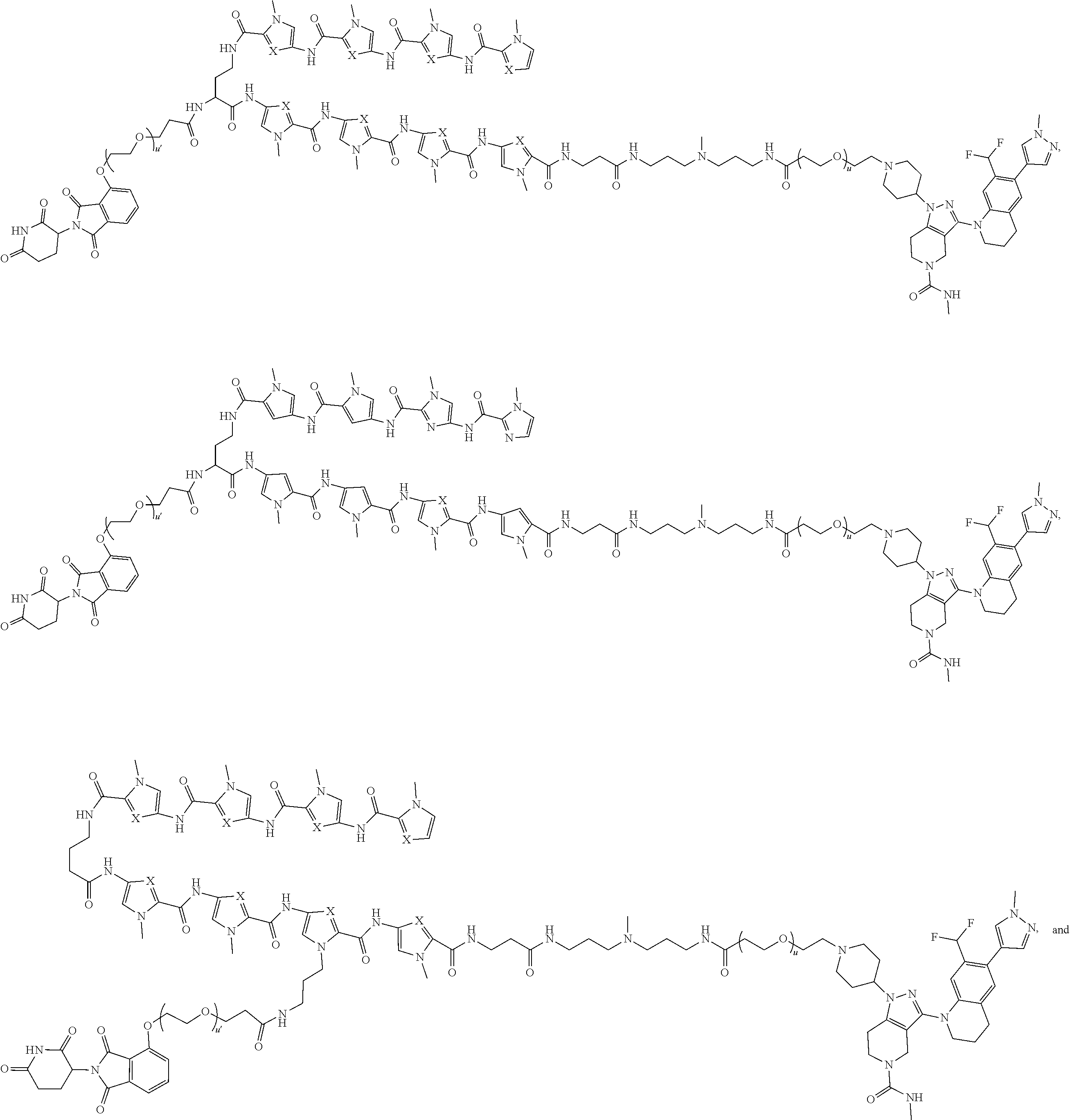

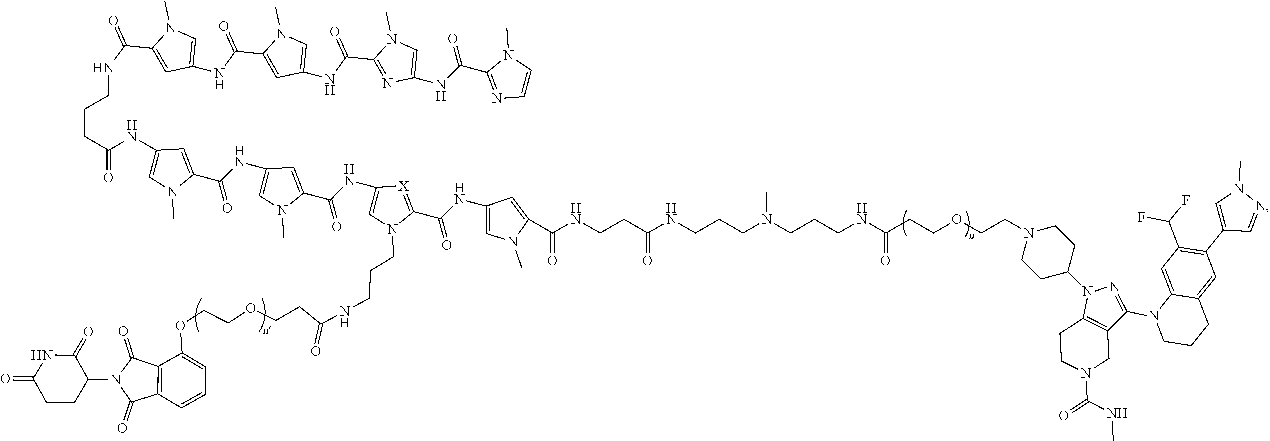

wherein each of u and u′ is independently an integer selected from 1 to 10; and wherein each occurrence of X is independently selected from N and CH.

In various aspects, the compound has a structure represented by a formula selected from:

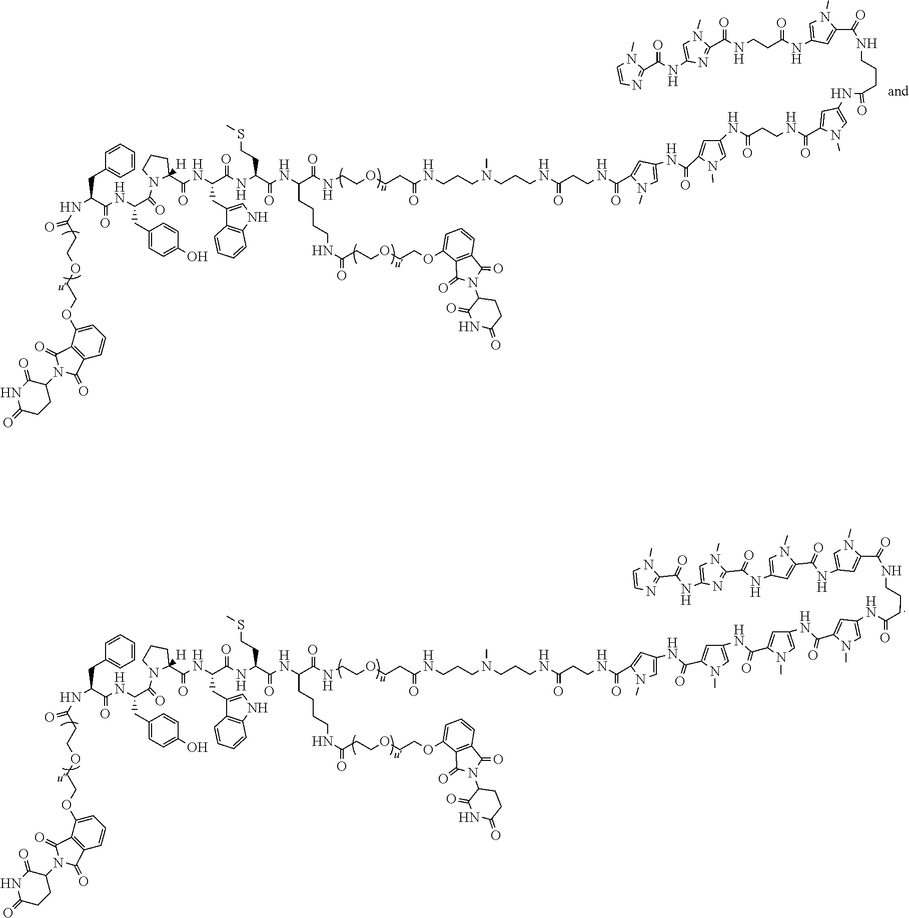

wherein each of u, u′, and u″ is independently an integer selected from 1 to 10.

In various aspects, the compound has a structure represented by a formula selected from:

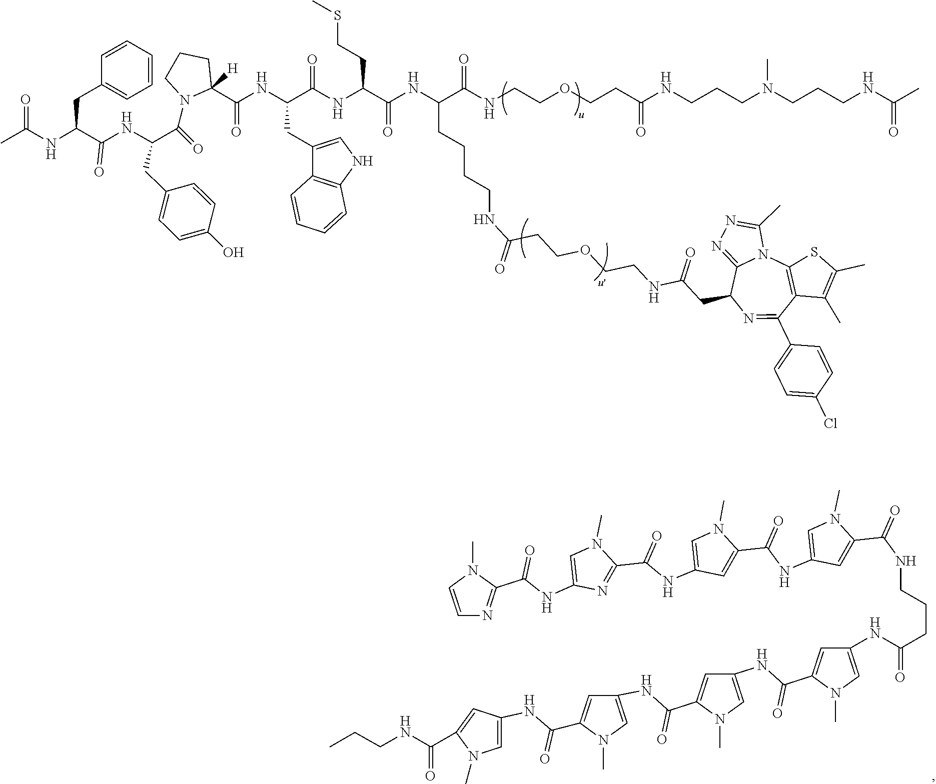

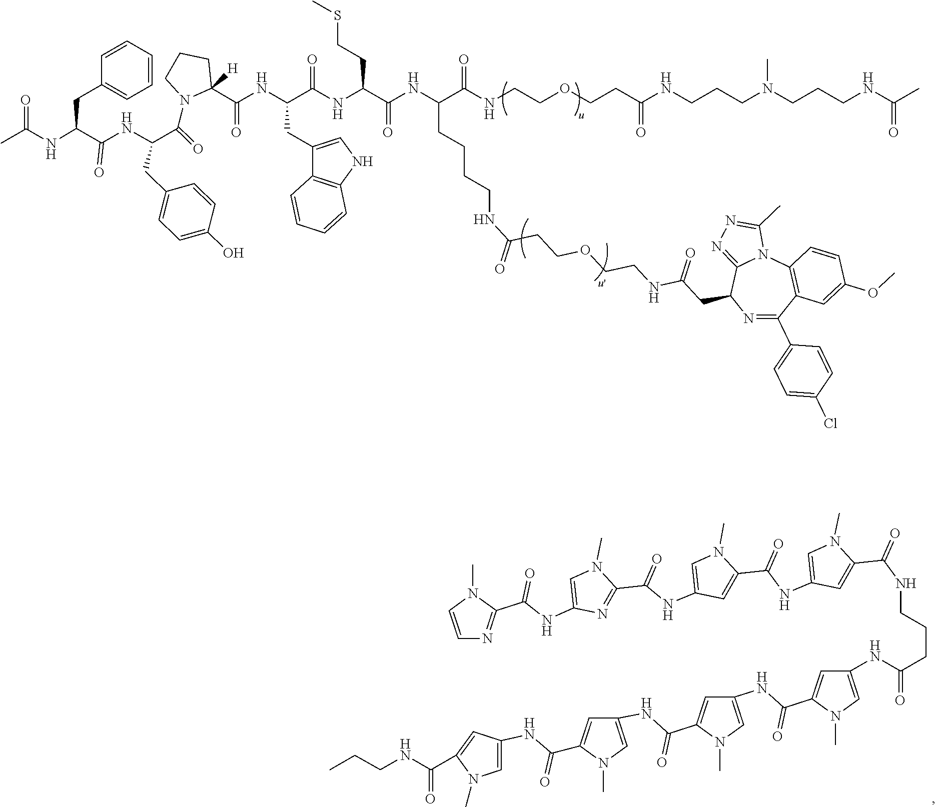

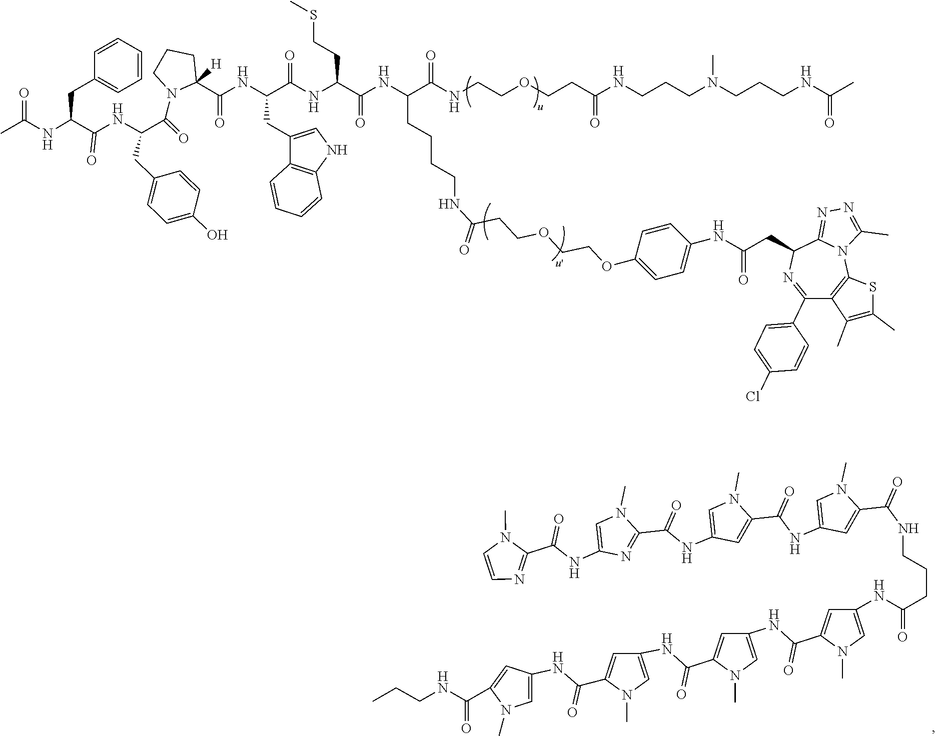

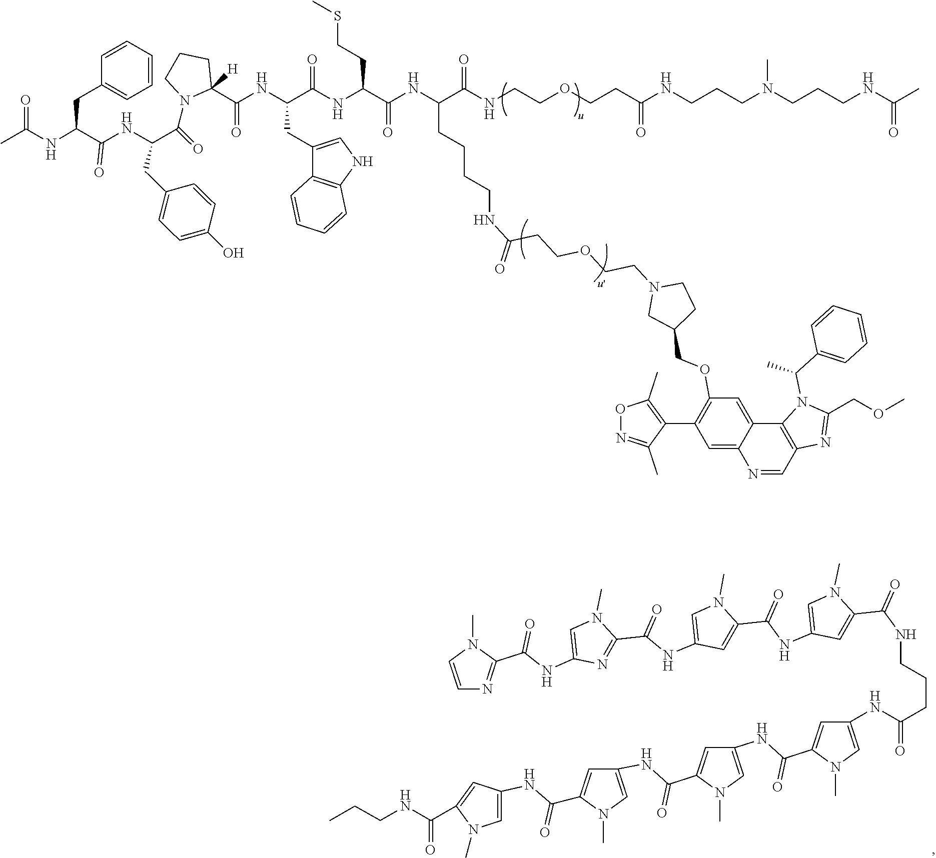

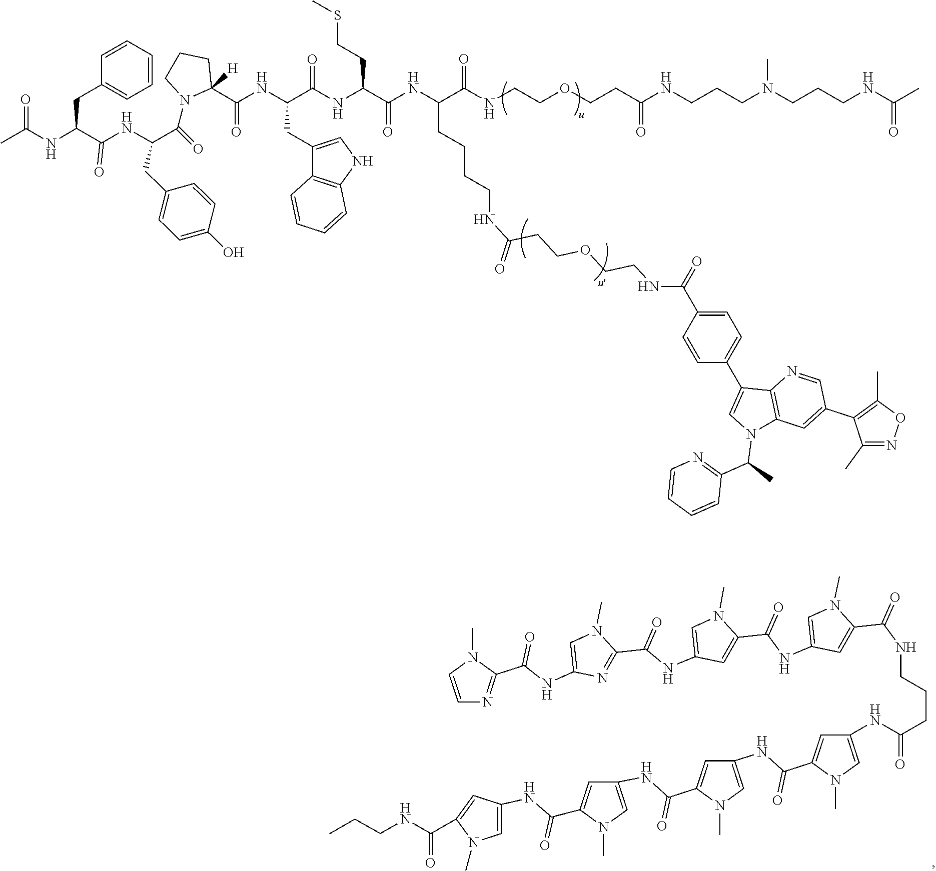

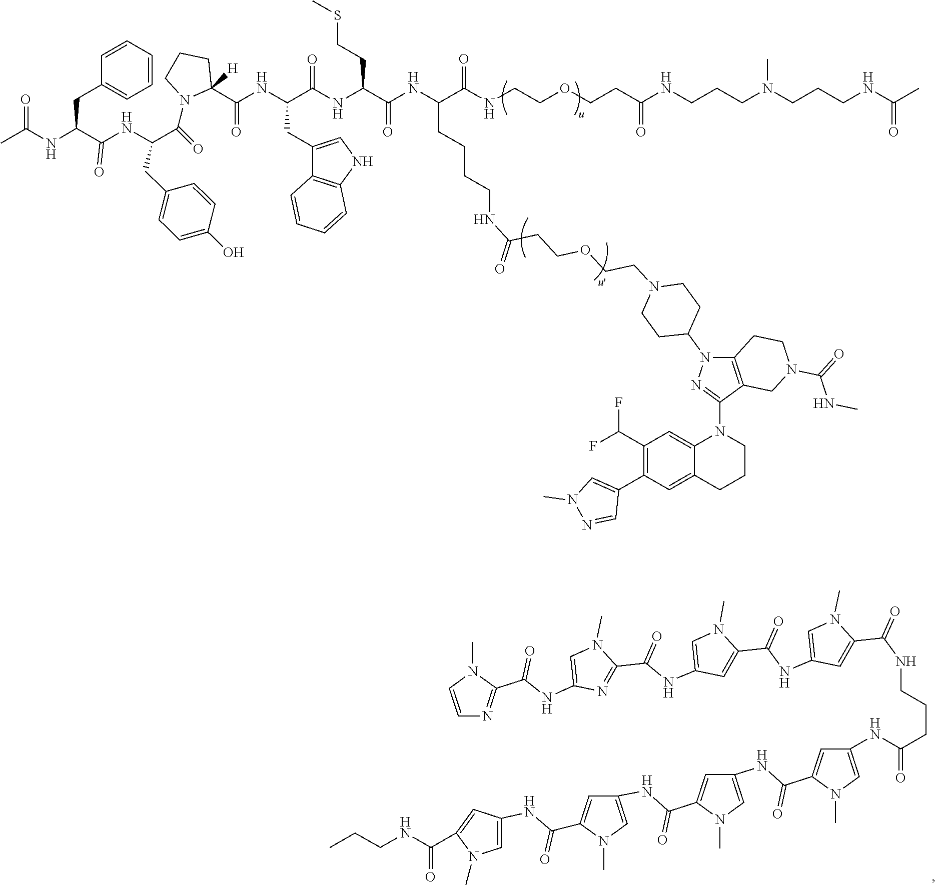

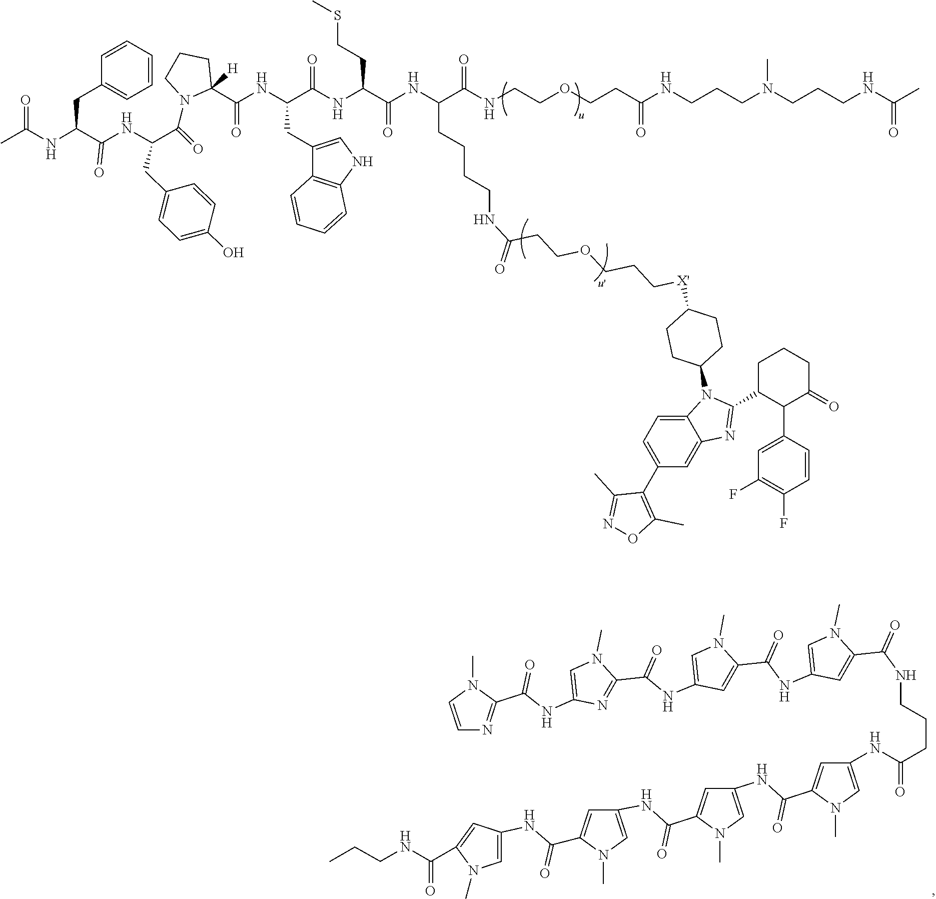

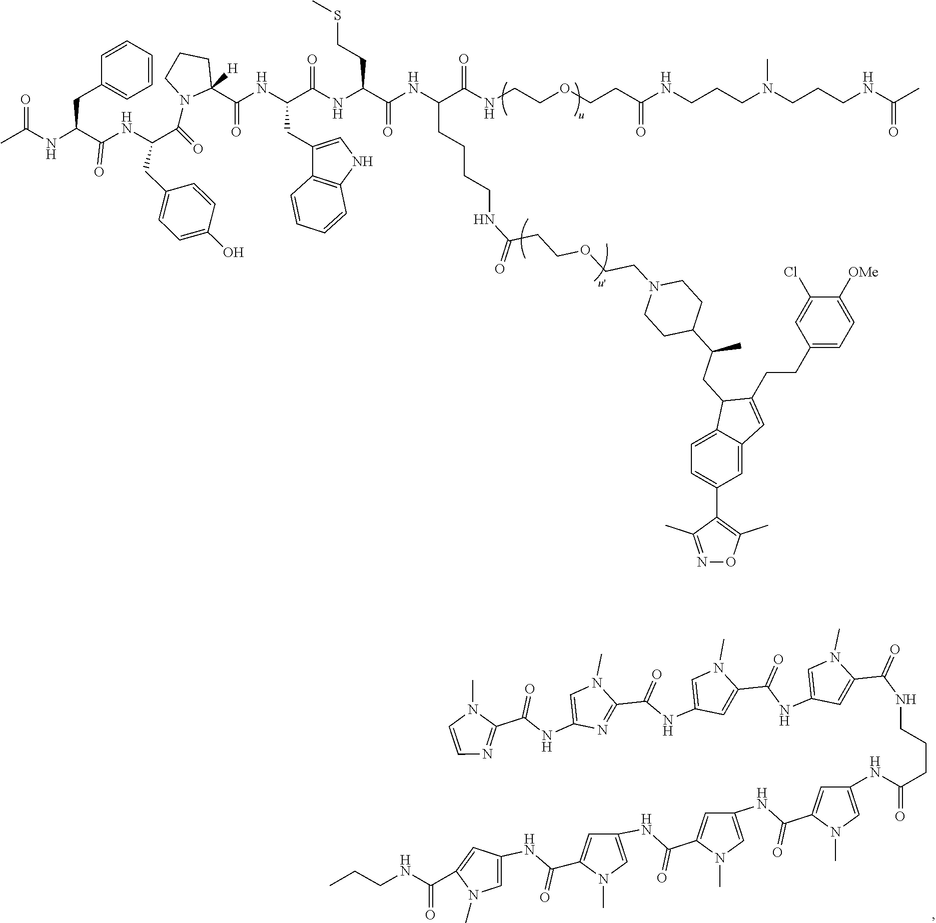

wherein each of u and u′ is independently an integer selected from 1 to 10; and wherein X′ is selected from NH and O.

In various aspects, the compound is selected from:

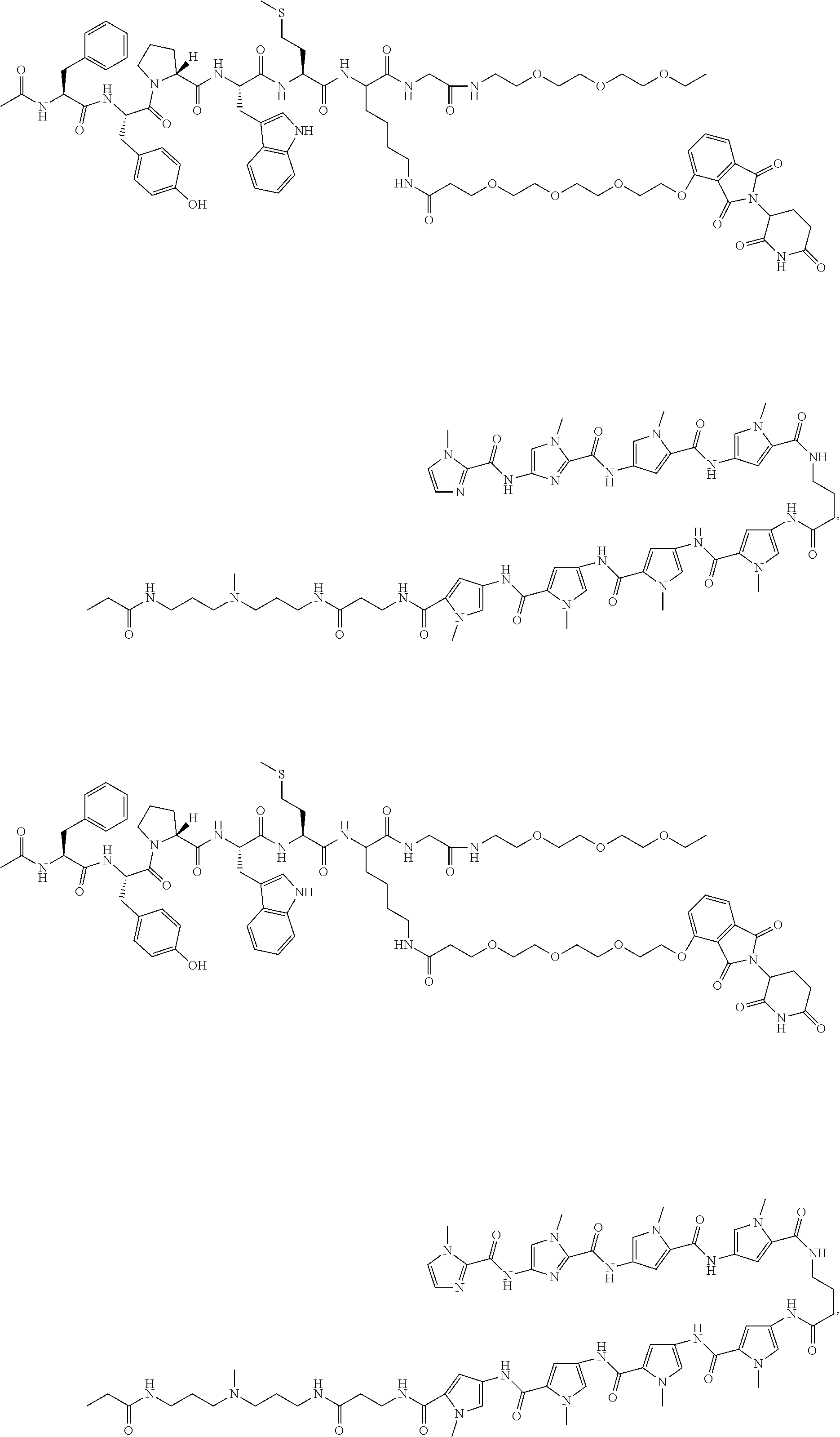

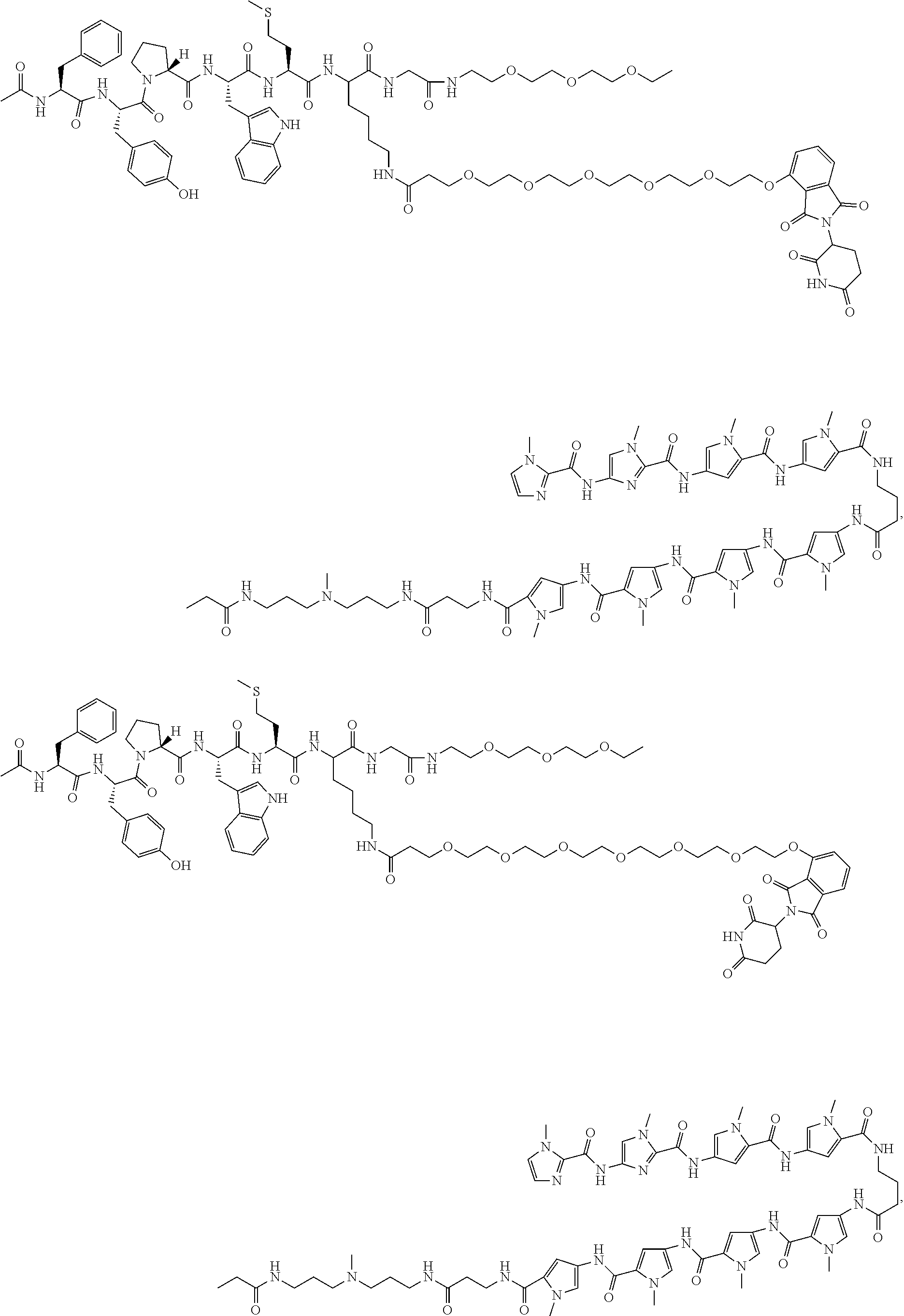

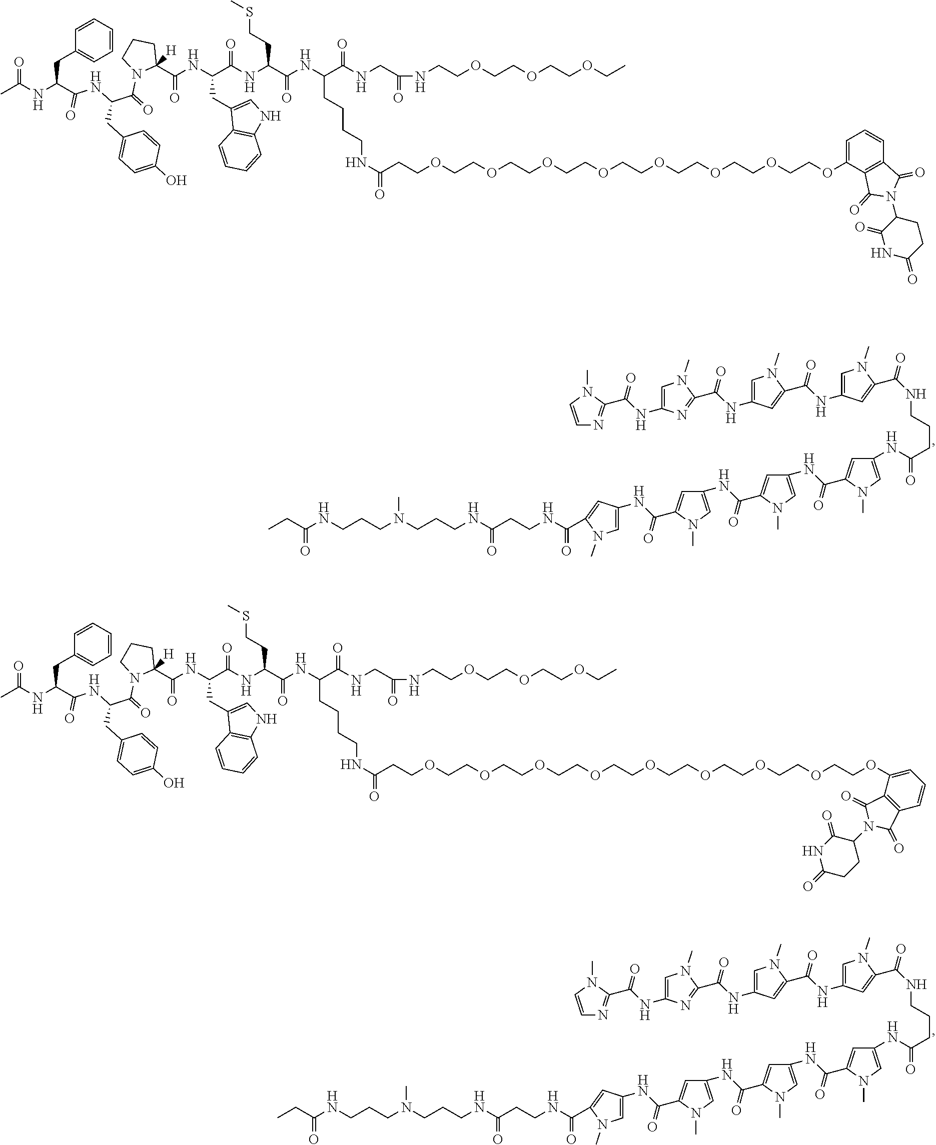

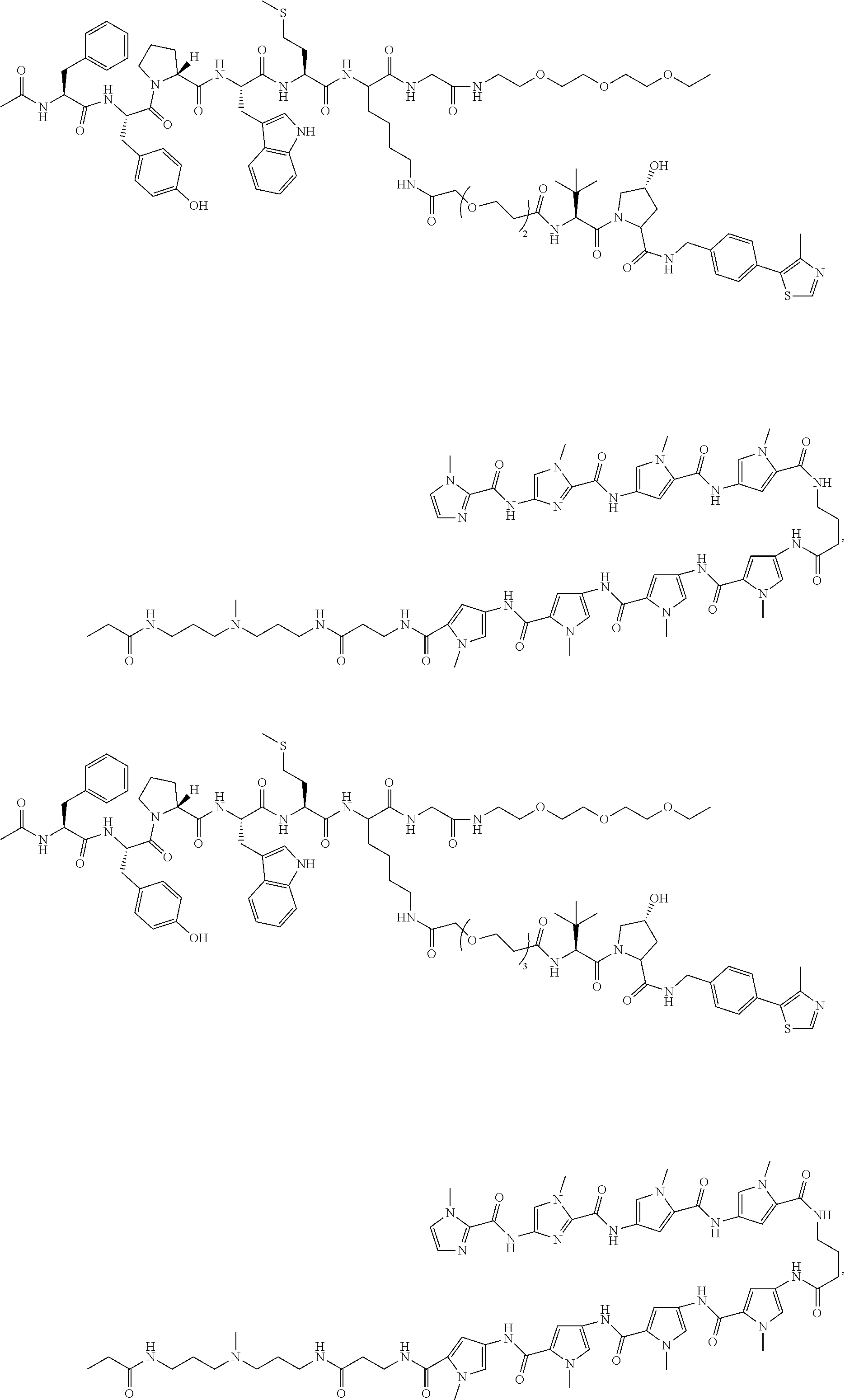

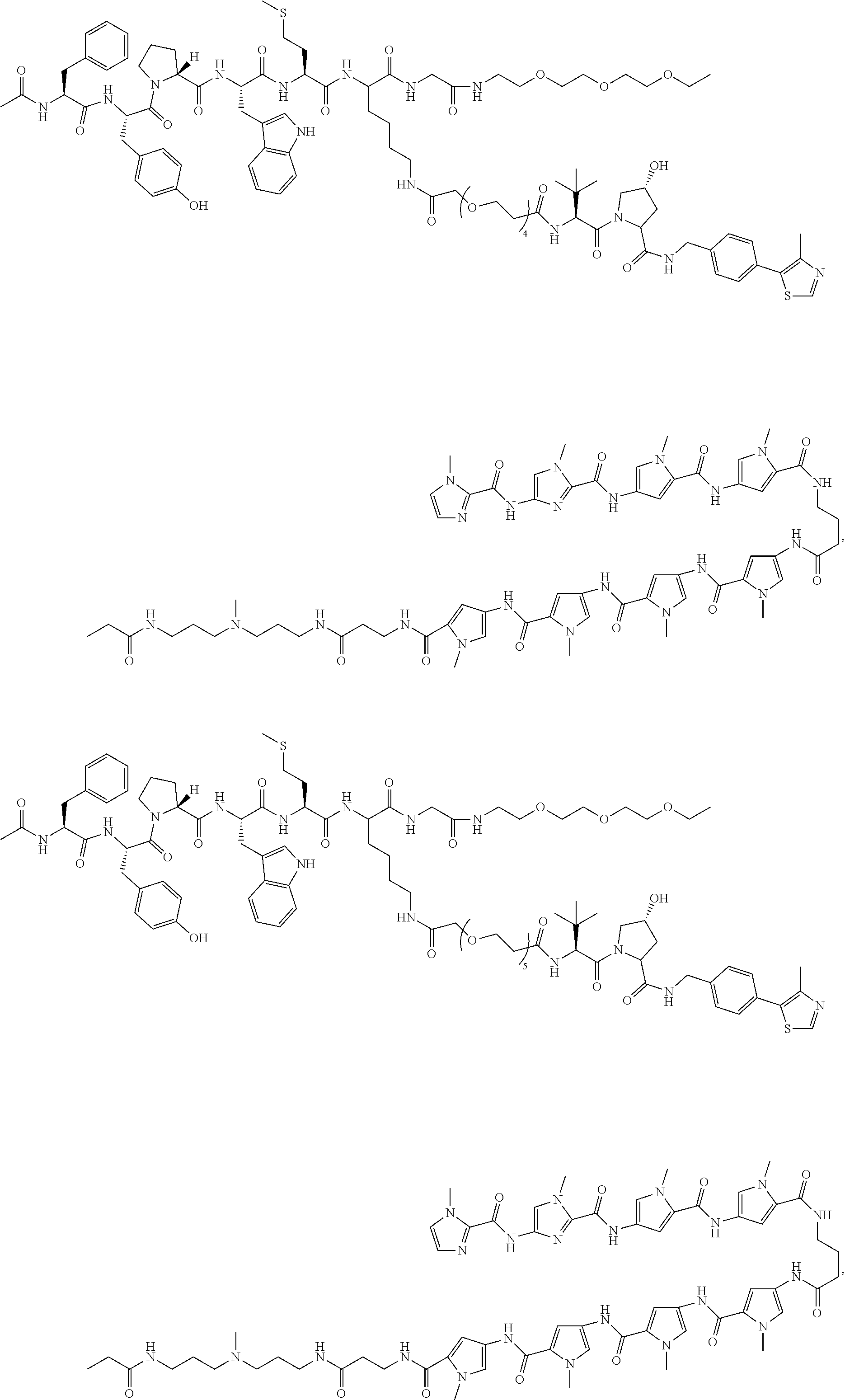

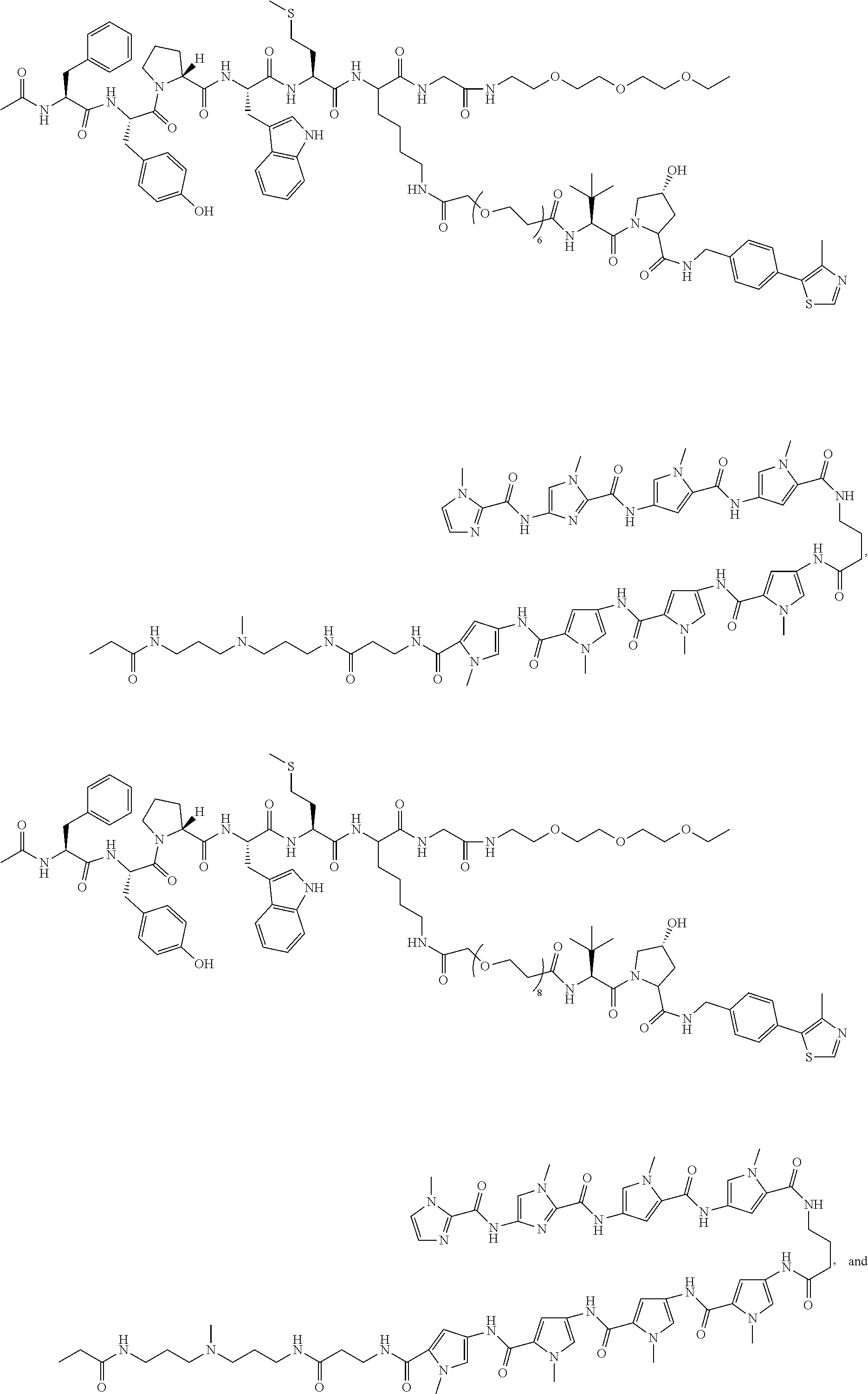

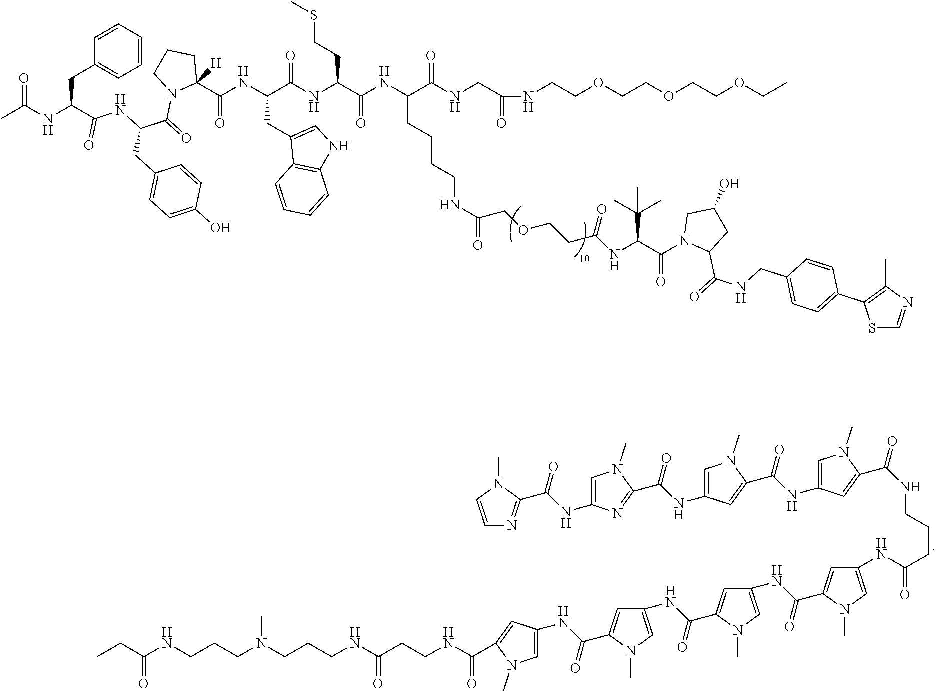

In one aspect, u is an integer selected from 1 to 13. In a further aspect, u is an integer selected from 1 to 10. In a still further aspect, u is an integer selected from 1 to 5. In yet a further aspect, u is an integer selected from 5 to 13. In an even further aspect, u is an integer selected from 10 to 13. In a still further aspect, u is an integer selected from 5 to 10.

In one aspect, each of u and u′ is independently an integer selected from 1 to 10. In a further aspect, each of u and u′ is independently an integer selected from 4 to 10. In a still further aspect, each of u and u′ is independently an integer selected from 8 to 10. In yet a further aspect, each of u and u′ is independently an integer selected from 1 to 8. In an even further aspect, each of u and u′ is independently an integer selected from 1 to 4. In a still further aspect, each of u and u′ is independently an integer selected from 4 to 8.

In one aspect, each of u, u′, and u″ is independently an integer selected from 1 to 10. In a further aspect, each of u, u′, and u″ is independently an integer selected from 4 to 10. In a still further aspect, each of u, u′, and u″ is independently an integer selected from 8 to 10. In yet a further aspect, each of u, u′, and u″ is independently an integer selected from 4 to 10. In an even further aspect, each of u, u′, and u″ is independently an integer selected from 8 to 10. In a still further aspect, each of u, u′, and u″ is independently an integer selected from 4 to 8.

a. Polyamide Moieties

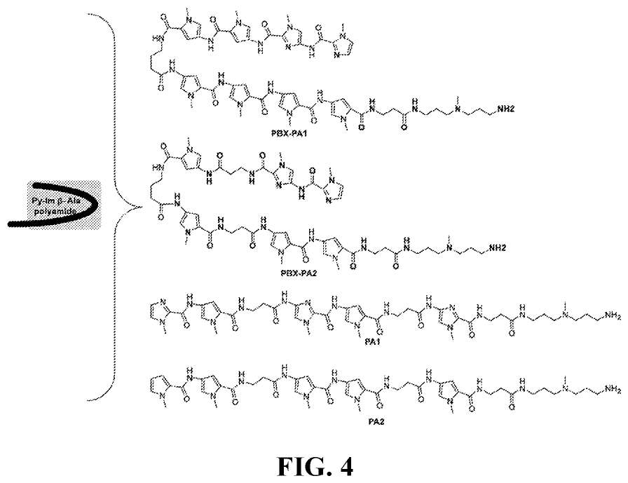

In one aspect, the disclosed sequence selective DNA binding compound comprises a polyamide moiety. The polyamide moiety can be tethered via a first chemical linker to the UPS targeting moiety, as further described herein. In various aspects, the polyamide moiety can also be tethered via a second chemical linker to the BET binding ligand. Exemplary polyamides are shown in FIG. 4.

In various aspects, the polyamide moiety is configured to bind the sequence ^{5′}AAGAAGAAG^{3′}.

In various aspects, the polyamide moiety is configured to bind the sequence ^{5′}WGGWWW^{3′}.

In various aspects, the polyamide moiety comprises a monomeric unit having a structure represented by a formula:

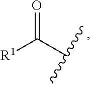

wherein R¹ is selected from:

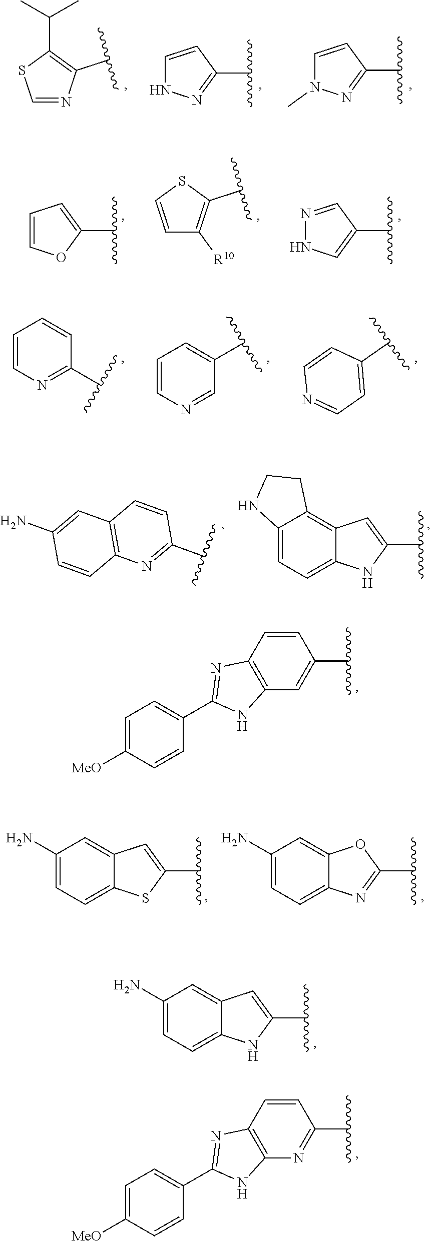

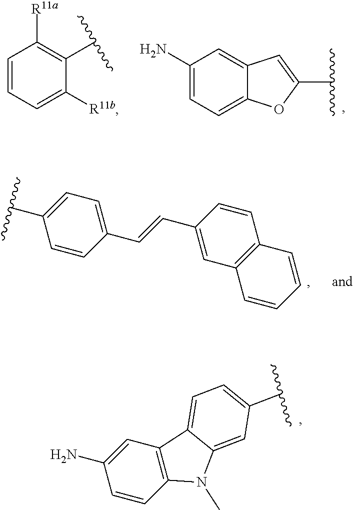

wherein R¹⁰ is selected from hydrogen, halogen, —OH, —NH₂, —NHC(O)CH₃, methyl, and methoxy; and wherein each of R^{11a} and R^{11b} is independently selected from hydrogen, —OH, methyl, and methoxy.

In various aspects, the polyamide moiety comprises a monomeric unit having a structure represented by a formula:

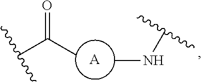

wherein \textcircled{A} is selected from:

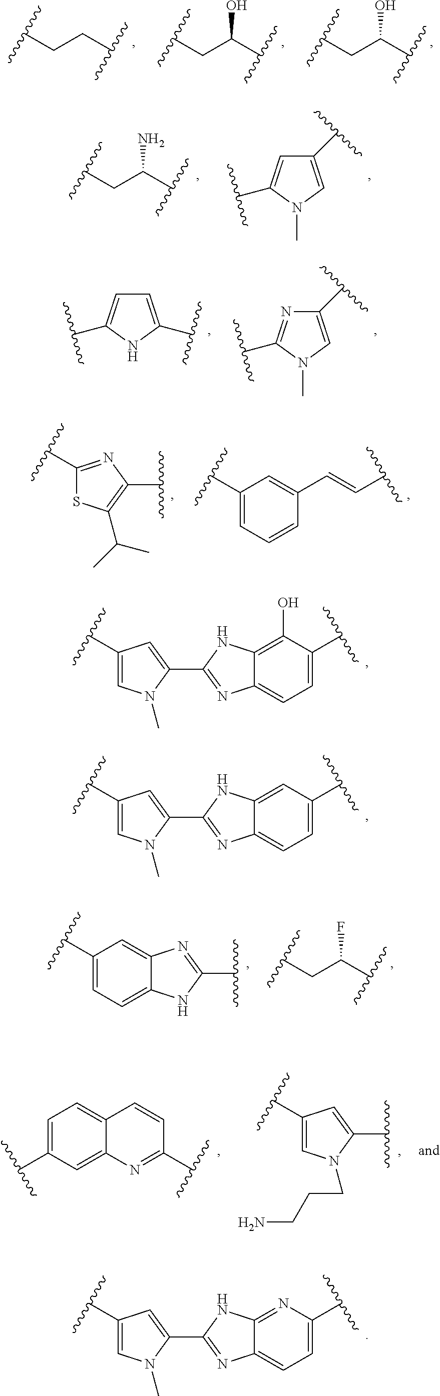

In various aspects, the polyamide moiety comprises a monomeric unit having a structure represented by a formula selected from:

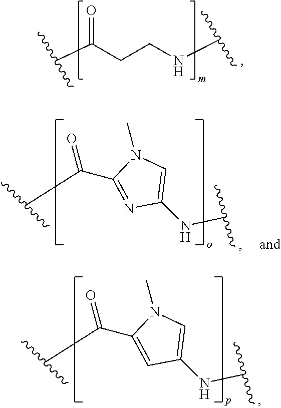

wherein each of m, o, and p is independently an integer selected from 1 to 20. In a further aspect, the polyamide moiety comprises at least two different monomeric units. In a still further aspect, the polyamide moiety comprises each of:

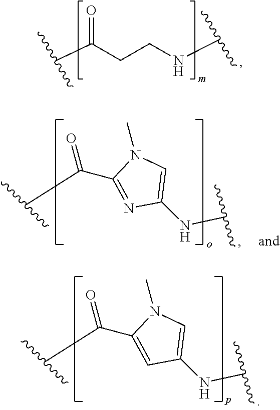

In various aspects, the polyamide moiety is a linear polyamide moiety. In a further aspect, the linear polyamide moiety is a residue of a structure represented by a formula:

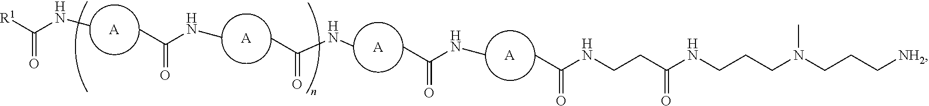

wherein n is an integer selected from 1 to 20; wherein R¹ is selected from:

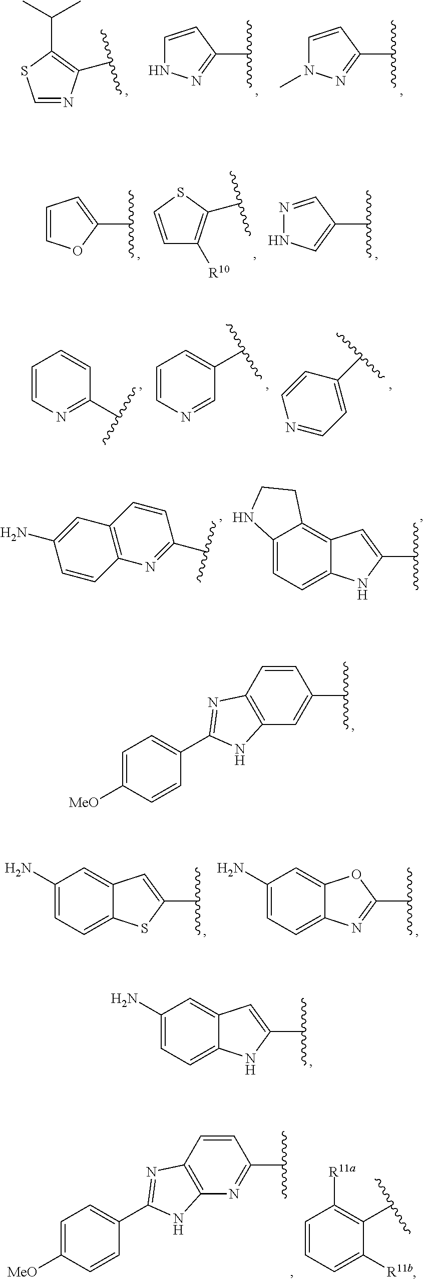

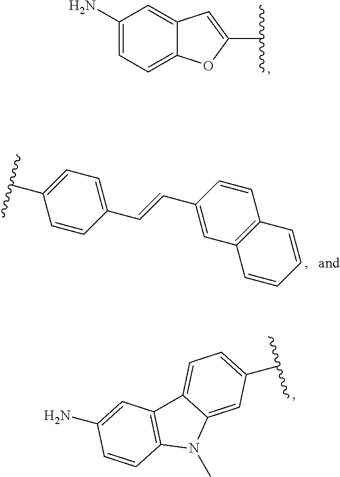

wherein R¹⁰ is selected from hydrogen, halogen, —OH, —NH₂, —NHC(O)CH₃, methyl, and methoxy; and wherein each of R^{11a} and R^{11b} is independently selected from hydrogen, —OH, methyl, and methoxy; and wherein each occurrence of \textcircled{A} is independently selected from:

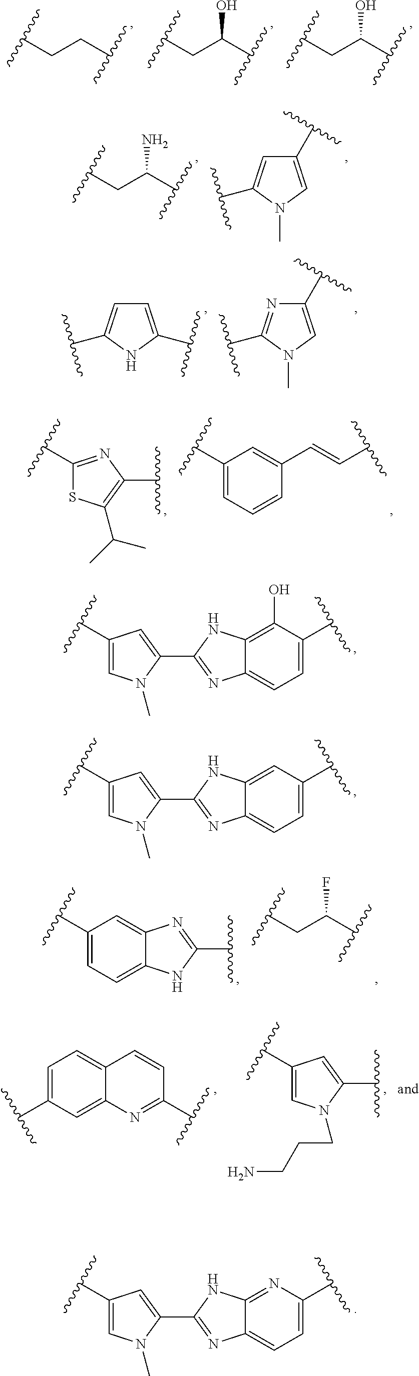

In various aspects, the polyamide moiety is a U-pin polyamide moiety. In a further aspect, the U-pin polyamide moiety is a residue of a structure represented by a formula:

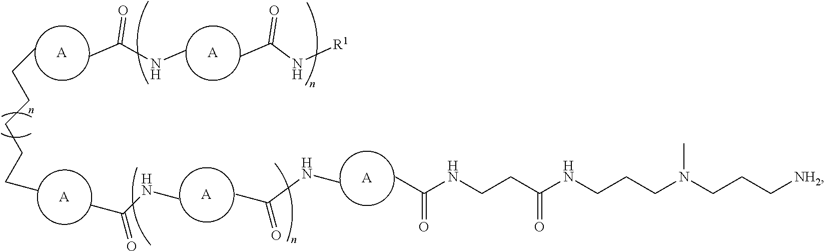

wherein each occurrence of n is independently an integer selected from 1 to 20; wherein R¹ is selected from:

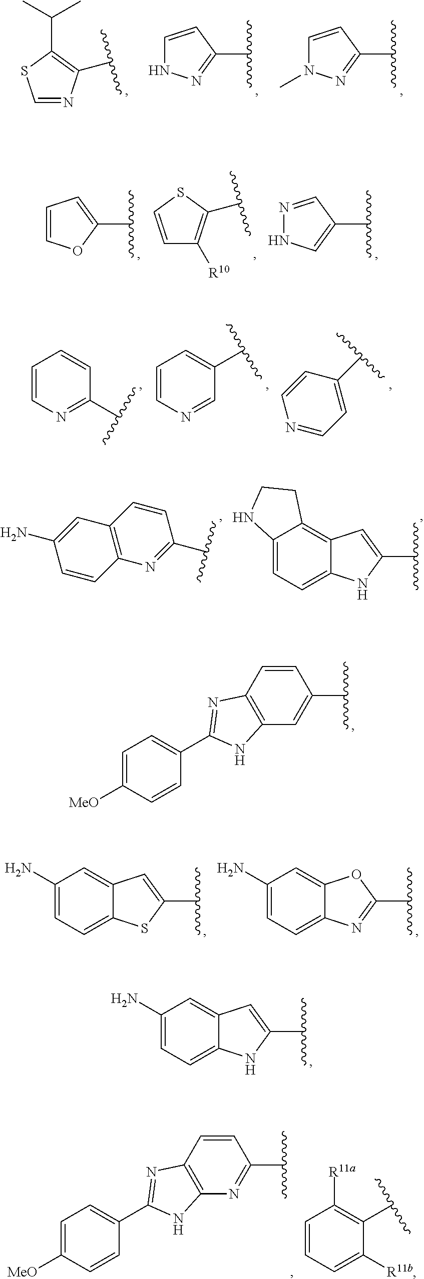

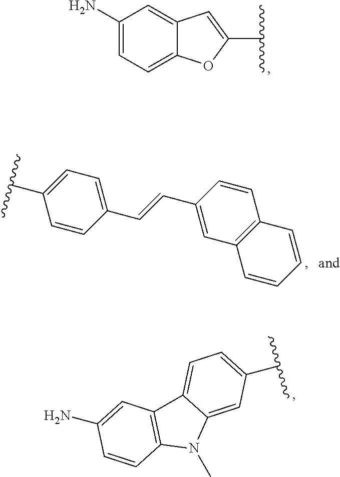

wherein R¹⁰ is selected from hydrogen, halogen, —OH, —NH₂, —NHC(O)CH₃, methyl, and methoxy; and wherein each of R^{11a} and R^{11b} is independently selected from hydrogen, —OH, methyl, and methoxy; and wherein each occurrence of \textcircled{A} is independently selected from:

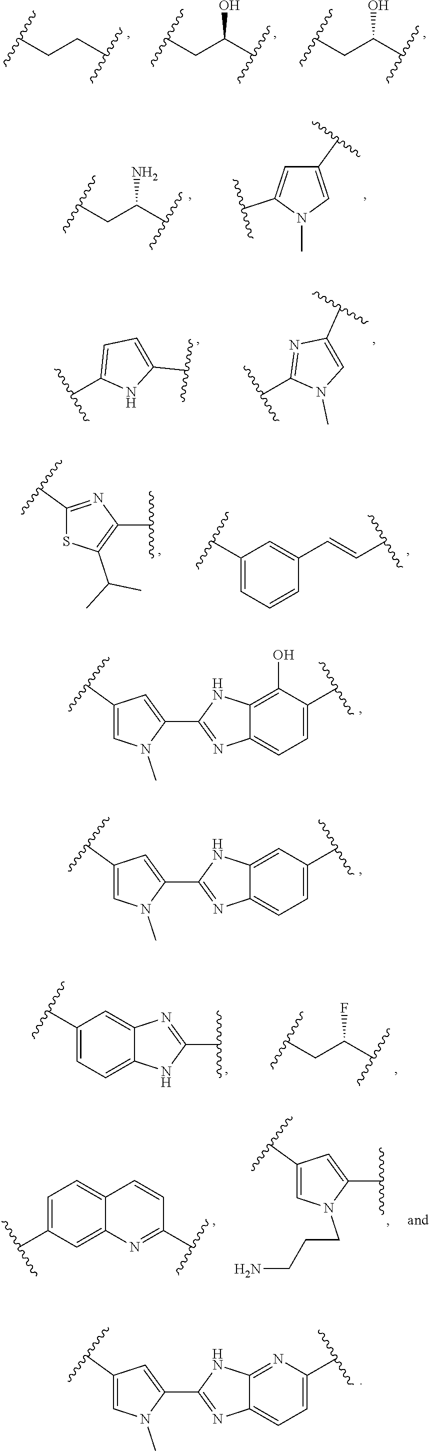

In various aspects, the polyamide moiety is an H-pin polyamide moiety. In a further aspect, the H-pin polyamide moiety is a residue of a structure represented by a formula:

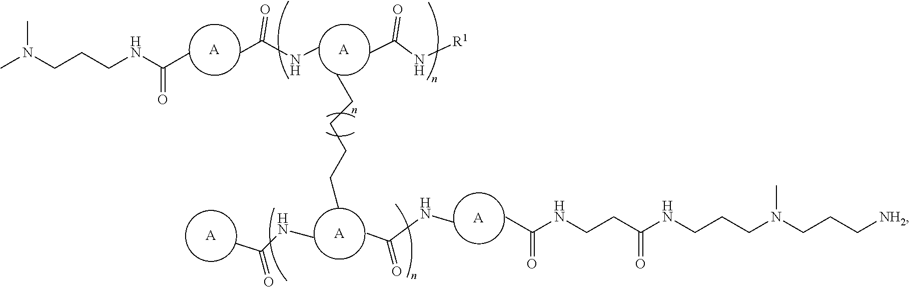

wherein each occurrence of n is independently an integer selected from 1 to 20; wherein R¹ is selected from:

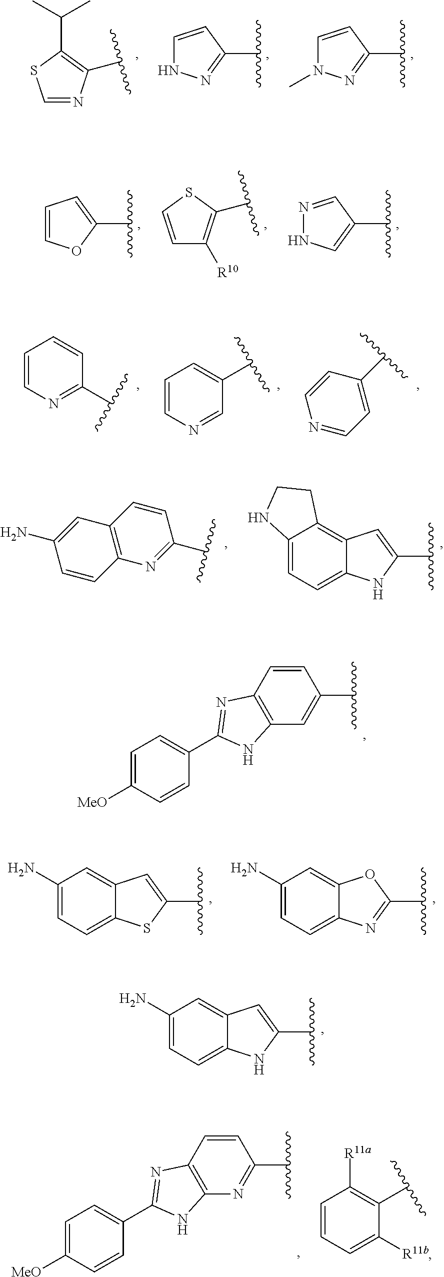

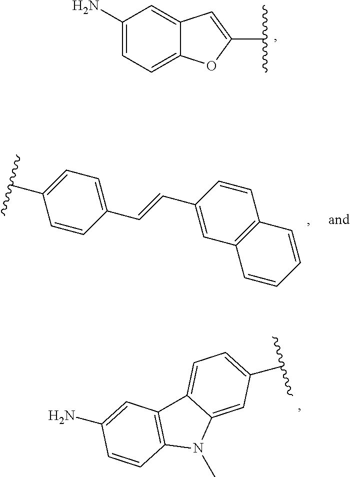

wherein R¹⁰ is selected from hydrogen, halogen, —OH, —NH₂, —NHC(O)CH₃, methyl, and methoxy; and wherein each of R^{11a} and R^{11b} is independently selected from hydrogen, —OH, methyl, and methoxy; and wherein each occurrence of \textcircled{A} is independently selected from:

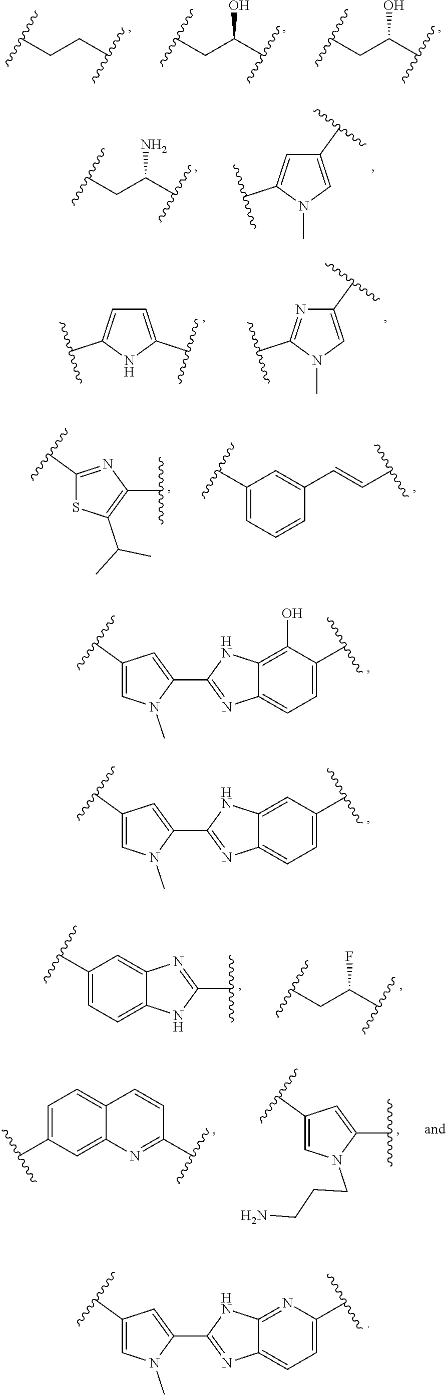

In various aspects, the polyamide moiety is a tandem hairpin polyamide moiety. In a further aspect, the tandem hairpin polyamide moiety is a residue of a structure represented by a formula:

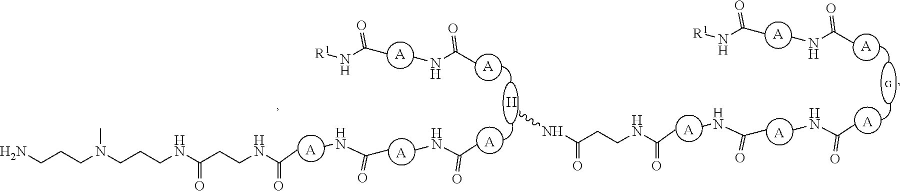

wherein each occurrence of n is independently an integer selected from 1 to 20; wherein each of G and H is independently a polyamide turn linker; wherein each occurrence of R¹ is independently selected from:

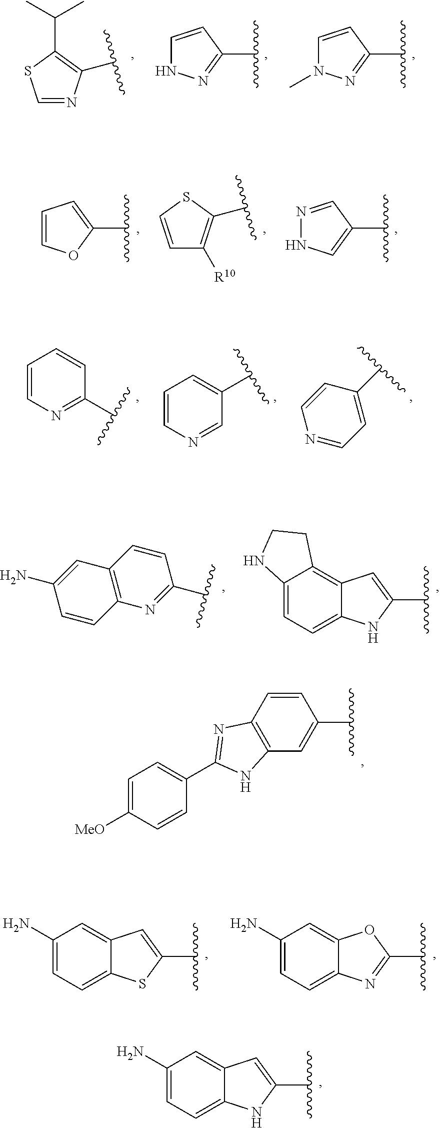

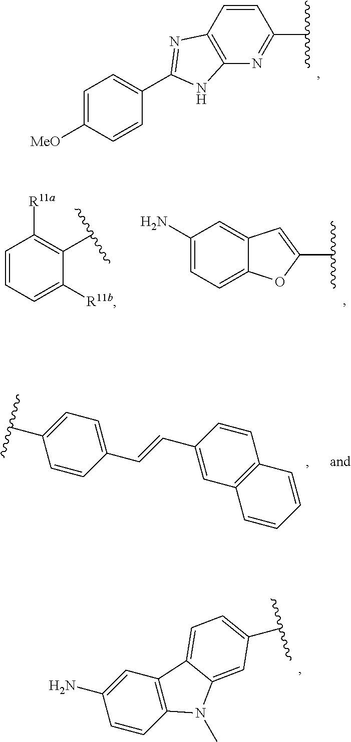

wherein R¹⁰ is selected from hydrogen, halogen, —OH, —NH₂, —NHC(O)CH₃, methyl, and methoxy; and wherein each of R^{11a} and R^{11b} is independently selected from hydrogen, —OH, methyl, and methoxy; and wherein each occurrence of \textcircled{A} is independently selected from:

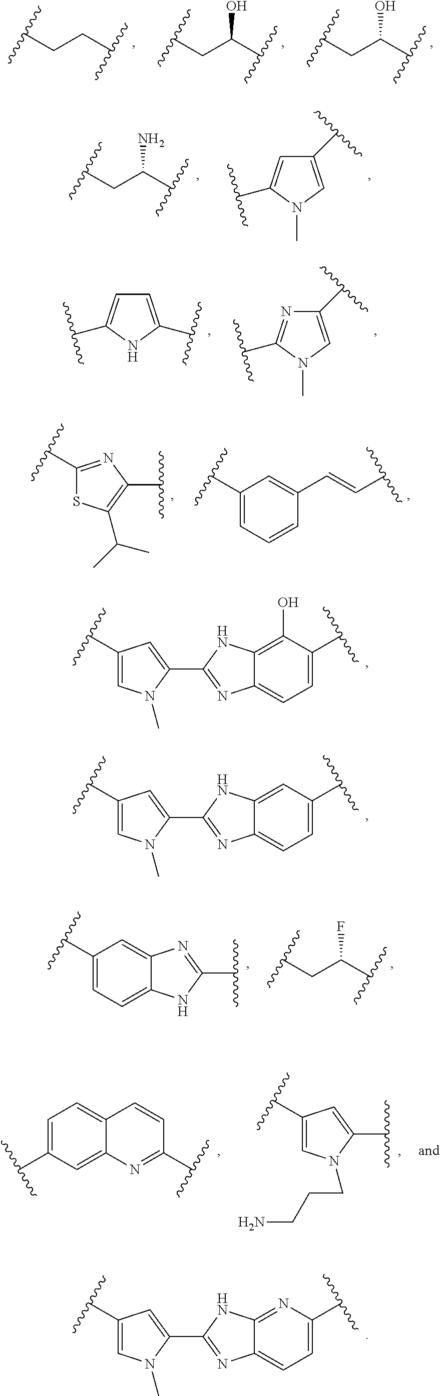

In various aspects, the polyamide moiety is a hairpin polyamide moiety. In a further aspect, the hairpin polyamide moiety is a residue of a structure represented by a formula:

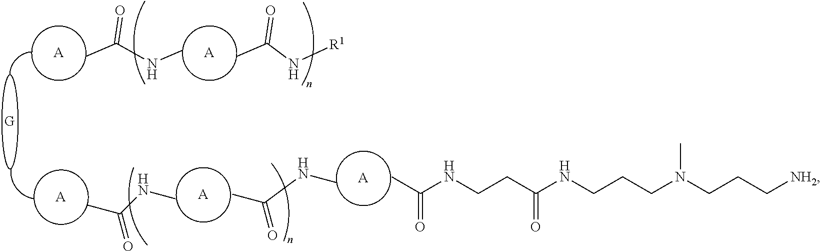

wherein n is an integer selected from 1 to 20; wherein G is a polyamide turn linker; wherein R¹ is selected from:

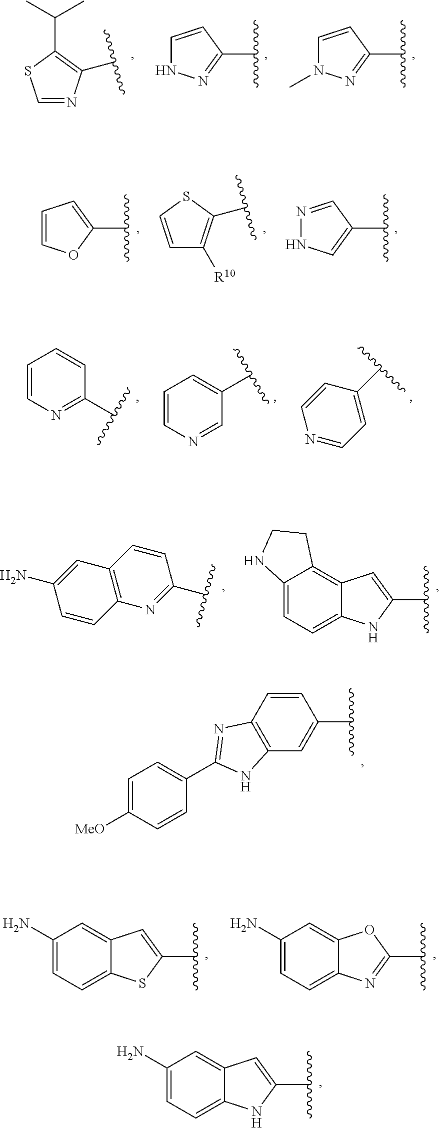

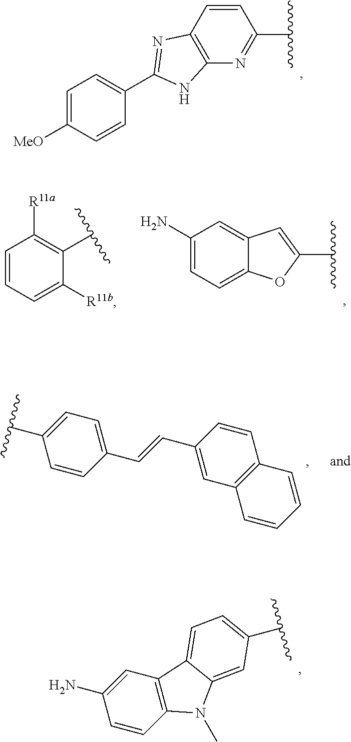

wherein R¹⁰ is selected from hydrogen, halogen, —OH, —NH₂, —NHC(O)CH₃, methyl, and methoxy; and wherein each of R^{11a} and R^{11b} is independently selected from hydrogen, —OH, methyl, and methoxy; and wherein each occurrence of \textcircled{A} is independently selected from:

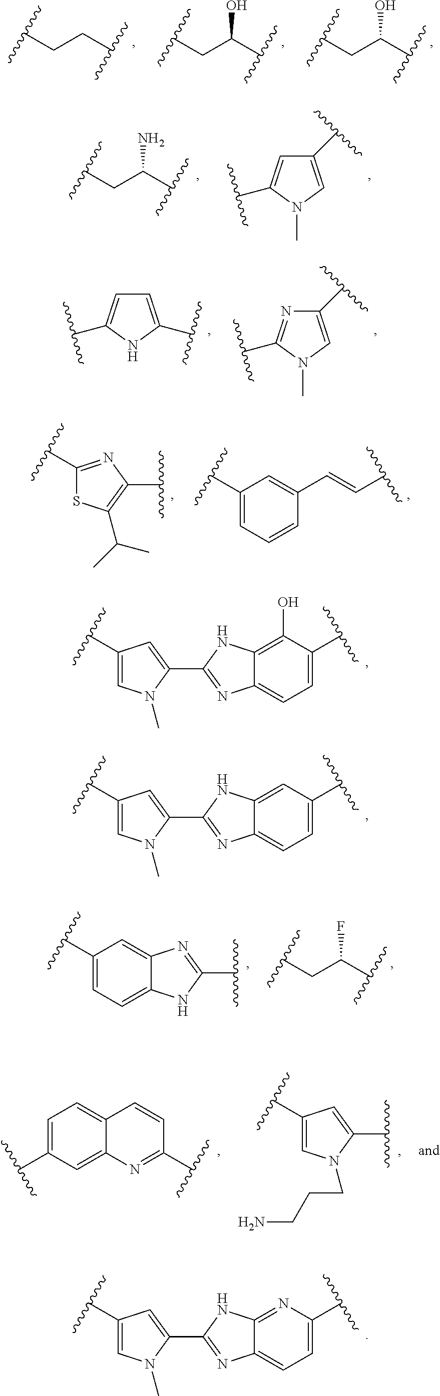

In various aspects, the polyamide moiety is a cyclic polyamide moiety. In a further aspect, the cyclic polyamide moiety is a residue of a structure represented by a formula:

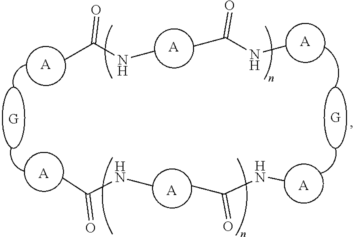

wherein n is an integer selected from 1 to 20; wherein each occurrence of G is independently a polyamide turn linker; wherein each occurrence of \textcircled{A} is independently selected from:

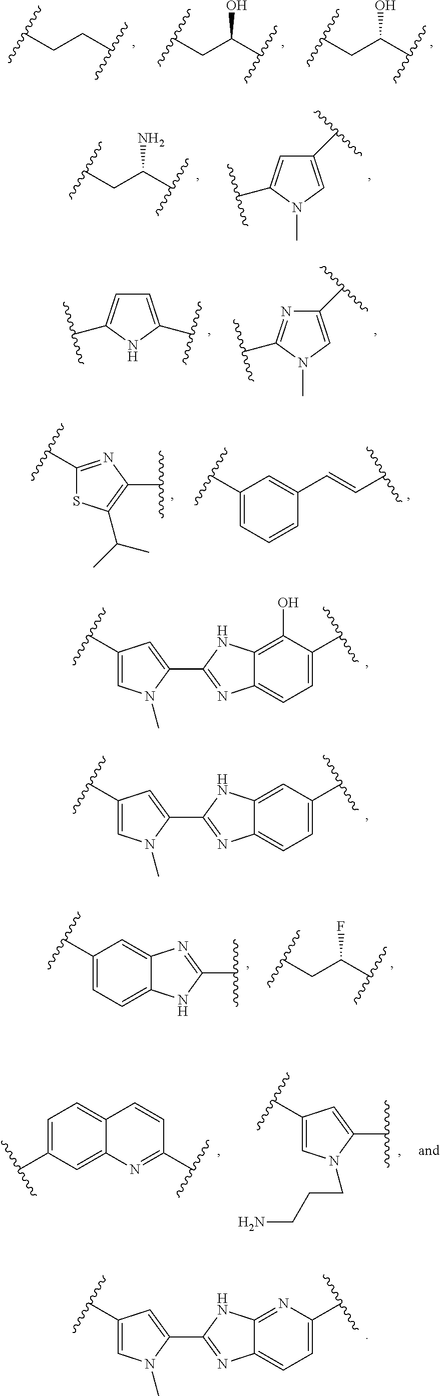

In various aspects, the polyamide moiety is selected from residues of:

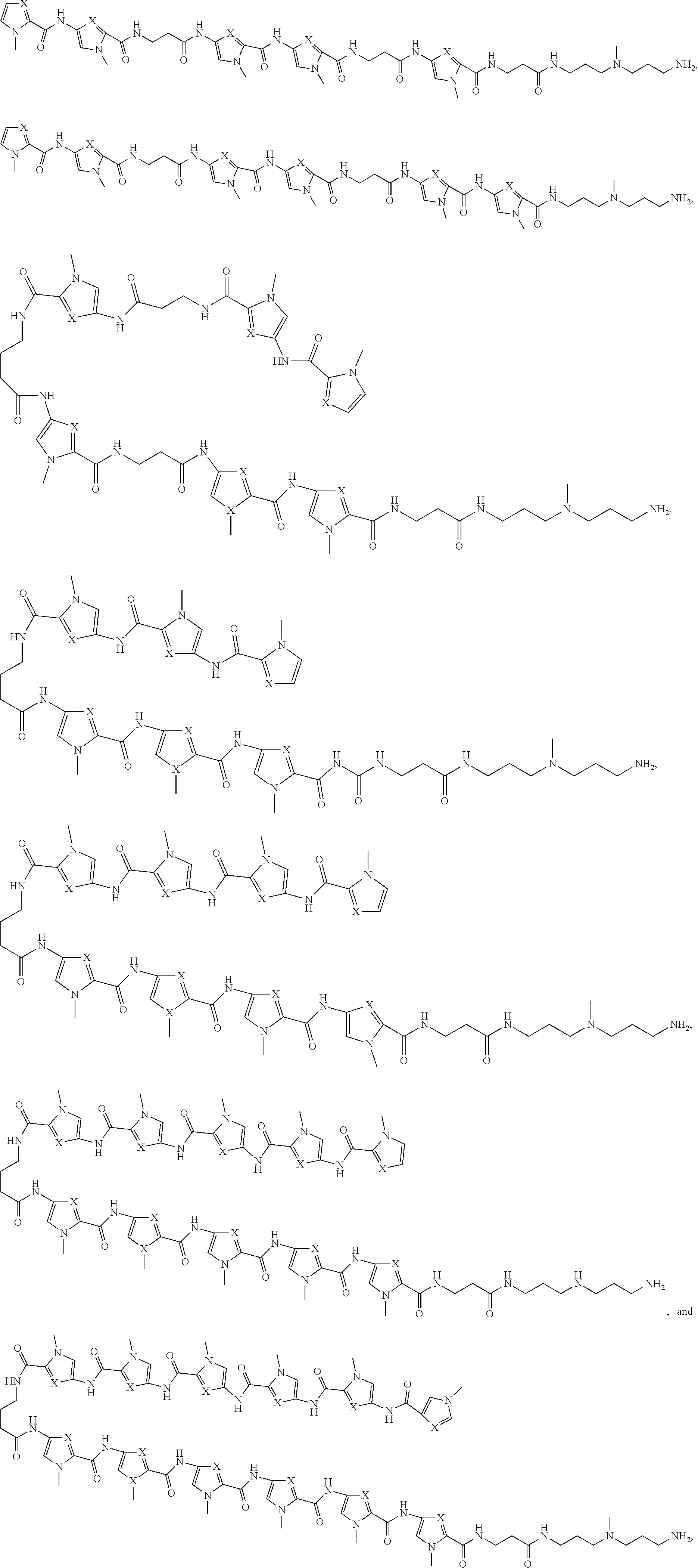

wherein each occurrence of X is independently selected from N and CH.

In various aspects, the polyamide moiety is selected from:

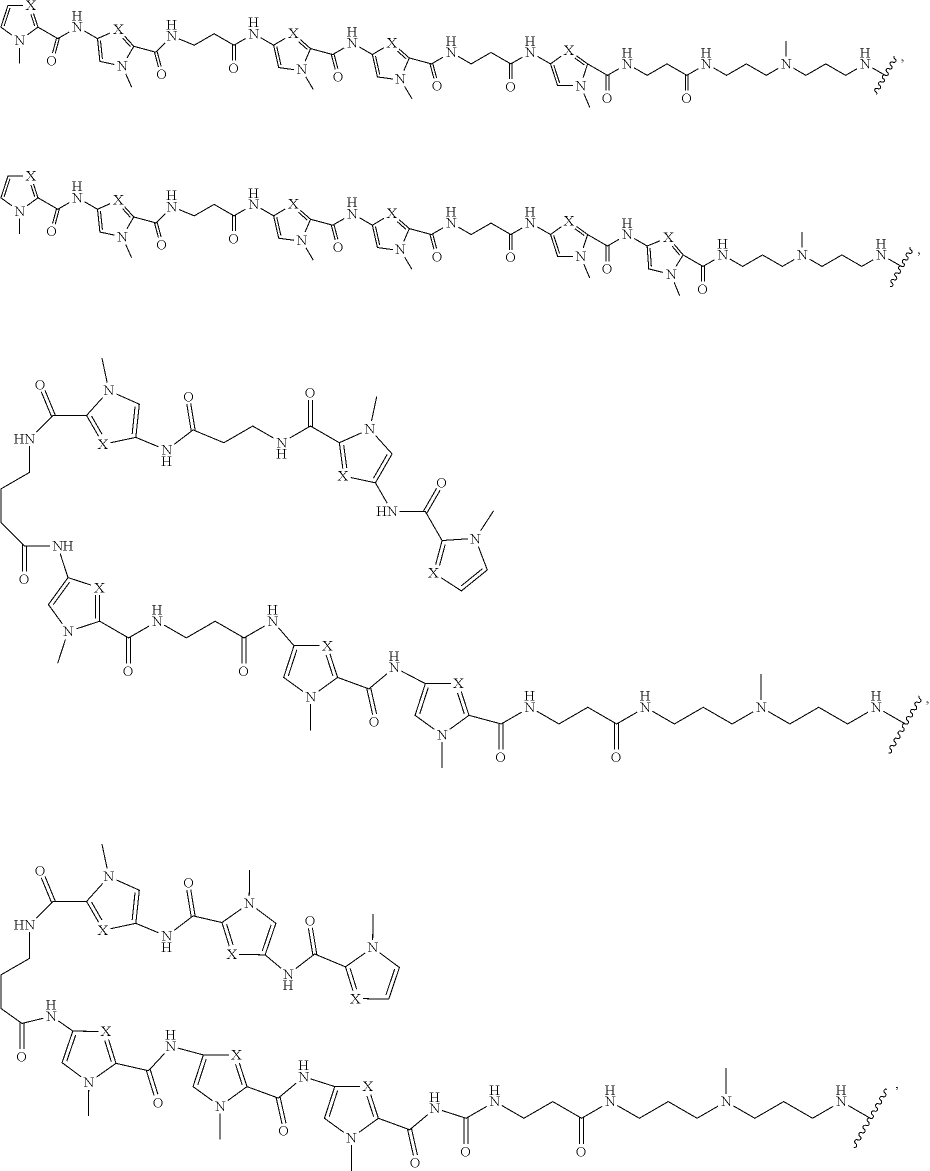

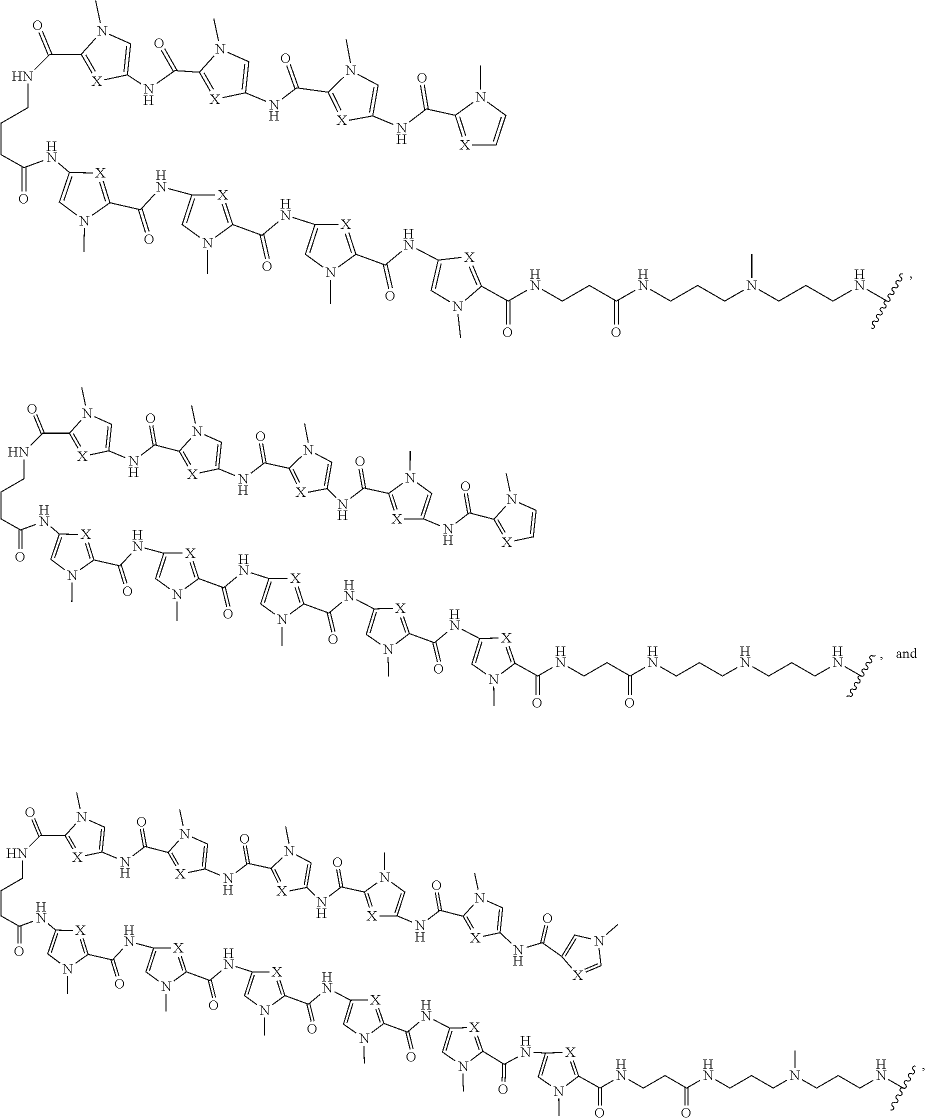

wherein each occurrence of X is independently selected from N and CH.

In various aspects, the polyamide moiety is selected from residues of:

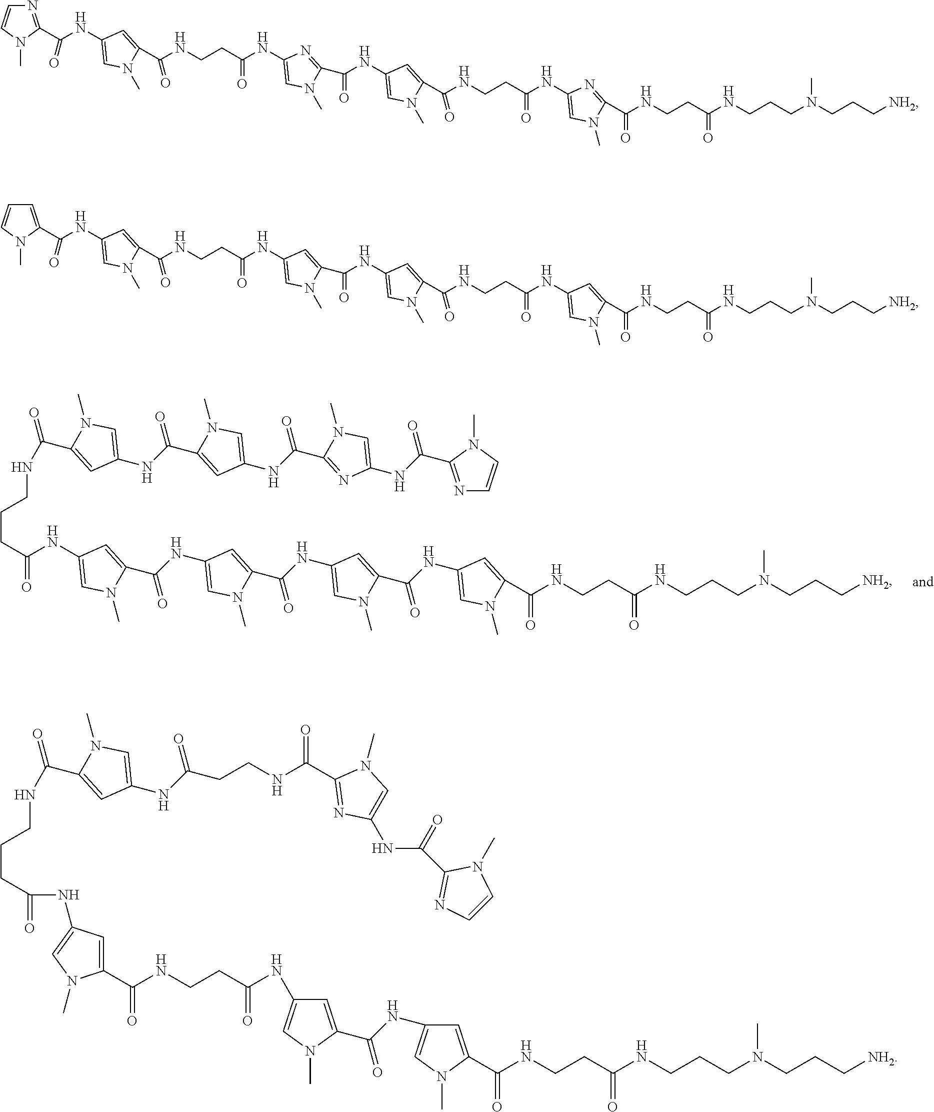

In various aspects, the polyamide moiety is selected from:

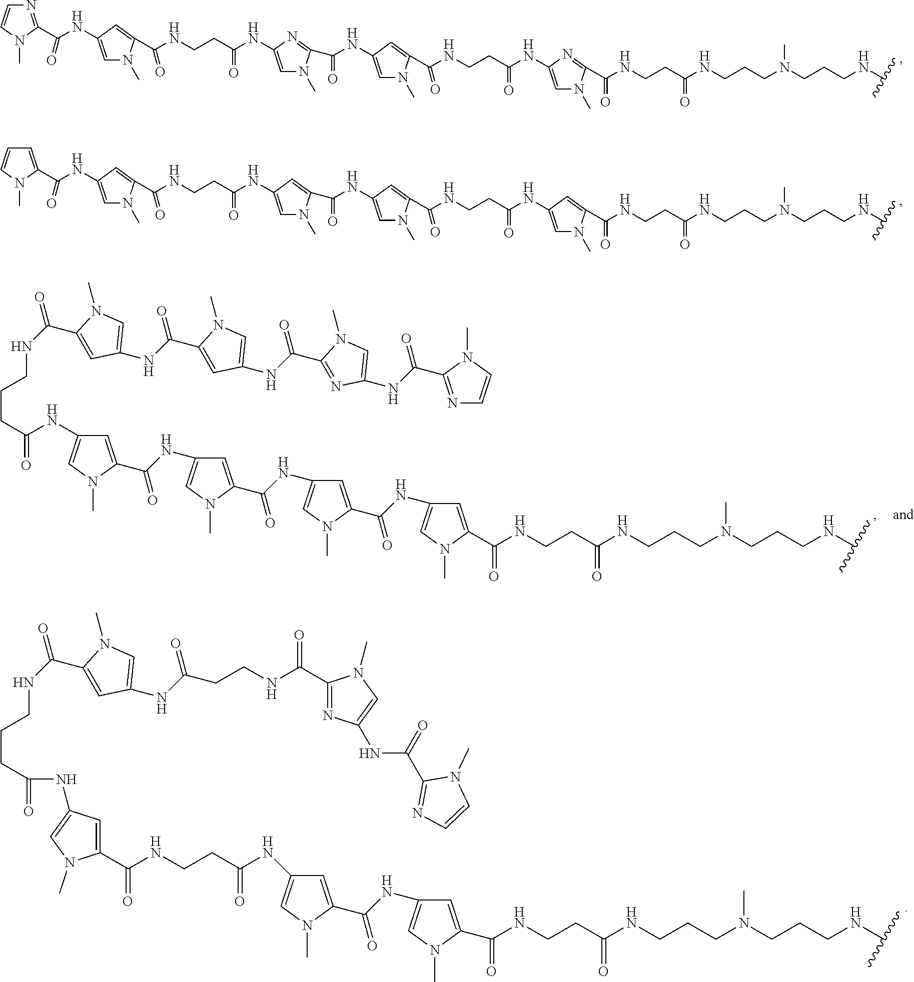

In various aspects, the polyamide moiety has a structure represented by a formula:

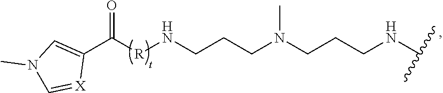

wherein t is an integer selected from 4 to 20; wherein X is selected from N and CH; and wherein each occurrence of R is independently selected from:

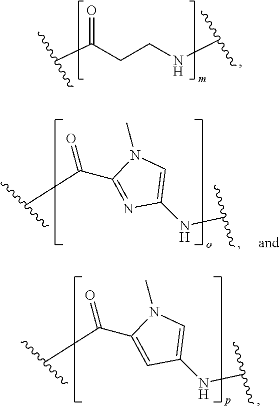

wherein each of m, o, and p is independently selected from 1, 2, 3, and 4; and wherein the sum of m, o, and p is equal to t.

In various aspects, the polyamide moiety is a residue of a structure:

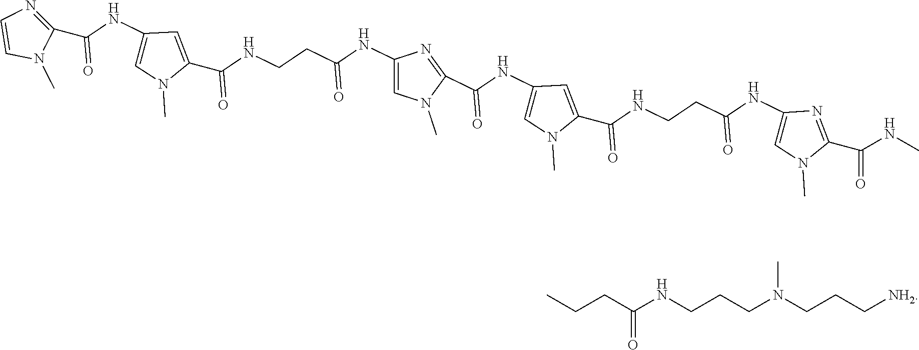

In various aspects, the polyamide moiety has a structure represented by a formula:

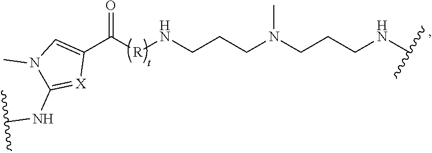

wherein t is an integer selected from 4 to 20; wherein X is selected from N and CH; and wherein each occurrence of R is independently selected from:

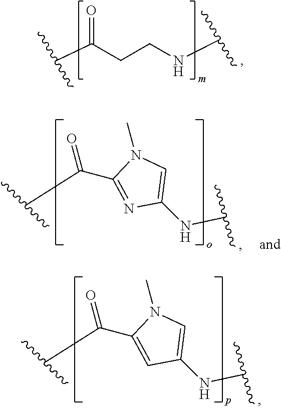

wherein each of m, o, and p is independently selected from 1, 2, 3, and 4; and wherein the sum of m, o, and p is equal to t.

In various aspects, the polyamide moiety is selected from:

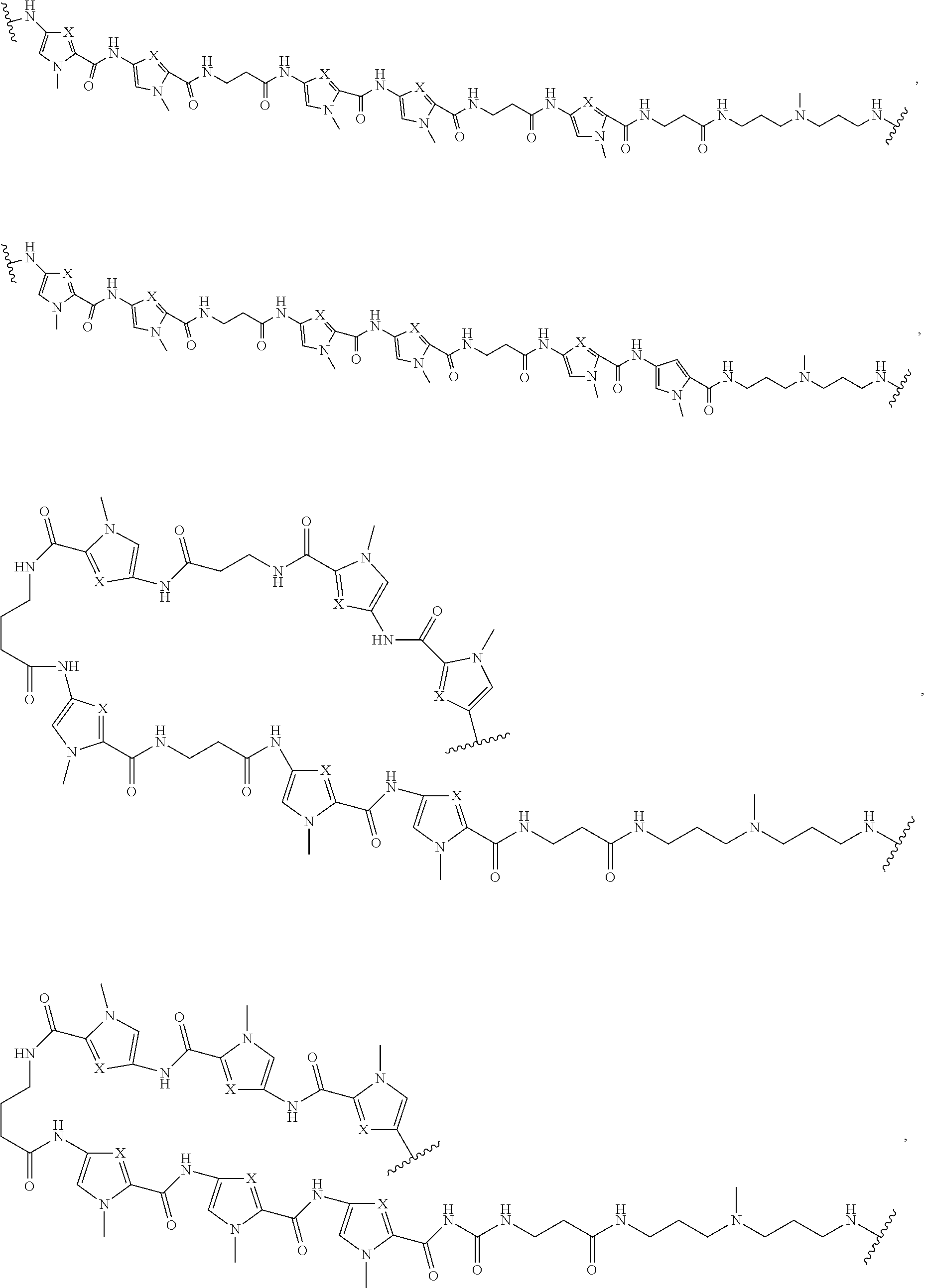

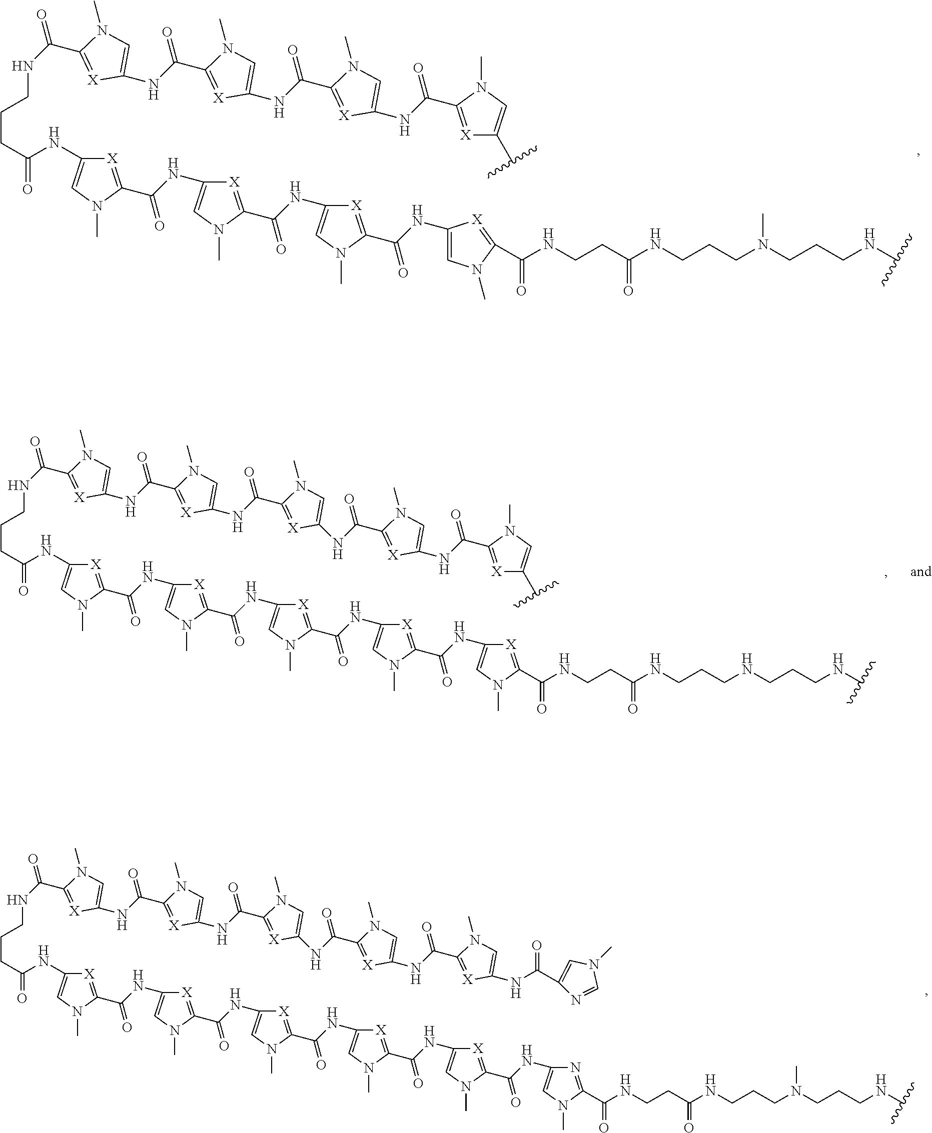

wherein each occurrence of X is independently selected from N and CH.

In various aspects, the polyamide moiety is selected from:

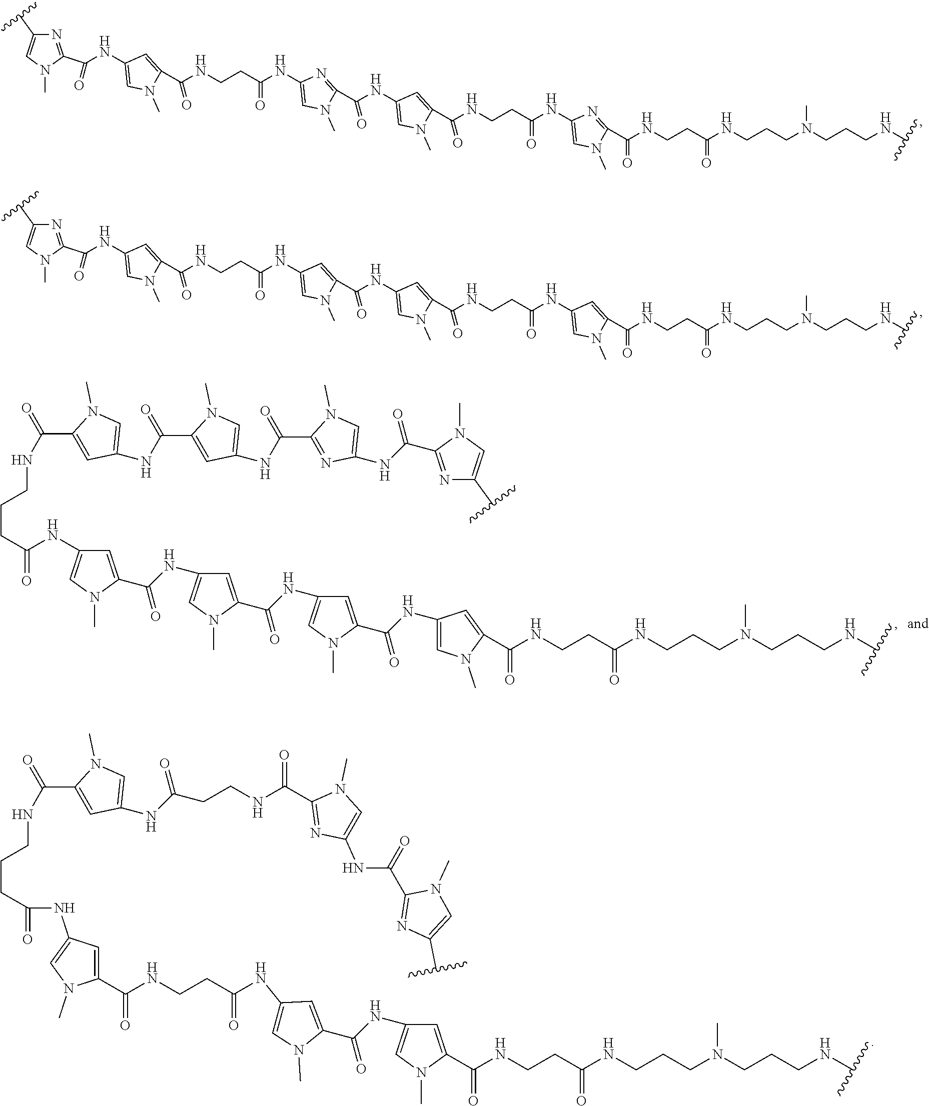

In various aspects, the polyamide moiety is a residue of a structure selected from:

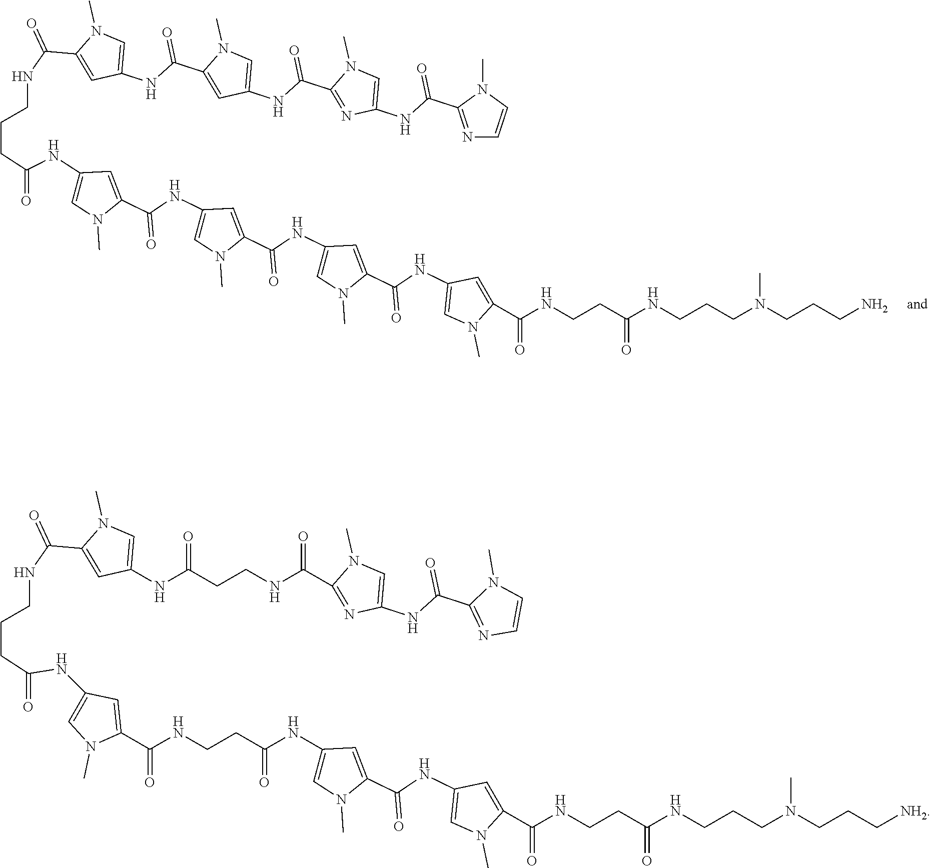 [SMILES: CN(CCCN)CCCNC(=O)CCNC(=O)c1cc(NC(=O)c2cc(NC(=O)c3cc(NC(=O)c4cc(NC(=O)CCCNC(=O)c5cc(NC(=O)c6cc(NC(=O)c7nc(NC(=O)c8nccn8C)cn7C)cn6C)cn5C)cn4C)cn3C)cn2C)cn1C ; CN(CCCN)CCCNC(=O)CCNC(=O)c1cc(NC(=O)c2cc(NC(=O)CCNC(=O)c3cc(NC(=O)CCCNC(=O)c4cc(NC(=O)CCNC(=O)c5nc(NC(=O)c6nccn6C)cn5C)cn4C)cn3C)cn2C)cn1C]

In various aspects, the polyamide moiety has a structure represented by a formula:

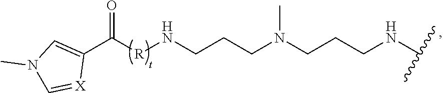

wherein t is an integer selected from 4 to 20; wherein X is selected from N and CH; and wherein each occurrence of R is independently selected from:

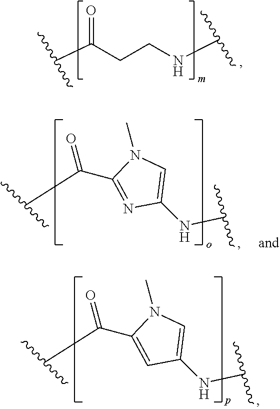

wherein each of m, o, and p is independently selected from 1, 2, 3, and 4; and wherein the sum of m, o, and p is equal to t.

In various aspects, the polyamide moiety is selected from:

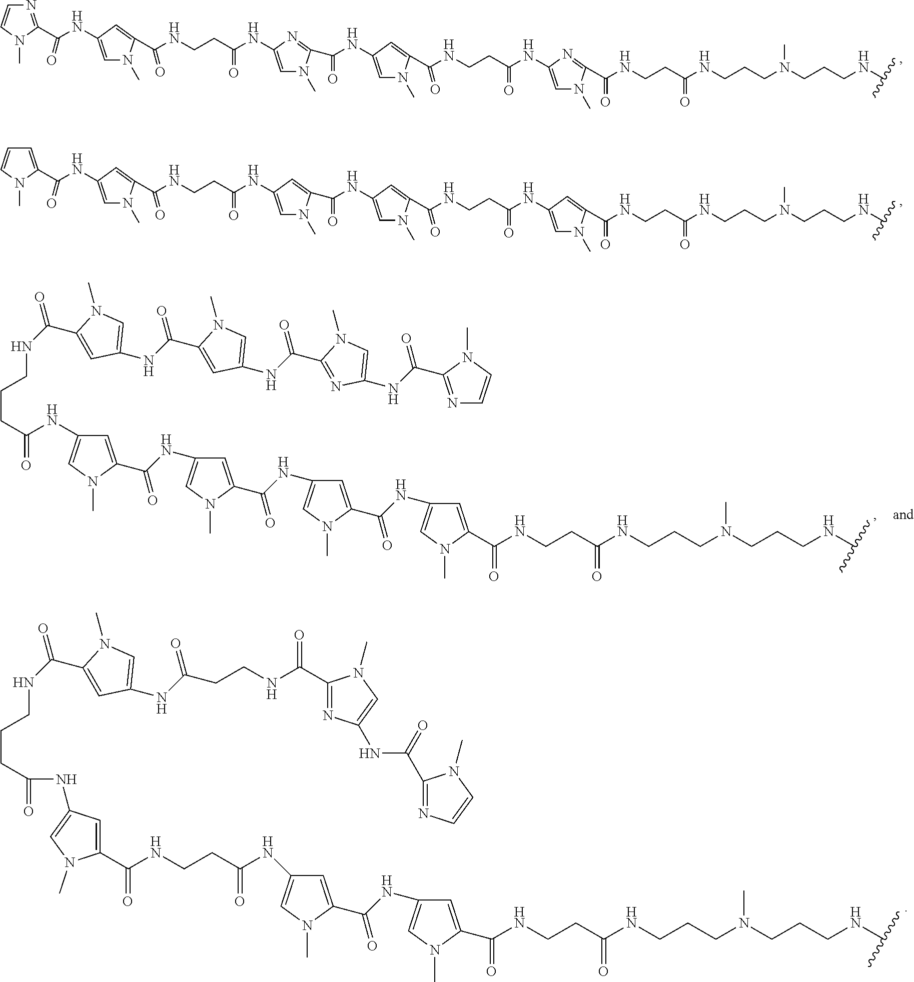

In one aspect, each of m, o, and p is independently an integer selected from 1 to 20. In a further aspect, each of m, o, and p is independently an integer selected from 1 to 15. In a still further aspect, each of m, o, and p is independently an integer selected from 1 to 10. In yet a further aspect, each of m, o, and p is independently an integer selected from 1 to 5. In an even further aspect, each of m, o, and p is independently an integer selected from 5 to 20. In a still further aspect, each of m, o, and p is independently an integer selected from 10 to 20. In yet a further aspect, each of m, o, and p is independently an integer selected from 15 to 20. In an even further aspect, each of m, o, and p is independently an integer selected from 5 to 15.

In one aspect, each of m, o, and p is independently selected from 1, 2, 3, and 4, and the sum of m, o, and p is equal to t. In a further aspect, each of m, o, and p is independently selected from 1, 2, and 3. In a still further aspect, each of m, o, and p is independently selected from 1 and 2. In yet a further aspect, each of m, o, and p is independently selected from 1 and 3. In an even further aspect, each of m, o, and p is independently selected from 1 and 4. In a still further aspect, each of m, o, and p is independently selected from 2 and 3. In yet a further aspect, each of m, o, and p is independently selected from 2 and 4. In an even further aspect, each of m, o, and p is independently selected from 3 and 4. In a still further aspect, each of m, o, and p is 4. In yet a further aspect, each of m, o, and p is 3. In an even further aspect, each of m, o, and p is 2.

In one aspect, n is an integer selected from 1 to 20. In a further aspect, n is an integer selected from 1 to 15. In a still further aspect, n is an integer selected from 1 to 10. In yet a further aspect, n is an integer selected from 1 to 5. In an even further aspect, n is an integer selected from 5 to 20. In a still further aspect, n is an integer selected from 10 to 20. In yet a further aspect, n is an integer selected from 15 to 20. In an even further aspect, n is an integer selected from 5 to 15.

In one aspect, each occurrence of n is independently an integer selected from 1 to 20. In a further aspect, each occurrence of n is independently an integer selected from 1 to 15. In a still further aspect, each occurrence of n is independently an integer selected from 1 to 10. In yet a further aspect, each occurrence of n is independently an integer selected from 1 to 5. In an even further aspect, each occurrence of n is independently an integer selected from 5 to 20. In a still further aspect, each occurrence of n is independently an integer selected from 10 to 20. In yet a further aspect, each occurrence of n is independently an integer selected from 15 to 20. In an even further aspect, each occurrence of n is independently an integer selected from 5 to 15.

In one aspect, t is an integer selected from 4 to 20. In a further aspect, t is an integer selected from 8 to 20. In a still further aspect, t is an integer selected from 12 to 20. In yet a further aspect, t is an integer selected from 16 to 20. In an even further aspect, t is an integer selected from 4 to 16. In a still further aspect, t is an integer selected from 4 to 12. In yet a further aspect, 4 to 8. In an even further aspect, t is an integer selected from 4 to 16. In a still further aspect, t is an integer selected from 8 to 12.

In one aspect, each of u and v is independently an integer selected from 1 to 20. In a further aspect, each of u and v is independently an integer selected from 1 to 15. In a still further aspect, each of u and v is independently an integer selected from 1 to 10. In yet a further aspect, each of u and v is independently an integer selected from 1 to 5. In an even further aspect, each of u and v is independently an integer selected from 5 to 20. In a still further aspect, each of u and v is independently an integer selected from 10 to 20. In yet a further aspect, each of u and v is independently an integer selected from 15 to 20. In an even further aspect, each of u and v is independently an integer selected from 5 to 15.

In one aspect, each of u′ and v′ is independently an integer selected from 1 to 20. In a further aspect, each of u′ and v′ is independently an integer selected from 1 to 15. In a still further aspect, each of u′ and v′ is independently an integer selected from 1 to 10. In yet a further aspect, each of u′ and v′ is independently an integer selected from 1 to 5. In an even further aspect, each of u′ and v′ is independently an integer selected from 5 to 20. In a still further aspect, each of u′ and v′ is independently an integer selected from 10 to 20. In yet a further aspect, each of u′ and v′ is independently an integer selected from 15 to 20. In an even further aspect, each of u′ and v′ is independently an integer selected from 5 to 15.

In one aspect, w is an integer selected from 1 to 20. In a further aspect, w is an integer selected from 1 to 15. In a still further aspect, w is an integer selected from 1 to 10. In yet a further aspect, w is an integer selected from 1 to 5. In an even further aspect, w is an integer selected from 5 to 20. In a still further aspect, w is an integer selected from 10 to 20. In yet a further aspect, w is an integer selected from 15 to 20. In an even further aspect, w is an integer selected from 5 to 15.

In one aspect, w′ is an integer selected from 1 to 20. In a further aspect, w is an integer selected from 1 to 15. In a still further aspect, w′ is an integer selected from 1 to 10. In yet a further aspect, w′ is an integer selected from 1 to 5. In an even further aspect, w′ is an integer selected from 5 to 20. In a still further aspect, w′ is an integer selected from 10 to 20. In yet a further aspect, w′ is an integer selected from 15 to 20. In an even further aspect, w′ is an integer selected from 5 to 15.

In one aspect, each of u, v, and w is independently an integer selected from 1 to 20. In a further aspect, each of u, v, and w is independently an integer selected from 1 to 15. In a still further aspect, each of u, v, and w is independently an integer selected from 1 to 10. In yet a further aspect, each of u, v, and w is independently an integer selected from 1 to 5. In an even further aspect, each of u, v, and w is independently an integer selected from 5 to 20. In a still further aspect, each of u, v, and w is independently an integer selected from 10 to 20. In yet a further aspect, each of u, v, and w is independently an integer selected from 15 to 20. In an even further aspect, each of u, v, and w is independently an integer selected from 5 to 15.

In one aspect, each of u′, v′, and w′ is independently an integer selected from 1 to 20. In a further aspect, each of u′, v′, and w′ is independently an integer selected from 1 to 15. In a still further aspect, each of u′, v′, and w′ is independently an integer selected from 1 to 10. In yet a further aspect, each of u′, v′, and w′ is independently an integer selected from 1 to 5. In an even further aspect, each of u′, v′, and w′ is independently an integer selected from 5 to 20. In a still further aspect, each of u′, v′, and w′ is independently an integer selected from 10 to 20. In yet a further aspect, each of u′, v′, and w′ is independently an integer selected from 15 to 20. In an even further aspect, each of u′, v′, and w′ is independently an integer selected from 5 to 15.

### (i) A Groups

In one aspect, each occurrence of A is selected from:

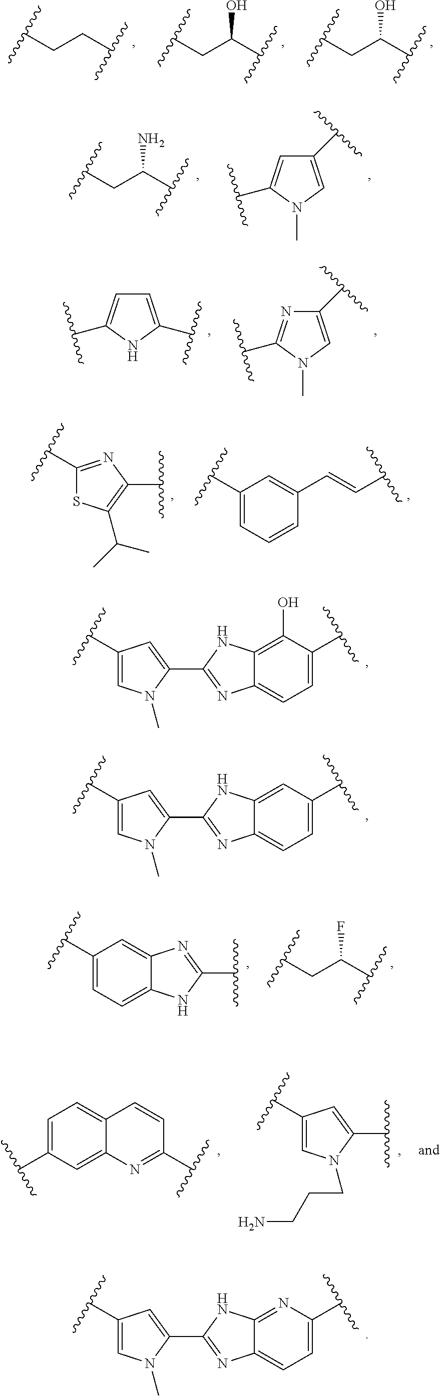

### (ii) G Groups

In one aspect, G is a polyamide turn linker such as, for example, a polyamide turn linker having two points of attachment to the remainder of the compound. Examples of such polyamide turn linkers include, but are not limited to:

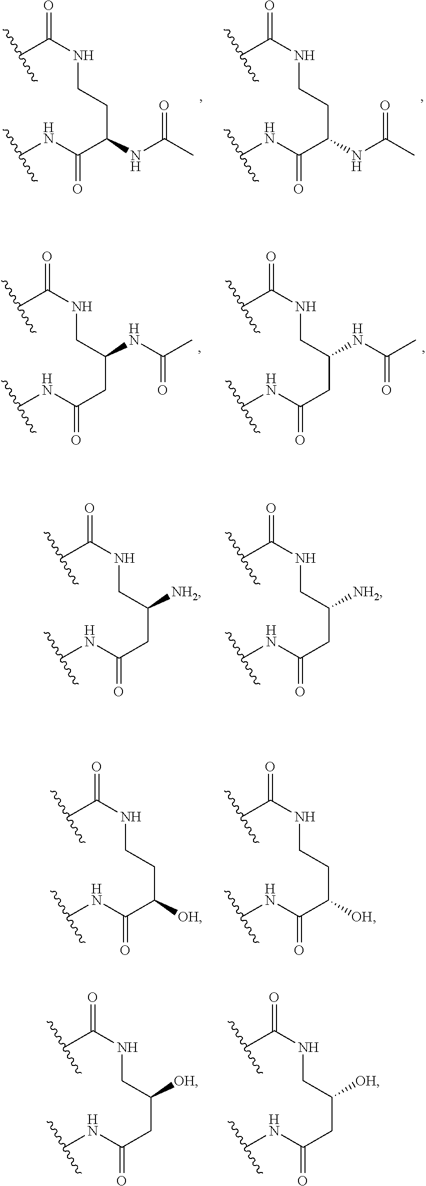

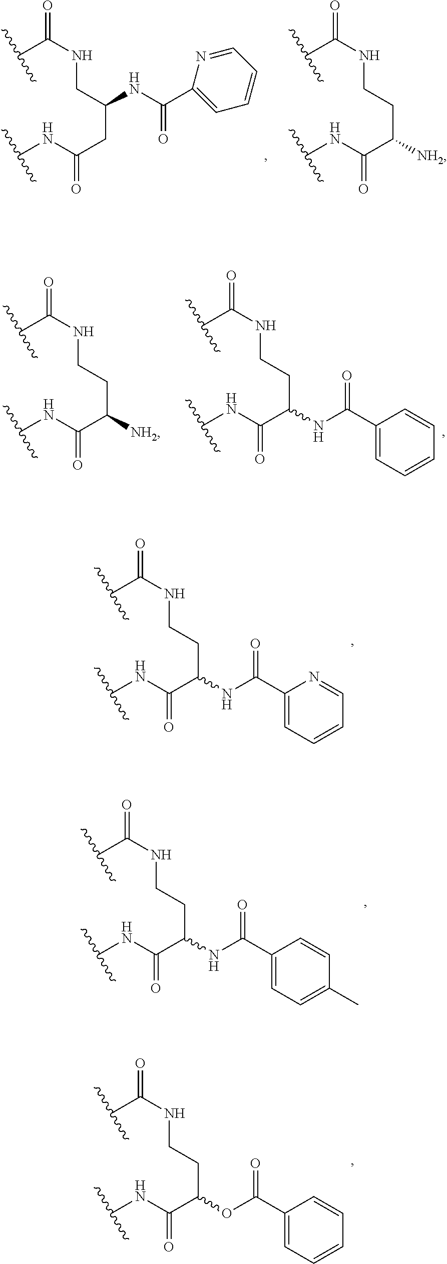

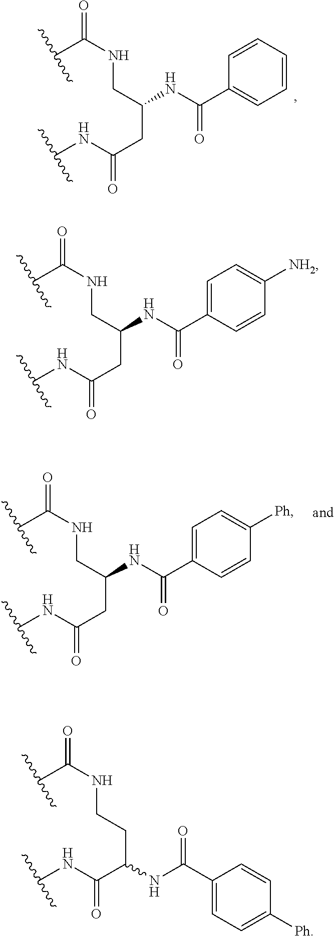 [SMILES: *NC(=O)C[C@H](CNC(*)=O)NC(=O)c1ccccc1 ; *NC(=O)C[C@@H](CNC(*)=O)NC(=O)c1ccc(N)cc1 ; *NC(=O)C[C@@H](CNC(*)=O)NC(=O)c1ccc(-c2ccccc2)cc1 ; *NC(=O)C(CCNC(*)=O)NC(=O)c1ccc(-c2ccccc2)cc1]

In various aspects, G is a structure selected from:

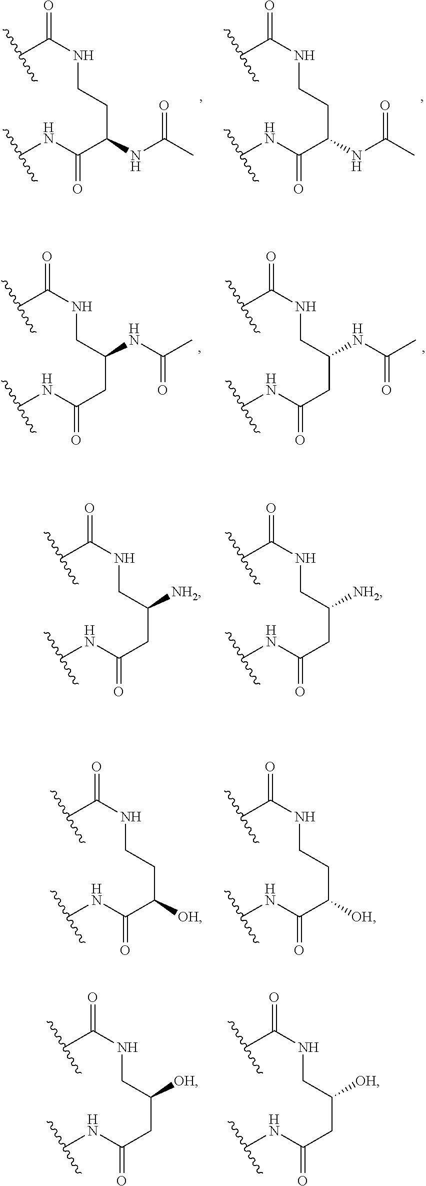

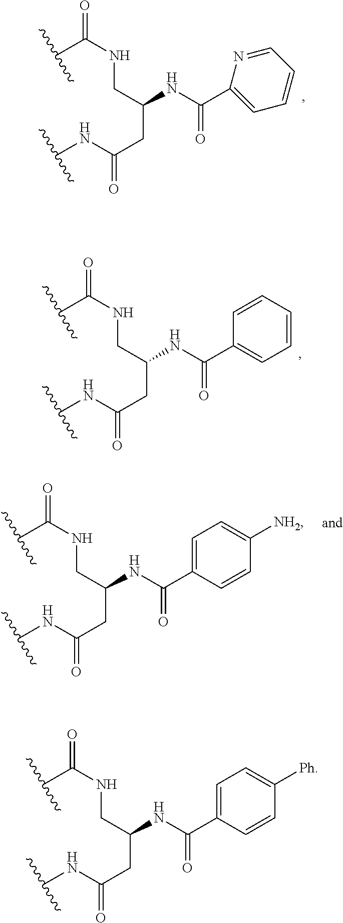 [SMILES: *NC(=O)C[C@@H](CNC(*)=O)NC(=O)c1ccccn1 ; *NC(=O)C[C@H](CNC(*)=O)NC(=O)c1ccccc1 ; *NC(=O)C[C@@H](CNC(*)=O)NC(=O)c1ccc(N)cc1 ; *NC(=O)C[C@@H](CNC(*)=O)NC(=O)c1ccc(-c2ccccc2)cc1]

(iii) H Groups

In one aspect, H is a polyamide turn linker such as, for example, a polyamide turn linker having three points of attachment. Examples of polyamide turn linkers include, but are not limited to, structures selected from:

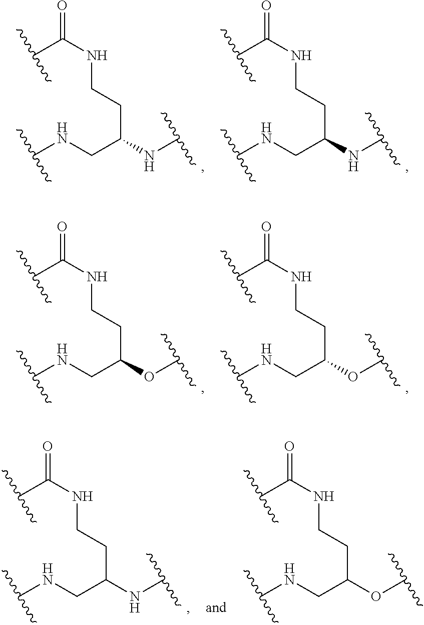 [SMILES: *NC[C@H](CCNC(*)=O)N* ; *NC[C@@H](CCNC(*)=O)N* ; *NC[C@@H](CCNC(*)=O)O* ; *NC[C@H](CCNC(*)=O)O* ; *NCC(CCNC(*)=O)N* ; *NCC(CCNC(*)=O)O*]

### (iv) X Groups

In one aspect, each occurrence of X is independently selected from N or CH. In a further aspect, X is N. In a still further aspect, X is CH.

### (v) R Groups

In one aspect, each occurrence of R is independently selected from:

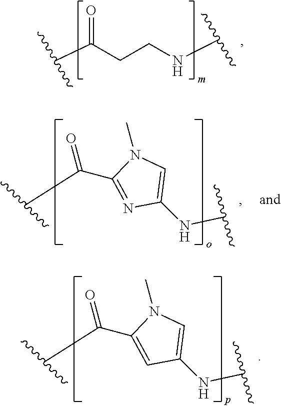

In a further aspect, each occurrence of R is independently selected from:

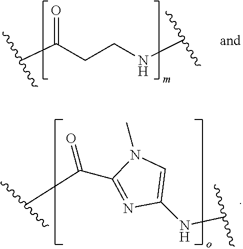

In a further aspect, each occurrence of R is independently selected from:

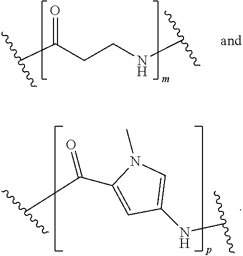

In a further aspect, each occurrence of R is independently selected from:

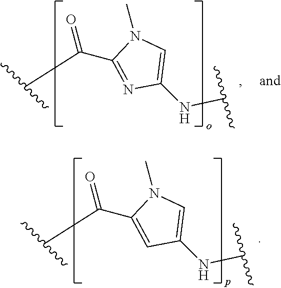

### (vi) R¹ Groups

In one aspect, R¹ is selected from:

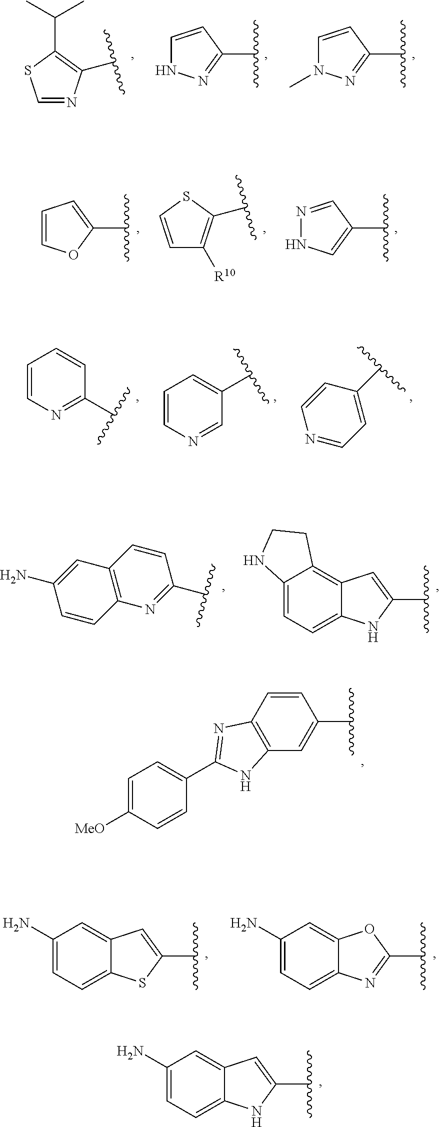

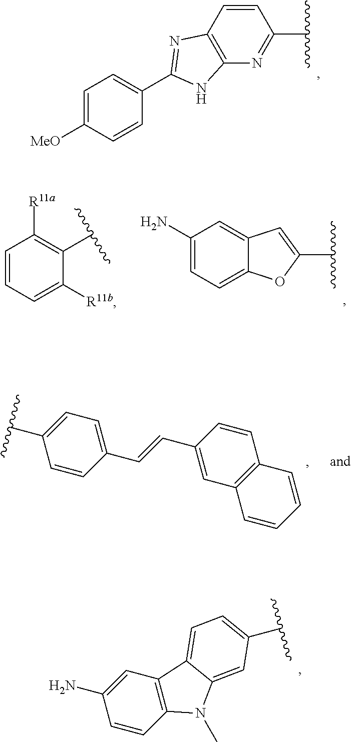

(vii) R¹⁰ Groups

In one aspect, R¹⁰ is selected from hydrogen, halogen, —OH, —NH₂, —NHC(O)CH₃, methyl, and methoxy. In a still further aspect, R¹⁰, when present, is selected from hydrogen, fluoro, chloro, —OH, —NH₂, —NHC(O)CH₃, methyl, and methoxy. In a still further aspect, R¹⁰, when present, is selected from hydrogen, fluoro, and chloro. In a still further aspect, R¹⁰, when present, is fluoro. In a still further aspect, R¹⁰, when present, is chloro.

In various aspects, R¹⁰, when present, is selected from hydrogen, —OH, —NH₂, —NHC(O)CH₃, methyl, and methoxy. In a further aspect, R¹⁰, when present, is selected from hydrogen, —OH, and —NH₂. In a further aspect, R¹⁰, when present, is selected from hydrogen and —OH. In a further aspect, R¹⁰, when present, is selected from hydrogen and —NH₂.

In various aspects, R¹⁰, when present, is selected from hydrogen, —NH₂, —NHC(O)CH₃. In a further aspect, R¹⁰, when present, is selected from hydrogen and —NH₂. In a further aspect, R¹⁰, when present, is selected from hydrogen and —NHC(O)CH₃. In a yet further aspect, R¹⁰, when present, is —NH₂. In a yet further aspect, R¹⁰, when present, is —NHC(O)CH₃.

In various aspects, R¹⁰, when present, is selected from hydrogen, —OH, methyl, and methoxy. In a further aspect, R¹⁰, when present, is selected from hydrogen, —OH, and methoxy. In a further aspect, R¹⁰, when present, is selected from hydrogen and —OH. In a further aspect, R¹⁰, when present, is selected from hydrogen and methoxy. In a further aspect, R¹⁰, when present, is selected from hydrogen and methyl. In a yet further aspect, R¹⁰, when present, is —OH. In a yet further aspect, R¹⁰, when present, is methoxy. In a yet further aspect, R¹⁰, when present, is methyl.

In various aspects, R¹⁰, when present, is hydrogen.

(viii) R^{11A} AND R^{11B} GROUPS

In one aspect, each of R^{11a} and R^{11b} is independently selected from hydrogen, —OH, methyl, and methoxy. In a further aspect, each of R^{11a} and R^{11b}, when present, is independently selected from hydrogen, methyl, and methoxy. In a further aspect, each of R^{11a} and R^{11b}, when present, is independently selected from hydrogen, —OH, and methoxy. In a further aspect, each of R^{11a} and R^{11b}, when present, is independently selected from hydrogen, —OH, and methyl. In a still further aspect, each of R^{11a} and R^{11b}, when present, is independently selected from —OH, methyl, and methoxy. In yet a further aspect, each of R^{11a} and R^{11b}, when present, is independently selected from hydrogen, and —OH. In yet a further aspect, each of R^{11a} and R^{11b}, when present, is independently selected from hydrogen and methyl. In yet a further aspect, each of R^{11a} and R^{11b}, when present, is independently selected from hydrogen and methoxy. In yet a further aspect, each occurrence of R¹¹, when present, is independently selected from —OH and methyl. In yet a further aspect, each of R^{11a} and R^{11b}, when present, is independently selected from —OH and methoxy. In yet a further aspect, each of R^{11a} and R^{11b} when present, is independently selected from methyl and methoxy.

In various aspects, each of R^{11a} and R^{11b}, when present, is hydrogen.

b. Ubiquitin-Proteosome System (UPS) Targeting Moieties

In one aspect, the disclosed sequence selective DNA binding compound comprises a Ubiquitin-Proteosome System (UPS) targeting moiety. In a further aspect, the UPS targeting moiety is an E3 ligase binding ligand. Examples of E3 ligase binding ligands include, but are not limited to:

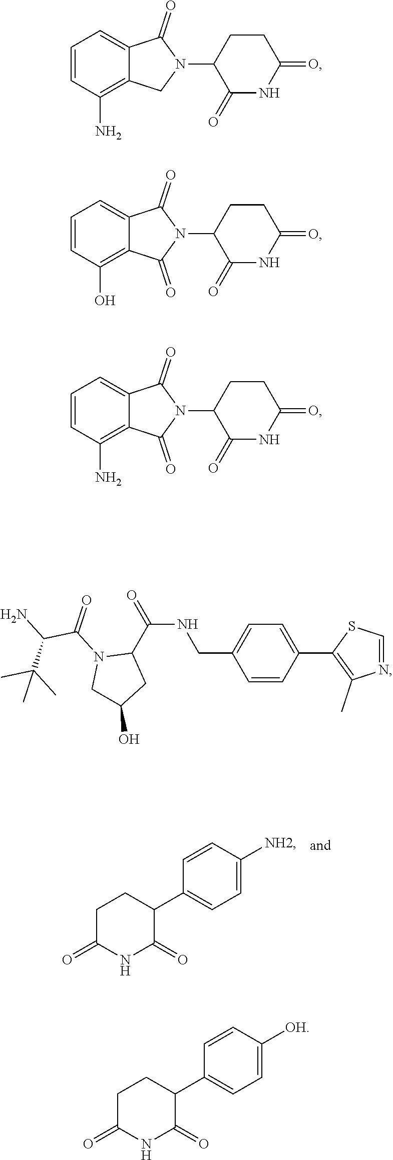 [SMILES: Nc1cccc2c1CN(C1CCC(=O)NC1=O)C2=O ; O=C1CCC(N2C(=O)c3cccc(O)c3C2=O)C(=O)N1 ; Nc1cccc2c1C(=O)N(C1CCC(=O)NC1=O)C2=O ; Cc1ncsc1-c1ccc(CNC(=O)C2C[C@@H](O)CN2C(=O)[C@@H](N)C(C)(C)C)cc1 ; Nc1ccc(C2CCC(=O)NC2=O)cc1 ; O=C1CCC(c2ccc(O)cc2)C(=O)N1]

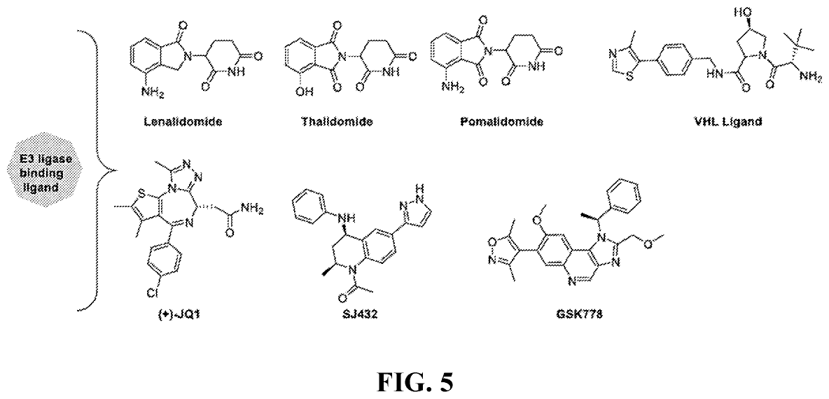

See also FIG. 5.

In various aspects, the E3 ligase binding ligand is selected from residues of:

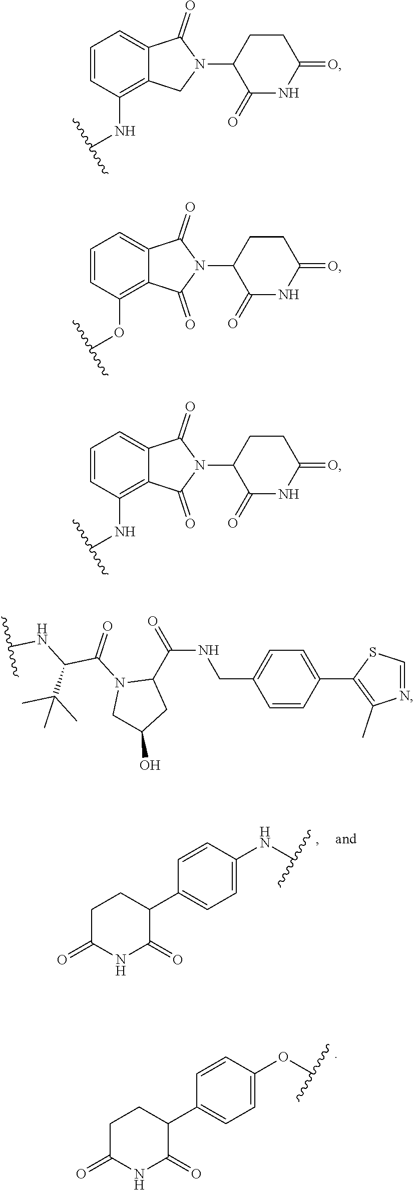 [SMILES: *Nc1cccc2c1CN(C1CCC(=O)NC1=O)C2=O ; *Oc1cccc2c1C(=O)N(C1CCC(=O)NC1=O)C2=O ; *Nc1cccc2c1C(=O)N(C1CCC(=O)NC1=O)C2=O ; *N[C@H](C(=O)N1C[C@H](O)CC1C(=O)NCc1ccc(-c2scnc2C)cc1)C(C)(C)C ; *Nc1ccc(C2CCC(=O)NC2=O)cc1 ; *Oc1ccc(C2CCC(=O)NC2=O)cc1]

In various aspects, the UPS targeting moiety is selected from residues of:

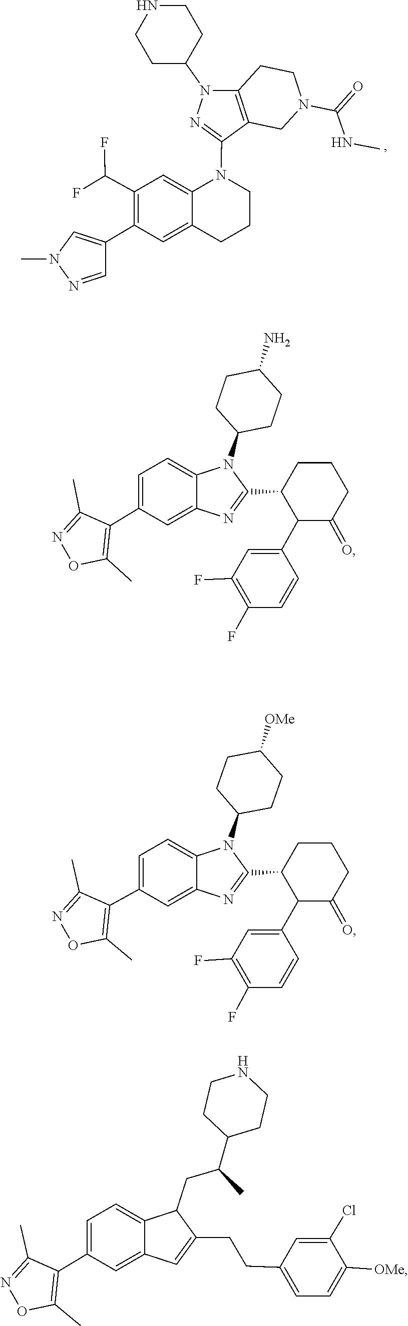

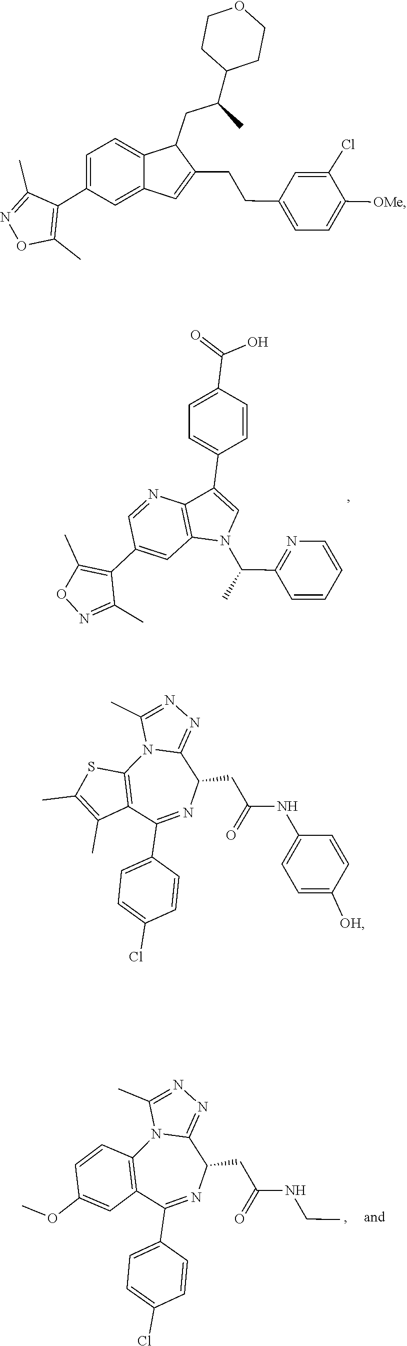 [SMILES: COc1ccc(CCC2=Cc3cc(-c4c(C)noc4C)ccc3C2C[C@H](C)C2CCOCC2)cc1Cl ; Cc1noc(C)c1-c1cnc2c(-c3ccc(C(=O)O)cc3)cn([C@@H](C)c3ccccn3)c2c1 ; Cc1sc2c(c1C)C(c1ccc(Cl)cc1)=N[C@@H](CC(=O)Nc1ccc(O)cc1)c1nnc(C)n1-2 ; CCNC(=O)C[C@@H]1N=C(c2ccc(Cl)cc2)c2cc(OC)ccc2-n2c(C)nnc21]

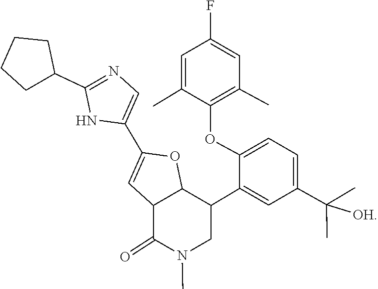 [SMILES: Cc1cc(F)cc(C)c1Oc1ccc(C(C)(C)O)cc1C1CN(C)C(=O)C2C=C(c3cnc(C4CCCC4)[nH]3)OC21]

c. Bromodomain Extraterminal Domain (BET) Binding Ligands (BRD Groups)

In various aspects, the disclosed sequence selective DNA binding compound comprises a bromodomain (BET) binding ligand. In a further aspect, the BET binding ligand is tethered to the polyamide moiety via a second chemical linker. In a still further aspect, the polyamide, the UPS targeting moiety, and the BET binding ligand are tethered via a first chemical linker and via a second chemical linker. In yet a further aspect, the polyamide, the transcription factor binding moiety, and the BET binding ligand are tethered via a first chemical linker and a second chemical linker.

In various aspects, the BET binding ligand is a residue of a structure selected from:

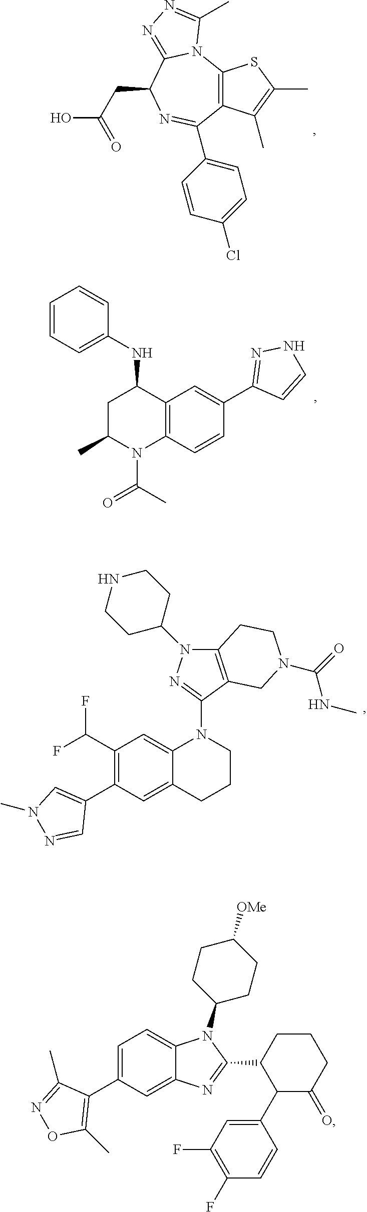

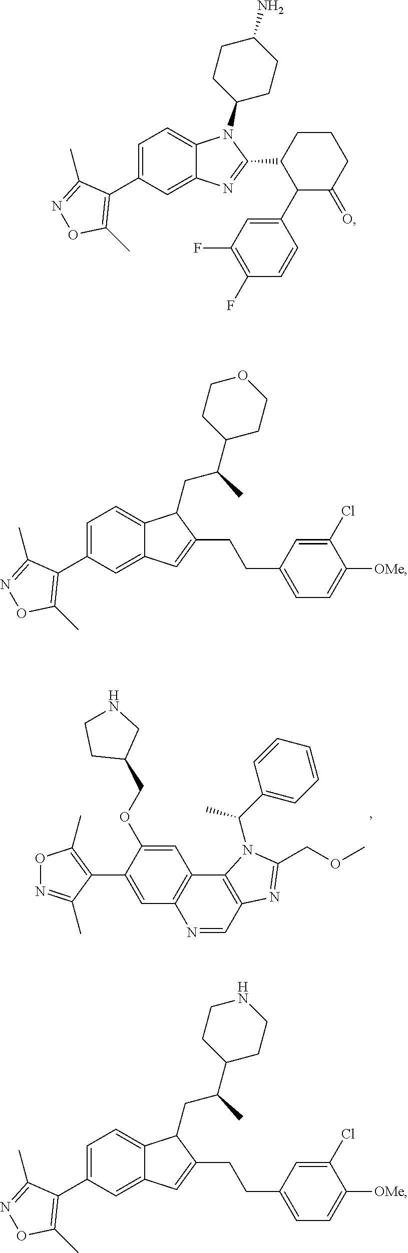

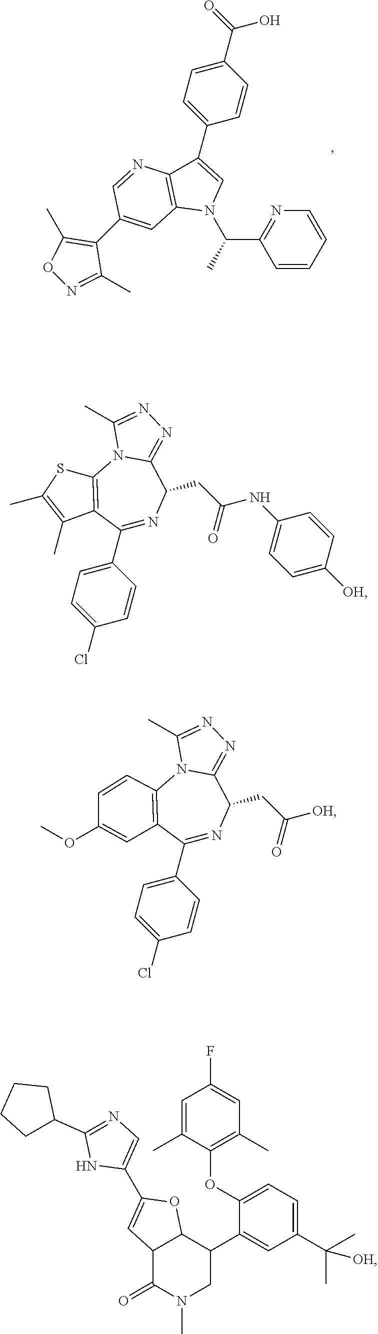 [SMILES: Cc1noc(C)c1-c1cnc2c(-c3ccc(C(=O)O)cc3)cn([C@@H](C)c3ccccn3)c2c1 ; Cc1sc2c(c1C)C(c1ccc(Cl)cc1)=N[C@@H](CC(=O)Nc1ccc(O)cc1)c1nnc(C)n1-2 ; COc1ccc2c(c1)C(c1ccc(Cl)cc1)=N[C@@H](CC(=O)O)c1nnc(C)n1-2 ; Cc1cc(F)cc(C)c1Oc1ccc(C(C)(C)O)cc1C1CN(C)C(=O)C2C=C(c3cnc(C4CCCC4)[nH]3)OC21]

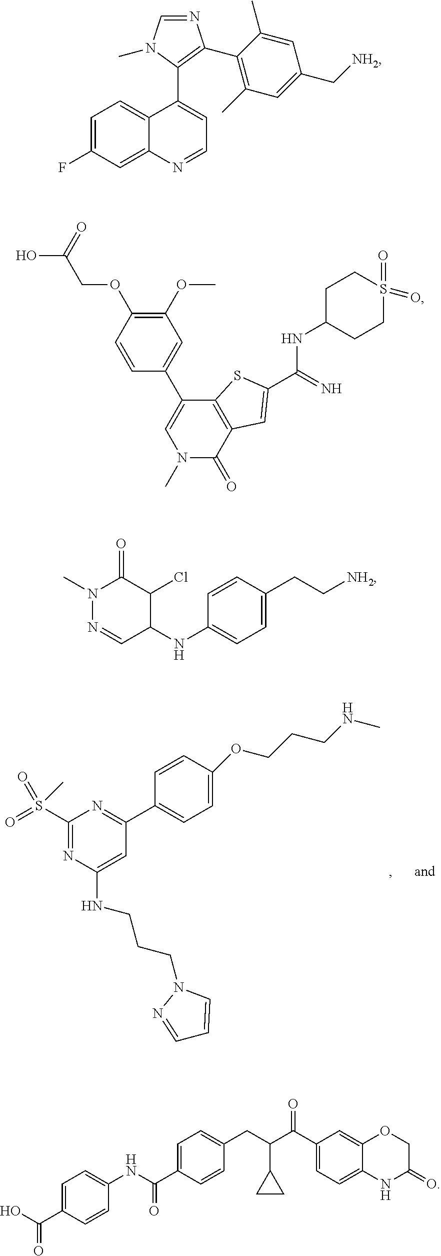 [SMILES: Cc1cc(CN)cc(C)c1-c1ncn(C)c1-c1ccnc2cc(F)ccc12 ; COc1cc(-c2cn(C)c(=O)c3cc(C(=N)NC4CCS(=O)(=O)CC4)sc23)ccc1OCC(=O)O ; CN1N=CC(Nc2ccc(CCN)cc2)C(Cl)C1=O ; CNCCCOc1ccc(-c2cc(NCCCn3cccn3)nc(S(C)(=O)=O)n2)cc1 ; O=C1COc2cc(C(=O)C(Cc3ccc(C(=O)Nc4ccc(C(=O)O)cc4)cc3)C3CC3)ccc2N1]

d. PBX or TCF3-PBX Transcription Factor Binding Moieties

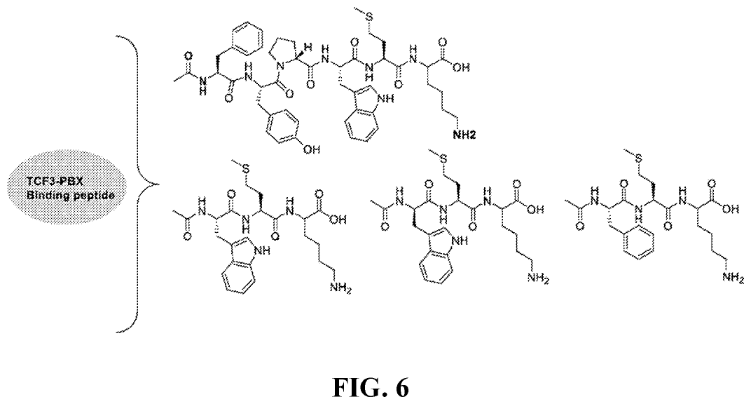

In various aspects, the disclosed sequence selective DNA binding compound comprises a PBX or TCF3-PBX transcription factor binding moiety. In a further aspect, the PBX or TCF3-PBX transcription factor binding moiety is tethered to the polyamide moiety via a first chemical linker. In a still further aspect, the polyamide, the UPS targeting moiety, and the PBX or TCF3-PBX transcription factor binding moiety are tethered via a first chemical linker and via a second chemical linker. In yet a further aspect, the polyamide, the transcription factor binding moiety, and the PBX or TCF3-PBX transcription factor binding moiety are tethered via a first chemical linker and a second chemical linker. In an even further aspect, the polyamide moiety and the transcription factor binding moiety are tethered via the second chemical linker. In a still further aspect, the polyamide moiety and the transcription factor binding moiety are tethered via the first chemical linker. Exemplary TCF3-PBX transcription factor binding moieties are shown in FIG. 6.

In various aspects, the transcription factor binding moiety is selected from:

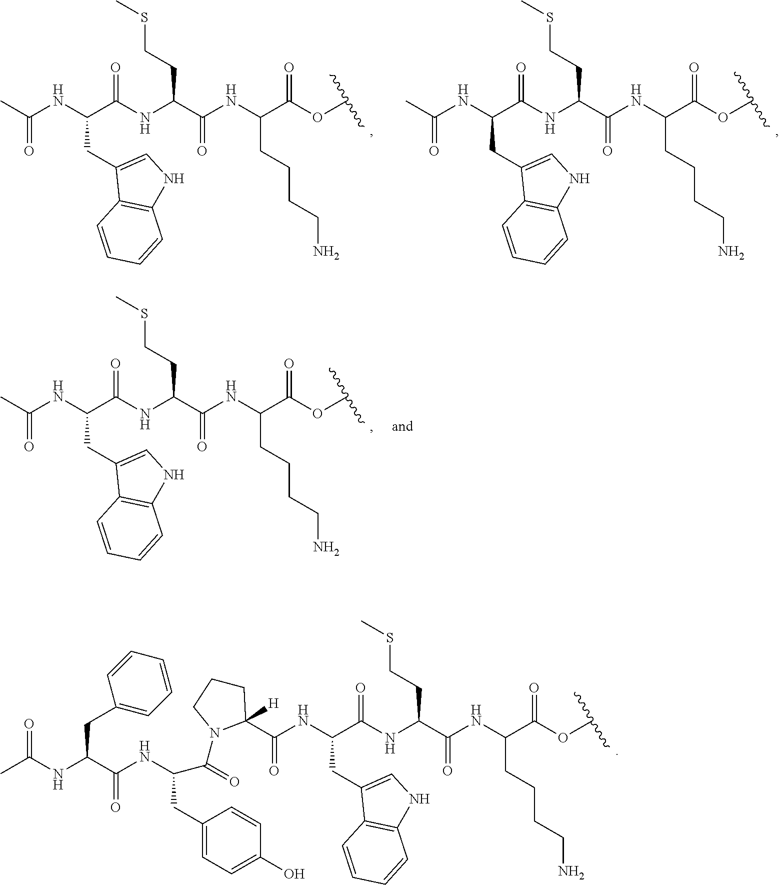

In various aspects, the transcription factor binding moiety is a residue of a structure selected from:

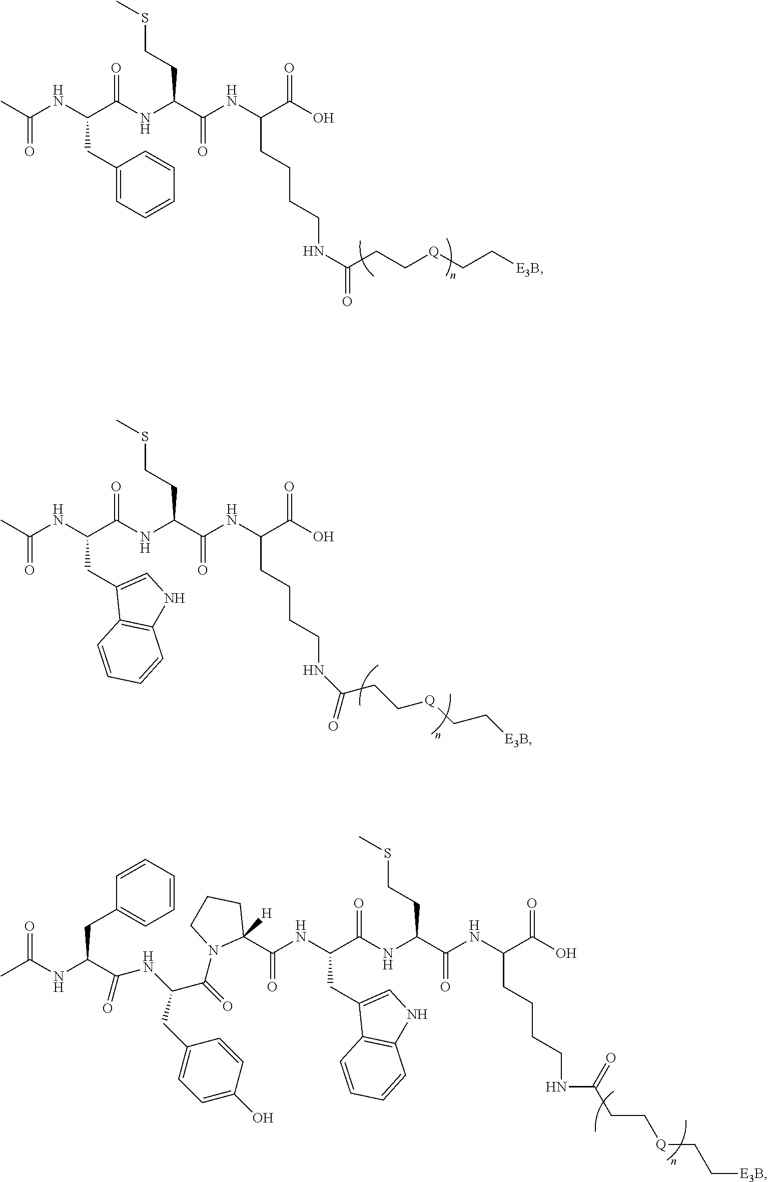

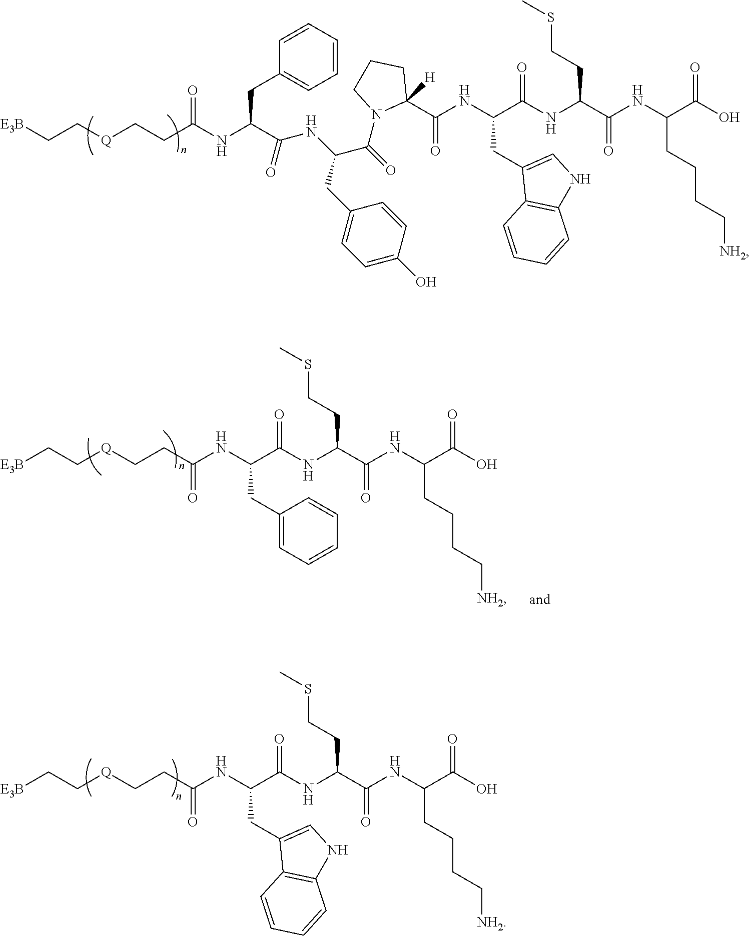

e. Chemical Linkers

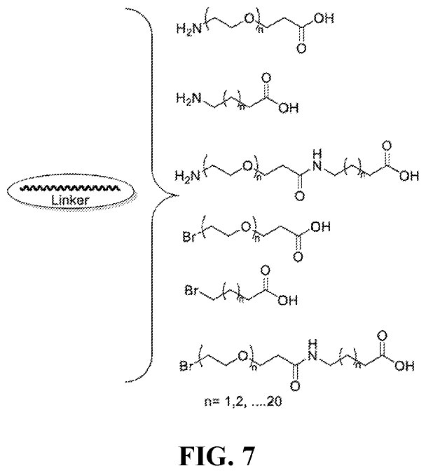

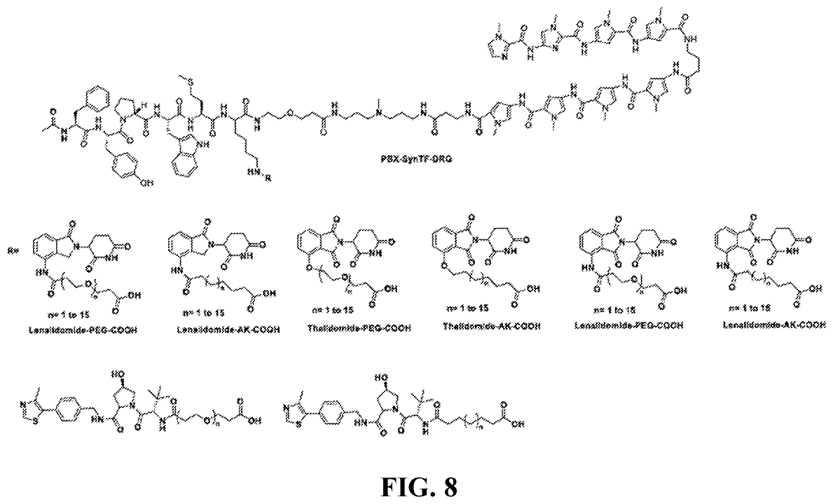

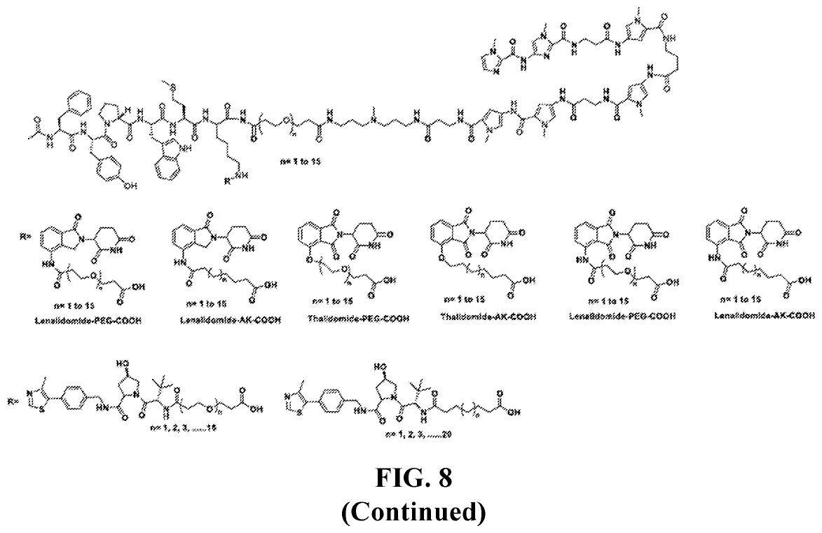

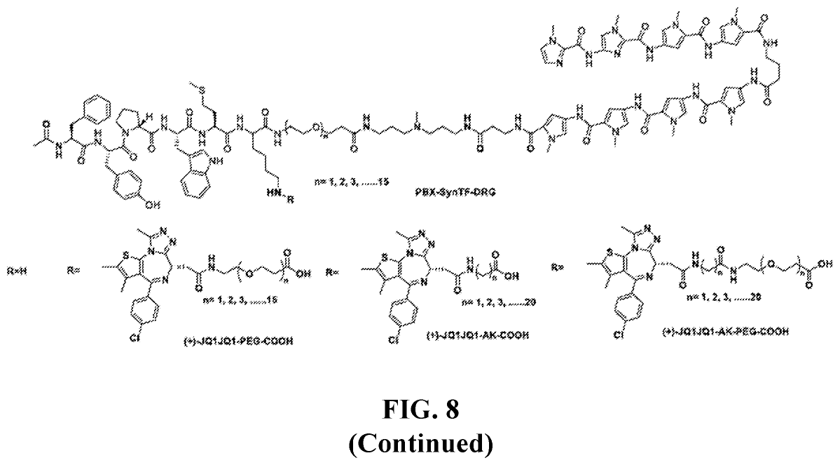

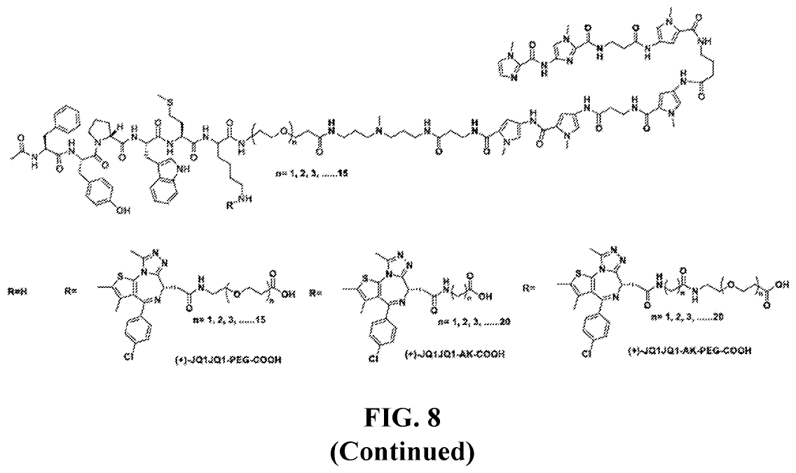

In one aspect, the disclosed sequence selective DNA binding compound comprises a first chemical linker. In a further aspect, the the disclosed sequence selective DNA binding compound also comprises a second chemical linker. In this way, the various components of the compound (e.g., the polyamide moiety, the UPS targeting moiety, the BET binding ligand, the PBX or TCF3-PBX transcription factor binding moiety) can be tethered together. Exemplary chemical linkers are shown in FIG. 7.

Thus, in various aspects, the compound comprises the first chemical linker. In a further aspect, the compound comprises the second chemical linker. In a still further aspect, the compound comprises the first and the second chemical linker.

In various aspects, the first and the second chemical linker are the same. In a further aspect, the first and the second chemical linker are different.

In various aspects, the first chemical linker is a polyethylene glycol (PEG) linker having a structure represented by a formula selected from:

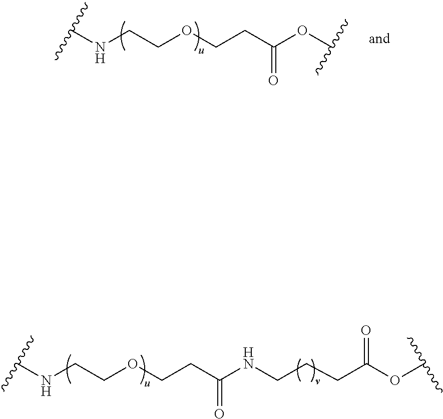

wherein each of u and v is independently an integer selected from 1 to 20.

In various aspects, the first chemical linker is an alkyl linker having a structure represented by a formula:

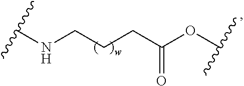

wherein w is an integer selected from 1 to 20.

In various aspects, the first chemical linker is selected from residues of:

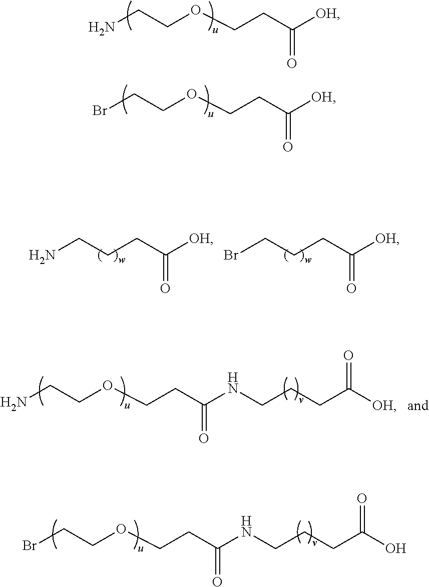

wherein each of u, v, and w is independently an integer selected from 1 to 20.

In various aspects, the first chemical linker is the only chemical linker in the compound.

In various aspects, the second chemical linker is a polyethylene glycol (PEG) linker having a structure represented by a formula selected from:

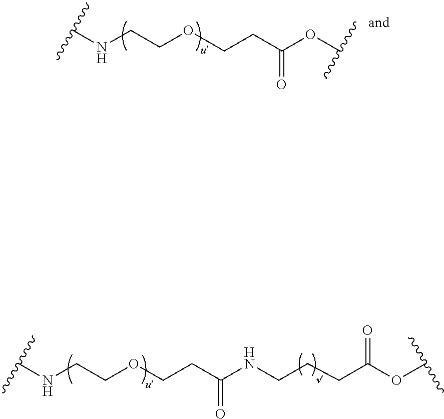

wherein each of u′ and v′ is independently an integer selected from 1 to 20.

In various aspects, the second chemical linker is an alkyl linker having a structure represented by a formula:

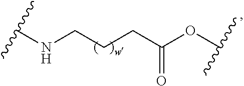

wherein w′ is an integer selected from 1 to 20.

In various aspects, the second chemical linker is selected from residues of:

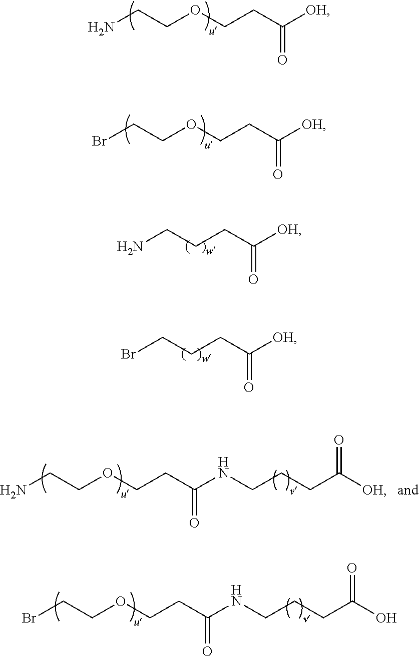

wherein each of u′, v′, and w′ is independently an integer selected from 1 to 20.

In one aspect, each of u and v is independently an integer selected from 1 to 20. In a further aspect, each of u and v is independently an integer selected from 1 to 15. In a still further aspect, each of u and v is independently an integer selected from 1 to 10. In yet a further aspect, each of u and v is independently an integer selected from 1 to 5. In an even further aspect, each of u and v is independently an integer selected from 5 to 20. In a still further aspect, each of u and v is independently an integer selected from 10 to 20. In yet a further aspect, each of u and v is independently an integer selected from 15 to 20. In an even further aspect, each of u and v is independently an integer selected from 5 to 15.

In one aspect, w is an integer selected from 1 to 20. In a further aspect, w is an integer selected from 1 to 15. In a still further aspect, w is an integer selected from 1 to 10. In yet a further aspect, w is an integer selected from 1 to 5. In an even further aspect, w is an integer selected from 5 to 20. In a still further aspect, w is an integer selected from 10 to 20. In yet a further aspect, w is an integer selected from 15 to 20. In an even further aspect, w is an integer selected from 5 to 15.

In one aspect, each of u, v, and w is independently an integer selected from 1 to 20. In a further aspect, each of u, v, and w is independently an integer selected from 1 to 15. In a still further aspect, each of u, v, and w is independently an integer selected from 1 to 10. In yet a further aspect, each of u, v, and w is independently an integer selected from 1 to 5. In an even further aspect, each of u, v, and w is independently an integer selected from 5 to 20. In a still further aspect, each of u, v, and w is independently an integer selected from 10 to 20. In yet a further aspect, each of u, v, and w is independently an integer selected from 15 to 20. In an even further aspect, each of u, v, and w is independently an integer selected from 5 to 15.

f. Q Groups

In one aspect, each occurrence of Q is independently selected from CH₂ and O. In a further aspect, Q is CH₂. In a still further aspect, Q is O.

g. X′ Groups

In one aspect, each occurrence of X′, when present, is independently selected from NH or O. In a further aspect, X′ is NH. In a still further aspect, X′ is O.

h. Z Groups

In one aspect, Z is selected from O and NH. In a further aspect, Z is O. In a still further aspect, Z is NH.

i. R² Groups

In one aspect, R² is selected from CH₂ and C(O). In a further aspect, R² is CH₂. In a further aspect, R² is C(O).

### 2. Example Compounds

In one aspect, a compound can be present as one or more of the following structures:

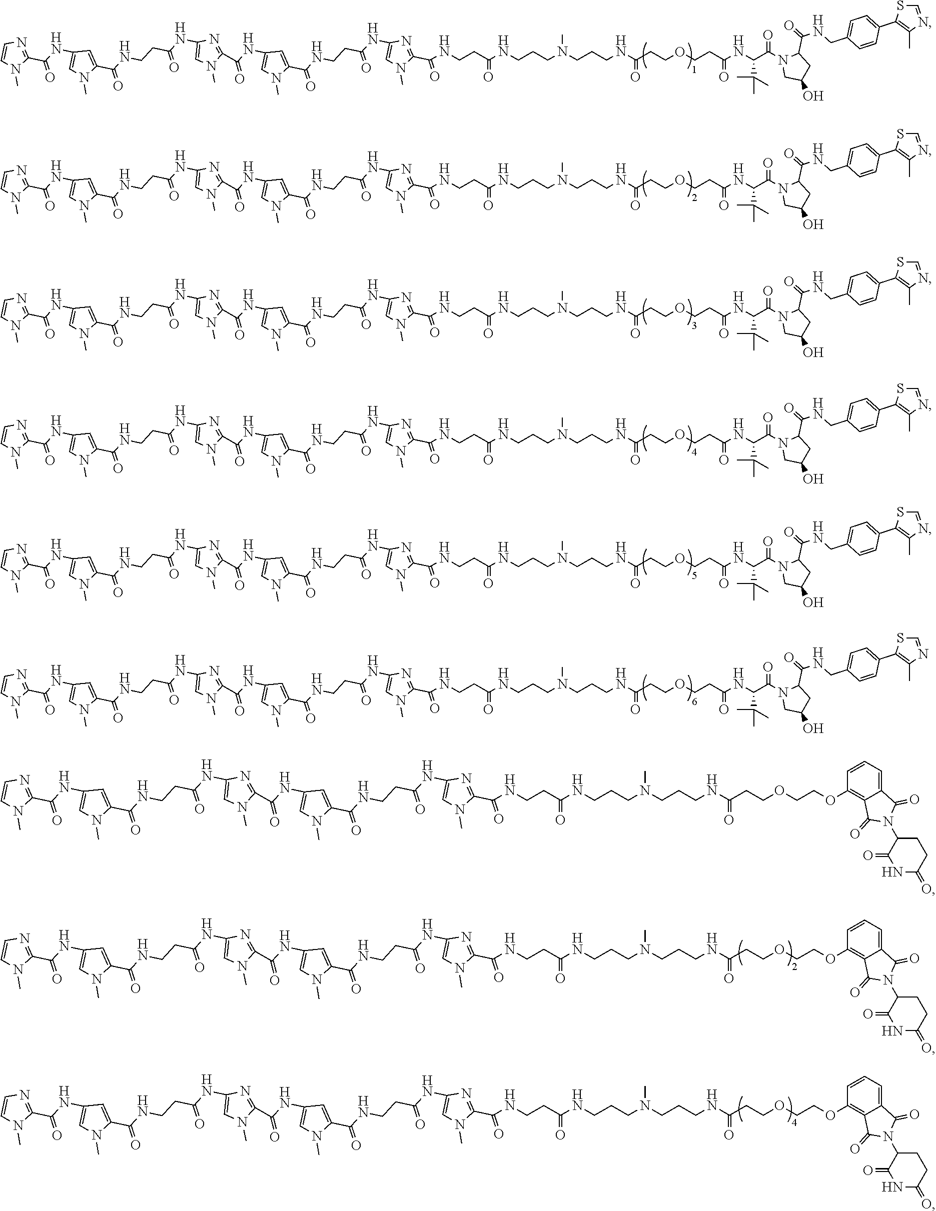

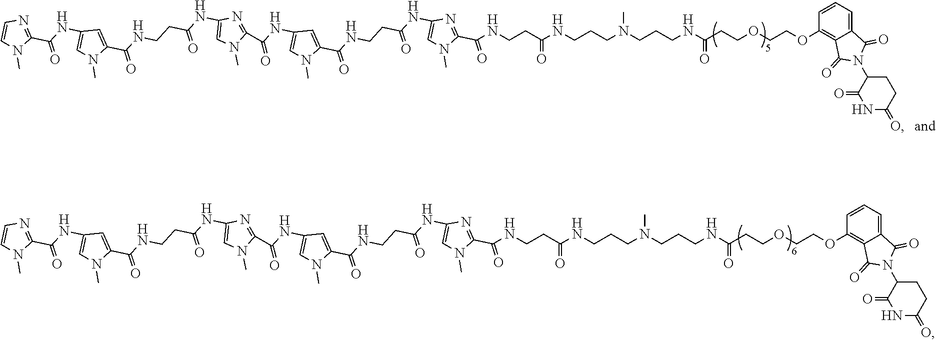

or a pharmaceutically acceptable salt thereof.

In one aspect, a compound can be present as one or more of the following structures:

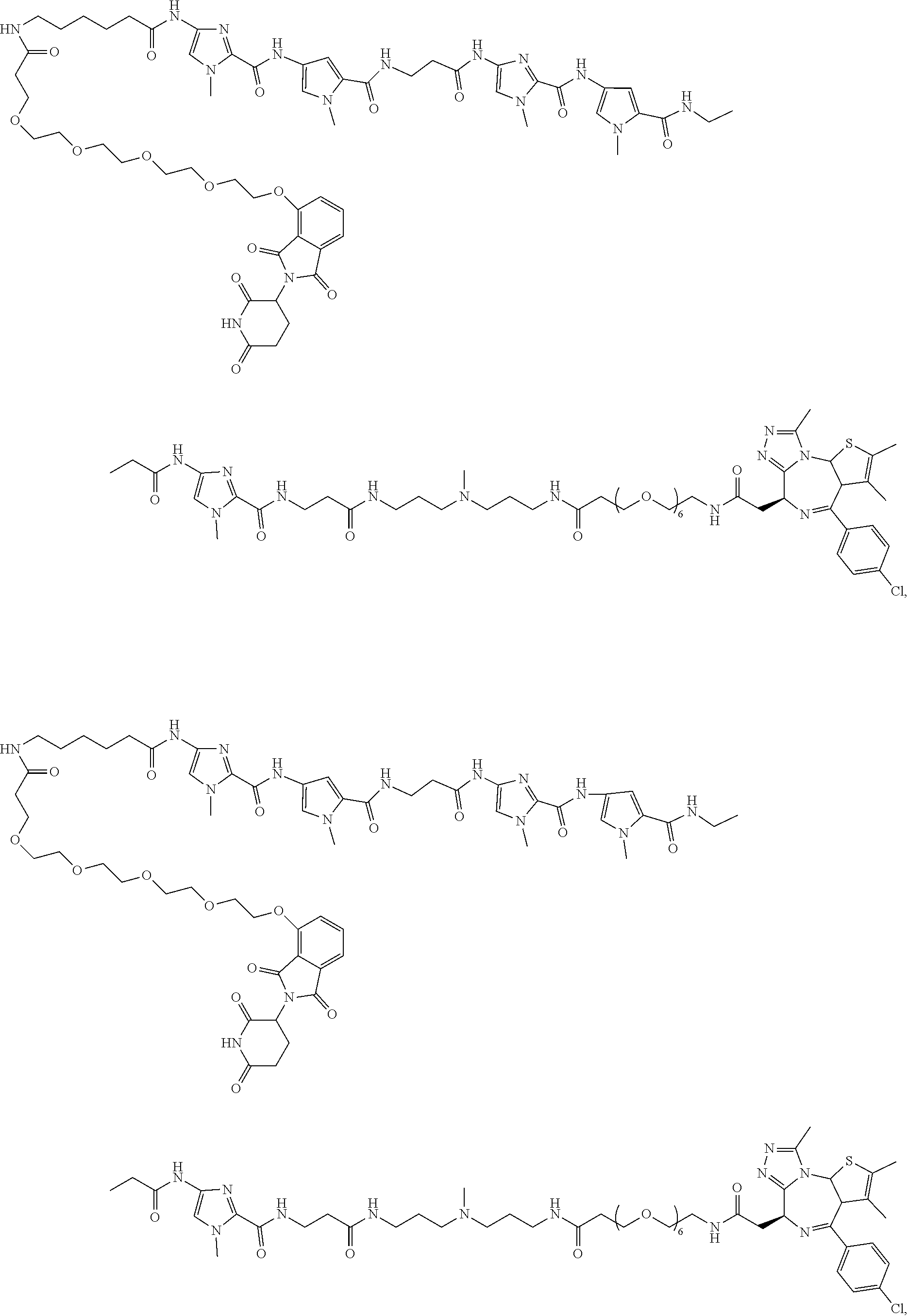

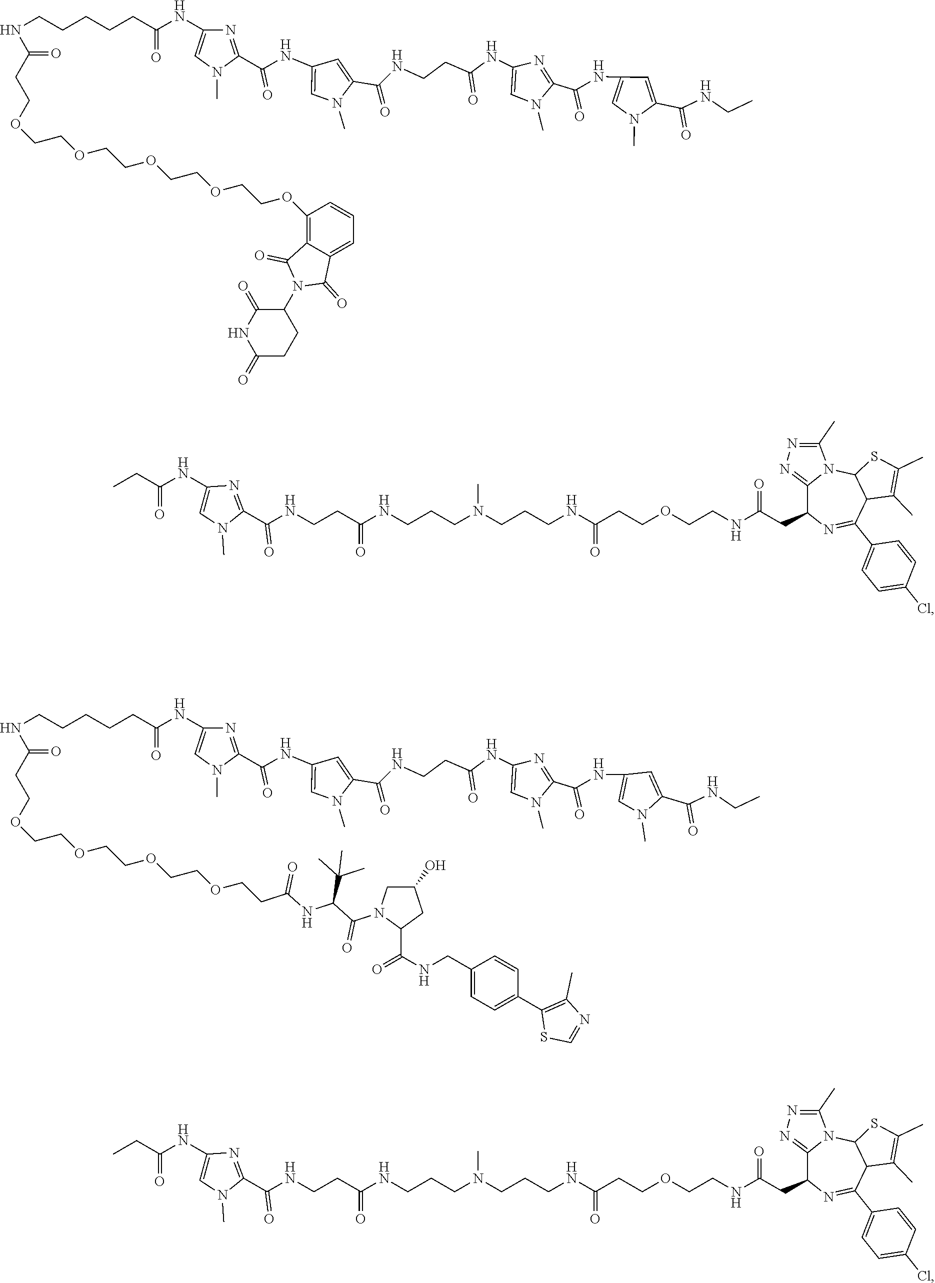

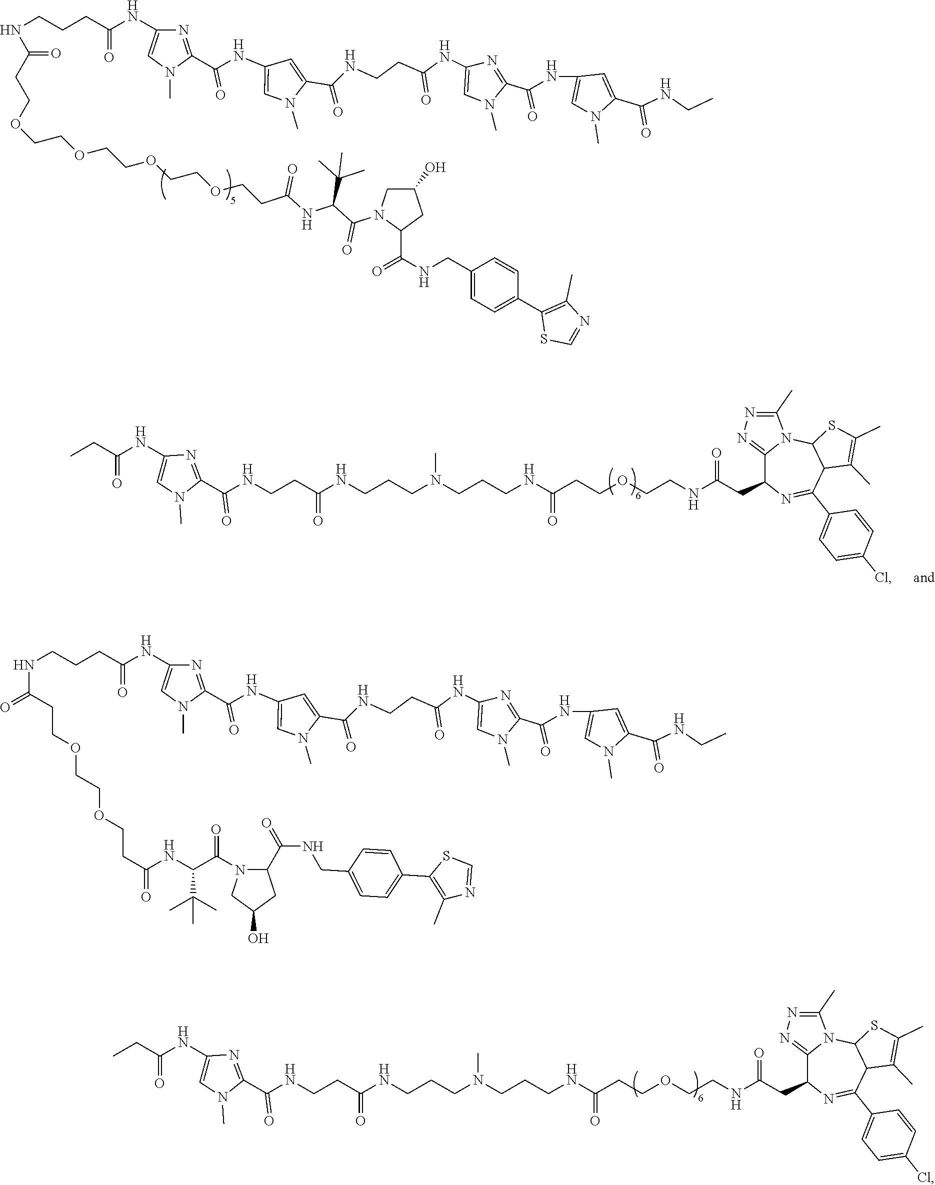

or a pharmaceutically acceptable salt thereof.

In one aspect, a compound can be present as one or more of the following structures:

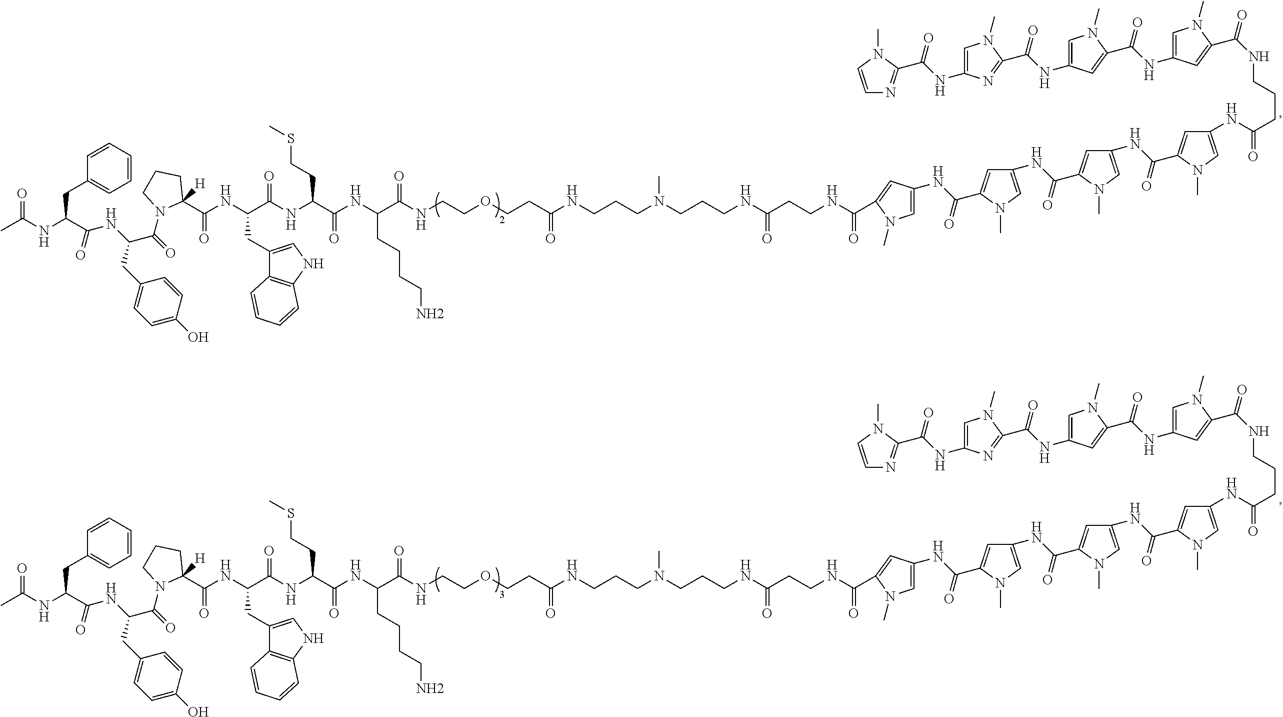

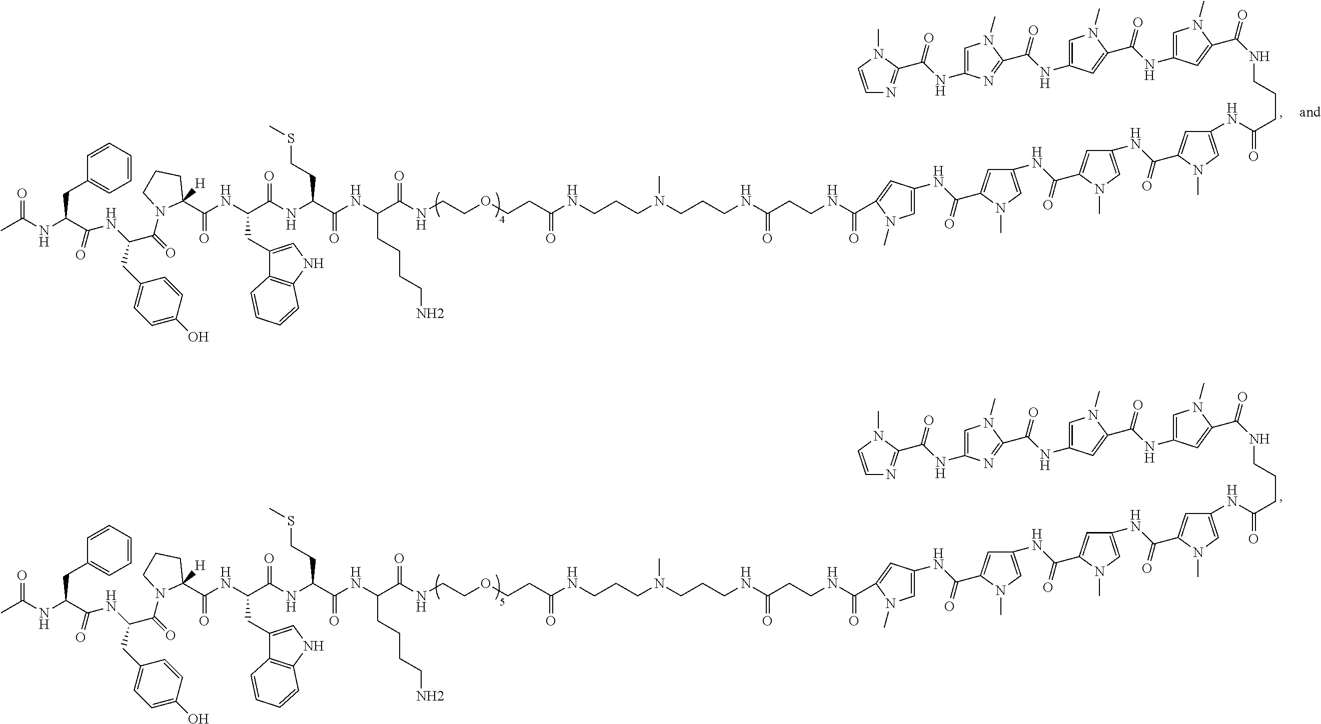

or a pharmaceutically acceptable salt thereof.

In one aspect, a compound can be present as one or more of the following structures:

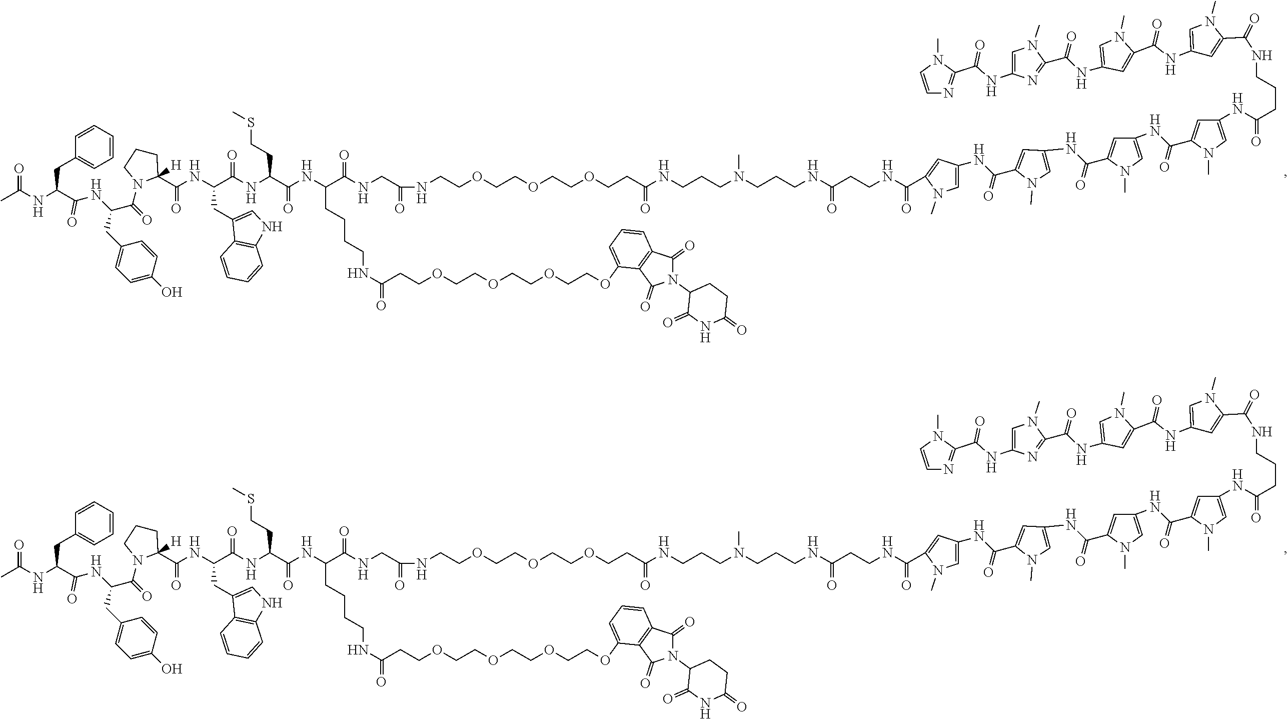

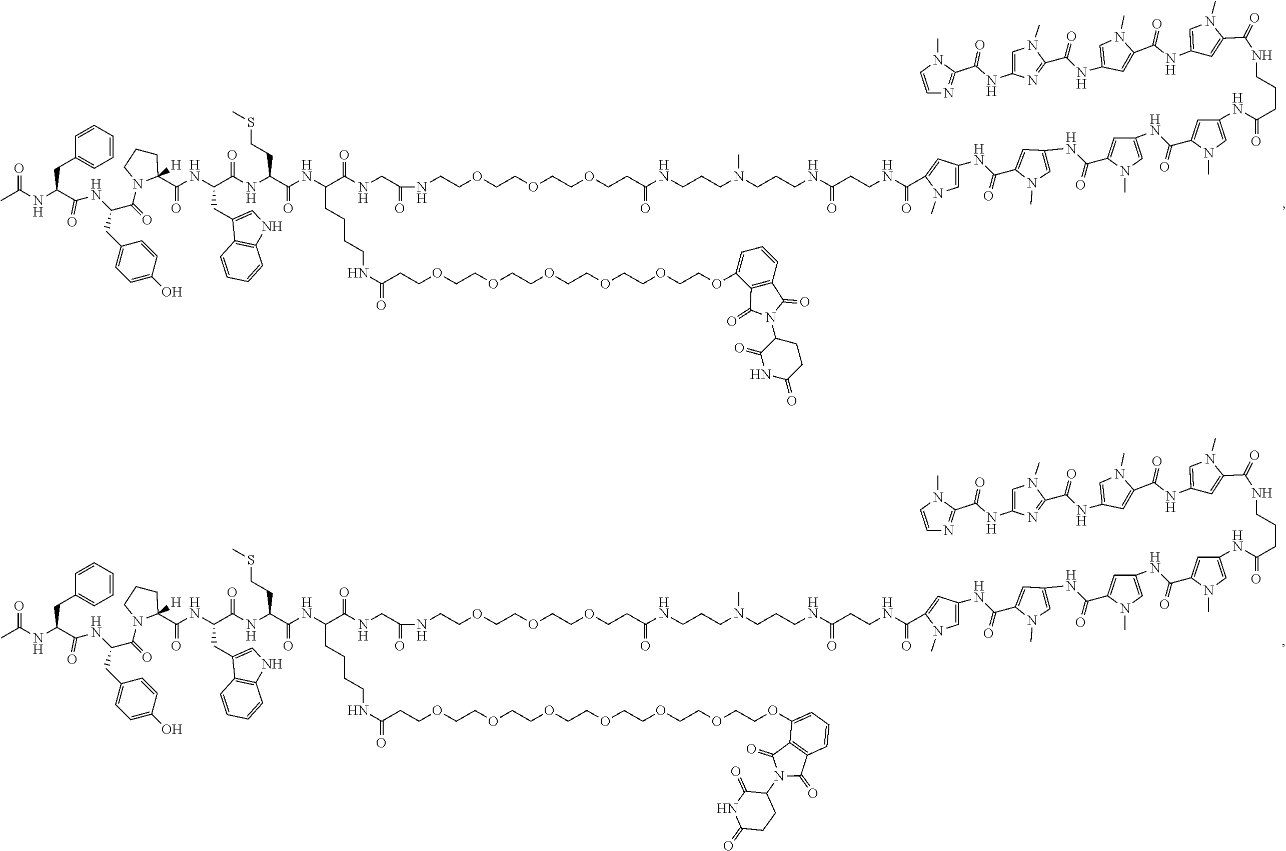

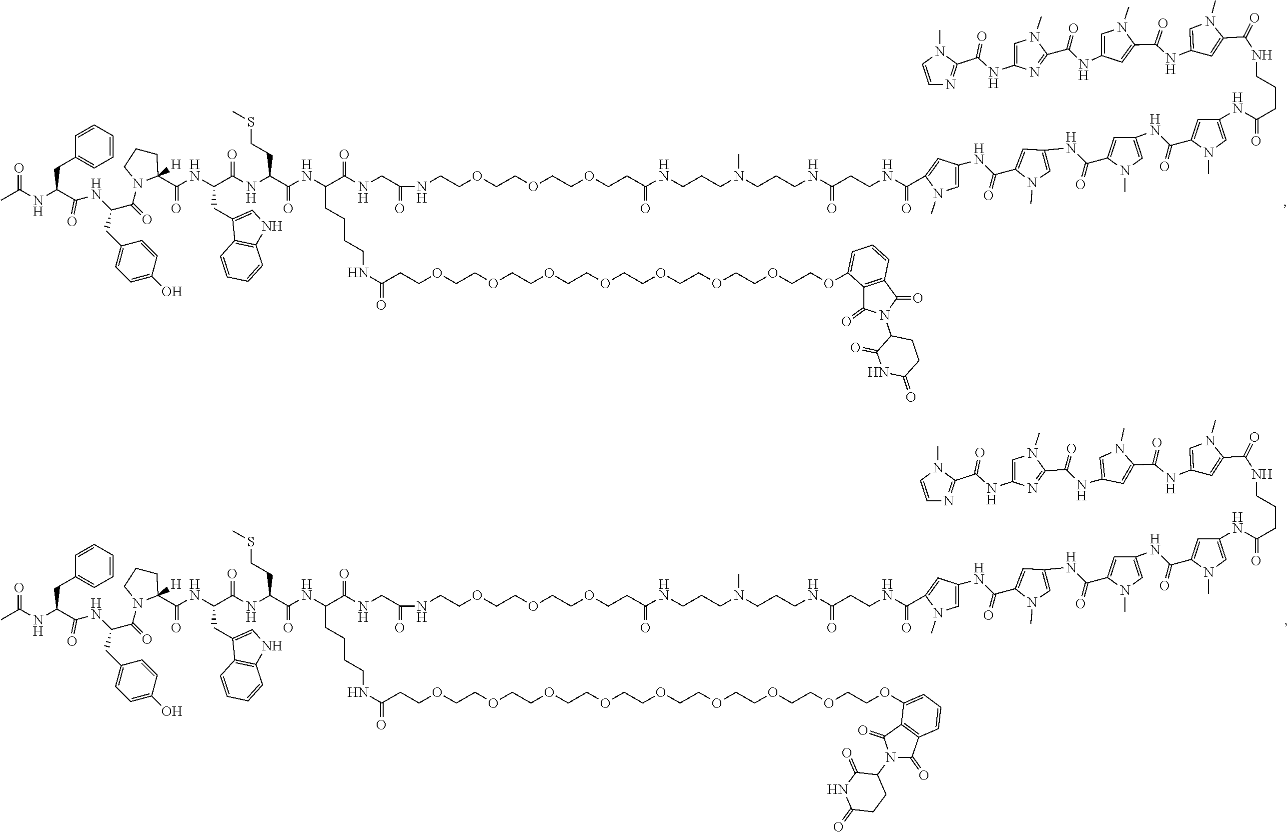

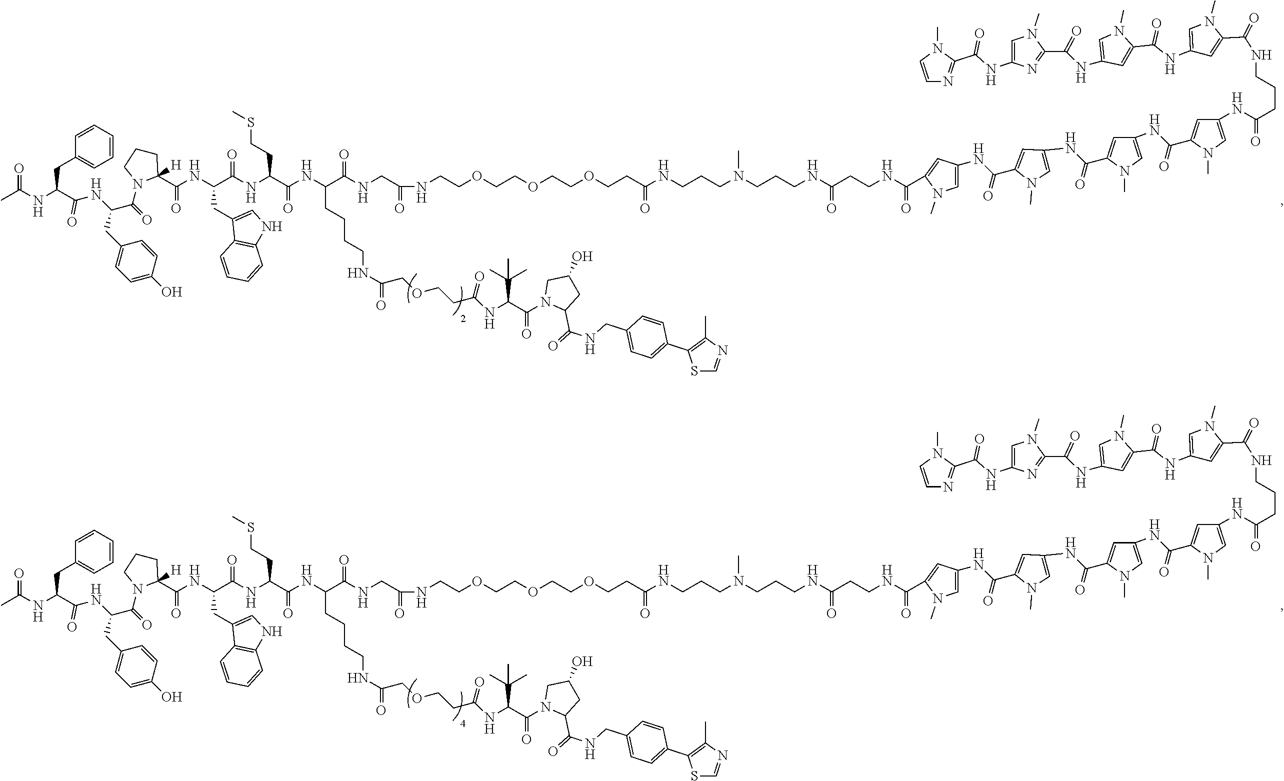

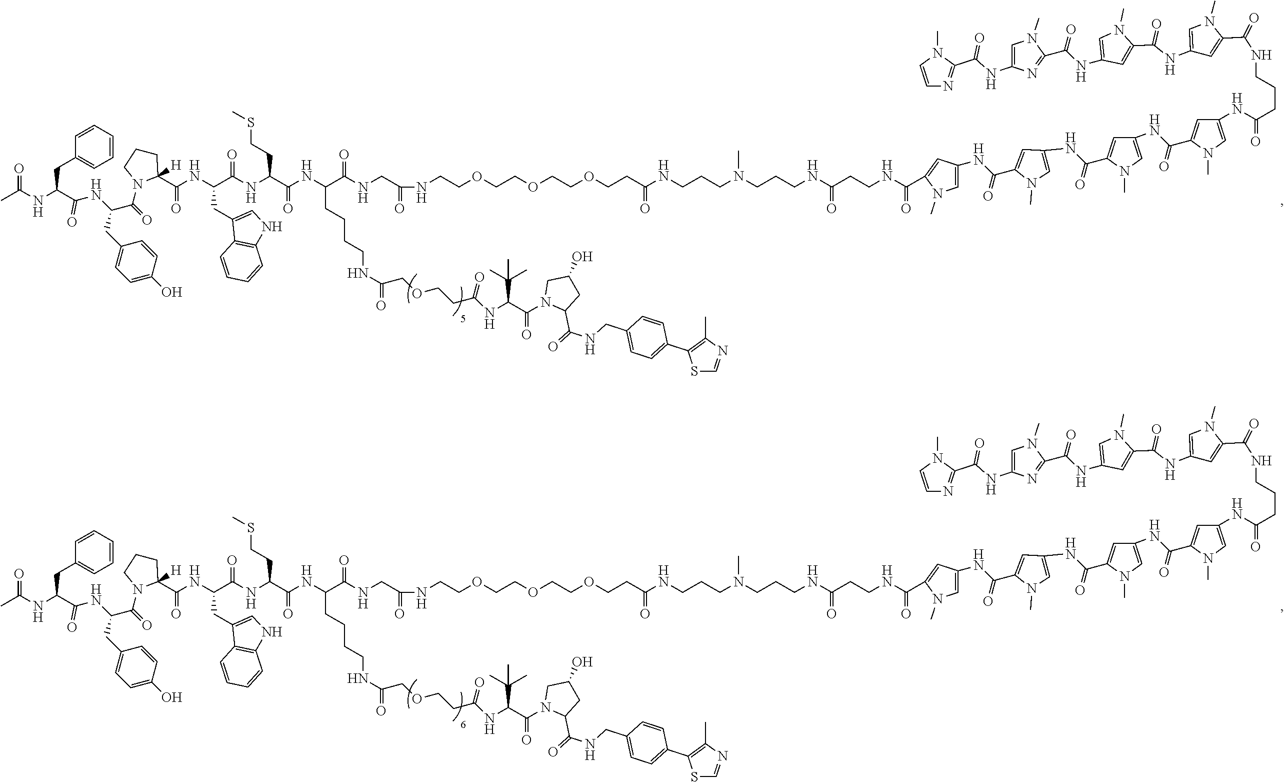

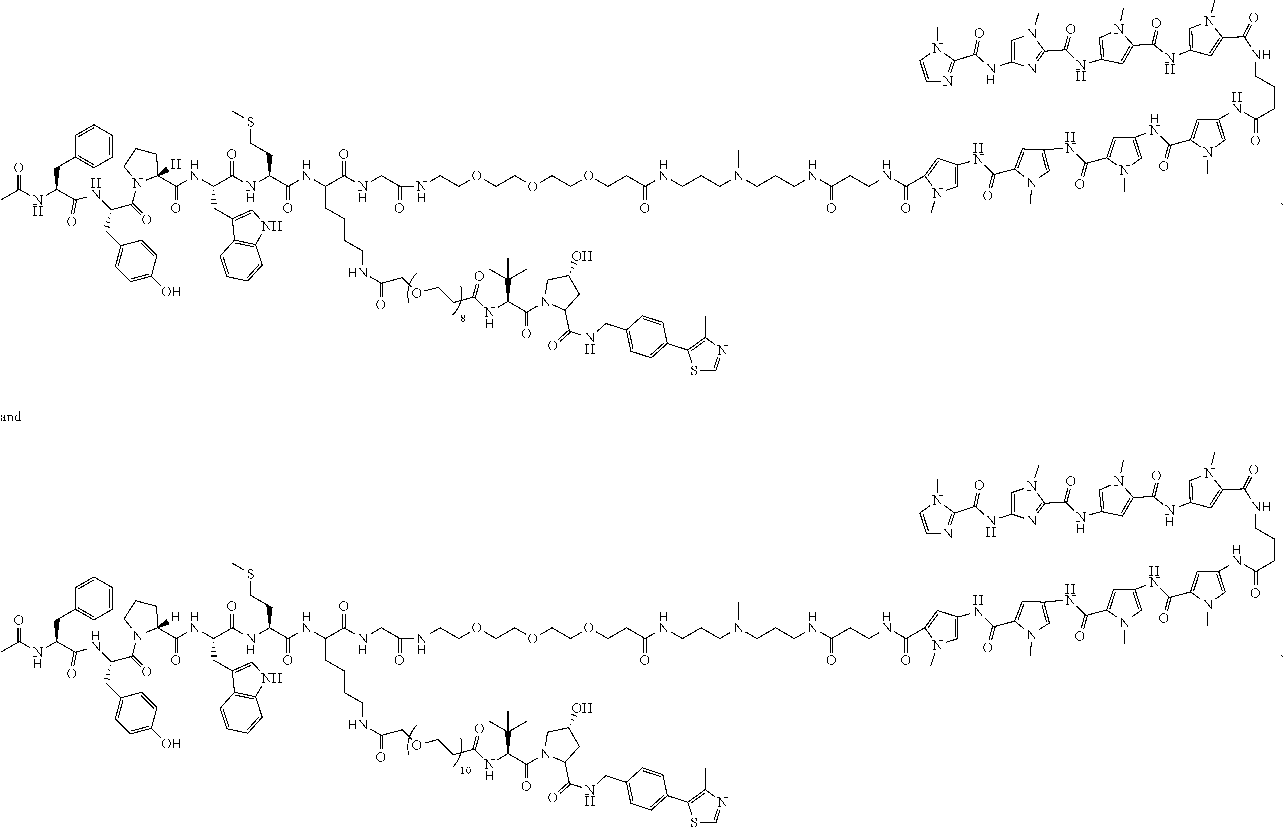

or a pharmaceutically acceptable salt thereof.

### C. METHODS OF MAKING A COMPOUND

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Routes I-VIII, as described and exemplified below. The following examples are provided so that the invention might be more fully understood, are illustrative only, and should not be construed as limiting.

### 1. Route 1

In one aspect, the disclosed polyamides (e.g., linear, U-pin, H-pin) can be prepared as shown below.

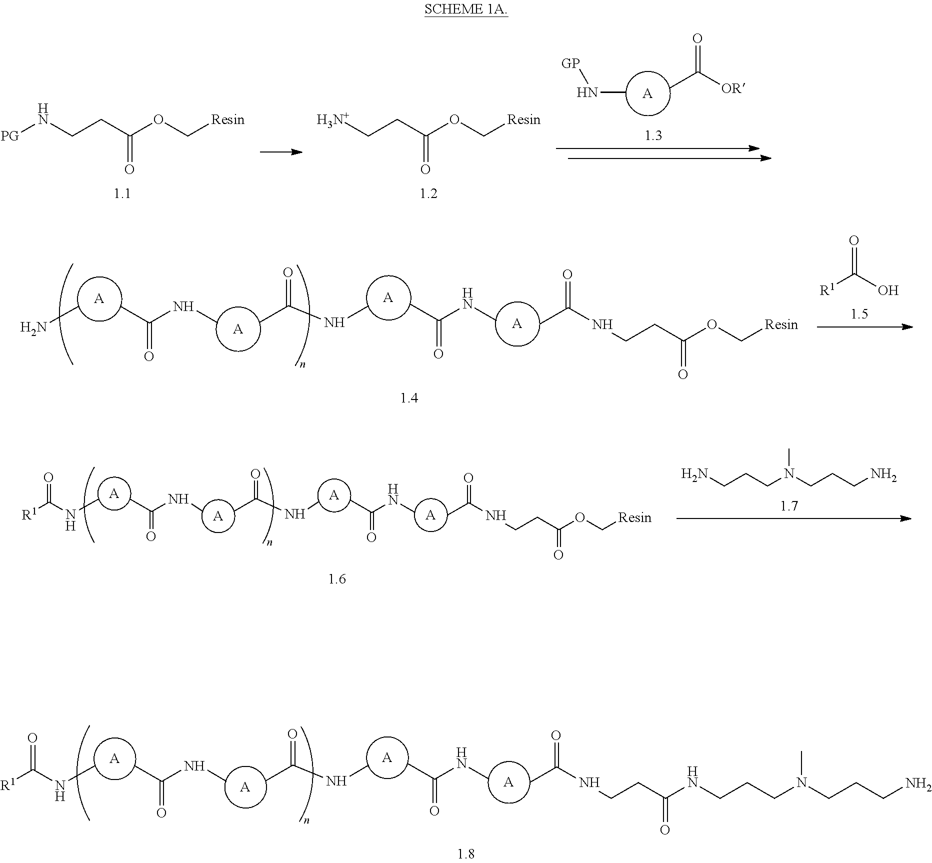

Compounds are represented in generic form, wherein each occurrence of PG is independently an amine protecting group (e.g., carbobenzyloxy, p-methoxybenzyl carbonyl, t-butyloxycarbonyl, 9-fluorenylmethyloxycarbonyl, acetyl, benzoyl, benzyl, carbamate, p-methoxybenzyl, 3,4-dimethoxybenzyl, p-methoxyphenyl, tosyl, 4-nitrobenzenesulfonyl), R′ is H, benzotriazole (Bt), or any other activated moiety, and with other substituents as noted in compound descriptions elsewhere herein. A specific non-limiting example of the synthesis shown in Scheme 1A is provided below.

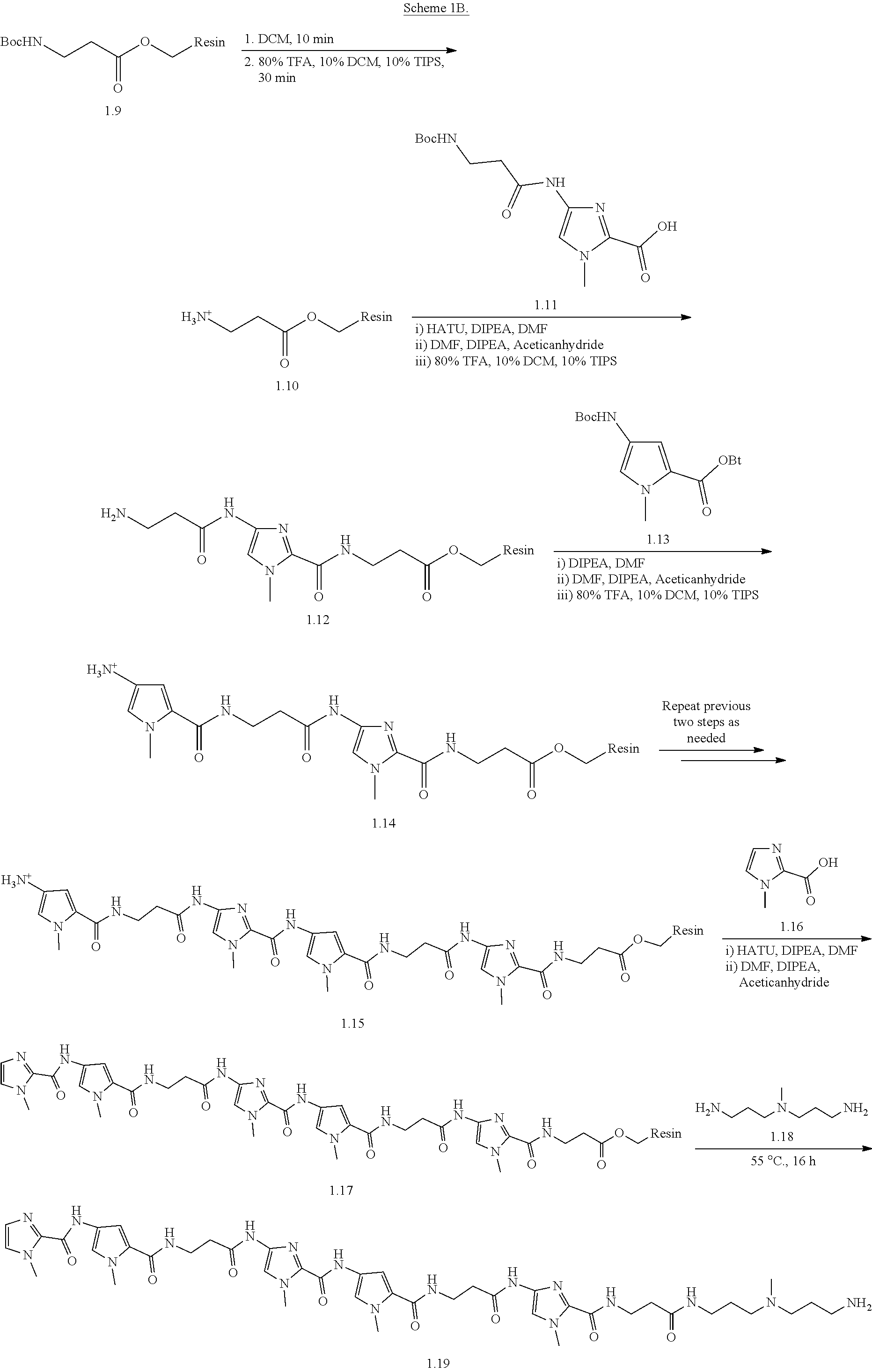

In one aspect, compounds of type 1.19, and similar compounds, can be prepared according to reaction Scheme 1B above. Thus, compounds of type 1.10 can be prepared by deprotection of an appropriate resin having an protected amine, e.g., 1.9 as shown above. The deprotection is carried out by suspending the resin in an appropriate solvent, e.g., dichloromethane (DCM), for an appropriate period of time, e.g., 10 minutes, followed by addition of an appropriate deprotecting agent, e.g., 80% triflouroacetic acid (TFA), an appropriate reducing agent, e.g., 10% triisopropyl silane (TIPS), in an appropriate solvent, e.g., 10% DCM, for an appropriate period of time, e.g., 30 minutes. Compounds of type 1.12 can be prepared by a coupling reaction between an appropriate amine, e.g., 1.10 as shown above, and an appropriate carboxylic acid, e.g., 1.11 as shown above. The coupling reaction can be carried out in the presence of an appropriate coupling agent, e.g., hexafluorophosphate azabenzotriazole tetramethyl uronoim (HATU), and an appropriate base, e.g., N,N-diisopropylethylamine (DIPEA), in an appropriate solvent, e.g., dimethylformamide (DMF), followed by suspension in an appropriate solvent, e.g., DMF, in the presence of an appropriate base, e.g., DIPEA, and capping with an appropriate acetate, e.g., acetic anhydride as shown above. The protecting group is then removed using deprotection conditions such as, for example, those detailed above for Step 1. Compounds of type 1.14 can be prepared by a coupling reaction between an appropriate amine, e.g., 1.12 as shown above, and an appropriate carboxylic acid, e.g., 1.13 as shown above. The coupling reaction can be carried out in the presence of an appropriate base, e.g., DIPEA, in an appropriate solvent, e.g., DMF, followed by suspension in an appropriate solvent, e.g., DMF, in the presence of an appropriate base, e.g., DIPEA, and capping with an appropriate acetate, e.g., acetic anhydride or aceticanhydride as shown above. The protecting group is then removed using deprotection conditions such as, for example, those detailed above for Step 1. Steps 2 and 3 can be repeated as needed such that the desired n groups are included in the peptide, e.g., peptide 1.15 as shown above. Compounds of type 1.17 can be prepared by a coupling reaction between an appropriate amine, e.g., 1.15 as shown above, and an appropriate carboxylic acid, e.g., 1.16 as shown above. The coupling reaction can be carried out in the presence of an appropriate coupling agent, e.g., HATU, and an appropriate base, e.g., N,N-DIPEA, in an appropriate solvent, e.g., DMF, followed by suspension in an appropriate solvent, e.g., DMF, in the presence of an appropriate base, e.g., DIPEA, and capping with an appropriate acetate, e.g., acetic anhydride or aceticanhydride as shown above. Compounds of type 1.19 can be prepared by cleavage of the resin using an appropriate amine, e.g., 1.18 as shown above. The cleavage can be achieved at an appropriate temperature, e.g., 55° C., for an appropriate period of time, e.g., 16 hours. As can be appreciated by one skilled in the art the above reaction provides an example of a generalized approach wherein compounds similar in structure to the specific reactions above (compounds similar to 1.1, 1.2, 1.3, 1.4, 1.5, 1.6, and 1.7) can be substituted in the reaction to provide polyamides similar to Formula 1.8.

### 2. Route 2

In one aspect, the disclosed polyamides (e.g., tandem hairpin, hairpin, cyclic) can be prepared as shown below.

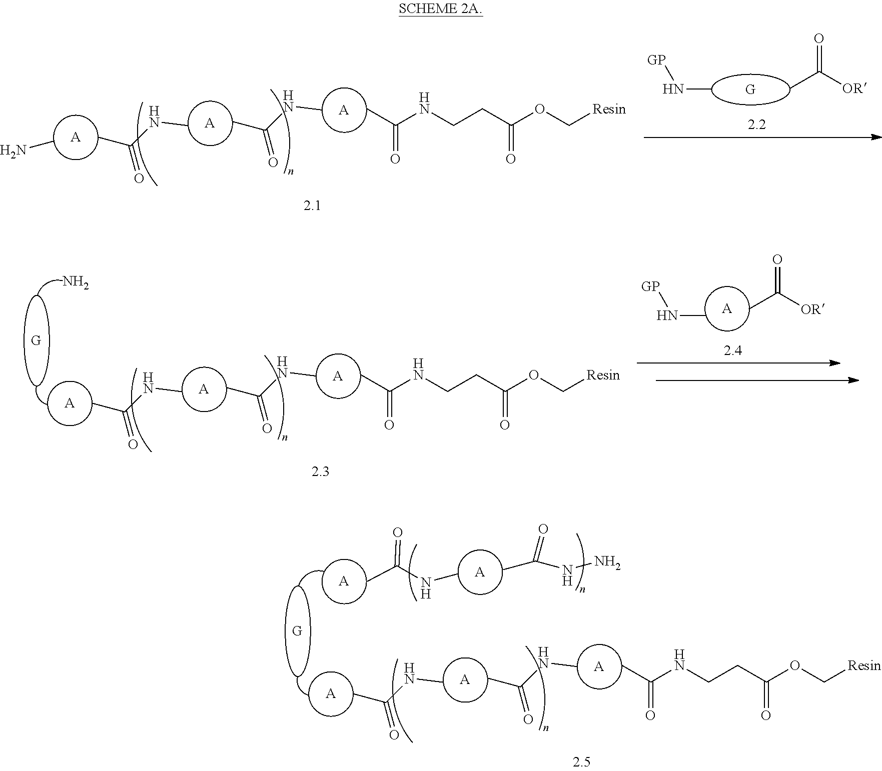

Compounds are represented in generic form, wherein each occurrence of PG independently is an amine protecting group (e.g., carbobenzyloxy, p-methoxybenzyl carbonyl, t-butyloxycarbonyl, 9-fluorenylmethyloxycarbonyl, acetyl, benzoyl, benzyl, carbamate, p-methoxy benzyl, 3,4-dimethoxybenzyl, p-methoxyphenyl, tosyl, 4-nitrobenzenesulfonyl), R′ is H, benzotriazole (Bt), and with other substituents as noted in compound descriptions elsewhere herein. A specific non-limiting example of the synthesis shown in Scheme 2A is provided below.

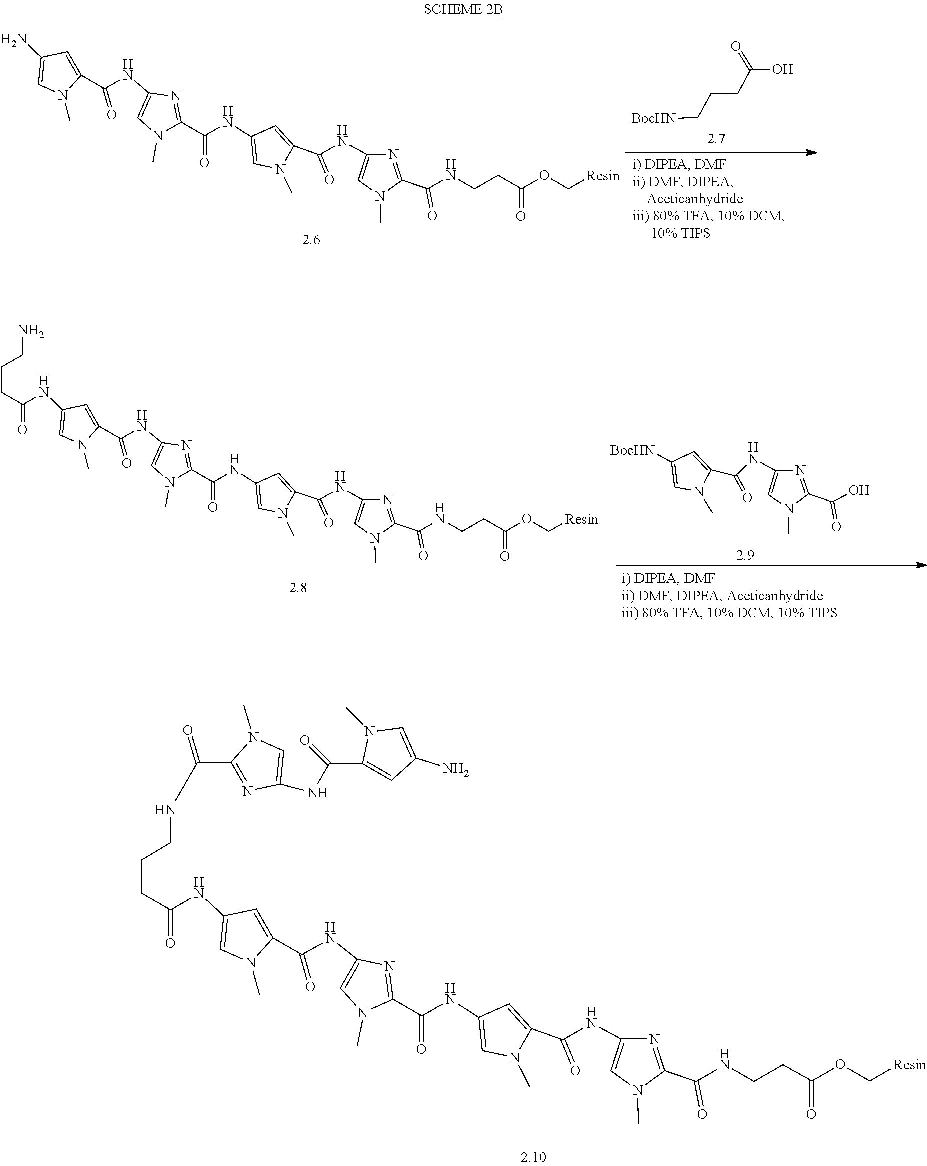

In one aspect, compounds of type 2.10, and similar compounds, can be prepared according to reaction Scheme 2B above. Thus, compounds of type 2.8 can be prepared by a coupling reaction between an appropriate amine, e.g., 2.6 as shown above, and an appropriate carboxylic acid, e.g., 2.9 as shown above. The coupling reaction can be carried out in the presence of an appropriate base, e.g., DIPEA, in an appropriate solvent, e.g., DMF, followed by suspension in an appropriate solvent, e.g., DMF, in the presence of an appropriate base, e.g., DIPEA, and capping with an appropriate acetate, e.g., acetic anhydride or aceticanhydride as shown above. The protecting group is then removed using an appropriate deprotecting agent, e.g., 80% triflouroacetic acid (TFA), and an appropriate reducing agent, e.g., 10% triisopropyl silane (TIPS), in an appropriate solvent, e.g., 10% DCM. Compounds of type 2.10 can be prepared by a coupling reaction between an appropriate amine, e.g., 2.8 as shown above, and an appropriate carboxylic acid, e.g., 2.9 as shown above. The coupling reaction can be carried out in the presence of an appropriate base, e.g., N,N-DIPEA, in an appropriate solvent, e.g., DMF, followed by suspension in an appropriate solvent, e.g., DMF, in the presence of an appropriate base, e.g., DIPEA, and capping with an appropriate acetate, e.g., acetic anhydride or aceticanhydride as shown above. The protecting group is then removed as described for Step 1. As would be understood by one of skill in the art, Step 2 can be repeated as needed such that the desired n groups are included in the peptide, e.g., peptide 2.10 as shown above. Capping of the terminal amine and resin cleavage can then be achieved as described for Scheme 1A and Scheme 1B above. As can be appreciated by one skilled in the art the above reaction provides an example of a generalized approach wherein compounds similar in structure to the specific reactions above (compounds similar to 2.1, 2.2, 2.3, and 2.4) can be substituted in the reaction to provide polyamides similar to Formula 2.5.

### 3. Route 3

In one aspect, the compounds disclosed herein can be prepared as shown below.

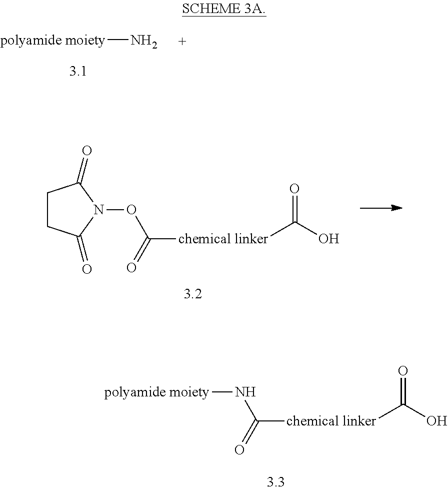

Compounds are represented in generic form, with substituents as noted in compound descriptions elsewhere herein. A specific non-limiting example of the synthesis shown in Scheme 3A is provided below.

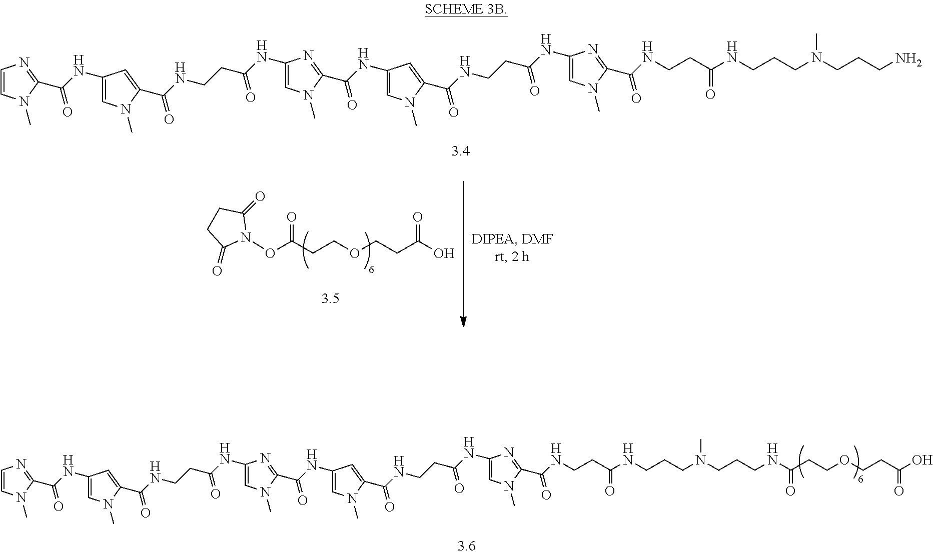

In one aspect, compounds of type 3.6, and similar compounds, can be prepared according to reaction Scheme 3B above. Thus, compounds of type 3.6 can be prepared by coupling a polyamide moiety, e.g., 3.4 as shown above, and an appropriate carboxyl-terminated chemical linker, e.g., 3.5 as shown above. The coupling reaction can be carried out in the presence of an appropriate base, e.g., DIPEA, in an appropriate solvent, e.g., DMF, at an appropriate temperature, e.g., room temperature, for an appropriate period of time, e.g., 2 hours. As can be appreciated by one skilled in the art, the above reaction provides an example of a generalized approach wherein compounds similar in structure to the specific reactants above (compounds similar to 3.1 and 3.2) can be substituted in the reaction to provide compounds similar to Formula 3.3.

### 4. Route 4

In one aspect, the sequence selective DNA binding compounds disclosed herein can be prepared as shown below.

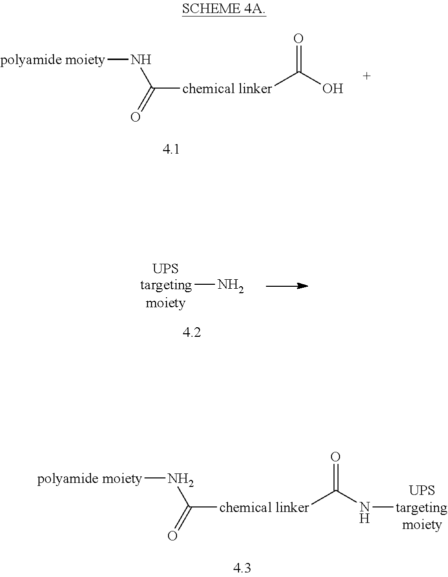

Compounds are represented in generic form, with substituents as noted in compound descriptions elsewhere herein. A specific non-limiting example of the synthesis shown in Scheme 4A is provided below.

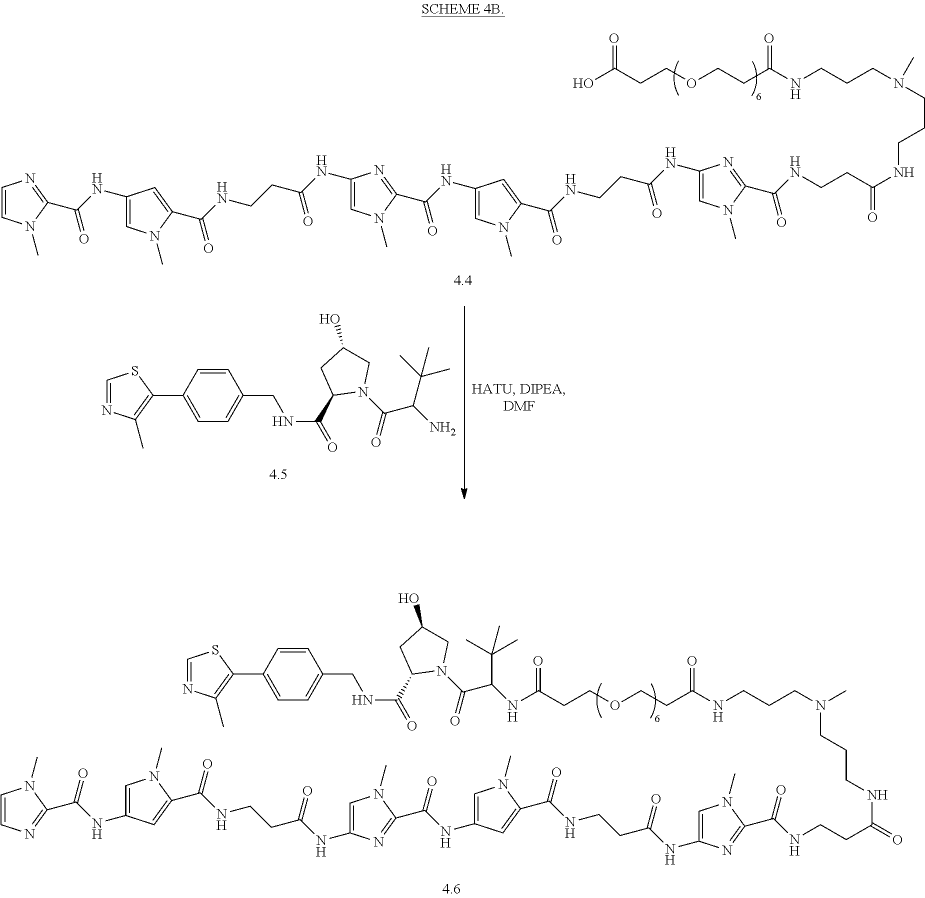

In one aspect, compounds of type 4.6 and similar compounds, can be prepared according to reaction Scheme 4B above. Thus, compounds of type 4.6 can be prepared by coupling an appropriate carboxylic acid, e.g., 4.4 as shown above, to an appropriate amine-terminated UPS targeting moiety, e.g., 4.5 as shown above. The coupling reaction is carried out in the presence of an appropriate coupling agent, e.g., HATU, and an appropriate base, e.g., DIPEA<in an appropriate solvent, e.g., DMF. As can be appreciated by one skilled in the art, the above reaction provides an example of a generalized approach wherein compounds similar in structure to the specific reactants above (compounds similar to 4.1 and 4.2) can be substituted in the reaction to provide sequence selective DNA binding compounds similar to Formula 4.3.

### 5. Route 5

In one aspect, the sequence selective DNA binding compounds disclosed herein can be prepared as shown below.

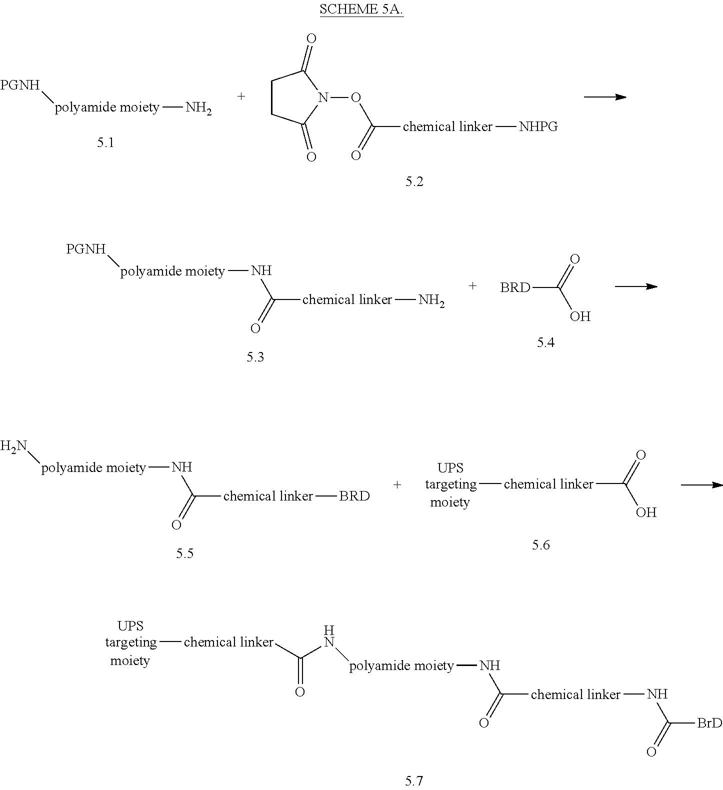

Compounds are represented in generic form, wherein each occurrence of PG independently is an amine protecting group (e.g., carbobenzyloxy, p-methoxybenzyl carbonyl, t-butyloxycarbonyl, 9-fluorenylmethyloxycarbonyl, acetyl, benzoyl, benzyl, carbamate, p-methoxybenzyl, 3,4-dimethoxybenzyl, p-methoxyphenyl, tosyl, 4-nitrobenzenesulfonyl), and with other substituents as noted in compound descriptions elsewhere herein. A specific non-limiting example of the synthesis shown in Scheme 5A is provided below.

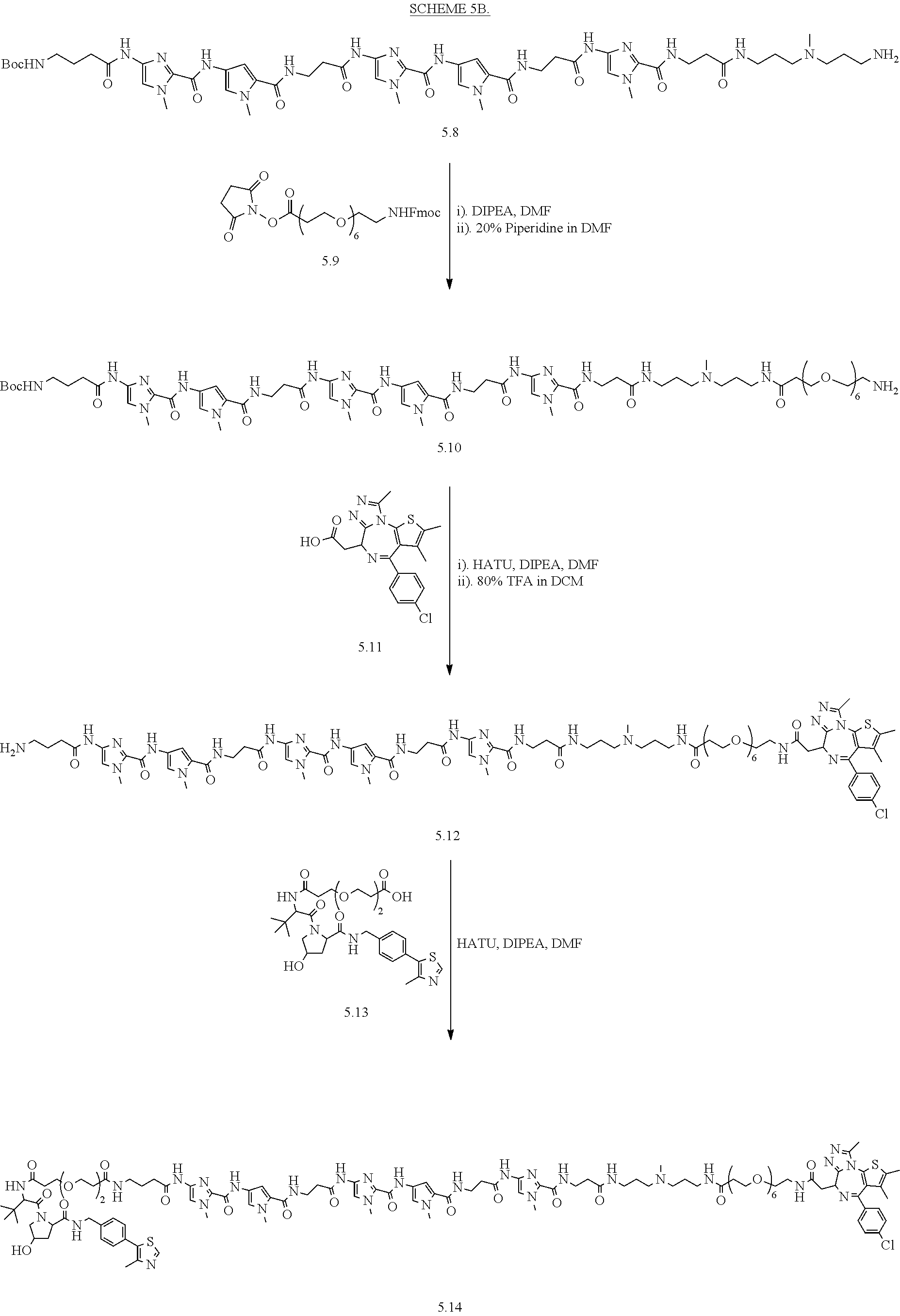

In one aspect, compounds of type 5.14 and similar compounds, can be prepared according to reaction Scheme 5B above. Thus, compounds of type 5.10 can be prepared by a coupling reaction between an appropriate polyamide, e.g., 5.8 as shown above, and a protected chemical linker, e.g., 5.9 as shown above. The coupling reaction is carried out in the presence of an appropriate base, e.g., DIPEA, in an appropriate solvent, e.g., DMF, followed by addition of an appropriate deprotecting agent, e.g., 20% piperidine in DMF. Compounds of type 5.12 can be prepared by a coupling reaction between an appropriate polyamide, e.g., 5.10 as shown above, and an appropriate carboxyl-terminated BET binding ligand, e.g., 5.11 as shown above. The coupling reaction is carried out in the presence of an appropriate coupling agent, e.g., HATU, and an appropriate base, e.g., DIPEA, in an appropriate solvent, e.g., DMF, followed by addition of an appropriate deprotecting agent, e.g., 80% TFA in DCM. Compounds of type 5.14 can be prepared by a coupling reaction between an appropriate polyamide tethered to a BET binding ligand, e.g., 5.12 as shown above, and an appropriate UPS targeting moiety tethered to a carboxyl-terminated chemical linker, e.g., 5.13 as shown above. The coupling reaction is carried out in the presence of an appropriate coupling agent, e.g., HATU, and an appropriate base, e.g., DIPEA, in an appropriate solvent, e.g., DMF. As can be appreciated by one skilled in the art, the above reaction provides an example of a generalized approach wherein compounds similar in structure to the specific reactants above (compounds similar to 5.1, 5.2, 5.3, 5.4, 5.5, and 5.6) can be substituted in the reaction to provide sequence selective DNA binding compounds similar to Formula 5.7.

### 6. Route 6

In one aspect, the transcription factor binding compounds disclosed herein can be prepared as shown below.

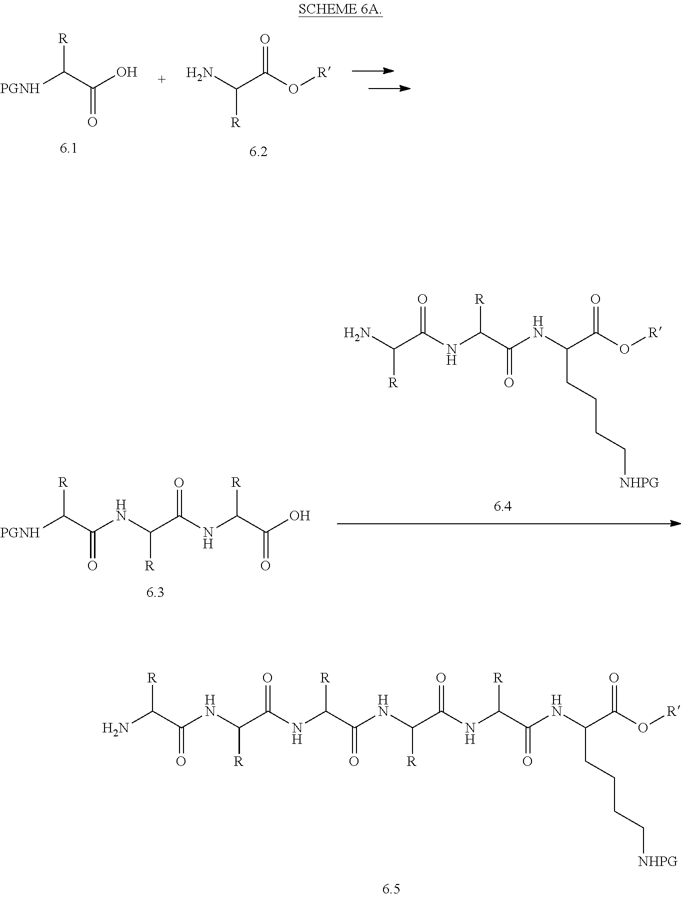

Compounds are represented in generic form, wherein each occurrence of R is independently a residue of a naturally or non-naturally occurring amino acid side chain, wherein each occurrence of R′ is independently C1-C4 alkyl, wherein each occurrence of PG is independently an amine protecting group (e.g., carbobenzyloxy, p-methoxybenzyl carbonyl, t-butyloxycarbonyl, 9-fluorenylmethyloxycarbonyl, acetyl, benzoyl, benzyl, carbamate, p-methoxy benzyl, 3,4-dimethoxybenzyl, p-methoxyphenyl, tosyl, 4-nitrobenzenesulfonyl), and wherein other substituents are as noted in compound descriptions elsewhere herein. A specific non-limiting example of the synthesis shown in Scheme 6A is provided below.

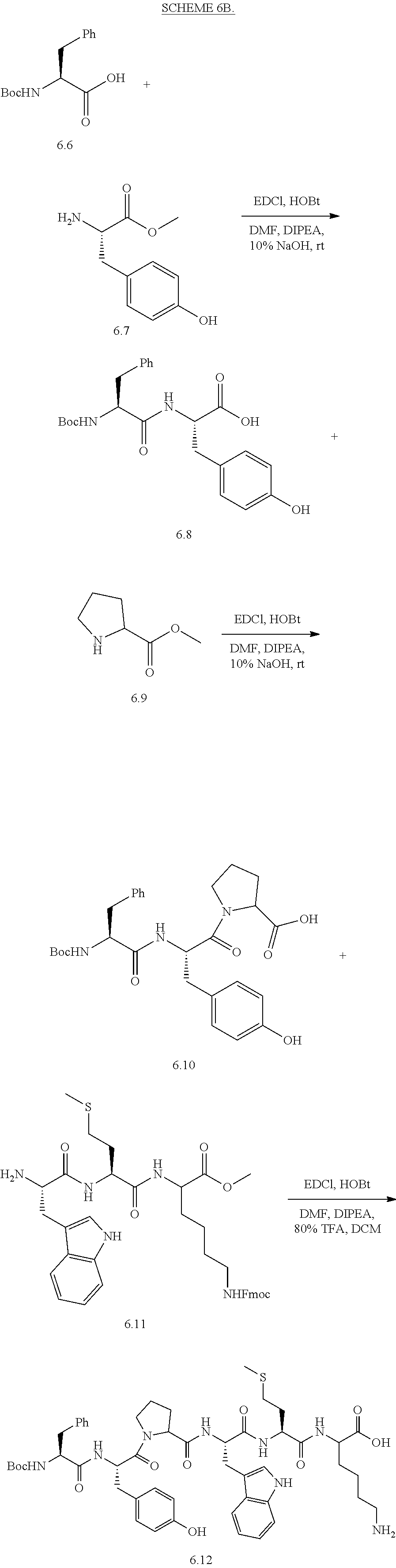

In one aspect, compounds of type 6.12 and similar compounds, can be prepared according to reaction Scheme 6B above. Thus, compounds of type 6.8 can be prepared by a coupling reaction between a first amino acid, e.g., 6.6 as shown above, and a second amino acid, e.g., 6.7 as shown above. The coupling reaction is carried out in the presence of an appropriate coupling agent, e.g., 1-ethyl-3-(3-dimethylaminopropyl) carbodiimide (EDCI), an appropriate activating agent, e.g., hydroxybenzotriazole (HOBt), and an appropriate base, e.g., DIPEA, in an appropriate solvent, e.g., DMF, followed by addition of an appropriate base, e.g., 10% sodium hydroxide. Compounds of type 6.10 can be similarly prepared by a coupling reaction between an appropriate dipeptide, e.g., 6.8 as shown above, and an appropriate amino acid, e.g., 6.9 as shown above. The coupling reaction is carried out using the same or similar conditions as shown in Step 1. Step 2 is then repeated as needed to achieve the desired peptide. Compounds of type 6.11 can be prepared by a coupling reaction between an appropriate first peptide, e.g., 6.10 as shown above, and an appropriate second peptide having a protected alkylamine group, e.g., 6.11 as shown above. The coupling reaction is carried out in the presence of an appropriate coupling agent, e.g., EDCI, an appropriate activating agent, e.g., HOBt, and an appropriate base, e.g., DIPEA, in an appropriate solvent, e.g., DMF, followed by addition of an appropriate acid, e.g., 80% TFA, in an appropriate solvent, e.g., DCM. As can be appreciated by one skilled in the art, the above reaction provides an example of a generalized approach wherein compounds similar in structure to the specific reactants above (compounds similar to 6.1, 6.2, 6.3, and 6.4) can be substituted in the reaction to provide transcription factor binding compounds similar to Formula 6.5.

### 7. Route 7

In one aspect, the sequence selective DNA binding compounds disclosed herein can be prepared as shown below.

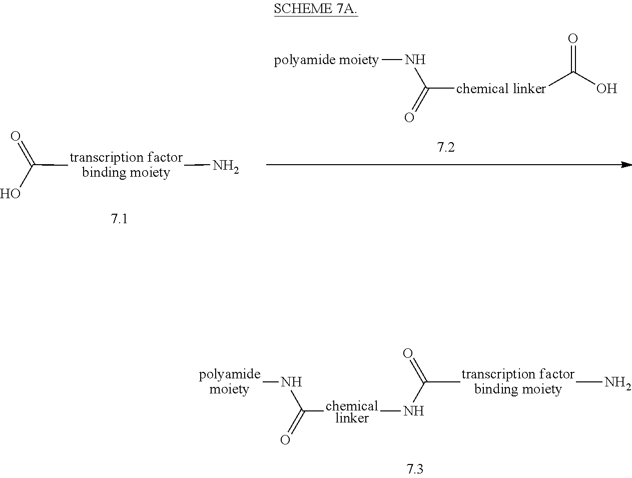

Compounds are represented in generic form, wherein substituents are as noted in compound descriptions elsewhere herein. A specific non-limiting example of the synthesis shown in Scheme 7A is provided below.

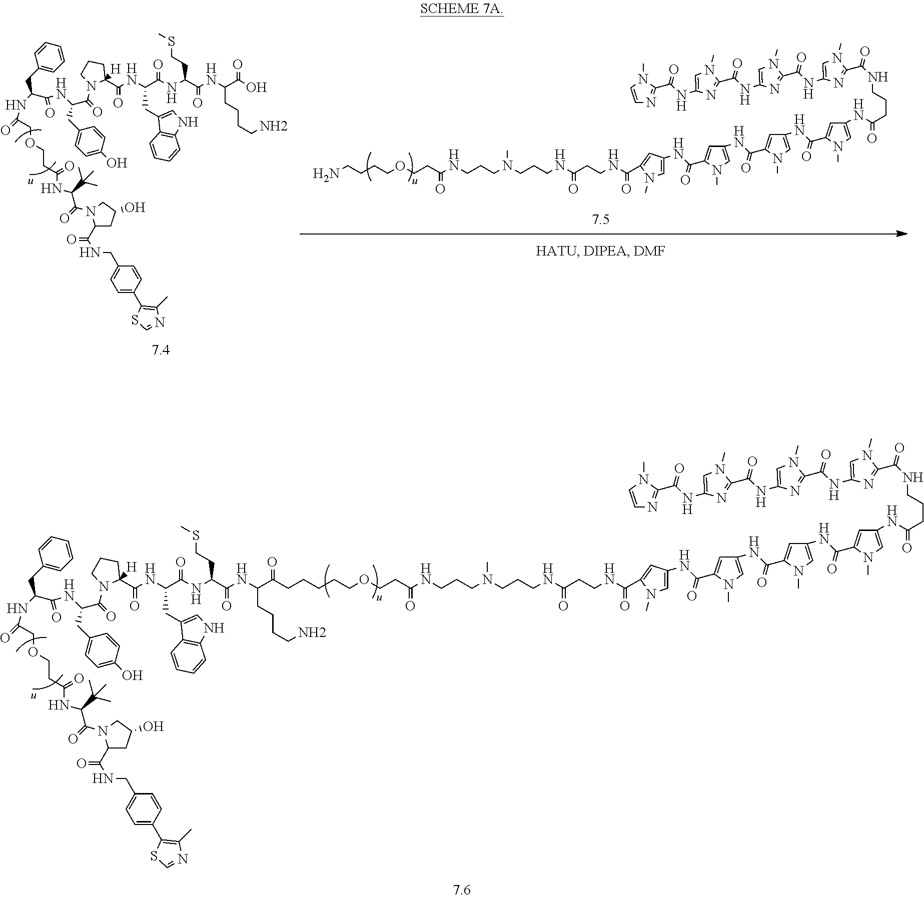

In one aspect, compounds of type 7.6 and similar compounds, can be prepared according to reaction Scheme 7B above. Thus, compounds of type 7.6 can be prepared by a coupling reaction between a carboxyl terminated transcription factor binding moiety, e.g., 7.4 as shown above, and an appropriate polyamide moiety tethered to an amino terminated chemical linker, e.g., 7.7 as shown above. The coupling reaction is carried out in the presence of an appropriate coupling agent, e.g., HATU, and an appropriate base, e.g., DIPEA, in an appropriate solvent, e.g., DMF. As can be appreciated by one skilled in the art, the above reaction provides an example of a generalized approach wherein compounds similar in structure to the specific reactants above (compounds similar to 7.1 and 7.2) can be substituted in the reaction to provide sequence selective DNA binding compounds similar to Formula 7.3.

### 8. Route 8

In one aspect, the sequence selective DNA binding compounds disclosed herein can be prepared as shown below.

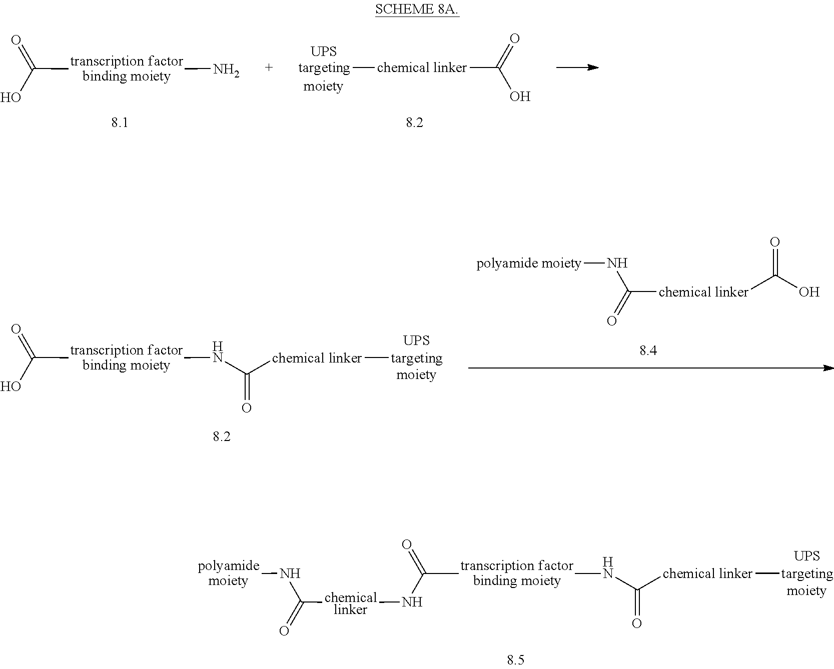

Compounds are represented in generic form, wherein substituents are as noted in compound descriptions elsewhere herein. As would be understood by one of ordinary skill in the art, the UPS targeting moiety or the transcription factor binding moiety, as shown above, can be replaced with a BET binding ligand as detailed elsewhere herein. Alternatively, the transcription factory binding moiety can be replaced with a UPS target moiety as detailed elsewhere herein. A specific non-limiting example of the synthesis shown in Scheme 8A is provided below.

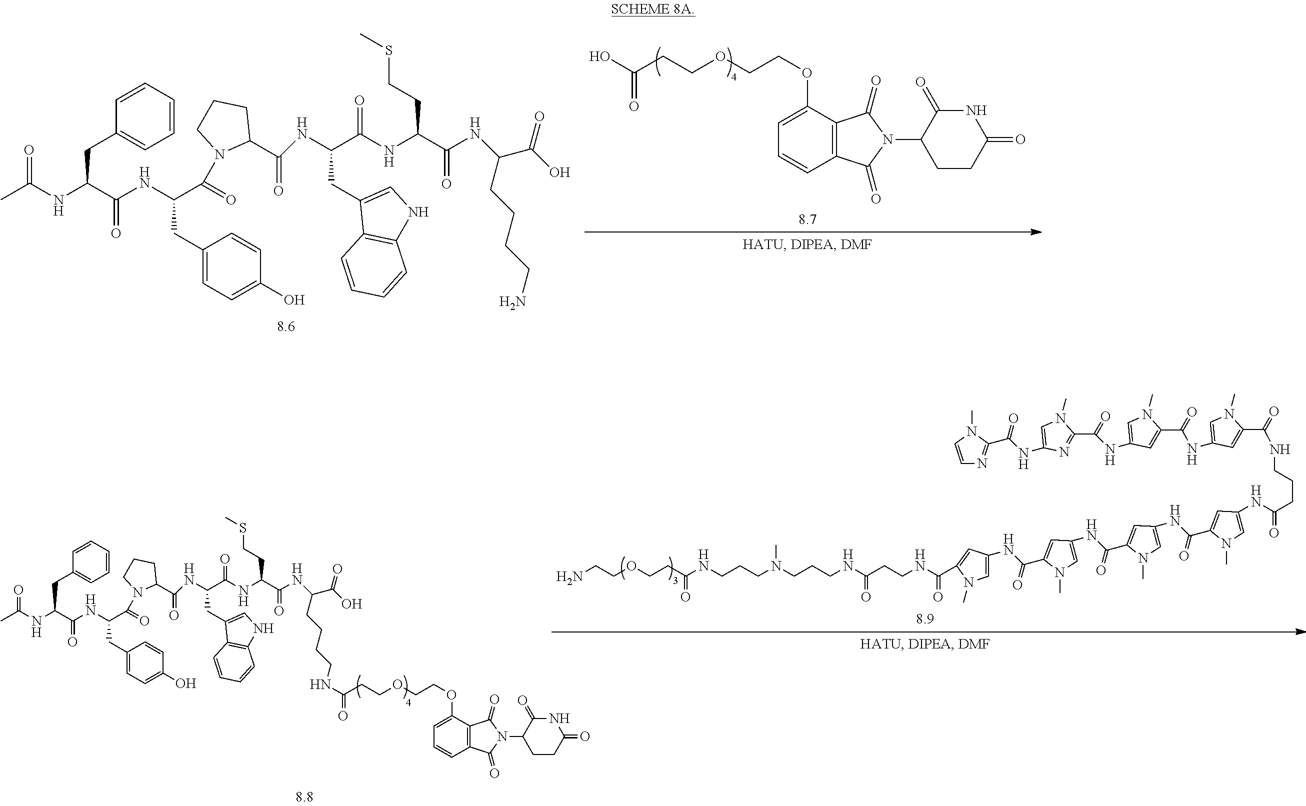

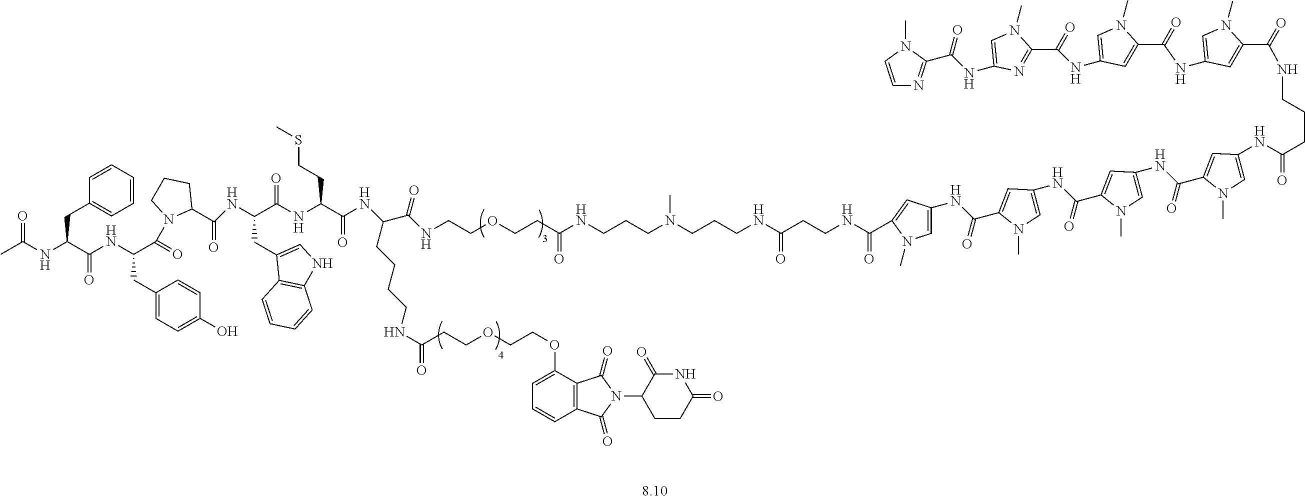

In one aspect, compounds of type 8.10 and similar compounds, can be prepared according to reaction Scheme 8B above. Thus, compounds of type 8.8 can be prepared by a coupling reaction between an amine terminated transcription factor binding moiety, e.g., 8.6 as shown above, and a UPS targeting moiety or a BET binding ligand tethered to a carboxyl terminated chemical linker, e.g., 8.7 as shown above. The coupling reaction is carried out in the presence of an appropriate coupling agent, e.g., HATU, and an appropriate base, e.g., DIPEA, in an appropriate solvent, e.g., DMF. Compounds of type 8.10 can be prepared by a coupling reaction between an appropriate carboxylic acid having two of a UPS targeting moiety, a BET binding ligand, and a transcription factor binding moiety, e.g., 8.8 as shown above, and an appropriate polyamide moiety tethered to an amino terminated chemical linker, e.g., 8.9 as shown above. The coupling reaction is carried out in the presence of an appropriate coupling agent, e.g., HATU, and an appropriate base, e.g., DIPEA, in an appropriate solvent, e.g., DMF. As can be appreciated by one skilled in the art, the above reaction provides an example of a generalized approach wherein compounds similar in structure to the specific reactants above (compounds similar to 8.1, 8.2, 8.3, and 8.4) can be substituted in the reaction to provide sequence selective DNA binding compounds similar to Formula 8.5.

### D. PHARMACEUTICAL COMPOSITIONS

In one aspect, disclosed are pharmaceutical compositions comprising an effective amount of a disclosed sequence selective DNA binding compound, or a pharmaceutically acceptable salt thereof, and a pharmaceutically acceptable carrier.

Thus, in one aspect, disclosed are pharmaceutical compositions comprising an effective amount of a sequence selective DNA binding compound comprising a polyamide moiety configured to bind a deoxyribonucleic acid (DNA) sequence, wherein the polyamide moiety is tethered via a first chemical linker to an Ubiquitin-Proteosome System (UPS) targeting moiety, or a pharmaceutically acceptable salt thereof, and a pharmaceutically acceptable carrier.

In one aspect, disclosed are pharmaceutical compositions comprising an effective amount of a sequence selective DNA binding compound comprising: (a) a polyamide moiety configured to bind a deoxyribonucleic acid (DNA) sequence; and (b) two of a Ubiquitin-Proteosome System (UPS) targeting moiety, a bromodomain extraterminal domain (BET) binding ligand, and a PBX or TCF3-PBX transcription factor binding moiety, wherein the polyamide moiety and two of the UPS targeting moiety, the BET binding ligand, and the transcription factor binding moiety are tethered via a first chemical linker and via a second chemical linker, or a pharmaceutically acceptable salt thereof, and a pharmaceutically acceptable carrier.

In various aspects, the compounds and compositions of the invention can be administered in pharmaceutical compositions, which are formulated according to the intended method of administration. The compounds and compositions described herein can be formulated in a conventional manner using one or more physiologically acceptable carriers or excipients. For example, a pharmaceutical composition can be formulated for local or systemic administration, e.g., administration by drops or injection into the ear, insufflation (such as into the ear), intravenous, topical, or oral administration.

The nature of the pharmaceutical compositions for administration is dependent on the mode of administration and can readily be determined by one of ordinary skill in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, PA 1990.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, an effective amount is a therapeutically effective amount. In a still further aspect, an effective amount is a prophylactically effective amount.

In a further aspect, the pharmaceutical composition is administered to a mammal. In a still further aspect, the mammal is a human. In an even further aspect, the human is a patient.

In a further aspect, the pharmaceutical composition is used to treat a disorder associated with uncontrolled cellular proliferation such as, for example, cancer (e.g., a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, plasma cell neoplasm (myeloma)).

It is understood that the disclosed compositions can be prepared from the disclosed compounds. It is also understood that the disclosed compositions can be employed in the disclosed methods of using.

### E. METHODS OF TREATING A DISORDER IN A SUBJECT

In one aspect, disclosed are methods for treating a disorder in a subject in need thereof, the method comprising administering to the subject an effective amount of a disclosed sequence selective binding compound, thereby treating the disorder. In a further aspect, the disorder is cancer (e.g., a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma)).

Thus, in one aspect, disclosed are methods of treating a disorder in a subject in need thereof, the method comprising administering to the subject a therapeutically effective amount of a sequence selective DNA binding compound comprising a polyamide moiety configured to bind a deoxyribonucleic acid (DNA) sequence, wherein the polyamide moiety is tethered via a first chemical linker to an Ubiquitin-Proteosome System (UPS) targeting moiety, or a pharmaceutically acceptable salt thereof.

In one aspect, disclosed are methods of treating a disorder in a subject in need thereof, the method comprising administering to the subject a therapeutically effective amount of a sequence selective DNA binding compound comprising: (a) a polyamide moiety configured to bind a deoxyribonucleic acid (DNA) sequence; and (b) two of a Ubiquitin-Proteosome System (UPS) targeting moiety, a bromodomain extraterminal domain (BET) binding ligand, and a PBX or TCF3-PBX transcription factor binding moiety, wherein the polyamide moiety and two of the UPS targeting moiety, the BET binding ligand, and the transcription factor binding moiety are tethered via a first chemical linker and via a second chemical linker, or a pharmaceutically acceptable salt thereof.

In a further aspect, the subject has been diagnosed with a need for treatment of the disorder prior to the administering step. In a still further aspect, the subject is at risk for developing the disorder prior to the administering step.

In a further aspect, the subject is a mammal. In a still further aspect, the mammal is a human.

In a further aspect, the method further comprises the step of identifying a subject in need of treatment of the disorder.

In a further aspect, the disorder is a cancer. In a still further aspect, the cancer is selected from a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma).

In a further aspect, the effective amount is a therapeutically effective amount. In a still further aspect, the effective amount is a prophylactically effective amount.

In a further aspect, the method further comprises the step of administering a therapeutically effective amount of at least one chemotherapeutic agent to the subject. In a still further aspect, the compound and the agent are administered sequentially. In yet a further aspect, the compound and the agent are administered simultaneously.

In a further aspect, the compound and the agent are co-formulated. In a still further aspect, the compound and the agent are co-packaged.

In a further aspect, the compound is administered as a single active agent.

### F. METHODS OF USING THE COMPOSITIONS

Provided are methods of using of a disclosed composition or medicament. In one aspect, the method of use is directed to the treatment of a disorder. In a further aspect, the disclosed compounds can be used as single agents or in combination with one or more other drugs in the treatment, prevention, control, amelioration, or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

The pharmaceutical compositions and methods of the present invention can further comprise other therapeutically active compounds as noted herein which are usually applied in the treatment of the above mentioned pathological conditions.

### 1. Manufacture of a Medicament

In one aspect, the invention relates to a method for the manufacture of a medicament for treating a disorder associated with uncontrolled cellular proliferation in a mammal, the method comprising combining a therapeutically effective amount of a disclosed sequence selective DNA binding compound or product of a disclosed method with a pharmaceutically acceptable carrier or diluent.

As regards these applications, the present method includes the administration to an animal, particularly a mammal, and more particularly a human, of a therapeutically effective amount of the compound effective in the inhibition of uncontrolled cellular proliferation, such as cancer, and the cancer's disclosed herein. The dose administered to an animal, particularly a human, in the context of the present invention should be sufficient to affect a therapeutic response in the animal over a reasonable time frame. One skilled in the art will recognize that dosage will depend upon a variety of factors including the condition of the animal, the body weight of the animal, as well as the severity and stage of the disorder.

Thus, in one aspect, the invention relates to the manufacture of a medicament comprising combining a disclosed sequence selective DNA binding compound or a product of a disclosed method of making, or a pharmaceutically acceptable salt, solvate, or polymorph thereof, with a pharmaceutically acceptable carrier or diluent.

### 2. Use of Compounds and Compositions

Also provided are the uses of the disclosed compounds and compositions. Thus, in one aspect, the invention relates to the uses of sequence selective DNA binding compounds. In a further aspect, the invention relates to the use of a disclosed sequence selective DNA binding compound or product of a disclosed method in the manufacture of a medicament for the treatment of a disorder associated with uncontrolled cellular proliferation, for example, cancer, (e.g., a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, plasma cell neoplasm (myeloma)).

In a further aspect, the use relates to a process for preparing a pharmaceutical composition comprising a therapeutically effective amount of a disclosed sequence selective DNA binding compound or a product of a disclosed method, and a pharmaceutically acceptable carrier, for use as a medicament.

In a further aspect, the use relates to a process for preparing a pharmaceutical composition comprising a therapeutically effective amount of a disclosed sequence selective DNA binding compound or a product of a disclosed method, wherein a pharmaceutically acceptable carrier is intimately mixed with a therapeutically effective amount of the disclosed compound or the product of a disclosed method.

In various aspects, the use relates to the treatment of uncontrolled cellular proliferation in a vertebrate animal. In a further aspect, the use relates to the treatment of uncontrolled cellular proliferation in a human subject.

In a further aspect, the use is the treatment of uncontrolled cellular proliferation, for example, cancer (e.g., a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma)).

It is understood that the disclosed uses can be employed in connection with the disclosed compounds, methods, compositions, and kits. In a further aspect, the invention relates to the use of a disclosed sequence selective DNA binding compound or composition of a medicament for the treatment of a disorder associated with uncontrolled cellular proliferation in a mammal.

In a further aspect, the invention relates to the use of a disclosed sequence selective DNA binding compound or composition in the manufacture of a medicament for the treatment of a disorder associated with uncontrolled cellular proliferation such as, for example, cancer (e.g., a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma)).

### 3. Subjects

In various aspects, the subject of the disclosed methods is a vertebrate, e.g., a mammal. Thus, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

In some aspects of the disclosed methods, the subject has been diagnosed with a need for treatment prior to the administering step. In some aspects of the disclosed method, the subject has been diagnosed with a disorder associated with uncontrolled cellular proliferation prior to the administering step. In some aspects of the disclosed methods, the subject has been identified with a need for treatment prior to the administering step. In one aspect, a subject can be treated prophylactically with a compound or composition disclosed herein, as discussed herein elsewhere.

a. Dosage

Toxicity and therapeutic efficacy of the agents and pharmaceutical compositions described herein can be determined by standard pharmaceutical procedures, using either cells in culture or experimental animals to determine the LD₅₀ (the dose lethal to 50% of the population) and the ED₅₀ (the dose therapeutically effective in 50% of the population). The dose ratio between toxic and therapeutic effects is the therapeutic index and can be expressed as the ratio LD₅₀/ED₅₀. Polypeptides or other compounds that exhibit large therapeutic indices are preferred.

Data obtained from cell culture assays and further animal studies can be used in formulating a range of dosage for use in humans. The dosage of such compounds lies preferably within a range of circulating concentrations that include the ED₅₀ with little or no toxicity, and with little or no adverse effect on a human's ability to hear. The dosage may vary within this range depending upon the dosage form employed and the route of administration utilized. For any agents used in the methods described herein, the therapeutically effective dose can be estimated initially from cell culture assays. A dose can be formulated in animal models to achieve a circulating plasma concentration range that includes the IC₅₀ (that is, the concentration of the test compound which achieves a half-maximal inhibition of symptoms) as determined in cell culture. Such information can be used to more accurately determine useful doses in humans. Exemplary dosage amounts of a differentiation agent are at least from about 0.01 to 3000 mg per day, e.g., at least about 0.00001, 0.0001, 0.001, 0.01, 0.1, 1, 2, 5, 10, 25, 50, 100, 200, 500, 1000, 2000, or 3000 mg per kg per day, or more.

The formulations and routes of administration can be tailored to the disease or disorder being treated, and for the specific human being treated. For example, a subject can receive a dose of the agent once or twice or more daily for one week, one month, six months, one year, or more. The treatment can continue indefinitely, such as throughout the lifetime of the human. Treatment can be administered at regular or irregular intervals (once every other day or twice per week), and the dosage and timing of the administration can be adjusted throughout the course of the treatment. The dosage can remain constant over the course of the treatment regimen, or it can be decreased or increased over the course of the treatment.

In various aspects, the dosage facilitates an intended purpose for both prophylaxis and treatment without undesirable side effects, such as toxicity, irritation, or allergic response. Although individual needs may vary, the determination of optimal ranges for effective amounts of formulations is within the skill of the art. Human doses can readily be extrapolated from animal studies (Katocs et al., (1990) Chapter 27 in Remington's Pharmaceutical Sciences, 18th Ed., Gennaro, ed., Mack Publishing Co., Easton, PA). In general, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., (1996) Chapter 3, In: Goodman & Gilman's The Pharmacological Basis of Therapeutics, 9th Ed., Hardman et al., eds., McGraw-Hill, New York, NY).

b. Routes of Administration

Also provided are routes of administering the disclosed sequence selective DNA binding compounds and compositions. The compounds and compositions of the present invention can be administered by direct therapy using systemic administration and/or local administration. In various aspects, the route of administration can be determined by a patient's health care provider or clinician, for example following an evaluation of the patient. In various aspects, an individual patient's therapy may be customized, e.g., the type of agent used, the routes of administration, and the frequency of administration can be personalized. Alternatively, therapy may be performed using a standard course of treatment, e.g., using pre-selected agents and pre-selected routes of administration and frequency of administration.

Systemic routes of administration can include, but are not limited to, parenteral routes of administration, e.g., intravenous injection, intramuscular injection, and intraperitoneal injection: enteral routes of administration e.g., administration by the oral route, lozenges, compressed tablets, pills, tablets, capsules, drops (e.g., ear drops), syrups, suspensions and emulsions; rectal administration, e.g., a rectal suppository or enema; a vaginal suppository; a urethral suppository; transdermal routes of administration; and inhalation (e.g., nasal sprays).

In various aspects, the modes of administration described above may be combined in any order.

### G. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. Chemistry Methods

### a. Synthesis of Polyamide 1 (PA1)

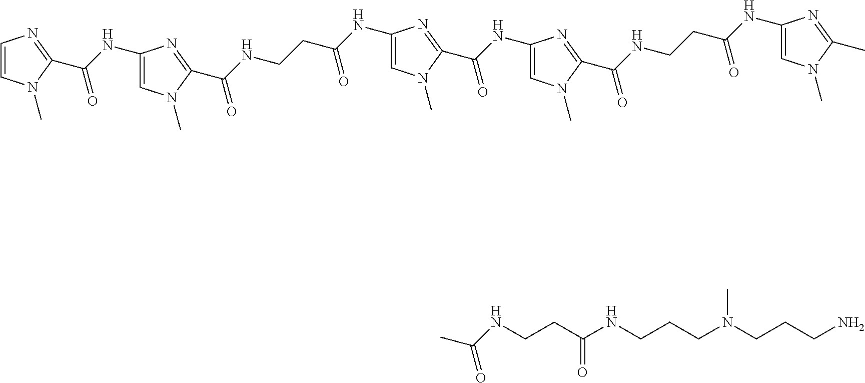

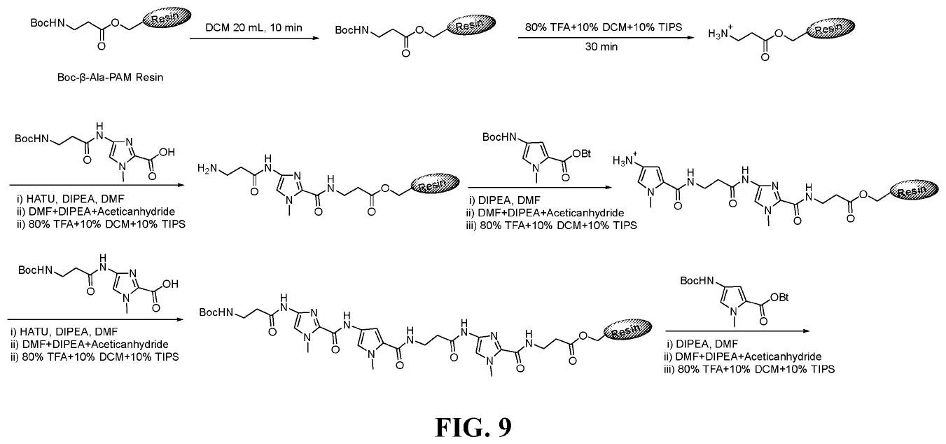

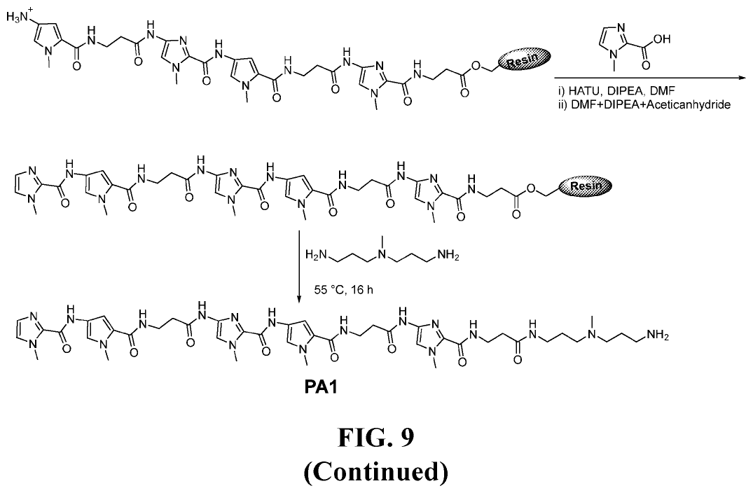

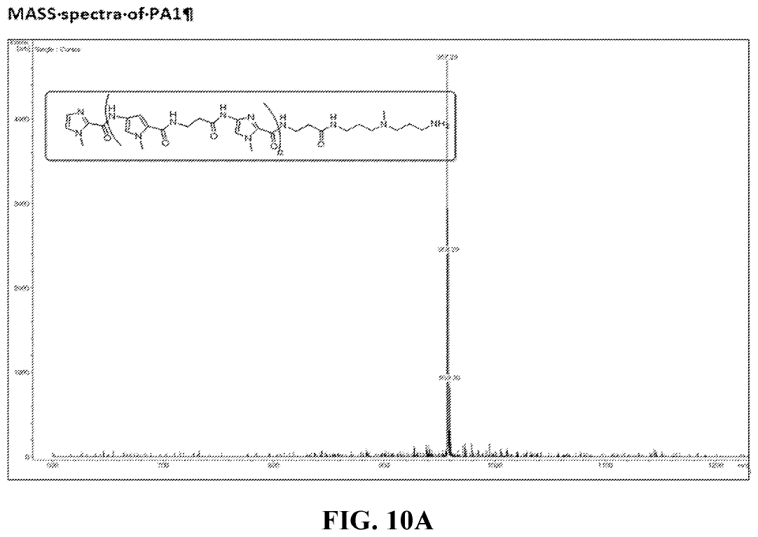

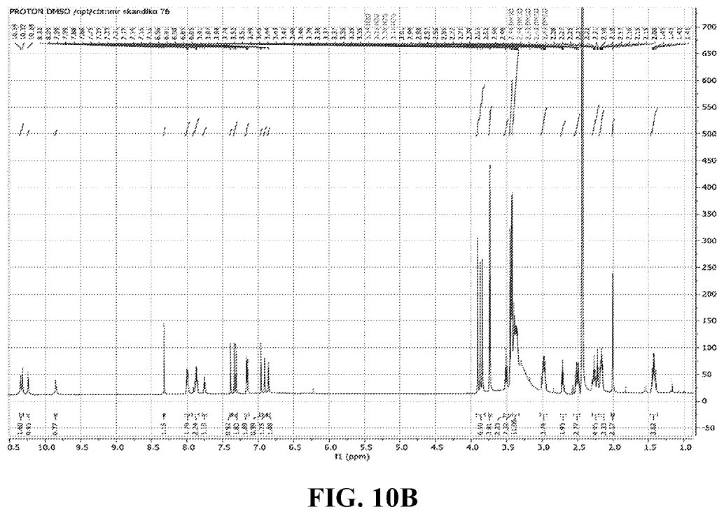

PA1 was synthesized as shown in FIG. 9. Briefly, polyamides were synthesized by manual solid-phase synthesis using Boc-beta-ala-PAM resin (1 g, 0.6 mm) following established procedures (Boc=tert-butoxycarbonyl). After synthesis was complete, the polyamide was cleaved from the solid support by aminolysis with 3,3′-diamino-N-methyldipropylamine (55° C., 14 h). Polyamide was precipitated twice with diethyl ether, dissolved in 15% acetonitrile/H2O+0.1% TFA, and purified by reverse-phase preparative HPLC on a C18 column. Fractions that showed pure polyamide were frozen in liquid nitrogen and lyophilized to afford a white or off-white powder. Identity and purity were confirmed by MALDI-TOF mass spectrometry (FIG. 10A), ¹HNMR (FIG. 10B), and analytical HPLC (not shown).

### b. Synthesis of Polyamide 2 (PA2)

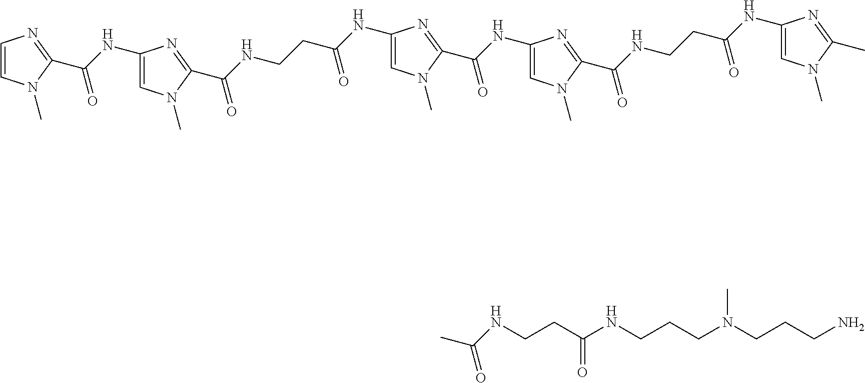

PA2 was synthesized using the same procedure as for PA1.

### c. Synthesis of PA1-(PEG)_{N}-NH₂

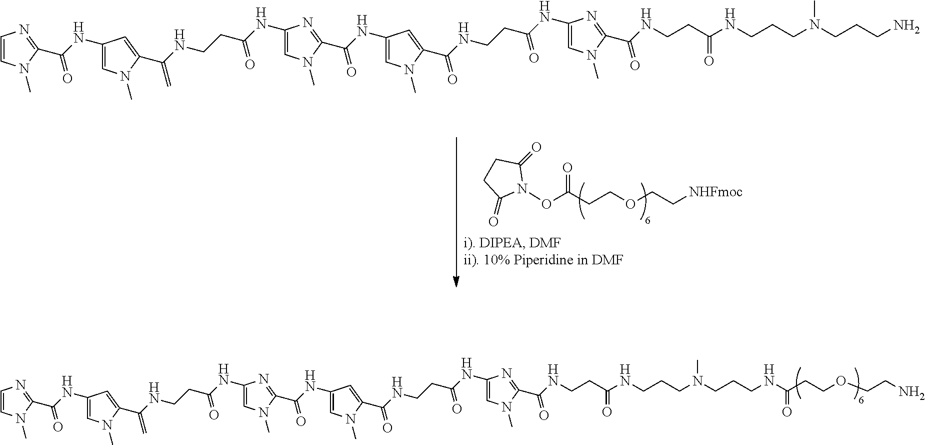

### (i) Representative Synthesis of PA1-(PEG)₆-NH₂

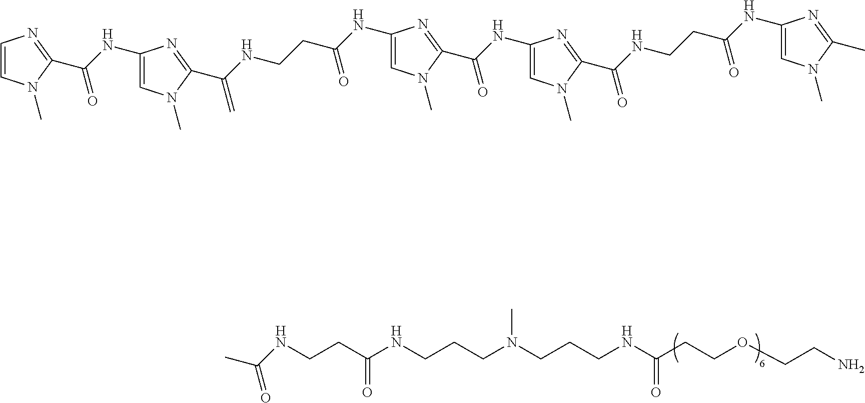

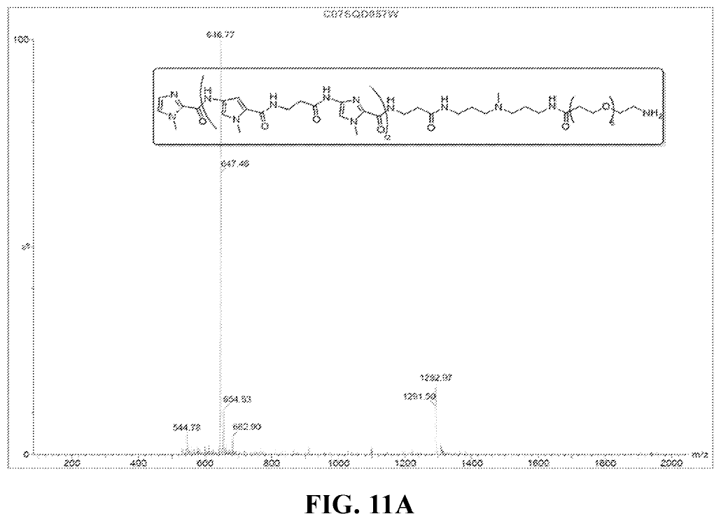

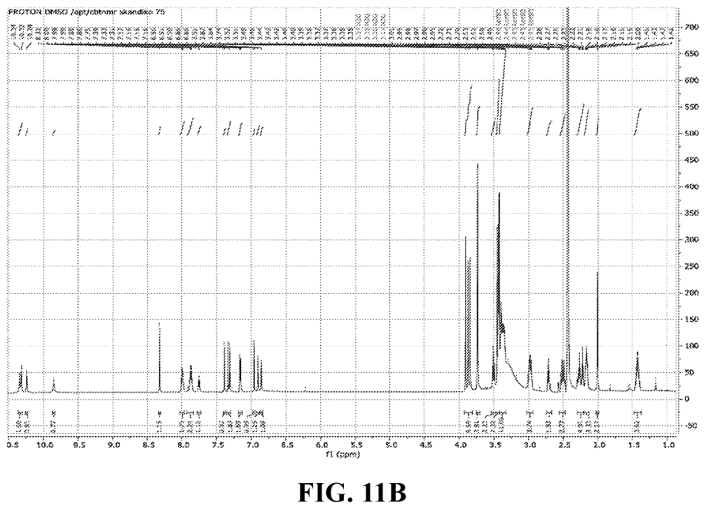

To a stirred solution of PA1 (30 mg, 0.03 mmol, 1 eq) and DIPEA (16 μL, 0.09 mmol, 3 eq) was added FmocNH-PEG₆-NHS ester (20 mg, 0.03 mmol). The reaction mixture was stirred at room temperature for 1 h. Reaction was monitored by LCMS. After the completion of reaction, diluted it in 15% of acetonitrile in water and purified by using Prep-HPLC. Fractions that showed pure polyamide were frozen in liquid nitrogen and lyophilized to afford a white or off-white powder. Identity and purity were confirmed by MALDI-TOF mass spectrometry (FIG. 11A), ¹H NMR (FIG. 11B) and analytical HPLC (data not shown).

### d. Synthesis of Syn-DGRs

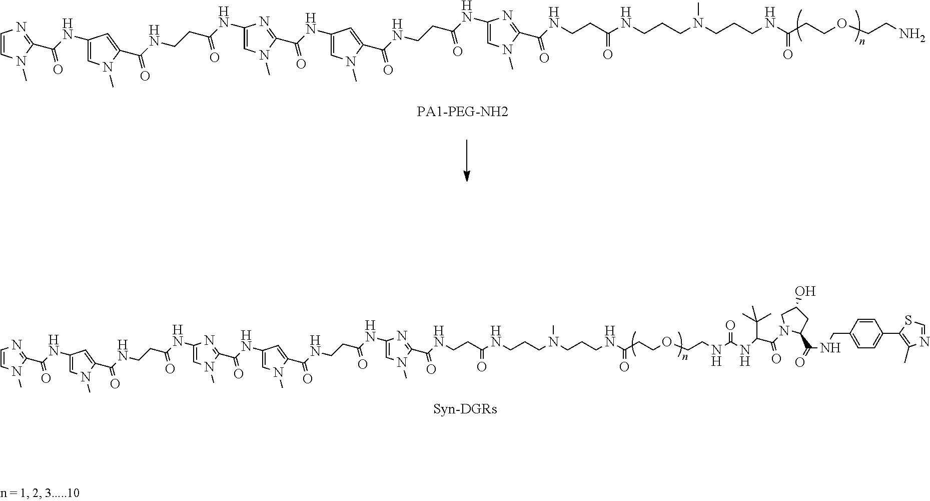

### e. Synthesis of NH₂-Syn-TEFs AND DRG-Syn-TEFs

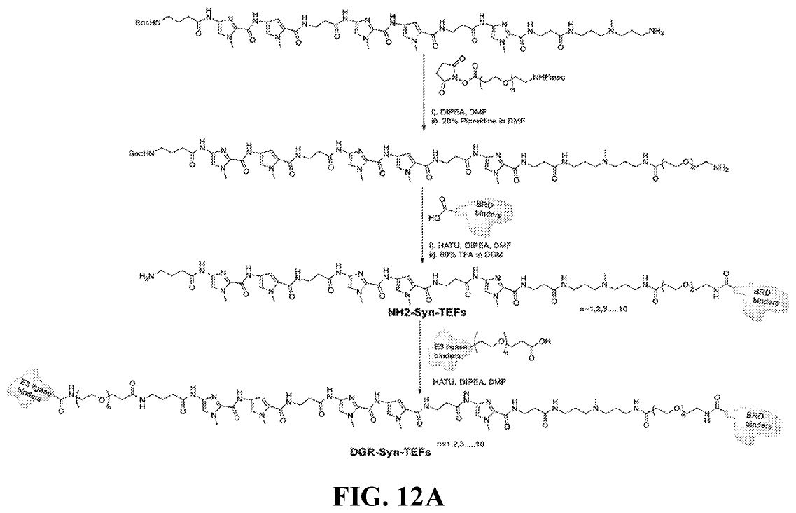

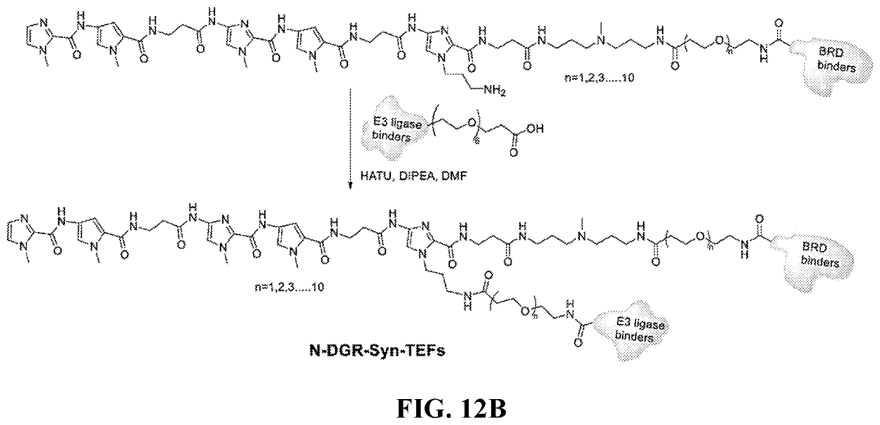

NH₂-Syn-TEFs and DGR-Syn-TEFs were synthesized as shown in FIG. 12A and FIG. 12B. As shown, the acid and amine couplings were done by using HATU, DIPEA, and DMF. Reactions were carried out at room temperature for 2 h.

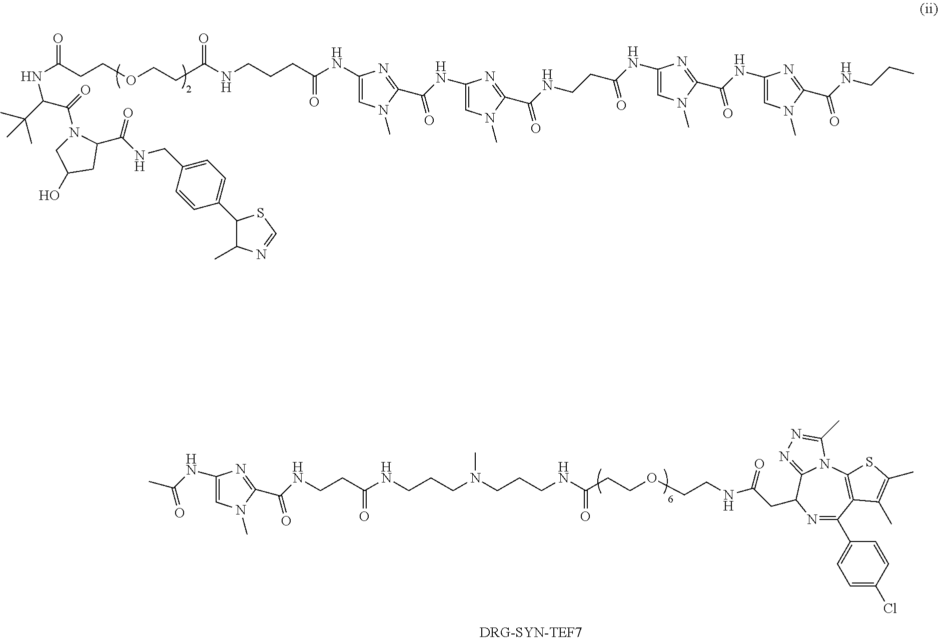

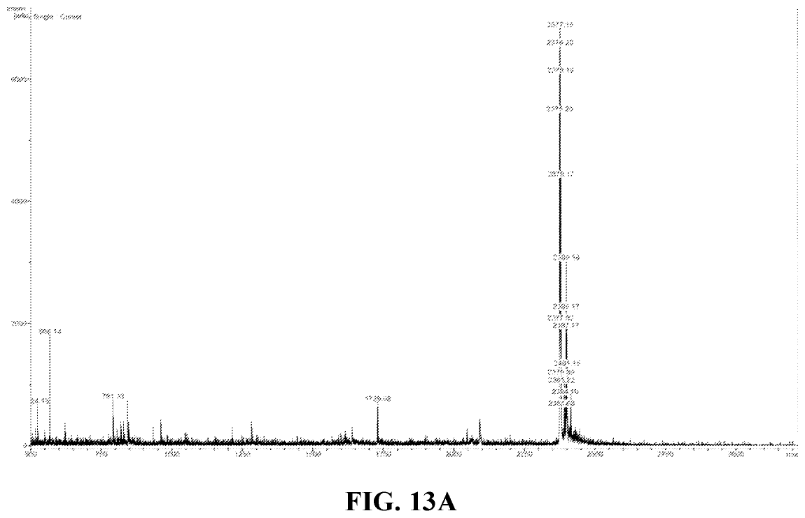

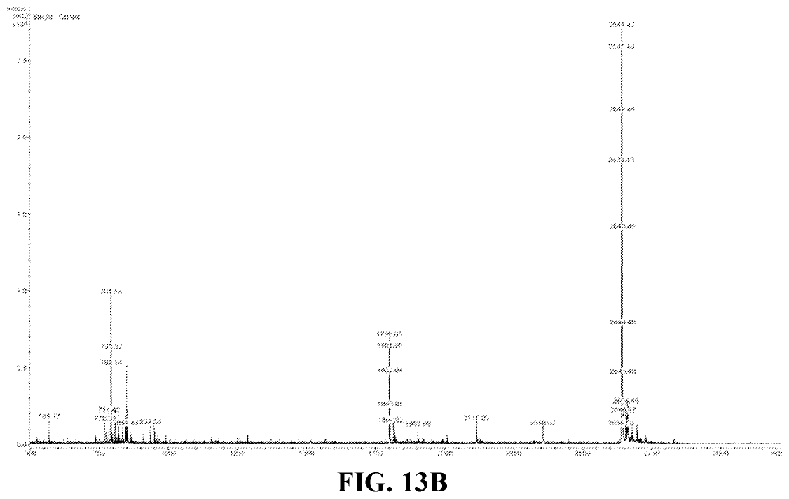

The mass spectra for DRG-Syn-TEF7 is shown in FIG. 13A.

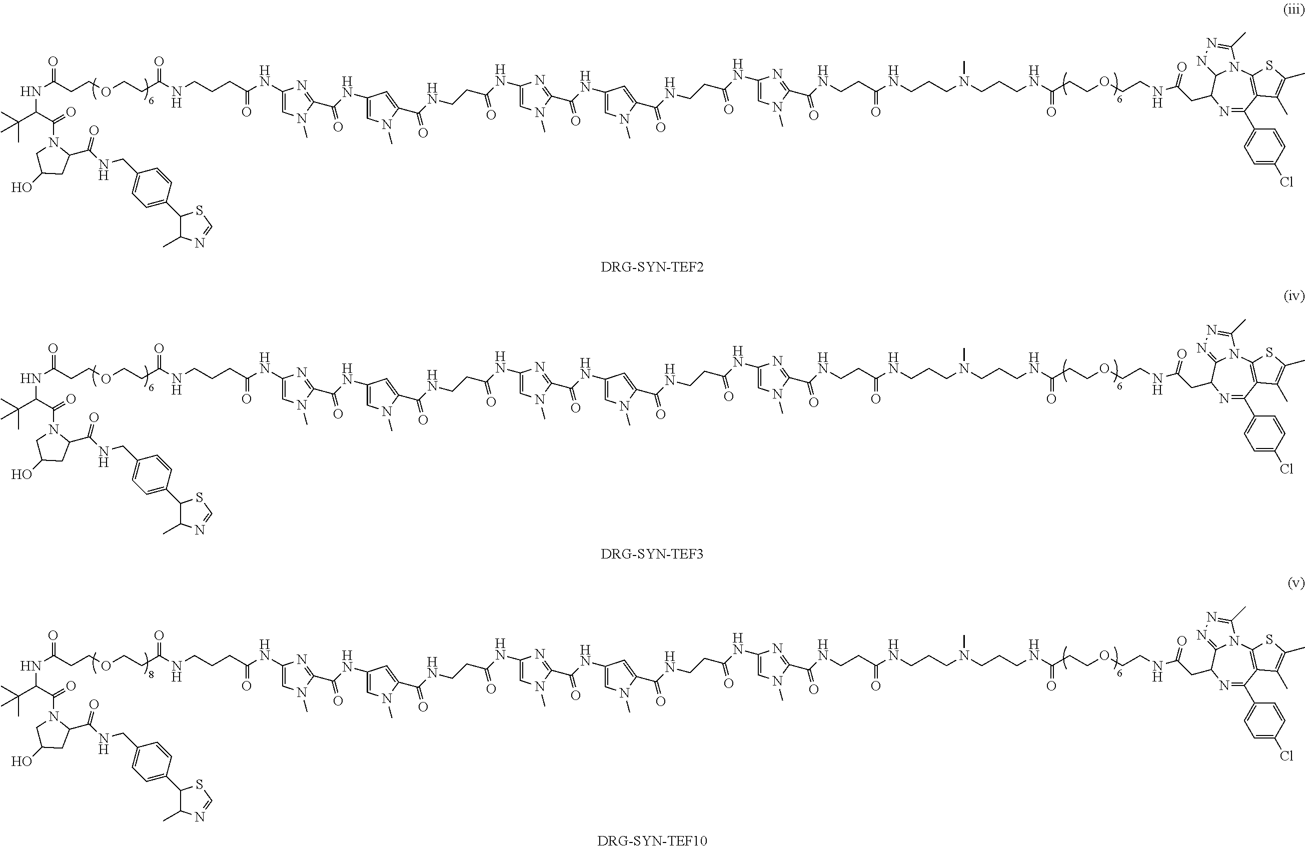

The mass spectra for DRG-Syn-TEF10 is shown in FIG. 13B.

### f. Synthesis of N-DGR-Syn-TEFs

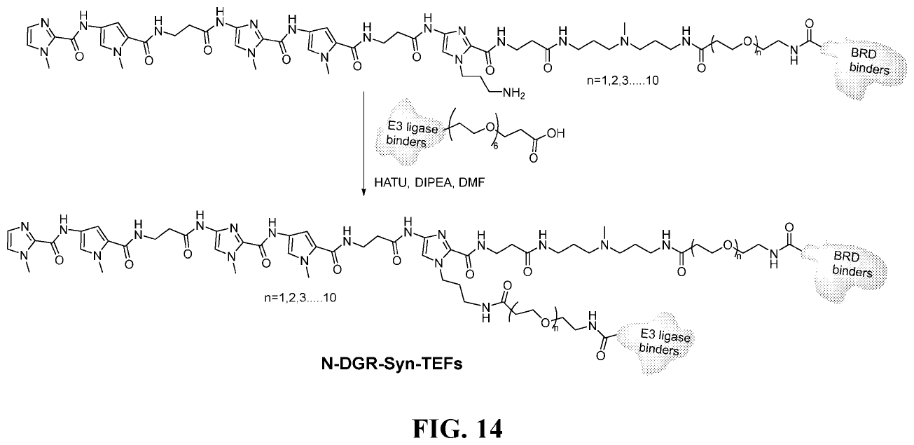

N-DGR-Syn-TEFs were synthesized as shown in FIG. 14. Briefly, PEG-containing E3 ligase binder and NH2-Syn-TEF were conjugated using HATU and DIPEA in DMF. Reaction was carried out at room temperature for 24 h.

### g. Synthesis of Hairpin Polyamides

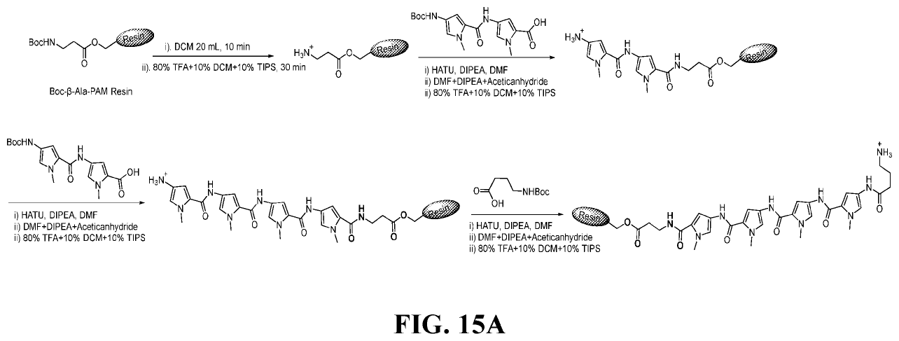

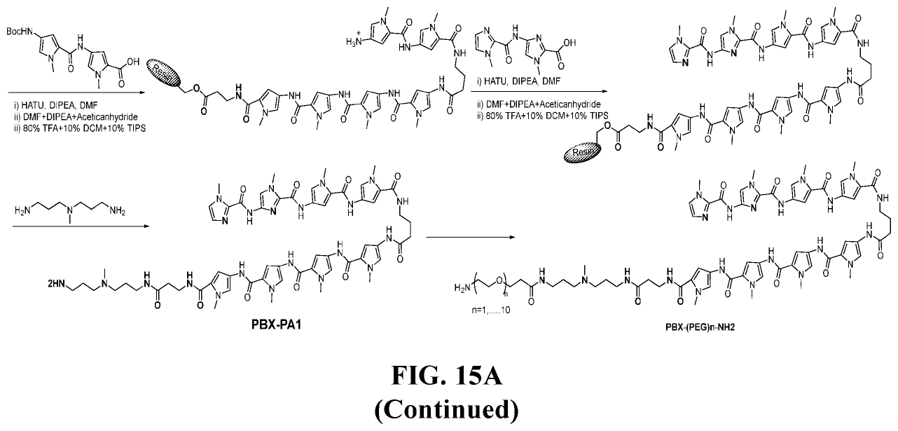

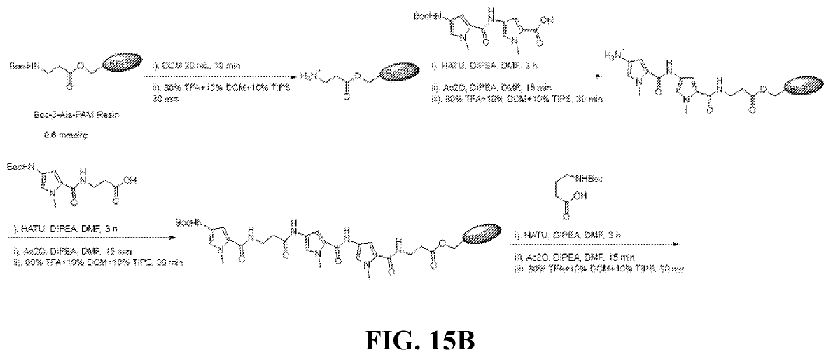

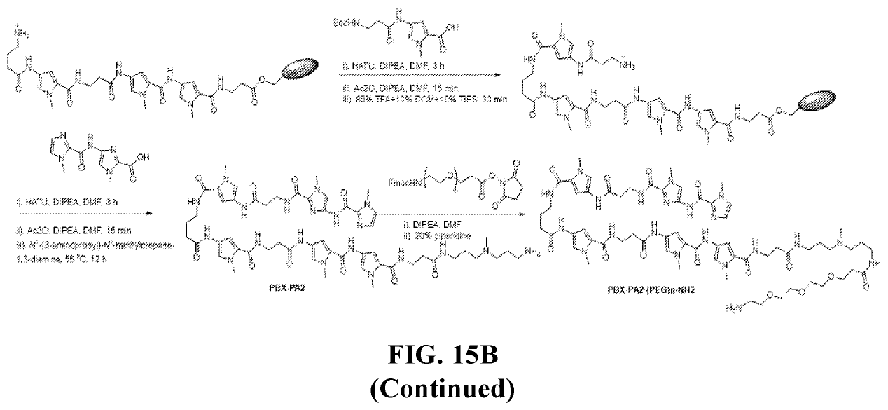

Hairpin polyamides were synthesized as shown in FIG. 15A and FIG. 15B. As shown, above standard solid phase synthesis method was used for these syntheses.

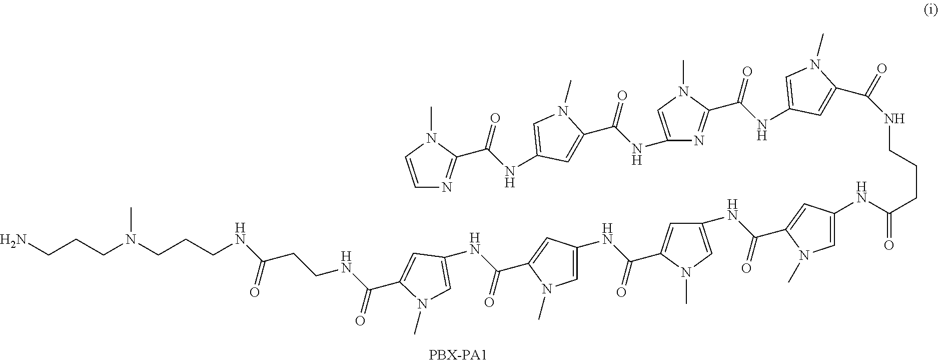 [SMILES: CN(CCCN)CCCNC(=O)CCNC(=O)c1cc(NC(=O)c2cc(NC(=O)c3cc(NC(=O)c4cc(NC(=O)CCCNC(=O)c5cc(NC(=O)c6nc(NC(=O)c7cc(NC(=O)c8nccn8C)cn7C)cn6C)cn5C)cn4C)cn3C)cn2C)cn1C]

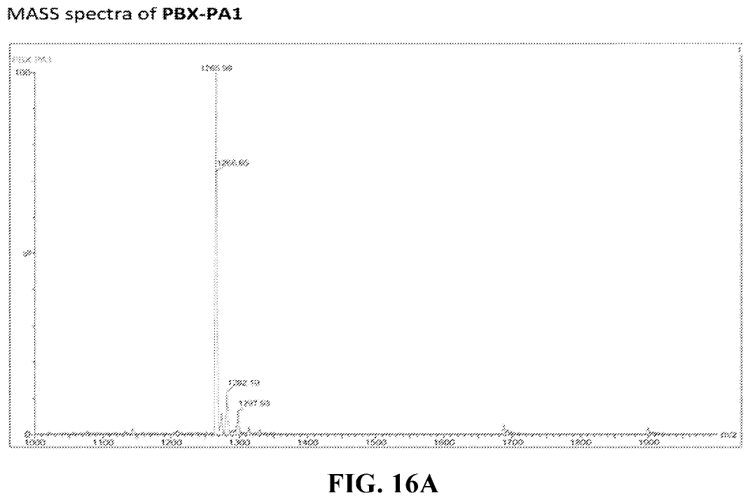

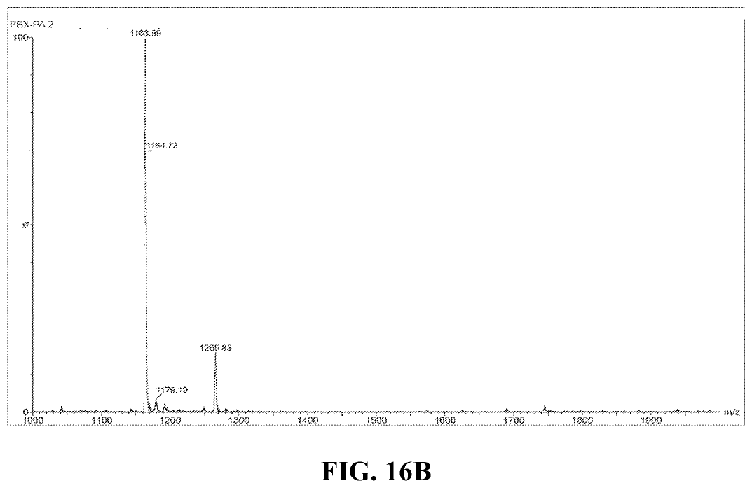

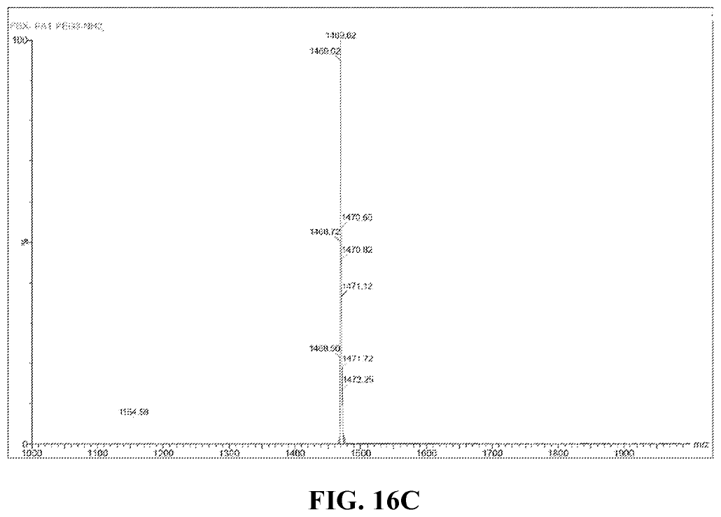

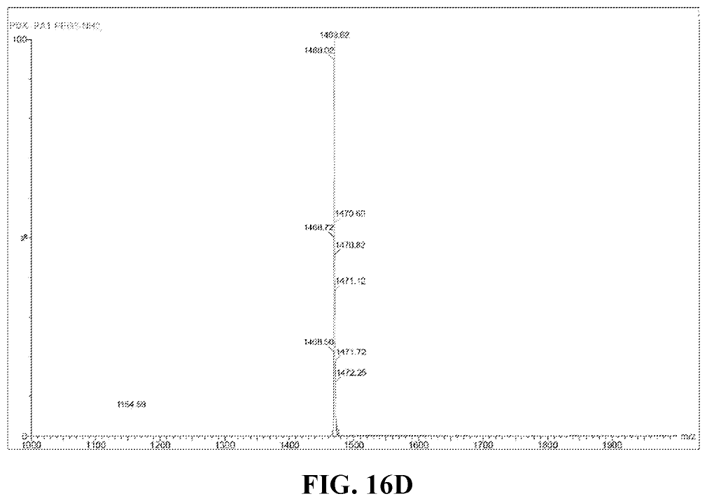

The mass spectra for PBX-PA1 is shown in FIG. 16A.

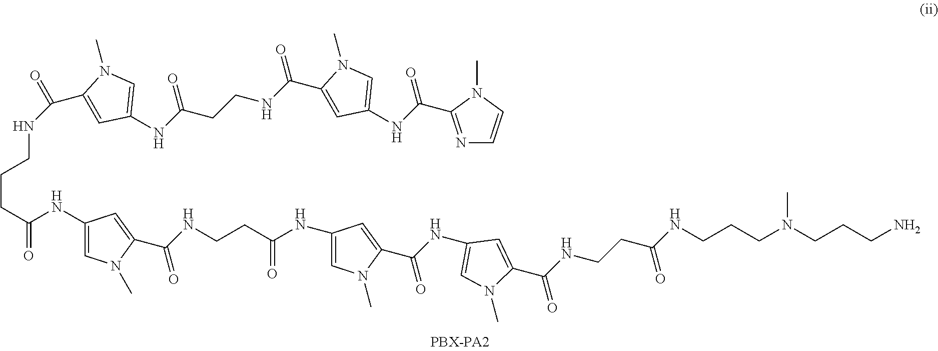 [SMILES: CN(CCCN)CCCNC(=O)CCNC(=O)c1cc(NC(=O)c2cc(NC(=O)CCNC(=O)c3cc(NC(=O)CCCNC(=O)c4cc(NC(=O)CCNC(=O)c5cc(NC(=O)c6nccn6C)cn5C)cn4C)cn3C)cn2C)cn1C]

The mass spectra for PBX-PA2 is shown in FIG. 16B.

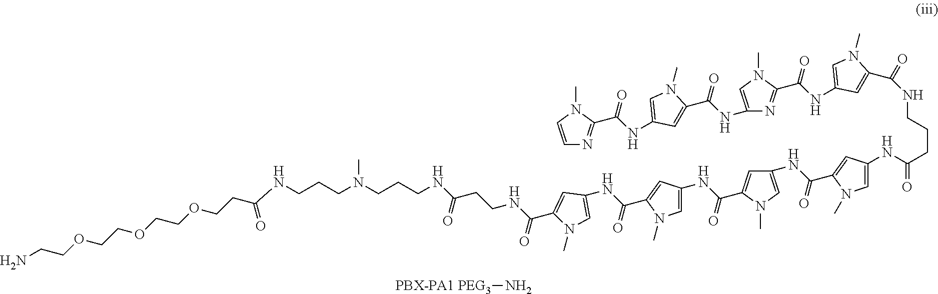

The mass spectra for PBX-PA1 PEG₃-NH₂ is shown in FIG. 16C.

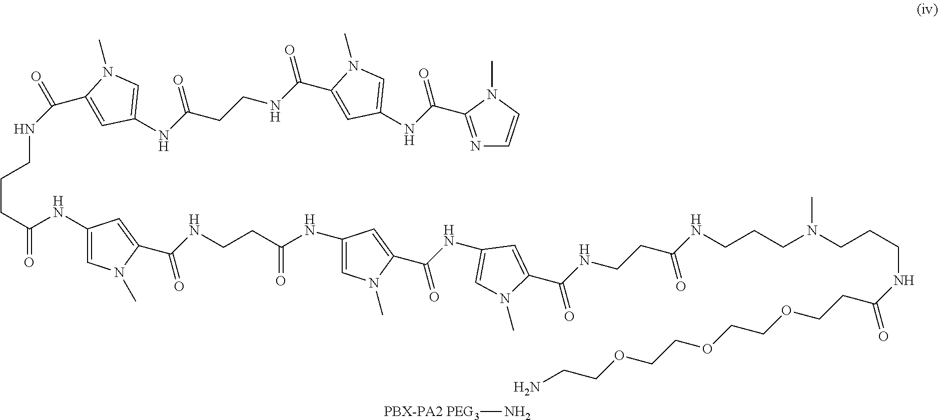

The mass spectra for PBX-PA2 PEG₃-NH₂ is shown in FIG. 16D.

### h. Peptide Synthesis

All peptides were synthesis by using standard coupling protocols (HATU, DIPEA, and DMF) and hydrolysis (10% NaOH) methods.

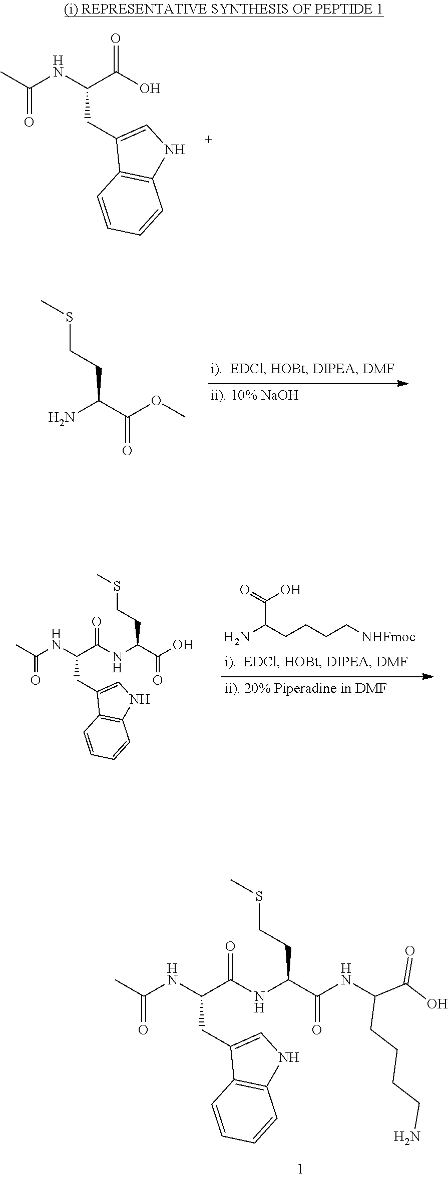

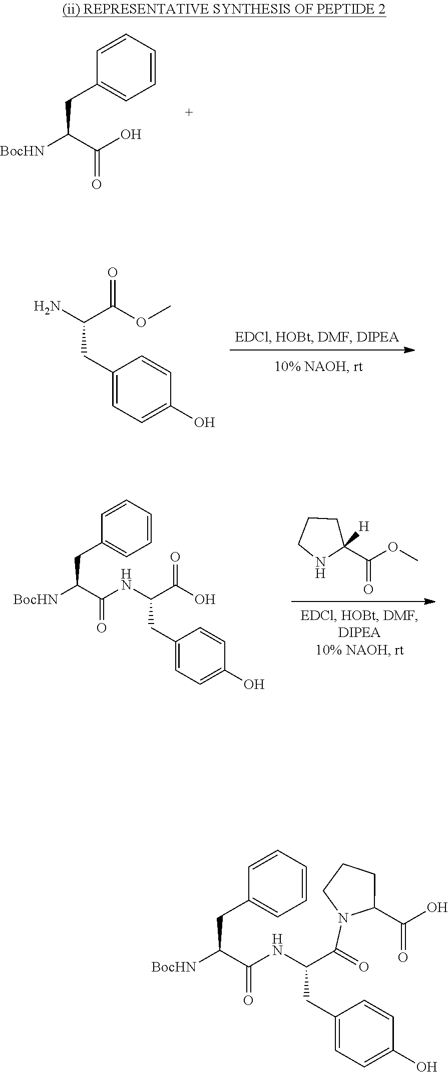

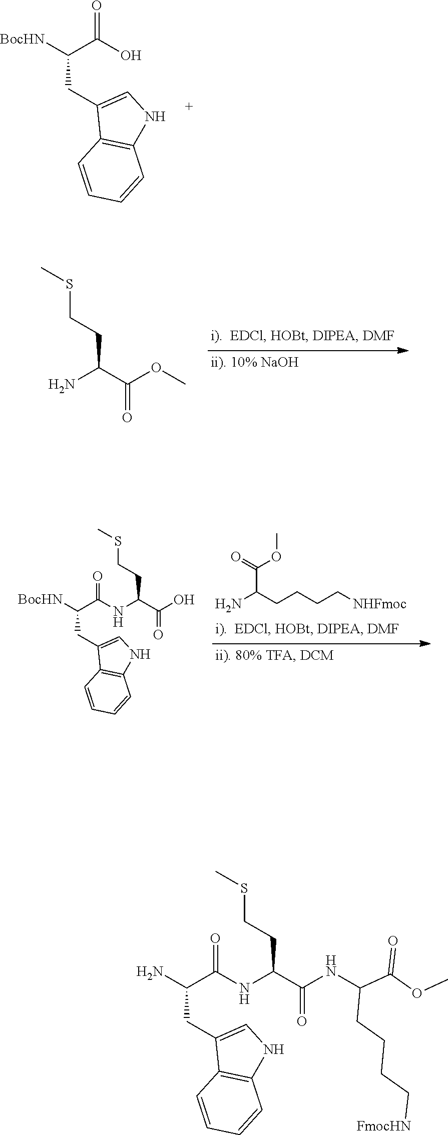

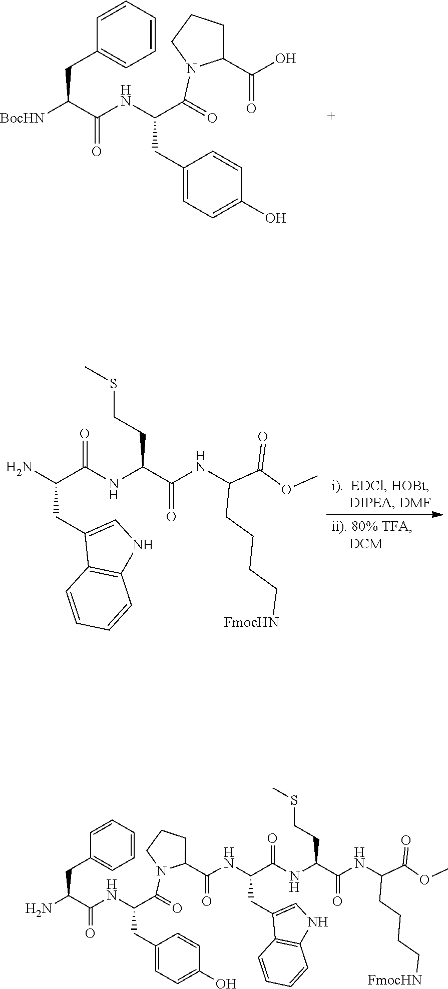

### (iii) Representative Synthesis of DRG-Peptide

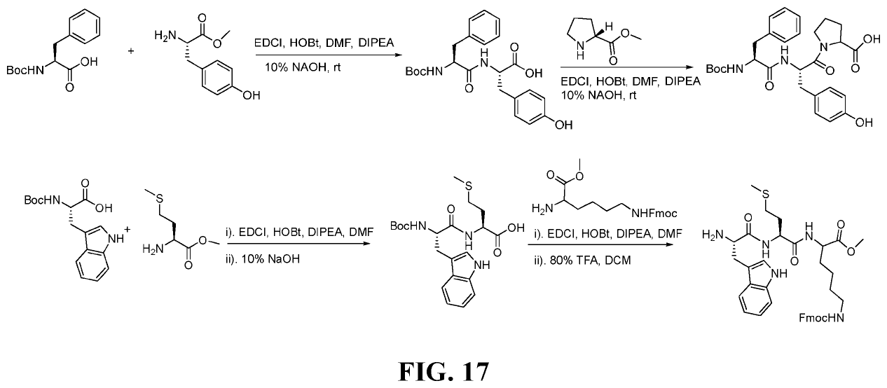

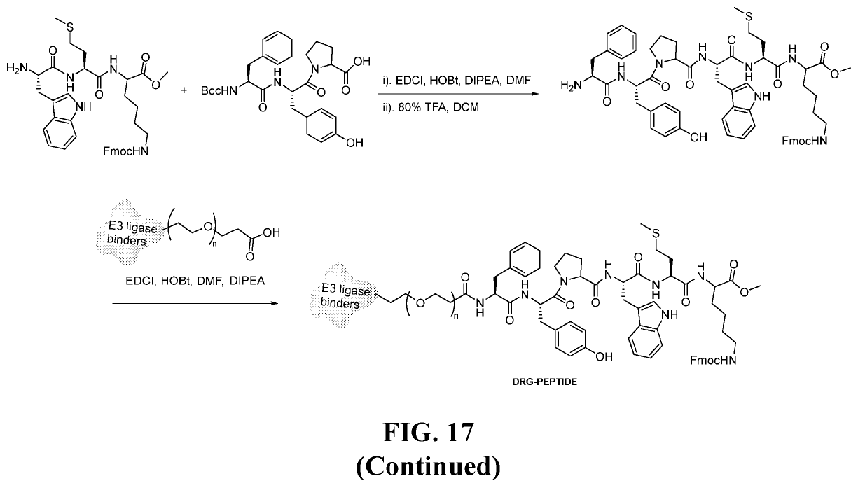

The synthesis of the DRG-Peptide is shown in FIG. 17.

### i. Synthesis of PBX-SynTEF-DGRs

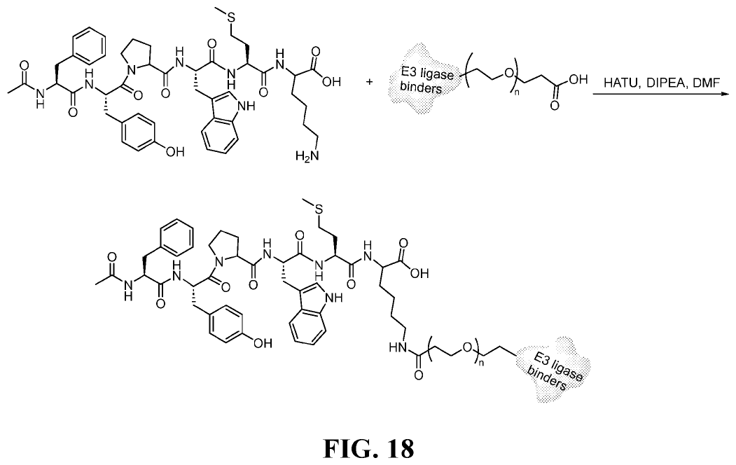

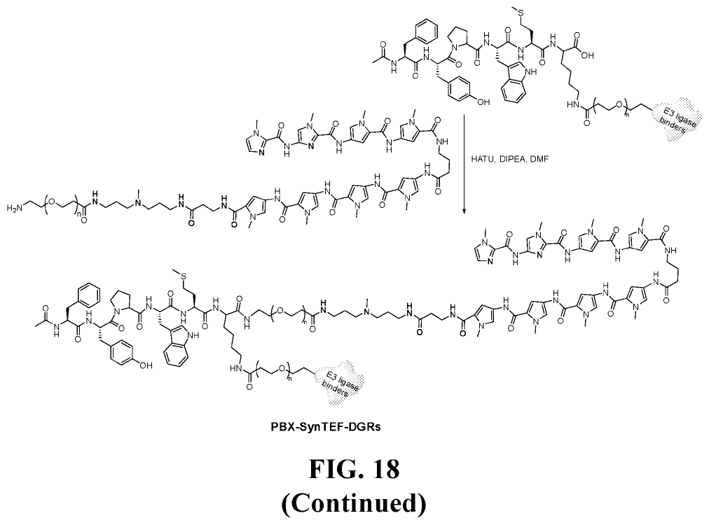

PBX-SynTEF-DGRs were synthesized as shown in FIG. 18.

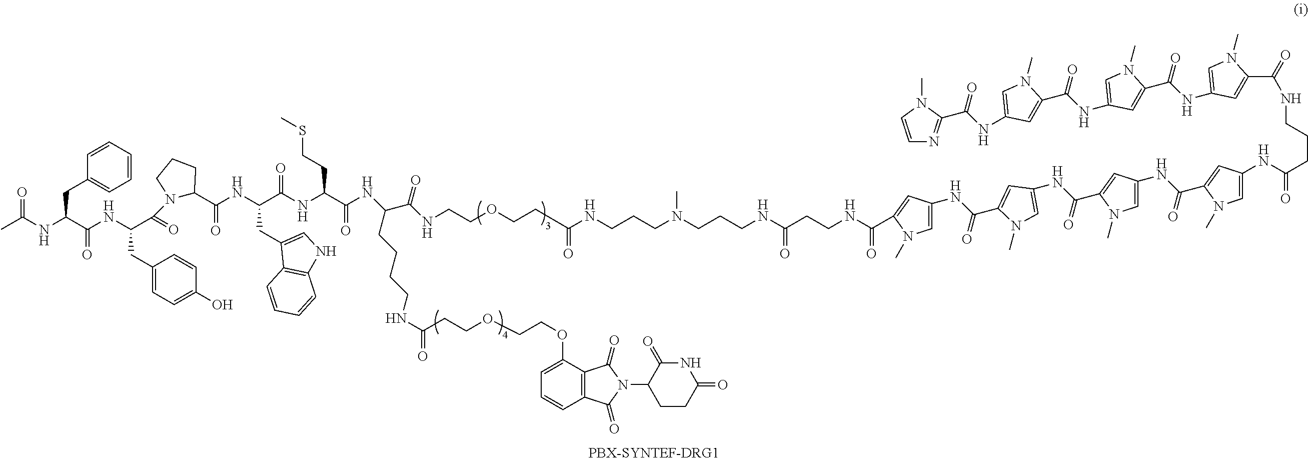

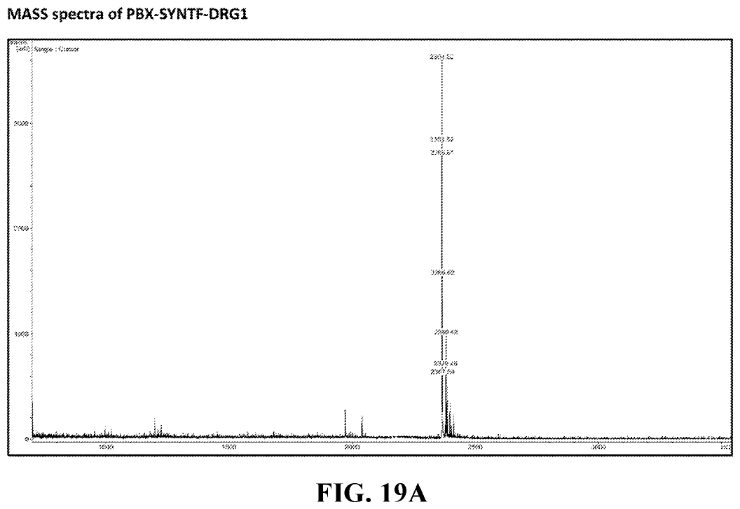

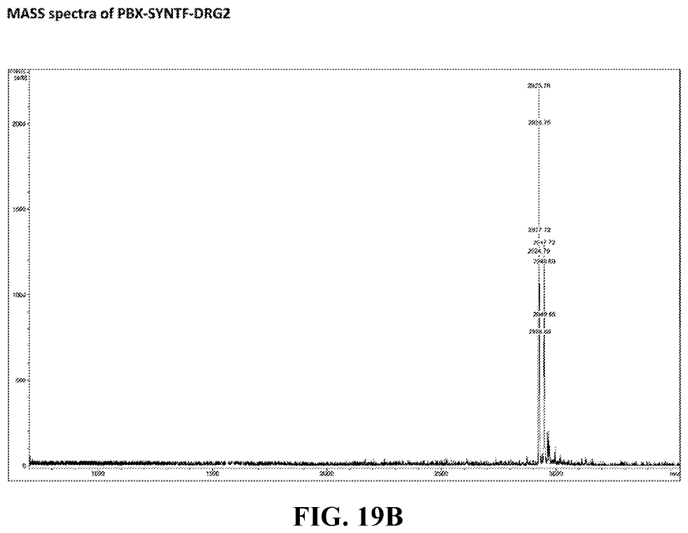

The mass spectra of PBX-SynTEF-DRG1 is shown in FIG. 19A.

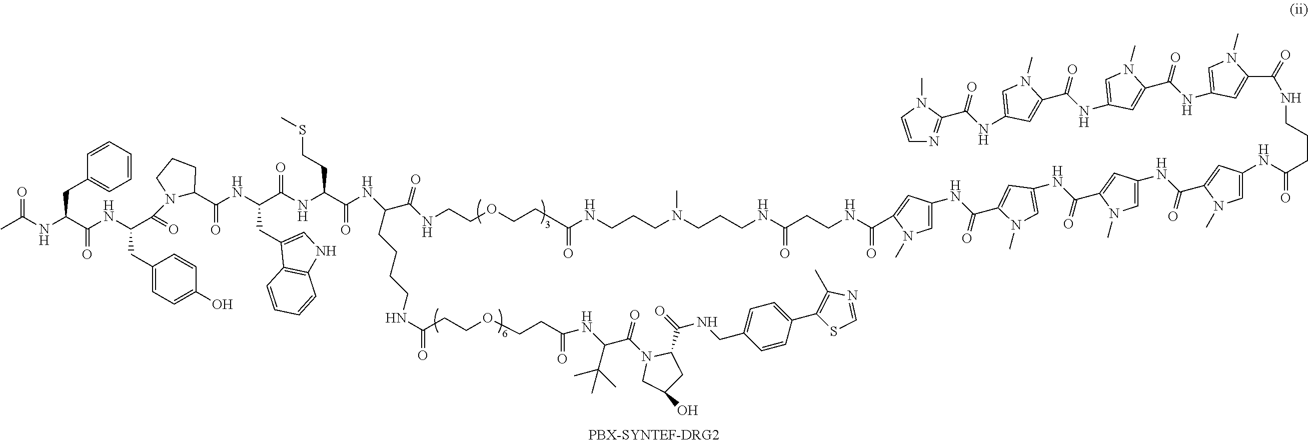

The mass spectra of PBX-SynTEF-DRG2 is shown in FIG. 19B.

### j. Synthesis of PBX-SynTEF-NDGRs

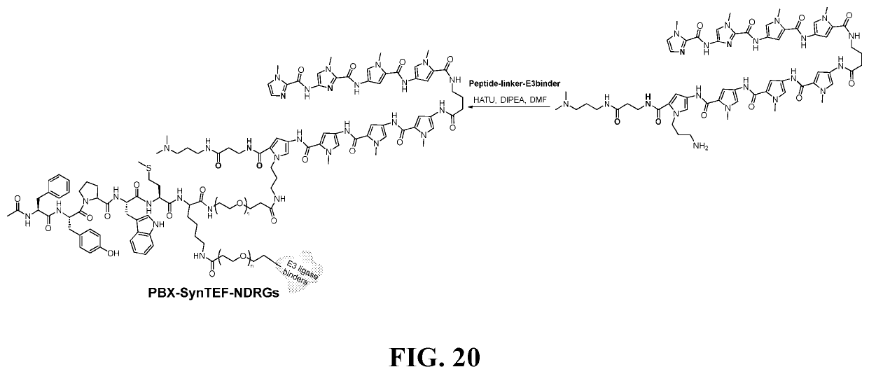

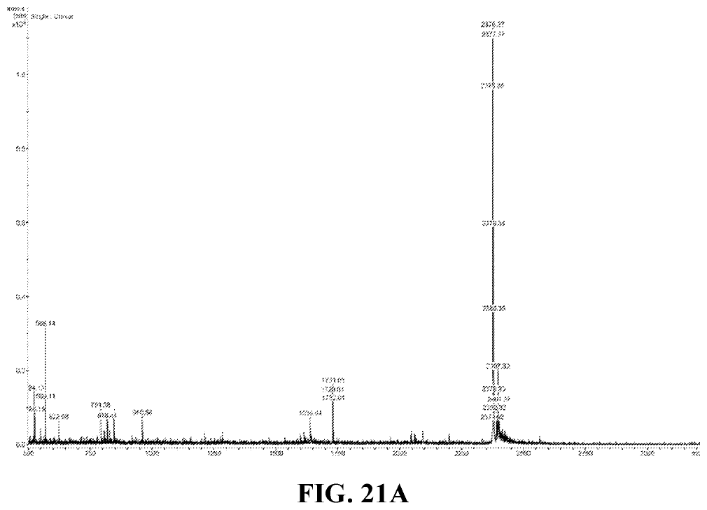

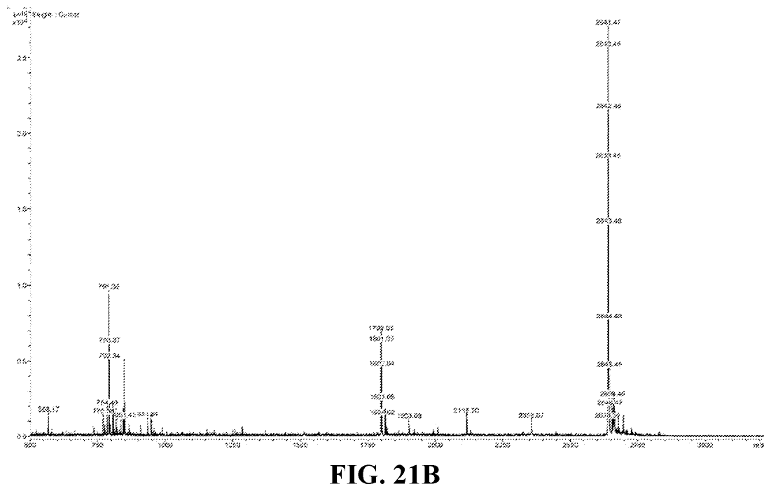

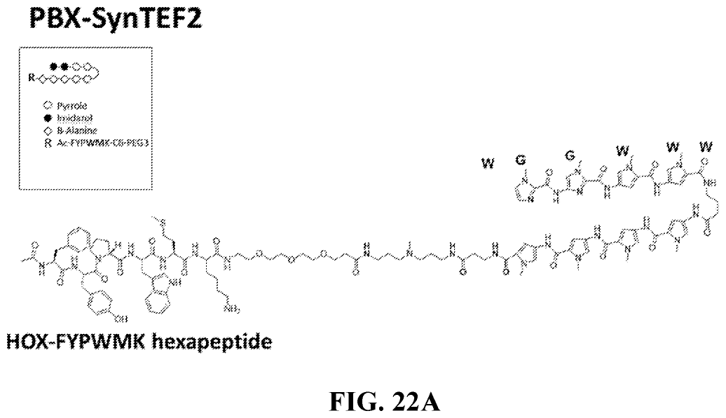

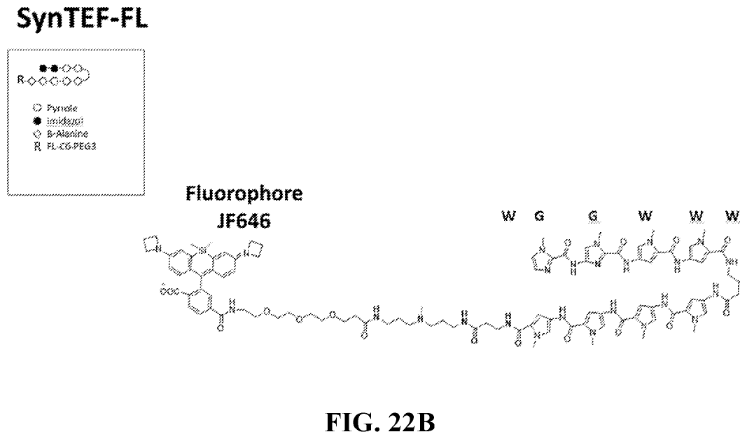

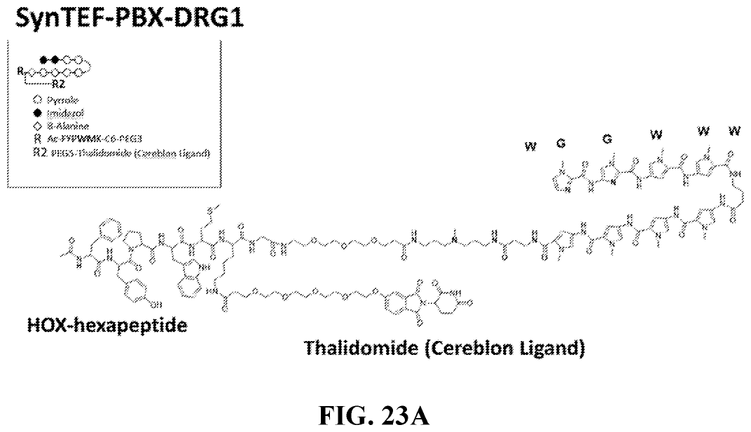

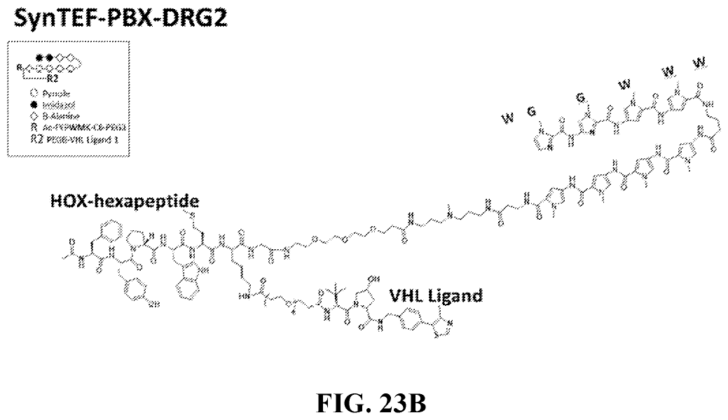

PBX-SynTEF-NDGRs were synthesized as shown in FIG. 20.

### 2. Biological Methods

a. Electrophoretic Mobility Shift Assays

Aliquots of PBX-SynTEF2 (200 nM) were incubated with 1 nM 5′ IRD680-labeled dsDNA (TGATTGATAAGGTAT) plus increasing amounts of TCF3-PBX1 (1.9-122 nM) and PBX1-HD-Ext (9.3-600 nM). After 1 hour incubation on ice, the samples were electrophoresed on 6% native acrylamide TBE gels (Novex) in chilled 0.5×TBE buffer at 100V. After 2-3 hours at 100V, the gels were imaged on an Odyssee-Fc Gel Imager (Li-Cor), and the IRD680 UV signal captured and analyzed.

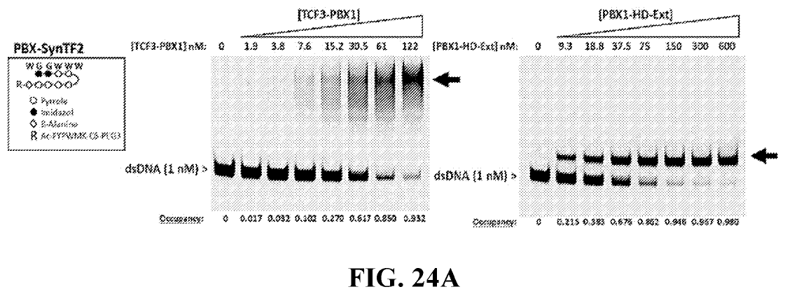

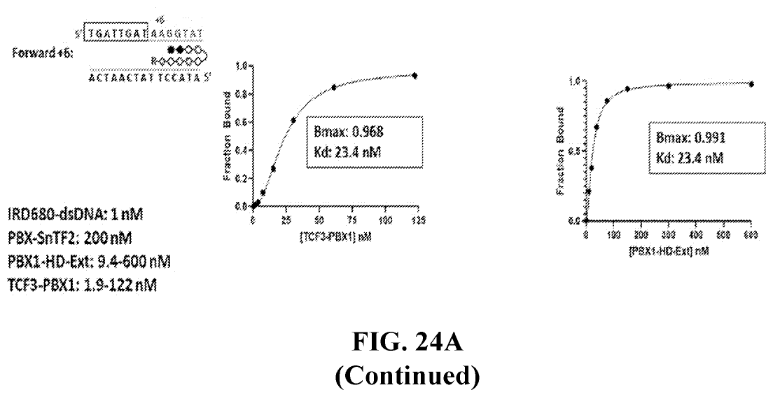

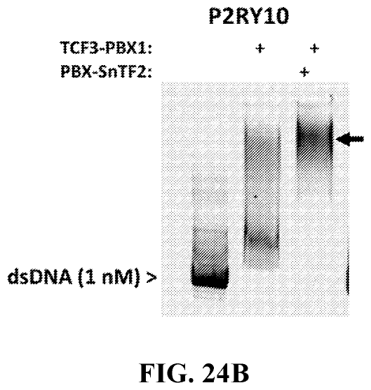

As shown in FIG. 24A, increasing amounts of both PBX1-HD-Ext and TCF3-PBX1 resulted in increased gel shifts of PBX1-HD-Ext-dsDNA and TCF3-PBX1-dsDNA complexes (arrows). The binding affinity of PBX-HD-Ext and TCF3-PBX1 for the dsDNA appeared to be similar.

As shown in FIG. 24B, 1 nM 5′ IRD680 labeled 282 bp dsDNA, a P2RY10 human gene promoter sequence in which we identified 9 PBX1 binding sites (TGAT or TGAC) and 5 WGGWWW PBX-SynTEF2 target sequences (unpublished), was incubated with 450 nM purified TCF3-PBX1, plus/minus 1 uM PBX-SynTEF2. After electrophoresis we detected gel shifted protein-DNA complexes in the presence of TCF3-PBX1 without PBX-SynTEF2 (middle lane), indicating that TCF3-PBX1 is capable in vitro of binding to the putative PBX1 and TCF3-PBX1 P2RY10 promoter. The addition of both TCF3-PBX1 and 1 uM PBX-SnTF2 resulted in the gel shift of all P2RY10 dsDNA to a more condensed higher molecular weight TCF3-PBX1-DNA complex (lane 3, arrow), indicating that the TCF3-PBX1/PBX-SynTEF2 complex has increased binding affinity for the P2RY10 promoter fragment.

b. P2RY10 Luciferase Reporter Assay

Cells were transfected with P2RY10-Luciferase reporter plasmid DNA which contains the 282 bp P2RY10 promoter sequence cloned downstream of a minimal TATA box promoter. After 24 hours, the cells were treated with 0.70 and 1.0 μM PBX-SynTEF2 (SnTF2), SynTEF-PBX-DRG1 (DRG1), and SynTEF-PBX-DRG2 (DRG2). The SynTEF-PBX-DRG1 compound carries the Thalidomide ligand for recruitment of the Cerbelon E3-Ligase, and SynTEF-PBX-DRG2 has a VHL ligand for the recruitment of the VHL E3-Ligase. Doxycycline (DOX) was added at 0.1 ug/mL concentration to induce TCF3-PBX1-HaloTag expression, and 48 hours after DOX addition, NanoGlo Luciferase expression (Promega) was measured in the medium (Luciferase protein is secreted by the cells). Luciferase activity was calculated as Fold Change compared to “DMSO without DOX” control cells (purple bar), Error bars: Standard deviation. N>6 biological replicates.

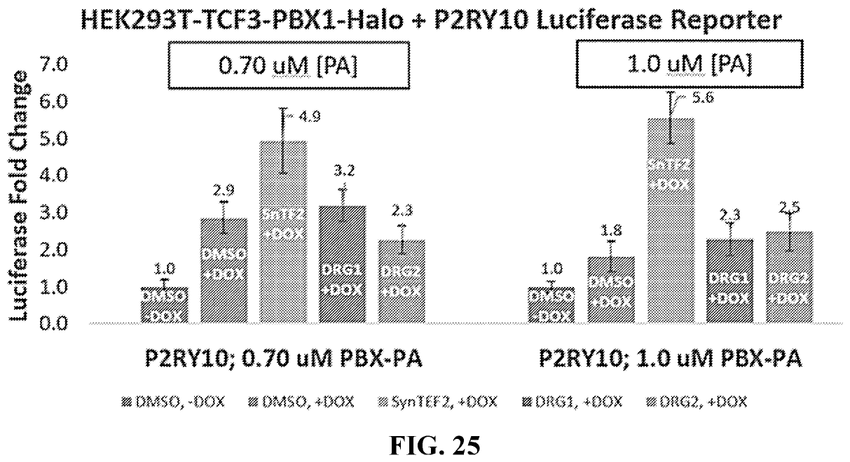

DOX-induction of TCF3-PBX1-HaloTag resulted in a ~2-3-fold increase in Luciferase expression (FIG. 25). This was not surprising, since TCF3-PBX1 is known to homodimerize or associate with endogenous HOX and TALE TFs, and as such is capable of transcriptional activation of gene targets. Uptake of PBX-SynTEF2 resulted in a higher 4.9 and 5.6-fold increase in Luciferase expression, at 0.70 and 1.0 uM PBX-PA concentration, respectively. This indicates that the WGGWWW-specific synthetic HOX mimic binds to the TCF3-PBX1-HaloTag TF and increases the capacity of this fusion TF to activate TCF3-PBX1 gene targets such as P2RY10. In contrast, the “PBX1 degrader compounds” SynTEF-PBX1-DRG1 and DRG2, whose polyamide and HOX-hexapeptide are identical to that of PBX-SynTEF2, but in addition carry E3-Ligase recruitment ligands, fail to further increase Luciferase expression (yellow and blue bars) above the “DMSO plus DOX” baseline levels at both 0.70 and 1.0 μM concentrations (red bars). Although this is indirect evidence, we can infer from these results that DRG1 and DRG2 E3-ligase recruitment to PBX-SynTEF-associated TCF3-PBX1-HaloTag, likely results in its Ubiquitylation and proteasomal degradation.

c. Cell Viability Assay

The N6-TCF3-PBX1-HaloTag cells were Nucleofected (Lonza) with P2RY10-Luciferase and CMV promoter Luciferase reporter plasmid DNAs. After 24 hours, cells were treated with either wildtype (WT) or Mutant (MT) Transferrin Receptor (TR) WGGWWW DNA-Aptamers pre-loaded with PBX-SynTEF-FL (SnTF-FL), SynTEF-PBX-DRG1 (DRG1), and SynTEF-PBX-DRG2 (DRG2). The WT and MT DNA-Aptamers contain WGGWWW tail duplex DNA sequences that bind the PBX-SynTEF payloads, but only WT DNA-Aptamers can bind to TR present on the cell surface of NALM6 ALL cells, resulting in TR-mediated cellular internalization of the polyamide compounds. The MT DNA-Aptamer, has a GG to AA dinucleotide substitution which prevents binding to the TR (*see Cheng et al, 2022). Thus, cells treated with the MT DNA-Aptamer-SynTEF complexes serve as negative controls, since without a functional Aptamer, it is unlikely that the polyamides are taken up by the NALM6 cells. Doxycycline (DOX) was added at 0.1 ug/mL concentration to induce TCF3-PBX1-HaloTag expression, and 48 hours after DOX addition, cell viability was measured using CellTiterGlo reagent (Promega) which detects ATP present only in live cells. CellTiterGlo activity was calculated as Fold Change compared to “DMSO no DOX” treated cells which had the highest cell viability (dark blue bars). Error bars: Standard deviation. N>6 biological replicates.

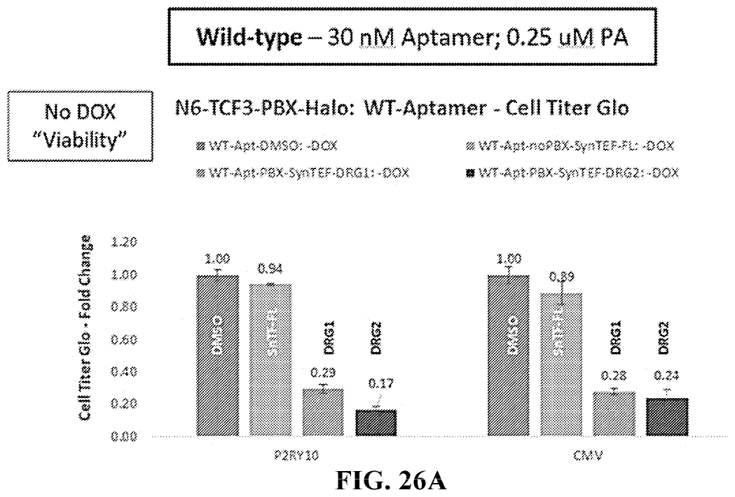

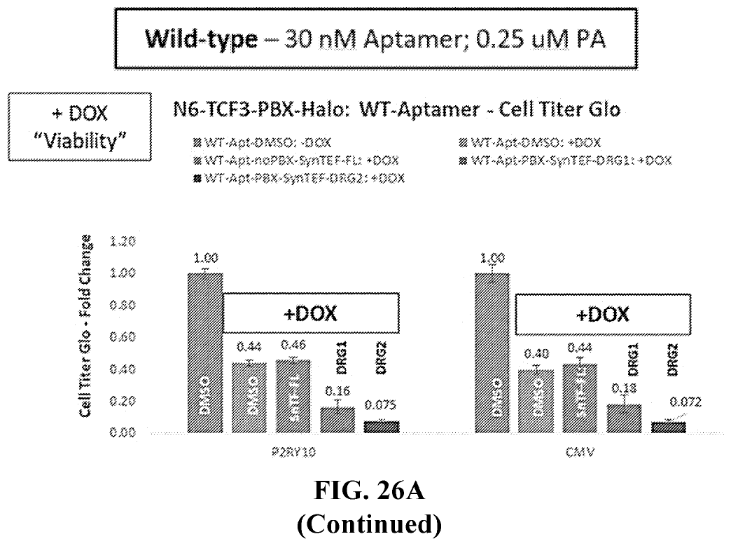

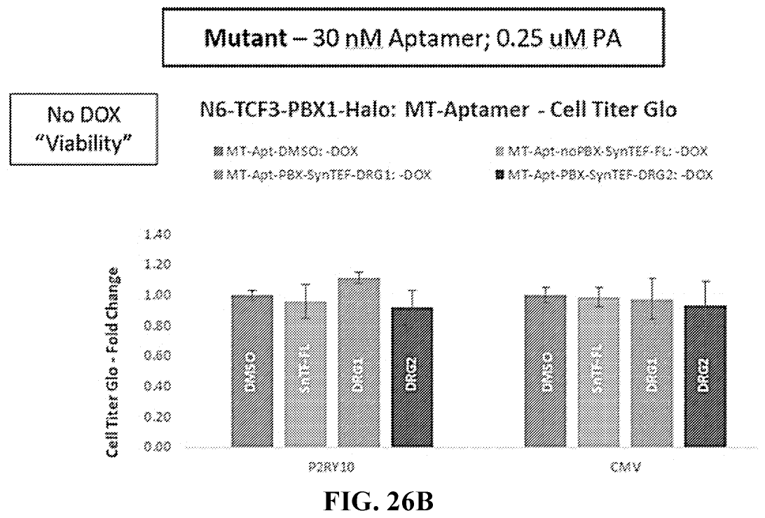

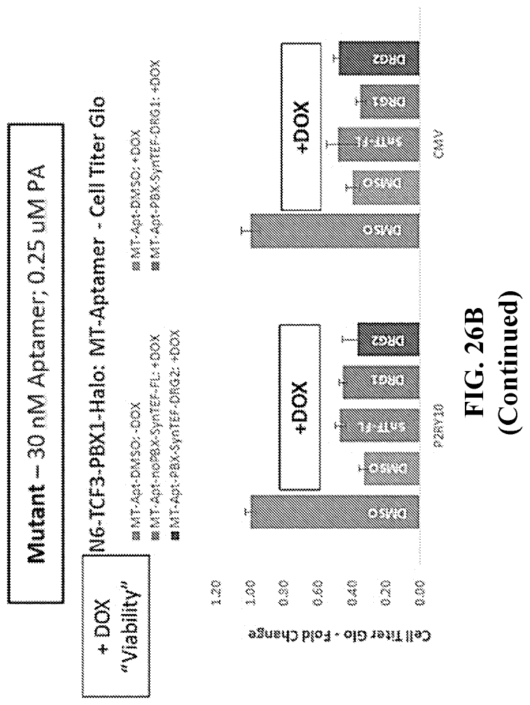

As shown in FIG. 26A, without DOX treatment (FIG. 26A, top), WT-Aptamer-SynTEF-FL treated cells had only a marginal loss in cell viability in P2RY10- and CMV nucleofected cells (grey bars), compared to DMSO-treated control cells (dark blue bars). However, in contrast, WT-Aptamer-SynTEF-DRG1 and DRG2 treated cells had a 70-80% loss in cell viability in both P2RY10 and CMV nucleofected cells (light blue and purple bars, respectively). In even starker contrast, none of the MT-Aptamer-SynTEF treated cells (FIG. 26B, top) showed any loss of cell viability, indicating that SynTEF-PBX-DRG1 and DRG2 are toxic to the cells but only when internalized. Moreover, because the WGGWWW SynTEF-FL polyamide compound is not toxic to the cells, we deduce that the toxicity of the PBX-SynTEF-DRG1 and DRG2 compounds must be caused by the covalently attached DRG1 and DRG2 ligands. With DOX treatment (FIG. 25A, bottom), both WT-Aptamer-SynTEF-FL and DMSO treated cells (orange and red bars, respectively) had a 40-60% loss in cell viability compared to “DMSO no DOX” treated control cells (dark blue bars). However, WT-Aptamer-SynTEF-DRG1 and DRG2 treated+DOX cells had an even much higher loss in cell viability, ~85% for DRG1 (green bars) and >90% for DRG2 (brown bars). Once again, and in stark contrast, of the “MT-Aptamer-SynTEF+DOX” treated cells (FIG. 25B, bottom), DRG1 (green bars) and DRG2 (brown bars) had comparable viability to “+DOX DMSO” (red bars) and “+DOX SynTF-FL” (yellow bars) cells. Thus, toxicity of SynTEF-PBX-DRG1 and DRG2 in non-DOX treated NALM6-TCF3-PBX1-HaloTag cells (Bottom left panel) which don't express the fusion TF (analyzed on Western, not shown), is probaly caused by the recruitment of E3-ligases to endogenous PBX proteins that also bind to the synthetic SynTEF HOX mimics via the HOX-hexapeptide, resulting in their subsequent proteasomal degradation. Precursor B cells such as NALM6 express PBX2, 3 and 4, which are very homologous to PBX1, and like PBX1, are forming heterodimeric complexes with other endogenous HOX and TALE TFs, transcriptionally activating downstream gene targets. DRG1 and DRG2-induced depletion of one or more of the PBX proteins and attached TF binding partners may very well result in B-cell differentiation into more mature B cells with reduced cell viability, which is even more enhanced when TCF3-PBX1-HaloTag expression is induced by DOX treatment of the NALM6 cells. This would also mean that the SynTEF-PBX-degrader compounds could be therapeutically relevant not only in ALL patients with the TCF3-PBX1 subtype, but also in ALL patients with other subtypes, and possibly extend to other malignancies in which overexpression of PBX1-4 HOX TFs are known to play roles in maintaining a stemness phenotype.

### 3. Development of SynGR-Degraders

As detailed herein, SynGRaders would enable targeted degradation or non-proteolytic inactivation of DNA binding proteins that drive or contribute to a range of pathologies. In a sense, these degraders represent a distinct class of PROTACs that function when assembled on a DNA, in a sequence-selective or non-selective manner. Unlike typical PROTACs, SynGRaders confer selectivity of action on proteins that are bound to the genome rather than in bulk cellular milieu. Moreover, due to DNA-templated assembly, ligands with moderate-weak affinity would become more potent and/or effective in engaging their target proteins and promoting their degradation or inactivation. Both these properties are embodied in the PBX-SynTF-DRG1 molecule that uses a YPWM bearing peptide to bind a shallow hydrophobic pocket on the surface of PBX or TCF3-PBX and leads to the reduced activity of the oncogenic TCF3-PBX1 fusion in cell-based reporters.

TCF3-PBX1 is an oncogenic transcription factor resulting from a chromosomal translocation fusing the transcription factors TCF3 (aka E2A) and PBX1. While able to oligomerize on its own, TCF3-PBX1 also interacts with the HOX and/or MEIS transcription factors to drive target gene expression. Ectopic expression of TCF3-PBX1 promotes proliferation and survival of pre-B cells and leads to pediatric Acute Lymphoblastic Leukemia (ALL). PBX-SynTF-DRG1 competes with endogenous HOX transcription factors to bind to TCF3-PBX1 protein and to specific DNA binding sites in the promoters of target genes, including the Purinergic Receptor P2Y (P2RY10), of TCF3-PBX1 in ALL. PBX-SynTF-DRG1 and derivatives have the potential, dependent on the attached DRG ligand, to (i) recruit an E3 Ligase, (ii) selectively ubiquitylate TCF3-PBX1, and (iii) promote its proteasomal degradation, (iv) thereby perturbing transcriptional activity of said target genes. In turn, degradation of TCF3-PBX1 has the potential to inhibit cell proliferation and induce cell death. In addition, this therapeutic modality could be combined with other chemotherapeutic drugs, potentially improving their efficacy.

a. VHL-PEG8-GMA-SYNTEF1

The disclosed VHL-PEG8-GMA-SynTEF1 bi-functional molecule bears a polyamide designed to target ^{5′}AAGAAGAAG^{3′} repeats that is tethered via chemical linkers to a small molecule (e.g., thalidomide) that engages the Ubiquitin-Proteosome System (UPS) at specific GAA-rich genomic loci. As detailed here, the DNA-binding polyamide moiety, the linker, and the UPS-targeting moiety can be replaced to target desired DNA sequences and to engage distinct members of the UPS machinery to achieve the goal of proximity-induced, DNA-templated protein degradation/inactivation.

b. PBC-SynTF-DRG1

The disclosed PBX-SynTF-DRG1 tri-functional molecule bears a polyamide engineered to target ^{5′}WGGWWW^{3′} DNA binding sites tethered via variable chemical linkers to a PBX or TCF3-PBX transcription factor binding moiety (e.g., YPWM peptide and its various peptidic and non-peptidic derivatives), and further tethered to a small molecule that engages the UPS machinery. The molecule is designed to promoter DNA-templated degradation of a targeted transcription factor such as PBX and the oncogenic TCF3-PBX. The modular design of this prototype lends itself to replacement of each unit and the tethering linkers to create molecules to engage and degrade/inactivate proximal proteins and is not limited to PBX/TCF3-PBX.

### 4. Exemplary Degraders

A summary of the exemplary bi-functional degraders having a polyamide linked to an E3 ligase binding ligand is shown in Table 1 below.

| No. | Structure |
|---|---|
| PA1- DRG1 | 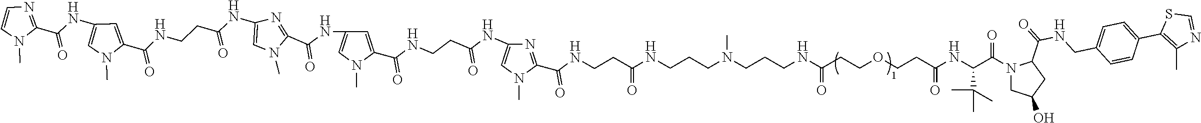 |
| PA1- DRG2 | 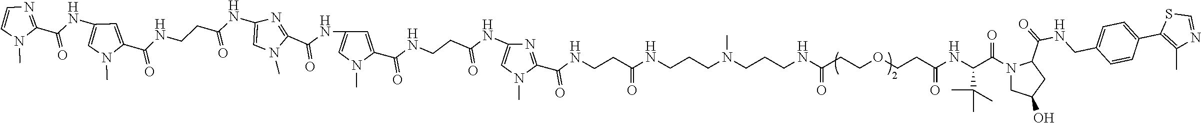 |
| PA1- DRG3 | 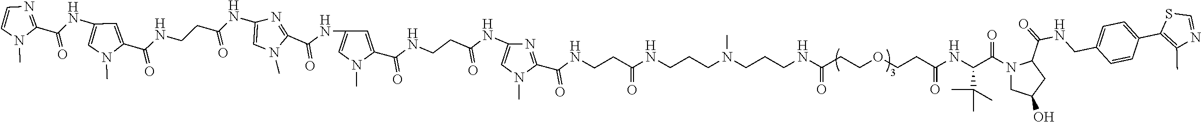 |
| PA1- DRG4 | 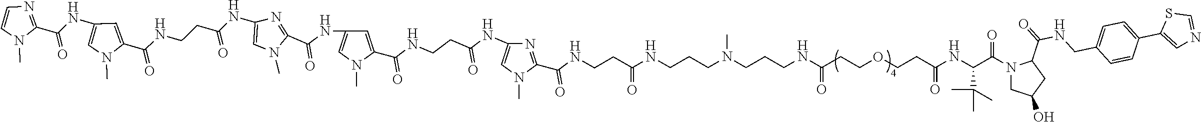 |
| PA1- DRG5 | 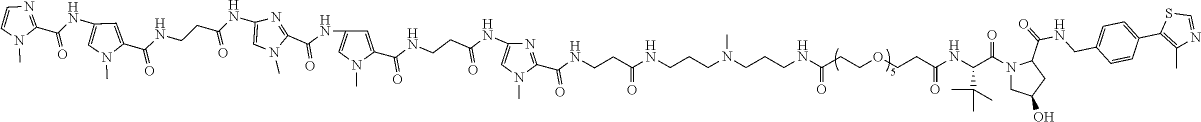 |
| PAI- DRG7 | 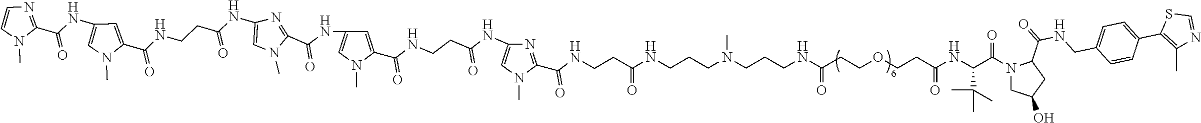 |
| PA1- DRG8 | 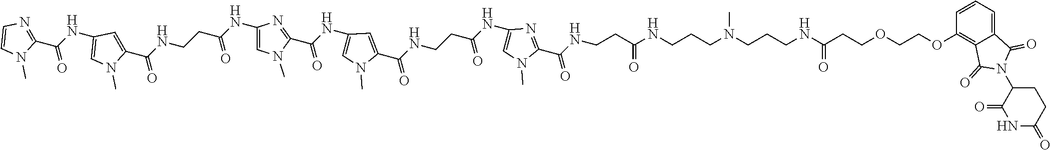 [SMILES: CN(CCCNC(=O)CCNC(=O)c1nc(NC(=O)CCNC(=O)c2cc(NC(=O)c3nc(NC(=O)CCNC(=O)c4cc(NC(=O)c5nccn5C)cn4C)cn3C)cn2C)cn1C)CCCNC(=O)CCOCCOc1cccc2c1C(=O)N(C1CCC(=O)NC1=O)C2=O] |
| PAI- DRG9 | 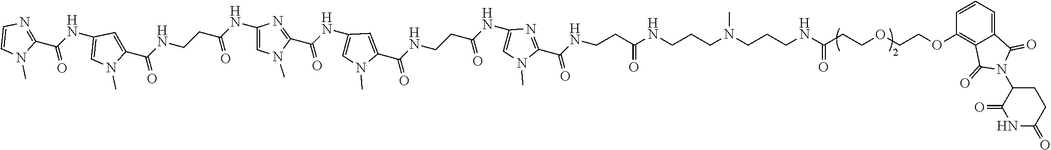 |
| PA1- DRG10 | 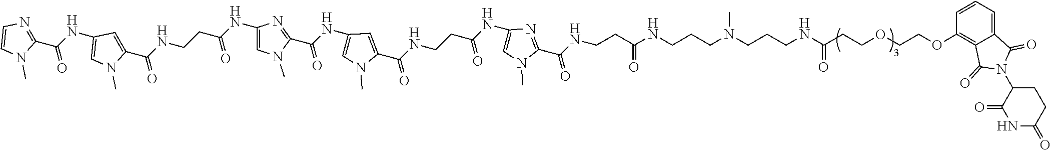 |
| PA1- DRG11 | 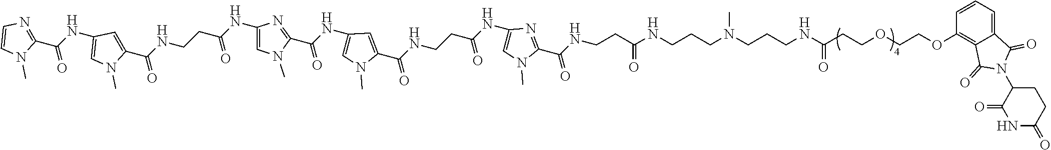 |
| PA1- DRG12 | 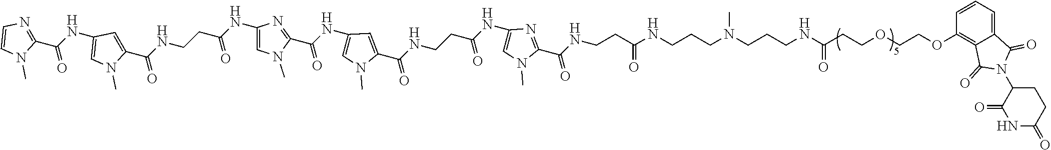 |
| PAI- DRG13 | 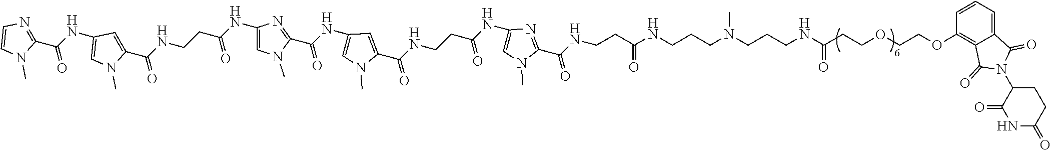 |
| PBX- SynTEF- DRG1 | 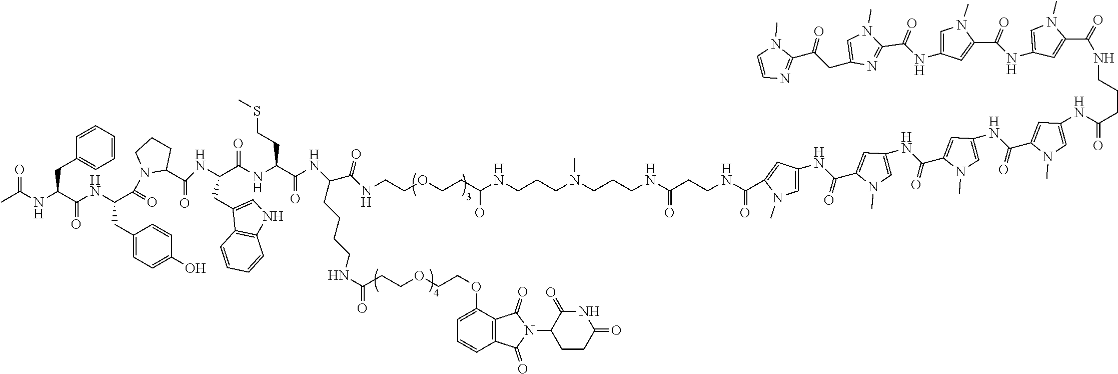 |
| PBX- SynTEF- DRG2 | 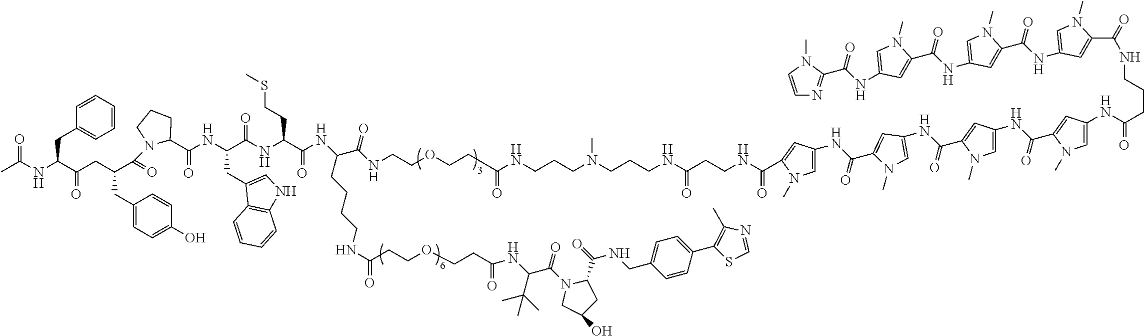 |

A summary of the exemplary tri-functional degraders having a BET binding ligand linked to a polyamide, which is further linked to an E3 ligase binding ligand is shown in Table 2 below.

| No. | Structure |
|---|---|
| SynTEF- DRG1 | 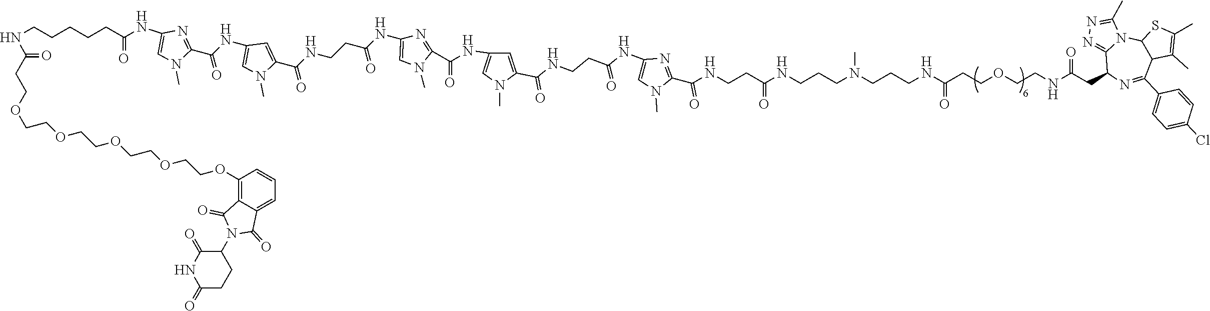 |
| SynTEF- DRG2 | 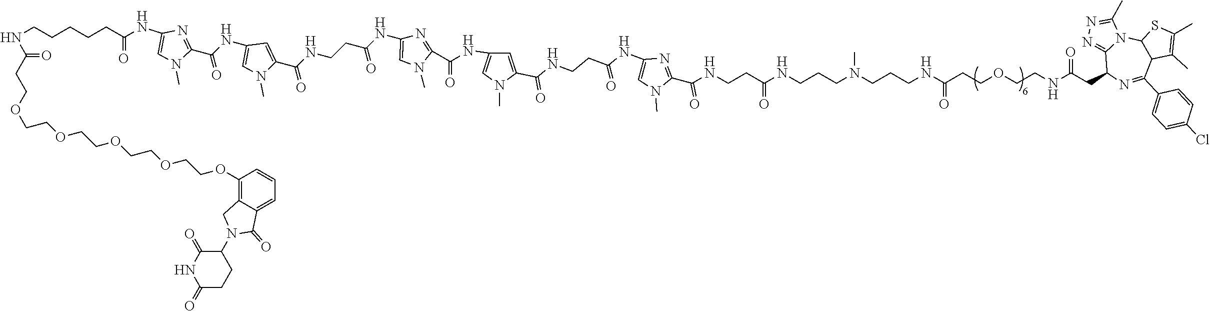 |
| SynTEF- DRG3 | 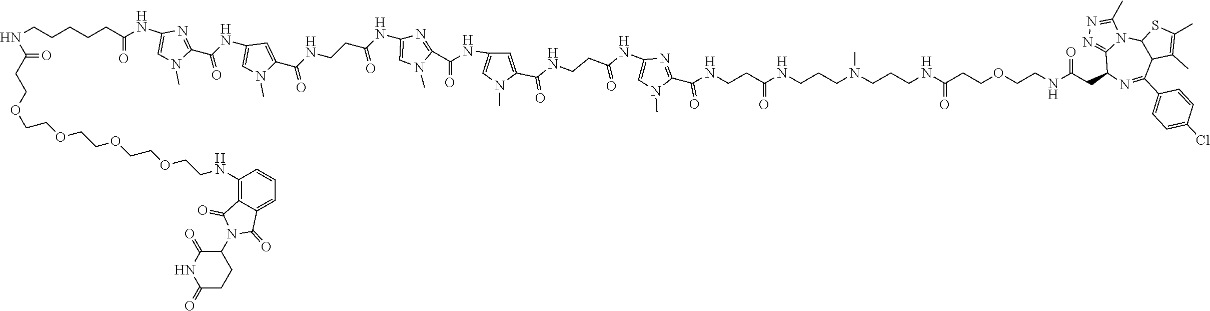 [SMILES: CC1=C(C)C2C(c3ccc(Cl)cc3)=N[C@@H](CC(=O)NCCOCCC(=O)NCCCN(C)CCCNC(=O)CCNC(=O)c3nc(NC(=O)CCNC(=O)c4cc(NC(=O)c5nc(NC(=O)CCNC(=O)c6cc(NC(=O)c7nc(NC(=O)CCCCCNC(=O)CCOCCOCCOCCOCCNc8cccc9c8C(=O)N(C8CCC(=O)NC8=O)C9=O)cn7C)cn6C)cn5C)cn4C)cn3C)c3nnc(C)n3C2S1] |
| SynTEF- DRG4 | 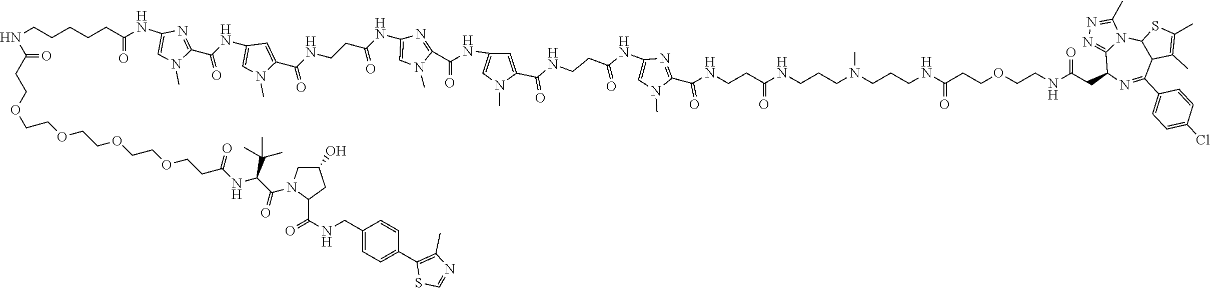 [SMILES: CC1=C(C)C2C(c3ccc(Cl)cc3)=N[C@@H](CC(=O)NCCOCCC(=O)NCCCN(C)CCCNC(=O)CCNC(=O)c3nc(NC(=O)CCNC(=O)c4cc(NC(=O)c5nc(NC(=O)CCNC(=O)c6cc(NC(=O)c7nc(NC(=O)CCCCCNC(=O)CCOCCOCCOCCOCCC(=O)N[C@H](C(=O)N8C[C@H](O)CC8C(=O)NCc8ccc(-c9scnc9C)cc8)C(C)(C)C)cn7C)cn6C)cn5C)cn4C)cn3C)c3nnc(C)n3C2S1] |
| SynTEF- DRG5 | 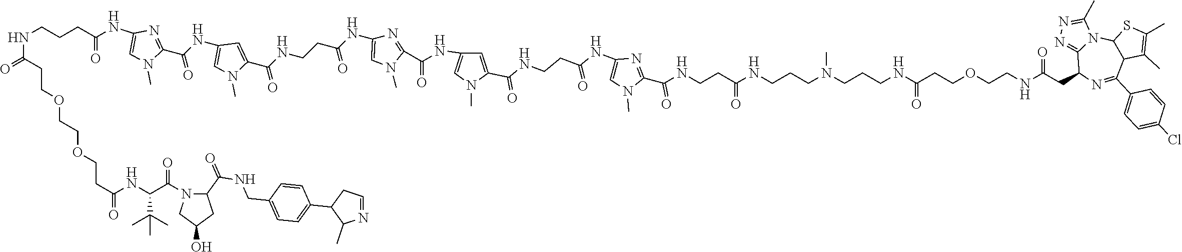 [SMILES: CC1=C(C)C2C(c3ccc(Cl)cc3)=N[C@@H](CC(=O)NCCOCCC(=O)NCCCN(C)CCCNC(=O)CCNC(=O)c3nc(NC(=O)CCNC(=O)c4cc(NC(=O)c5nc(NC(=O)CCNC(=O)c6cc(NC(=O)c7nc(NC(=O)CCCNC(=O)CCOCCOCCC(=O)N[C@H](C(=O)N8C[C@H](O)CC8C(=O)NCc8ccc(C9CC=NC9C)cc8)C(C)(C)C)cn7C)cn6C)cn5C)cn4C)cn3C)c3nnc(C)n3C2S1] |
| SynTEF- DRG6 | 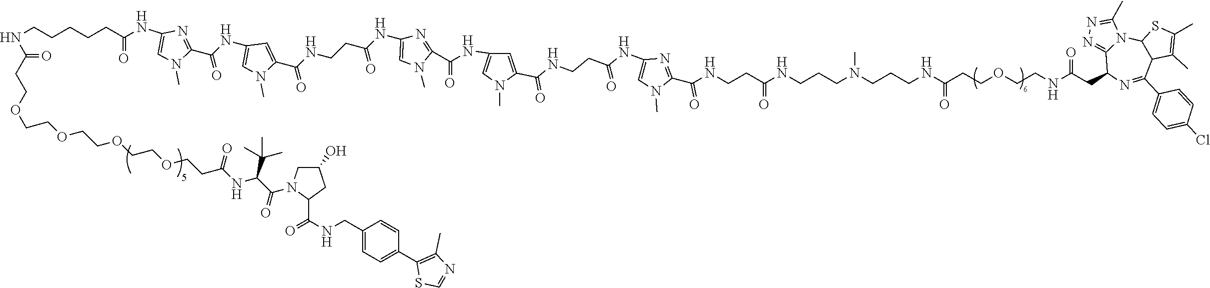 |
| SynTEF- DRG7 | 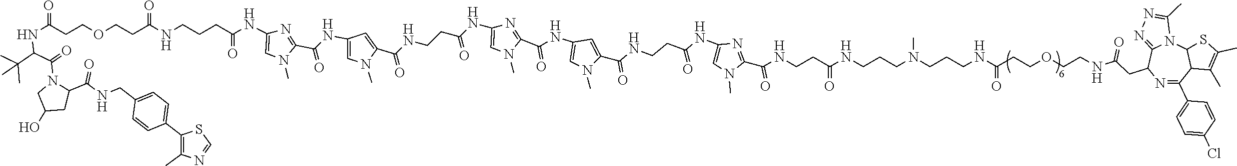 |
| SynTEF- DRG8 | 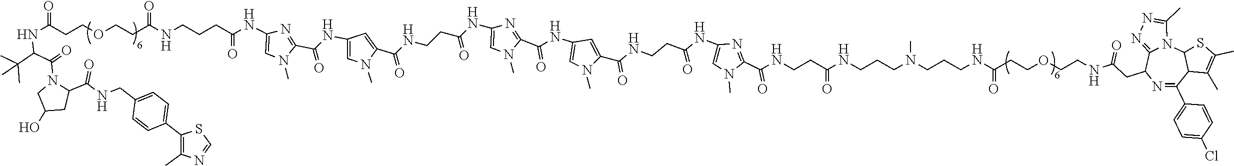 |
| SynTEF- DRG9 | 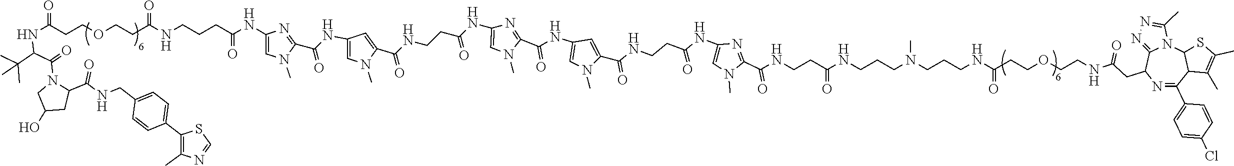 |
| SynTEF- DRG10 | 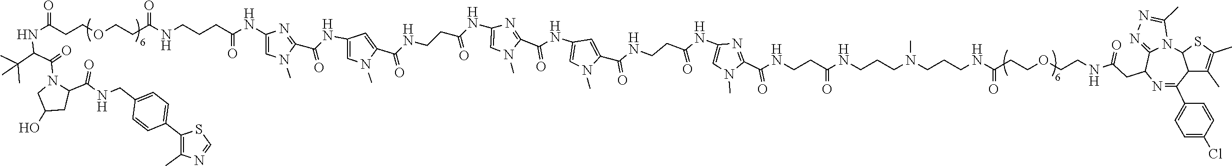 |

A summary of the exemplary bi-functional degraders having a polyamide linked to a TCF3-PBX binding peptide, which can be used to prepare tri-functional degraders, as further described herein, is shown in Table 3 below.

| No. | Structure |
|---|---|
| PBX- SynTF1 | 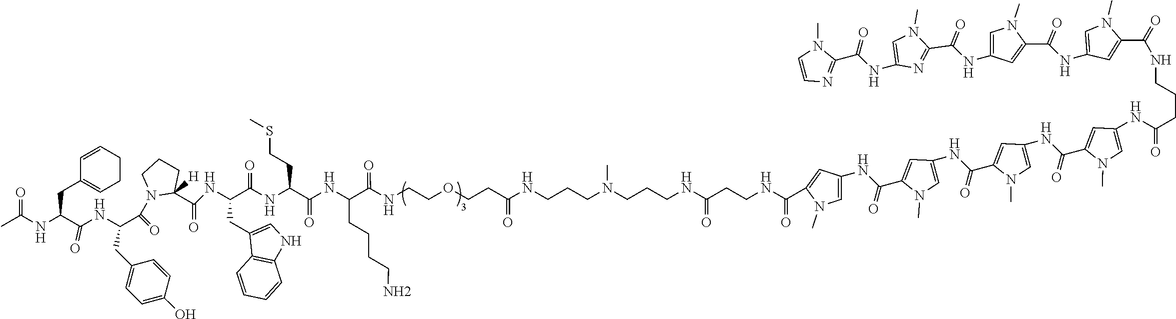 |
| PBX- SynTF2 | 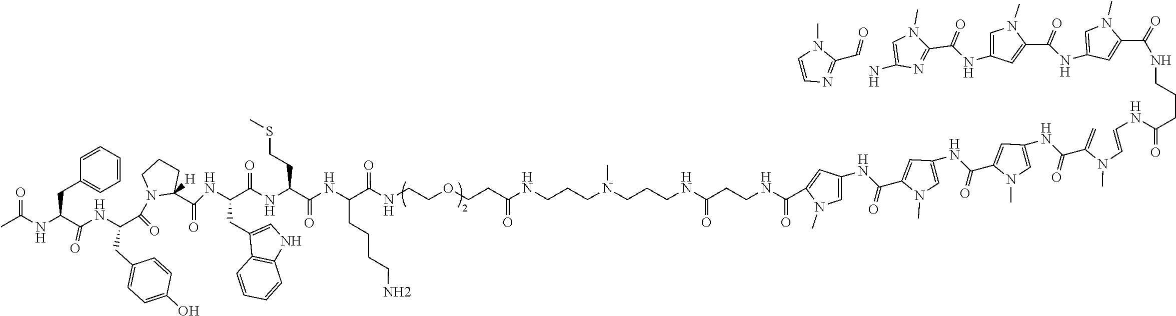 |
| PBX- SynTF3 | 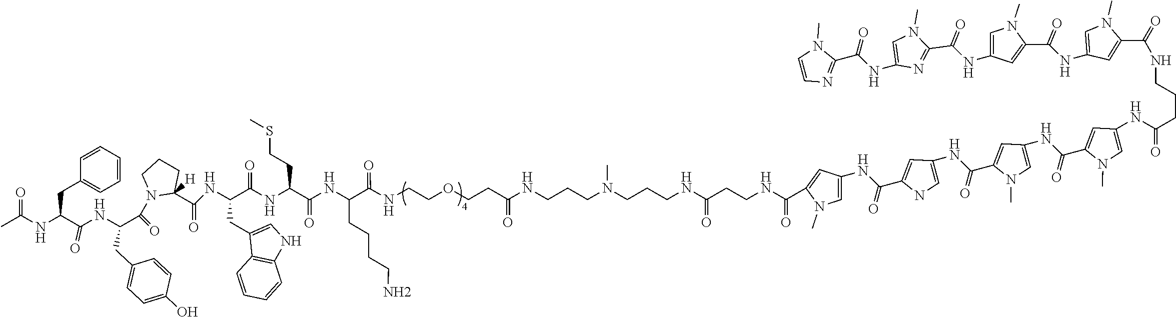 |
| PBX- SynTF4 | 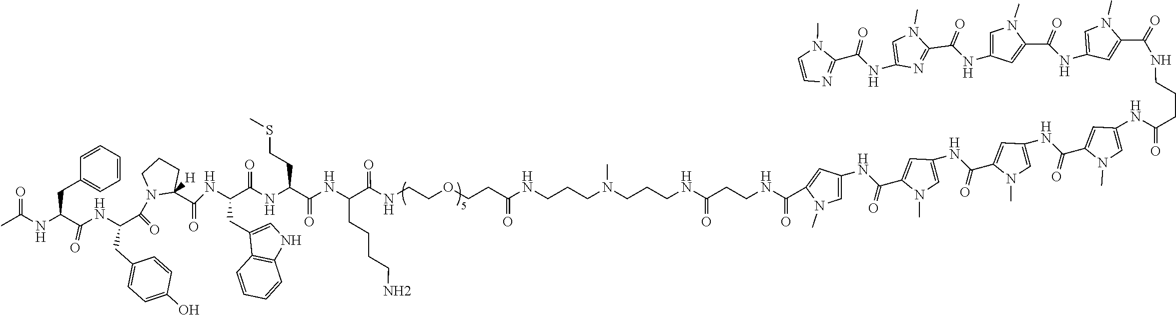 |

A summary of the exemplary tri-functional degraders having a polyamide linked to a TCF3-PBX binding peptide, which is further linked to an E3 ligase binding ligand is shown in Table 4 below.

| No. | Structure |
|---|---|
| PBX- SynTEF- DRG1 | 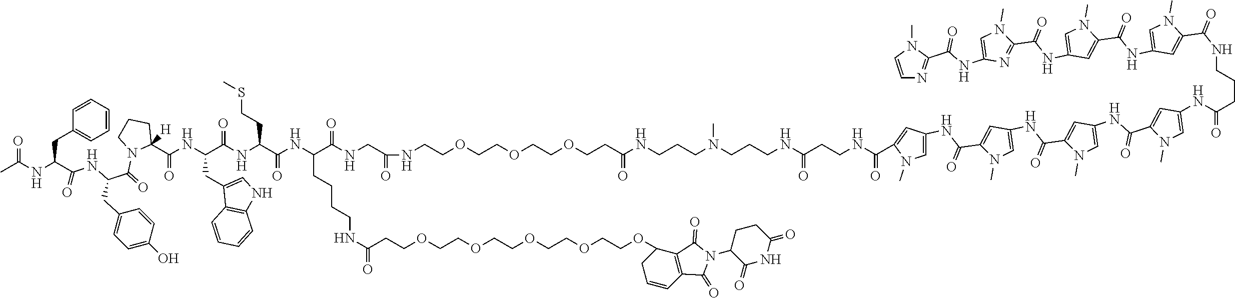 [SMILES: CSCC[C@H](NC(=O)[C@H](Cc1c[nH]c2ccccc12)NC(=O)[C@H]1CCCN1C(=O)[C@H](Cc1ccc(O)cc1)NC(=O)[C@H](Cc1ccccc1)NC(C)=O)C(=O)NC(CCCCNC(=O)CCOCCOCCOCCOCCOC1CC=CC2=C1C(=O)N(C1CCC(=O)NC1=O)C2=O)C(=O)NCC(=O)NCCOCCOCCOCCC(=O)NCCCN(C)CCCNC(=O)CCNC(=O)c1cc(NC(=O)c2cc(NC(=O)c3cc(NC(=O)c4cc(NC(=O)CCCNC(=O)c5cc(NC(=O)c6cc(NC(=O)c7nc(NC(=O)c8nccn8C)cn7C)cn6C)cn5C)cn4C)cn3C)cn2C)cn1C] |
| PBX- SynTEF- DRG2 | 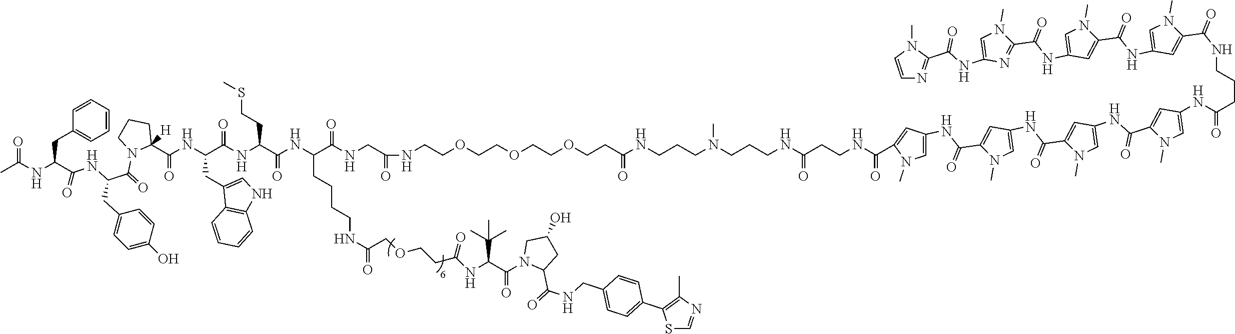 |
| PBX- SynTEF- DRG3 | 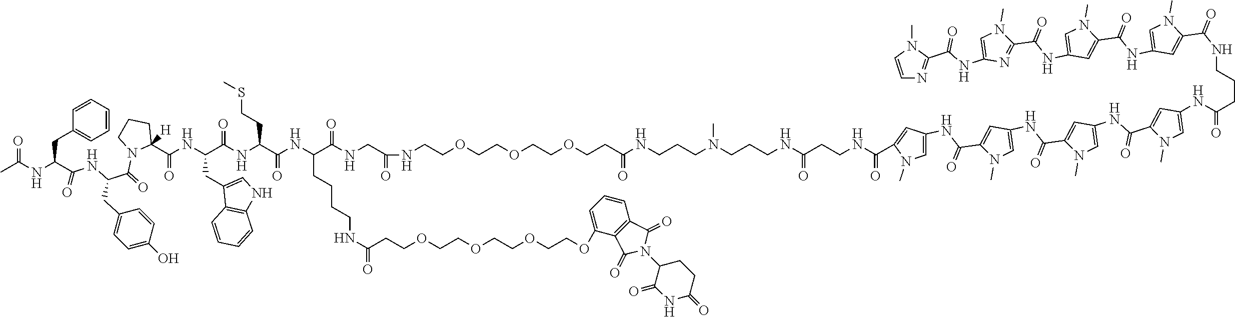 [SMILES: CSCC[C@H](NC(=O)[C@H](Cc1c[nH]c2ccccc12)NC(=O)[C@H]1CCCN1C(=O)[C@H](Cc1ccc(O)cc1)NC(=O)[C@H](Cc1ccccc1)NC(C)=O)C(=O)NC(CCCCNC(=O)CCOCCOCCOCCOc1cccc2c1C(=O)N(C1CCC(=O)NC1=O)C2=O)C(=O)NCC(=O)NCCOCCOCCOCCC(=O)NCCCN(C)CCCNC(=O)CCNC(=O)c1cc(NC(=O)c2cc(NC(=O)c3cc(NC(=O)c4cc(NC(=O)CCCNC(=O)c5cc(NC(=O)c6cc(NC(=O)c7nc(NC(=O)c8nccn8C)cn7C)cn6C)cn5C)cn4C)cn3C)cn2C)cn1C] |
| PBX- SynTEF- DRG4 | 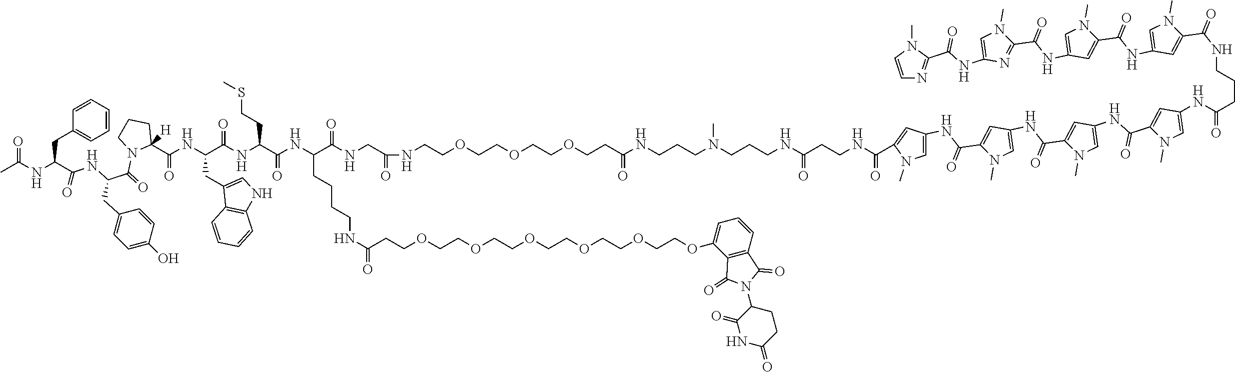 [SMILES: CSCC[C@H](NC(=O)[C@H](Cc1c[nH]c2ccccc12)NC(=O)[C@H]1CCCN1C(=O)[C@H](Cc1ccc(O)cc1)NC(=O)[C@H](Cc1ccccc1)NC(C)=O)C(=O)NC(CCCCNC(=O)CCOCCOCCOCCOCCOCCOc1cccc2c1C(=O)N(C1CCC(=O)NC1=O)C2=O)C(=O)NCC(=O)NCCOCCOCCOCCC(=O)NCCCN(C)CCCNC(=O)CCNC(=O)c1cc(NC(=O)c2cc(NC(=O)c3cc(NC(=O)c4cc(NC(=O)CCCNC(=O)c5cc(NC(=O)c6cc(NC(=O)c7nc(NC(=O)c8nccn8C)cn7C)cn6C)cn5C)cn4C)cn3C)cn2C)cn1C] |
| PBX- SynTEF- DRG5 | 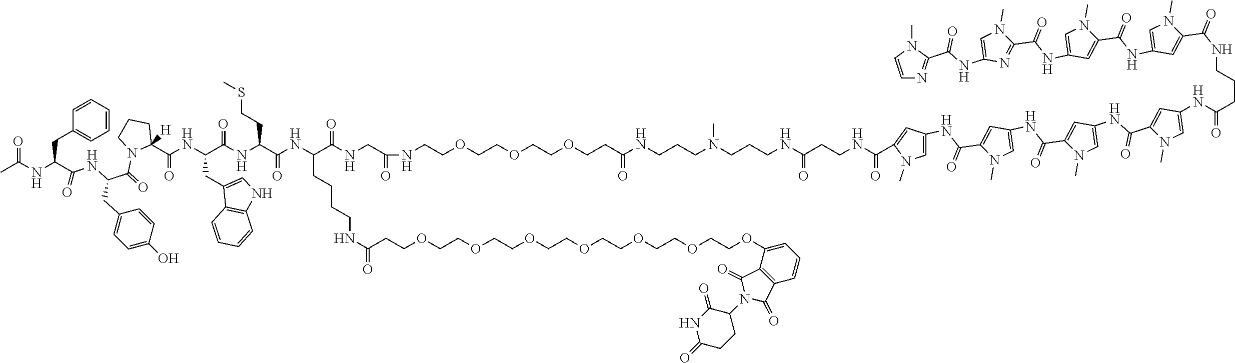 [SMILES: CSCC[C@H](NC(=O)[C@H](Cc1c[nH]c2ccccc12)NC(=O)[C@H]1CCCN1C(=O)[C@H](Cc1ccc(O)cc1)NC(=O)[C@H](Cc1ccccc1)NC(C)=O)C(=O)NC(CCCCNC(=O)CCOCCOCCOCCOCCOCCOCCOc1cccc2c1C(=O)N(C1CCC(=O)NC1=O)C2=O)C(=O)NCC(=O)NCCOCCOCCOCCC(=O)NCCCN(C)CCCNC(=O)CCNC(=O)c1cc(NC(=O)c2cc(NC(=O)c3cc(NC(=O)c4cc(NC(=O)CCCNC(=O)c5cc(NC(=O)c6cc(NC(=O)c7nc(NC(=O)c8nccn8C)cn7C)cn6C)cn5C)cn4C)cn3C)cn2C)cn1C] |
| PBX- SynTEF- DRG6 | 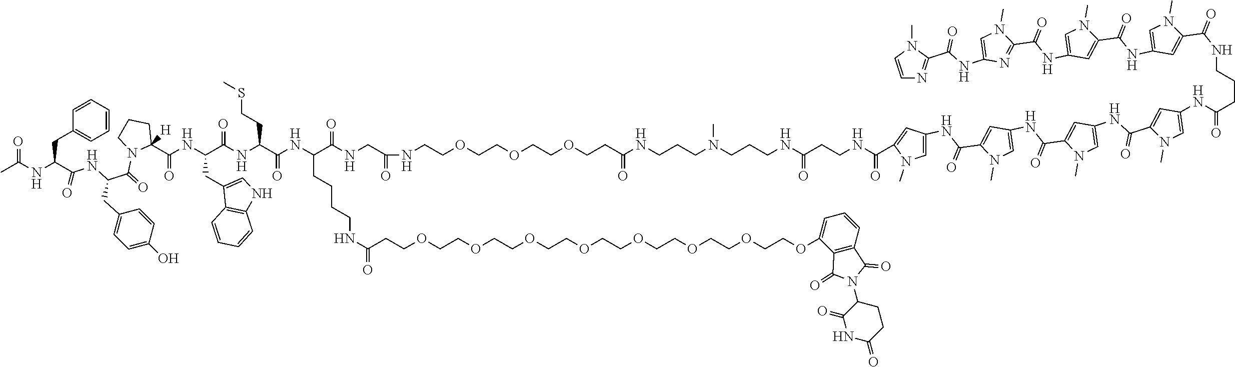 [SMILES: CSCC[C@H](NC(=O)[C@H](Cc1c[nH]c2ccccc12)NC(=O)[C@H]1CCCN1C(=O)[C@H](Cc1ccc(O)cc1)NC(=O)[C@H](Cc1ccccc1)NC(C)=O)C(=O)NC(CCCCNC(=O)CCOCCOCCOCCOCCOCCOCCOCCOc1cccc2c1C(=O)N(C1CCC(=O)NC1=O)C2=O)C(=O)NCC(=O)NCCOCCOCCOCCC(=O)NCCCN(C)CCCNC(=O)CCNC(=O)c1cc(NC(=O)c2cc(NC(=O)c3cc(NC(=O)c4cc(NC(=O)CCCNC(=O)c5cc(NC(=O)c6cc(NC(=O)c7nc(NC(=O)c8nccn8C)cn7C)cn6C)cn5C)cn4C)cn3C)cn2C)cn1C] |
| PBX SynTEF-6 DRG7 | 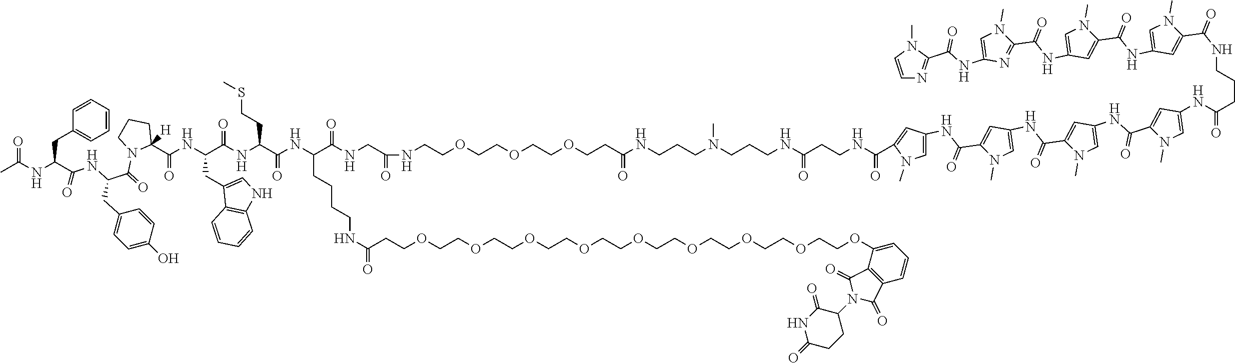 [SMILES: CSCC[C@H](NC(=O)[C@H](Cc1c[nH]c2ccccc12)NC(=O)[C@H]1CCCN1C(=O)[C@H](Cc1ccc(O)cc1)NC(=O)[C@H](Cc1ccccc1)NC(C)=O)C(=O)NC(CCCCNC(=O)CCOCCOCCOCCOCCOCCOCCOCCOCCOc1cccc2c1C(=O)N(C1CCC(=O)NC1=O)C2=O)C(=O)NCC(=O)NCCOCCOCCOCCC(=O)NCCCN(C)CCCNC(=O)CCNC(=O)c1cc(NC(=O)c2cc(NC(=O)c3cc(NC(=O)c4cc(NC(=O)CCCNC(=O)c5cc(NC(=O)c6cc(NC(=O)c7nc(NC(=O)c8nccn8C)cn7C)cn6C)cn5C)cn4C)cn3C)cn2C)cn1C] |
| PBX- SynTEF- DRG8 | 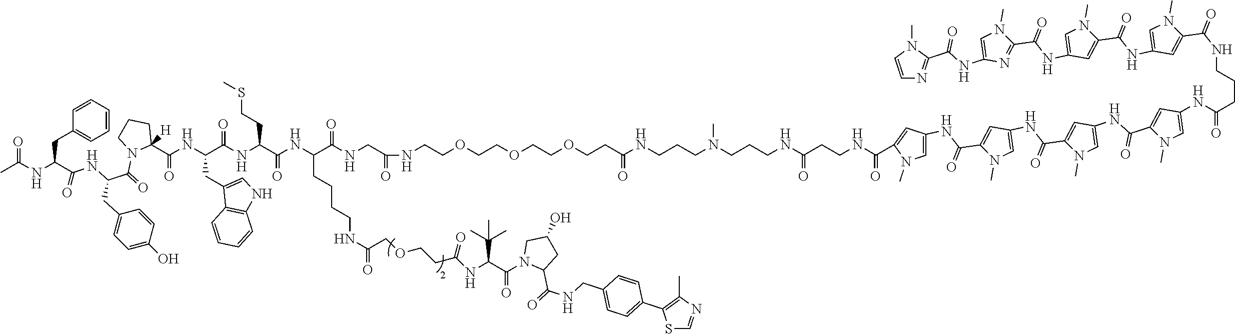 |
| PBX- SynTEF- DRG9 | 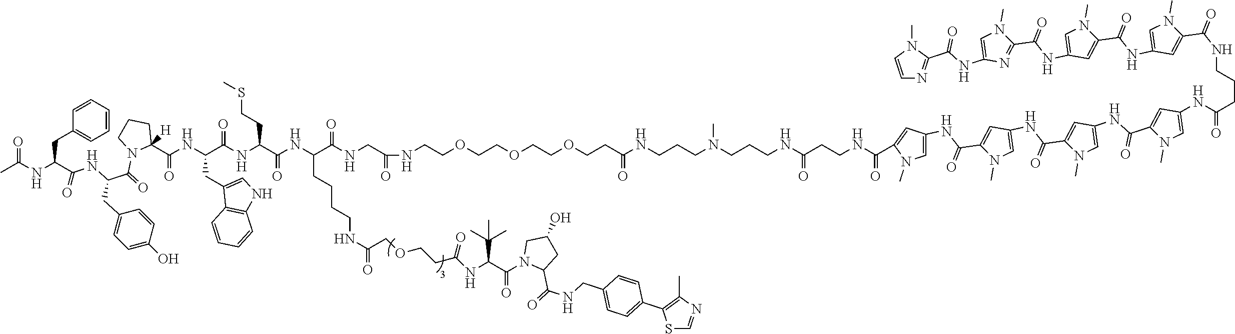 |
| PBX- SynTEF- DRG10 | 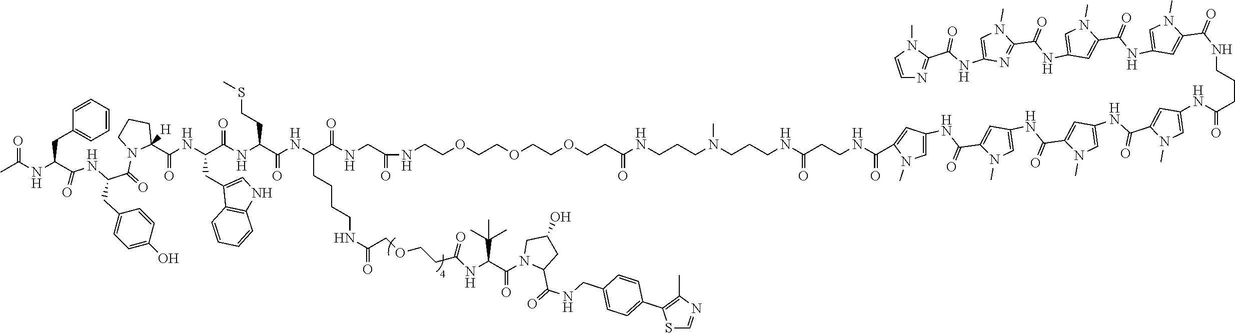 |
| PBX- SynTEF- DRG11 | 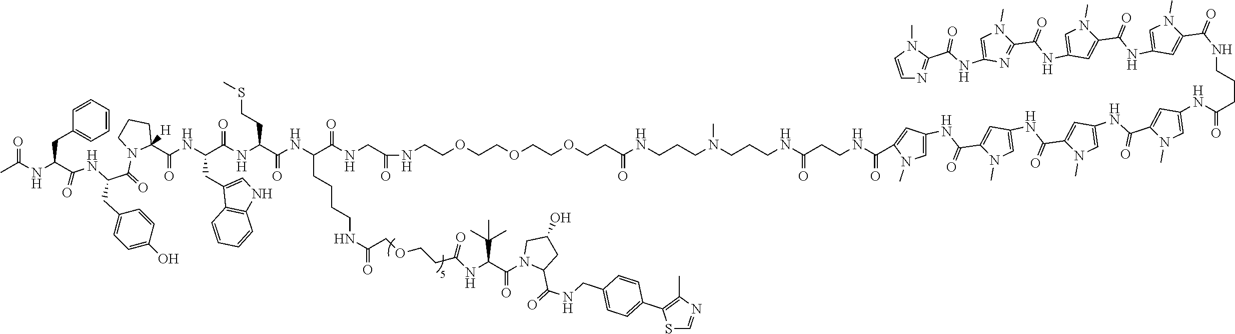 |
| PBX- SynTEF- DRG12 | 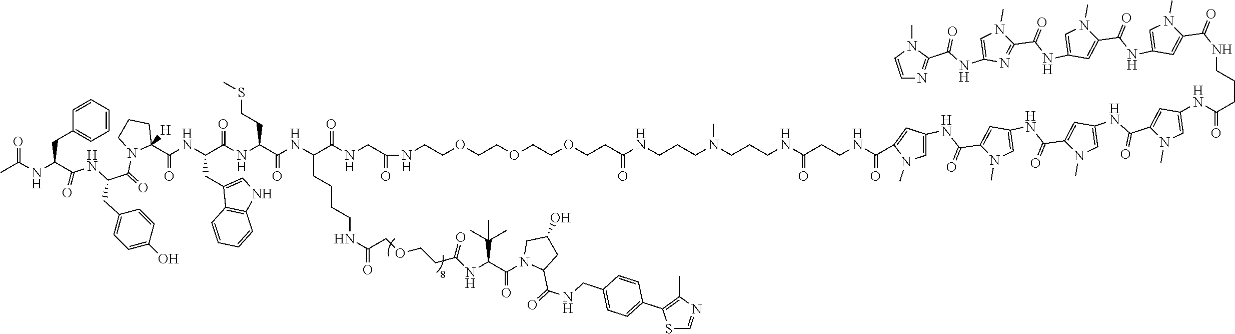 |
| PBX- SynTEF- DRG13 | 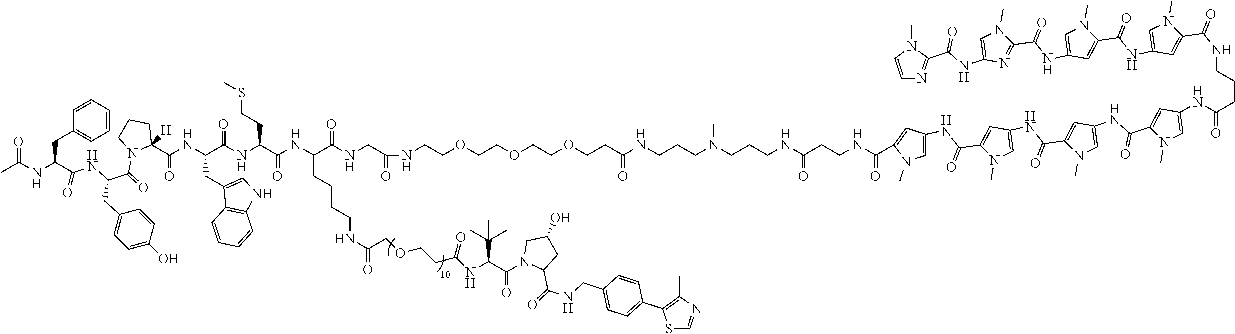 |

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1-76. (canceled)

77. A sequence selective DNA binding compound comprising:
(a) a polyamide moiety configured to bind a deoxyribonucleic acid (DNA) sequence ^{5′}WGGWWW^{3′};
(b) two of a Ubiquitin-Proteosome System (UPS) targeting moiety, a bromodomain extraterminal domain (BET) binding ligand, and a PBX or TCF3-PBX transcription factor binding moiety,
wherein the polyamide moiety and two of the UPS targeting moiety, the BET binding ligand, and the transcription factor binding moiety are tethered via a first chemical linker and via a second chemical linker,
wherein the UPS targeting moiety is an E3 ligase binding ligand selected from: 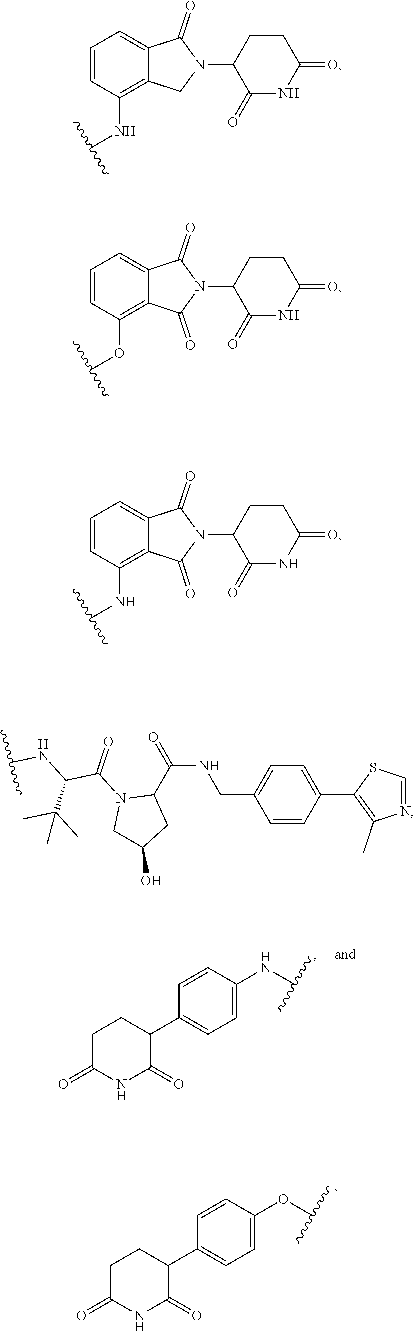 [SMILES: *Nc1cccc2c1CN(C1CCC(=O)NC1=O)C2=O ; *Oc1cccc2c1C(=O)N(C1CCC(=O)NC1=O)C2=O ; *Nc1cccc2c1C(=O)N(C1CCC(=O)NC1=O)C2=O ; *N[C@H](C(=O)N1C[C@H](O)CC1C(=O)NCc1ccc(-c2scnc2C)cc1)C(C)(C)C ; *Nc1ccc(C2CCC(=O)NC2=O)cc1 ; *Oc1ccc(C2CCC(=O)NC2=O)cc1]
wherein the BET binding ligand is selected from: 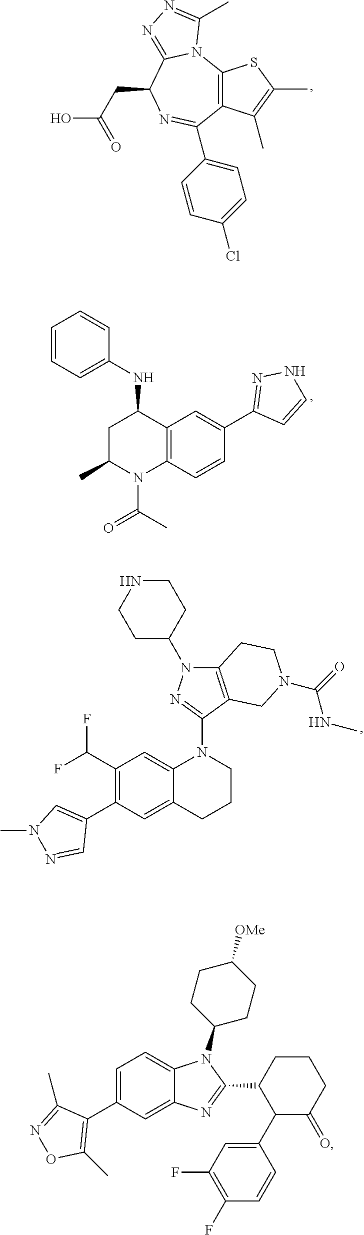 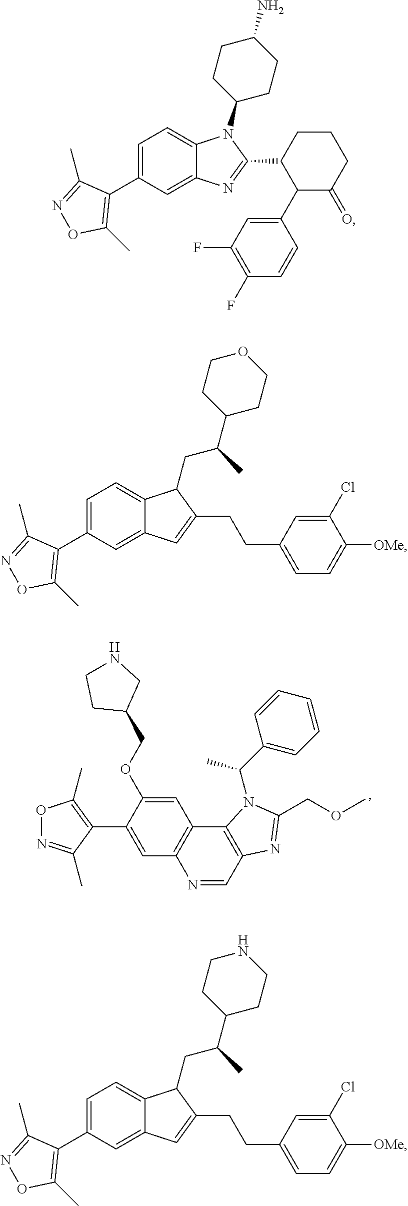 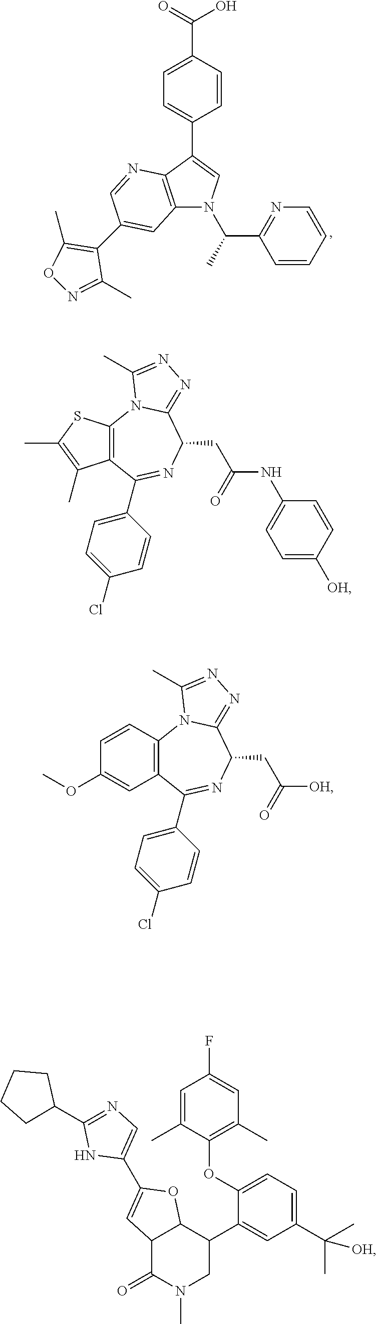 [SMILES: Cc1noc(C)c1-c1cnc2c(-c3ccc(C(=O)O)cc3)cn([C@@H](C)c3ccccn3)c2c1 ; Cc1sc2c(c1C)C(c1ccc(Cl)cc1)=N[C@@H](CC(=O)Nc1ccc(O)cc1)c1nnc(C)n1-2 ; COc1ccc2c(c1)C(c1ccc(Cl)cc1)=N[C@@H](CC(=O)O)c1nnc(C)n1-2 ; Cc1cc(F)cc(C)c1Oc1ccc(C(C)(C)O)cc1C1CN(C)C(=O)C2C=C(c3cnc(C4CCCC4)[nH]3)OC21] 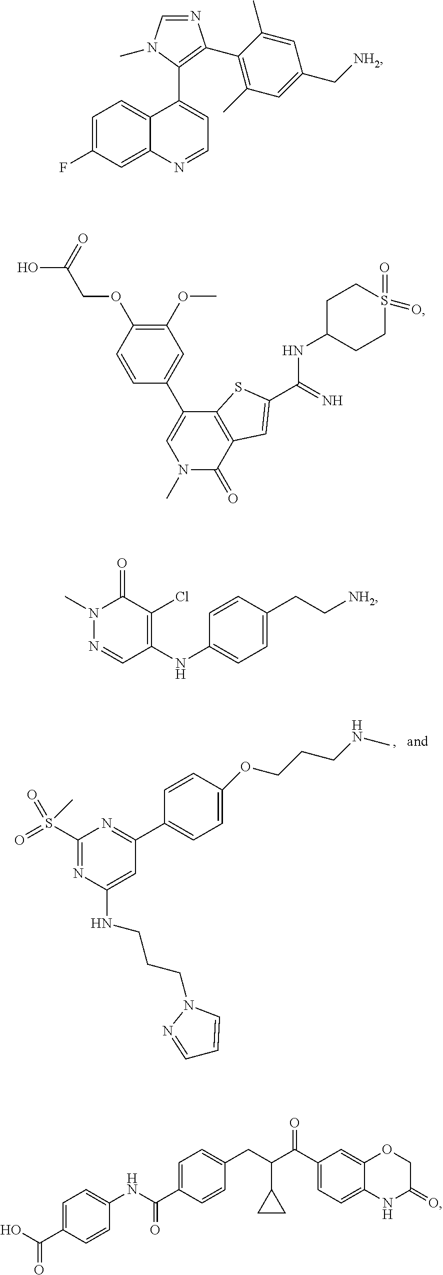 [SMILES: Cc1cc(CN)cc(C)c1-c1ncn(C)c1-c1ccnc2cc(F)ccc12 ; COc1cc(-c2cn(C)c(=O)c3cc(C(=N)NC4CCS(=O)(=O)CC4)sc23)ccc1OCC(=O)O ; Cn1ncc(Nc2ccc(CCN)cc2)c(Cl)c1=O ; CNCCCOc1ccc(-c2cc(NCCCn3cccn3)nc(S(C)(=O)=O)n2)cc1 ; O=C1COc2cc(C(=O)C(Cc3ccc(C(=O)Nc4ccc(C(=O)O)cc4)cc3)C3CC3)ccc2N1]
and
wherein the transcription factor binding moiety is selected from:

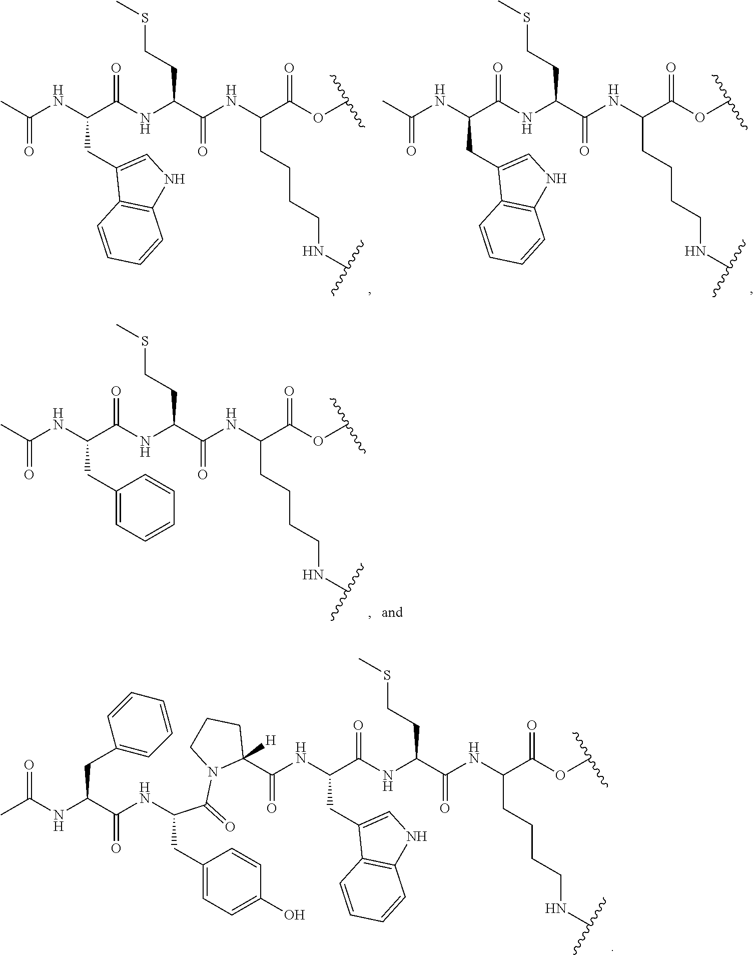

78-137. (canceled)

138. The sequence selective DNA binding compound of claim 77, wherein the polyamide moiety comprises a monomeric unit having a structure represented by a formula: 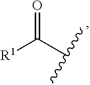
wherein R¹ is selected from: 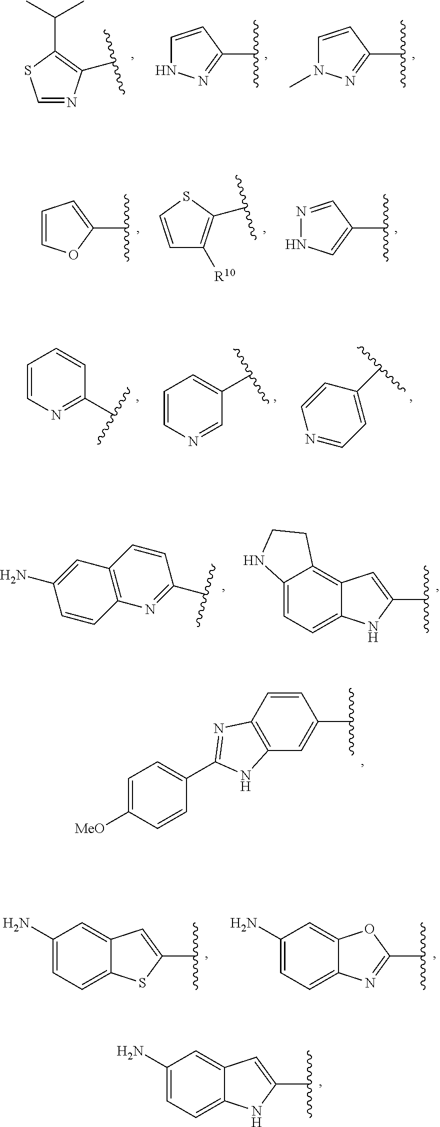 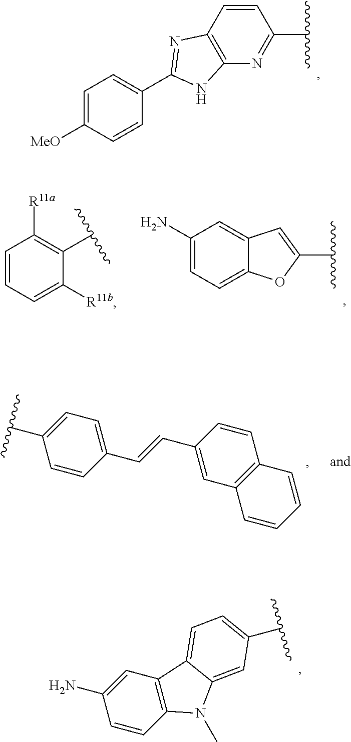
wherein R¹⁰ is selected from hydrogen, halogen, —OH, —NH₂, —NHC(O)CH₃, methyl, and methoxy; and
wherein each of R^{11a} and R^{11b} is independently selected from hydrogen, —OH, methyl, and methoxy.

139. The sequence selective DNA binding compound of claim 77, wherein the polyamide moiety comprises a monomeric unit having a structure represented by a formula: 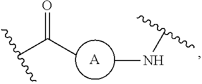
wherein \textcircled{A} is selected from:

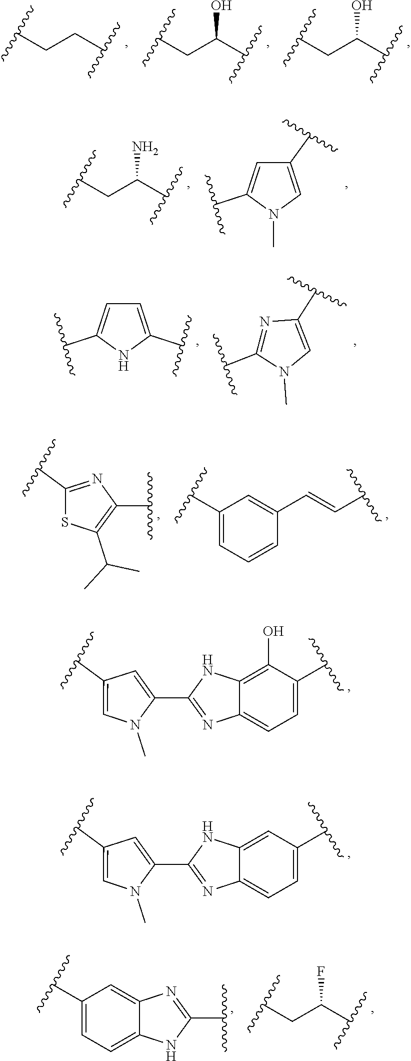

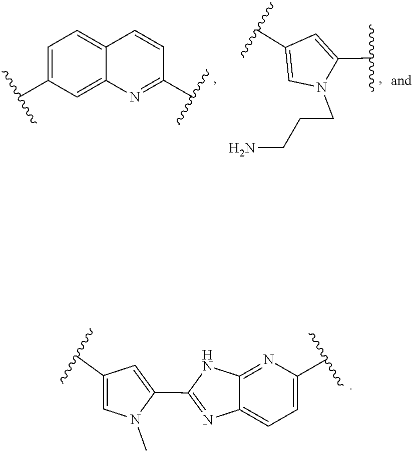 [SMILES: *c1ccc2ccc(*)nc2c1 ; *c1cc(*)n(CCCN)c1 ; *c1cc(-c2nc3ccc(*)nc3[nH]2)n(C)c1]

140. The sequence selective DNA binding compound of claim 77, wherein the polyamide moiety comprises a monomeric unit having a structure represented by a formula selected from: 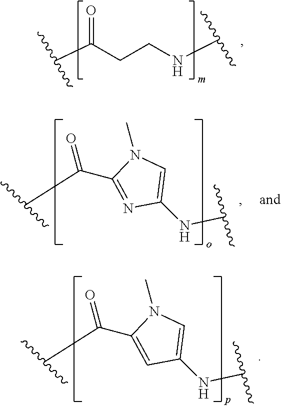
wherein each of m, o, and p is independently an integer selected from 1 to 20.

141. The sequence selective DNA binding compound of claim 77, wherein the polyamide moiety is selected from:

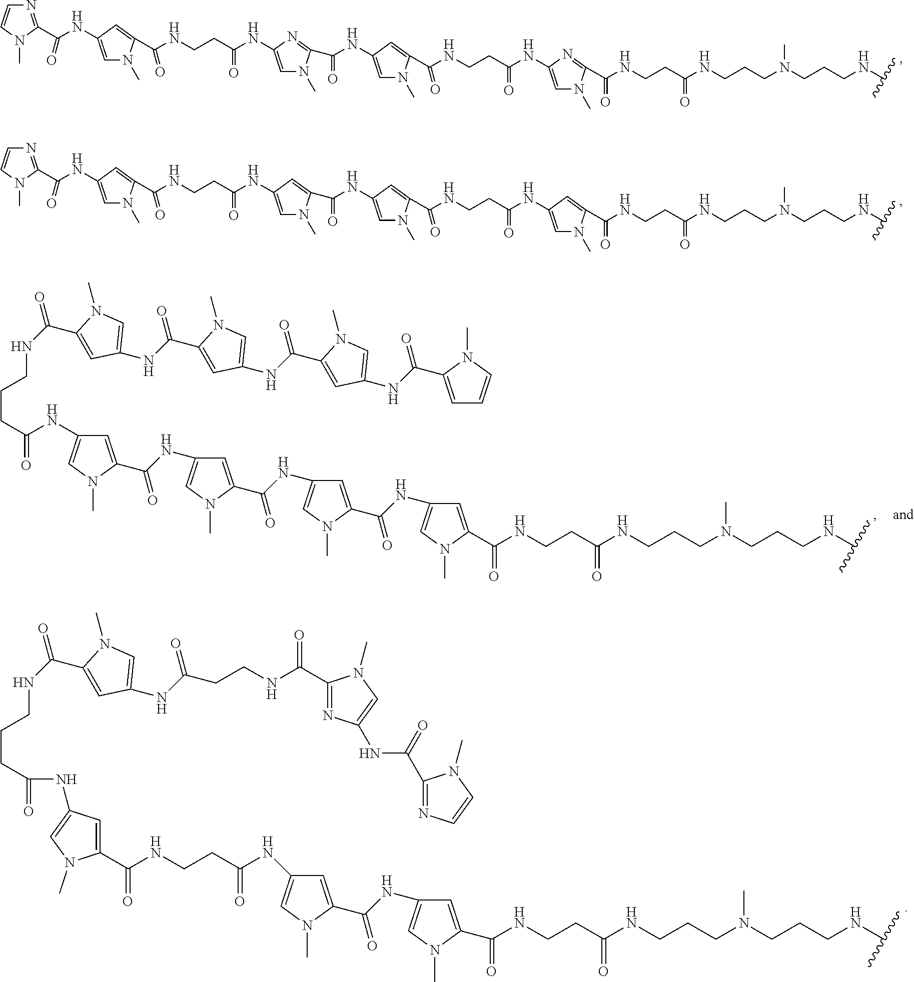

142. The sequence selective DNA binding compound of claim 77, wherein the polyamide moiety is selected from:

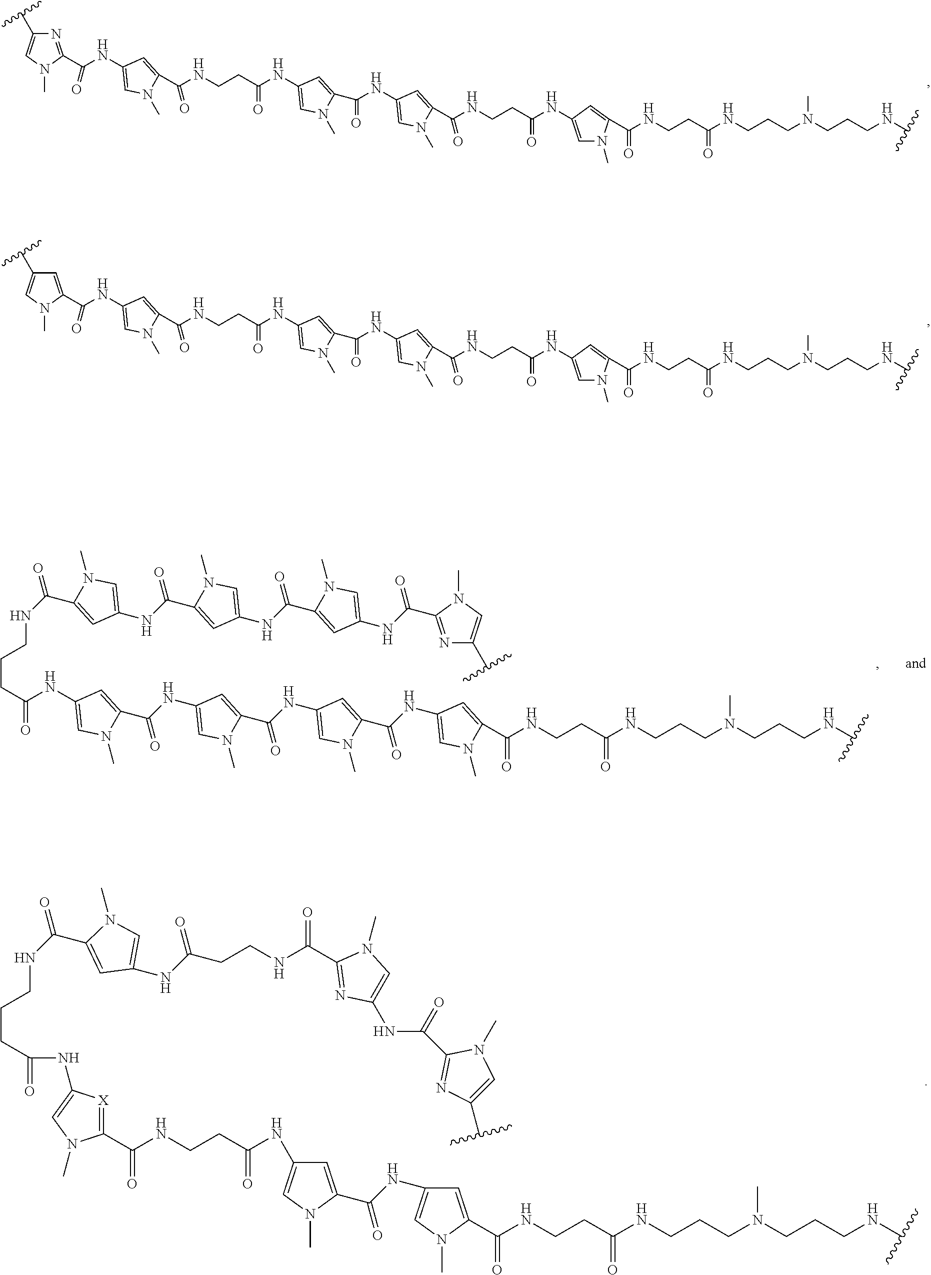

143. The sequence selective DNA binding compound of claim 77, wherein the first chemical linker is a polyethylene glycol (PEG) linker having a structure represented by a formula selected from: 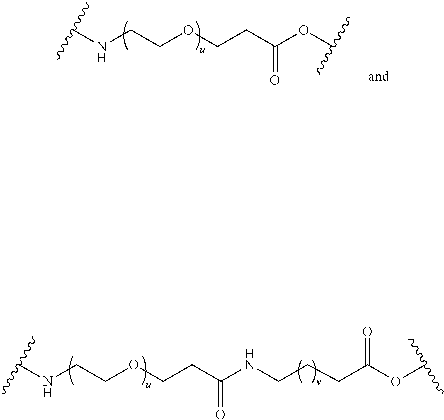
wherein each of u and v is independently an integer selected from 1 to 20.

144. The sequence selective DNA binding compound of claim 77, wherein the first chemical linker is an alkyl linker having a structure represented by a formula: 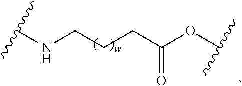
wherein w is an integer selected from 1 to 20.

145. The sequence selective DNA binding compound of claim 77, wherein the second chemical linker is a polyethylene glycol (PEG) linker having a structure represented by a formula selected from: 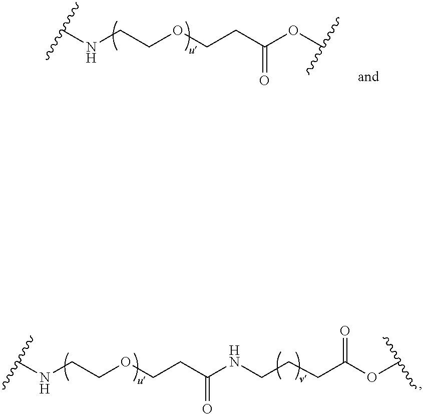
wherein each of u′ and v′ is independently an integer selected from 1 to 20.

146. The sequence selective DNA binding compound of claim 77, wherein the second chemical linker is an alkyl linker having a structure represented by a formula: 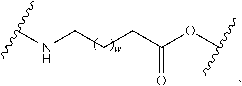
wherein w′ is an integer selected from 1 to 20.

147. The sequence selective DNA binding compound of claim 77, wherein the compound comprises the transcription factor binding moiety.

148. The sequence selective DNA binding compound of claim 77, wherein the compound comprises the UPS targeting moiety.

149. The sequence selective DNA binding compound of claim 77, wherein the compound comprises the BET binding ligand.

150. The sequence selective DNA binding compound of claim 77, wherein the compound has a structure represented by a formula selected from: 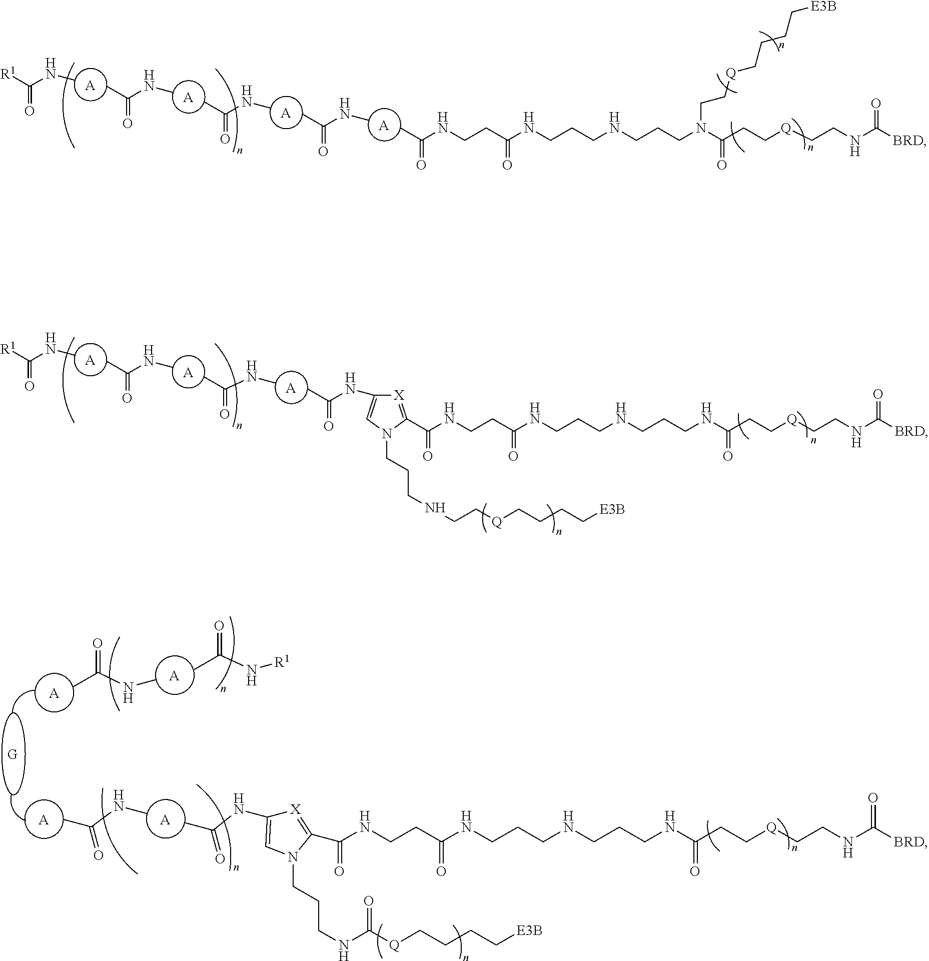 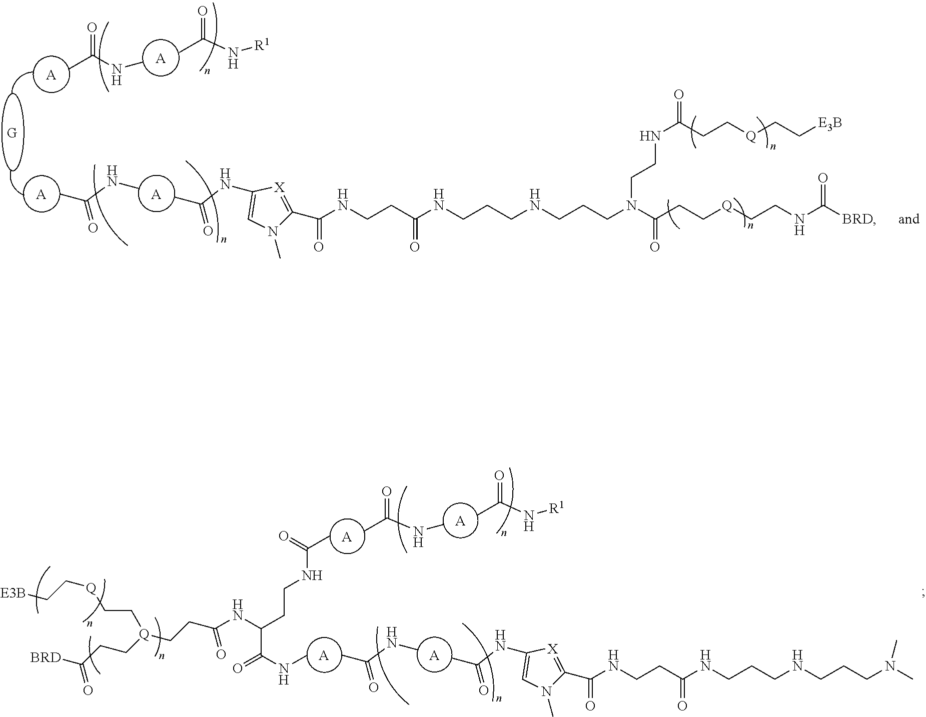
wherein E3B is the E3 ligase binding ligand;
wherein BRD is the BET binding ligand;
wherein each occurrence of n is independently an integer selected from 1 to 20;
wherein G is a polyamide turn linker;
wherein each occurrence of Q is independently selected from CH₂ and O;
wherein X is selected from N and CH;
wherein R¹ is selected from: 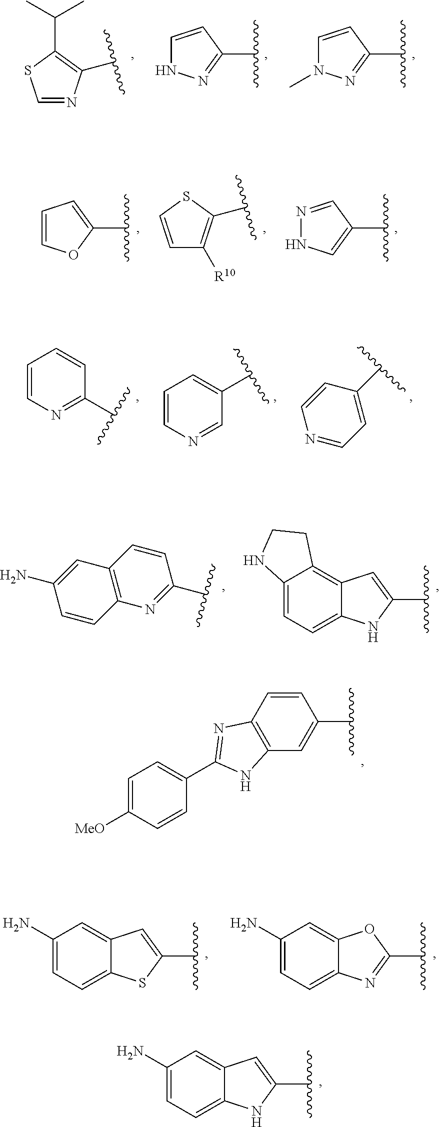 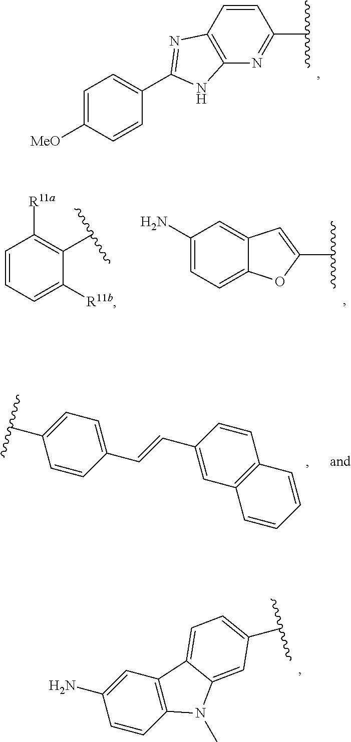
wherein R¹⁰ is selected from hydrogen, halogen, —OH, —NH₂, —NHC(O)CH₃, methyl, and methoxy; and
wherein each of R^{11a} and R^{11b} is independently selected from hydrogen, —OH, methyl, and methoxy; and
wherein each occurrence of \textcircled{A} is independently selected from:

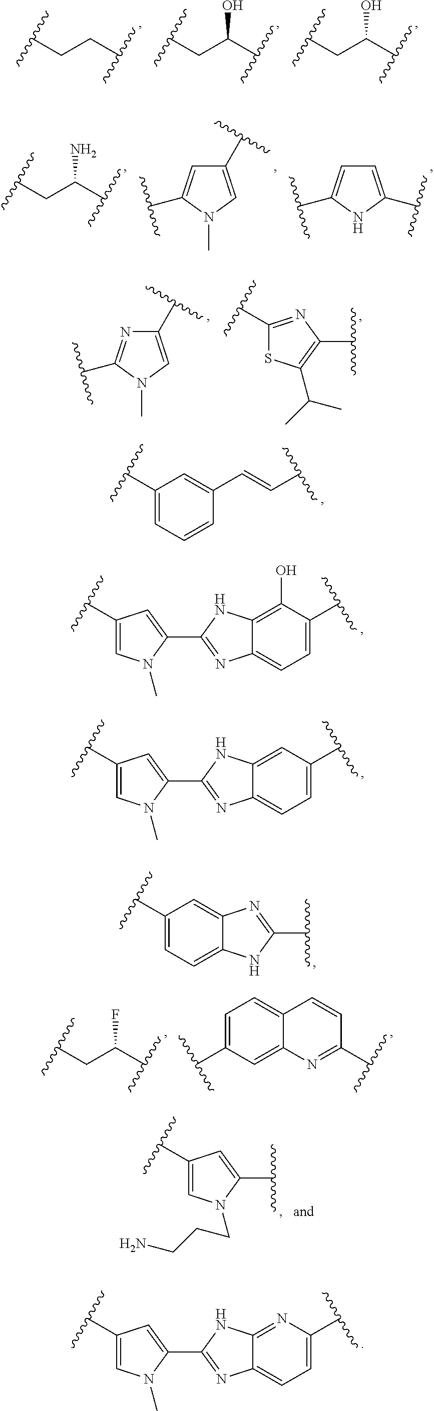

151. The sequence selective DNA binding compound of claim 77, wherein the compound has a structure represented by a formula selected from: 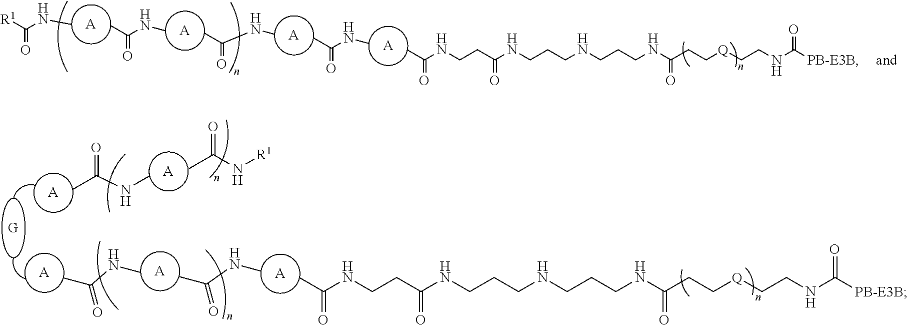
wherein E3B is an E3 ligase binding ligand;
wherein PB is a transcription factor binding moiety;
wherein each occurrence of n is independently an integer selected from 1 to 20;
wherein G is a polyamide turn linker;
wherein Q is selected from CH₂ and O;
wherein R¹ is selected from: 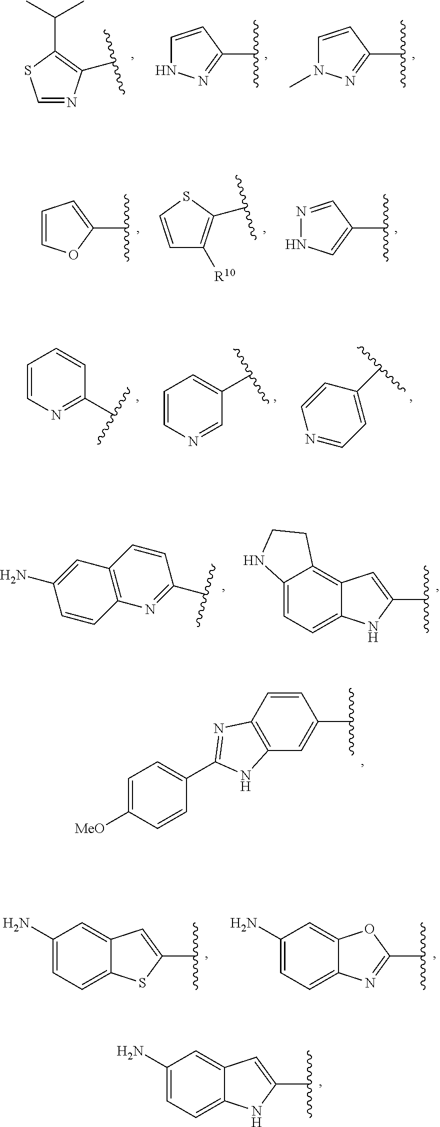 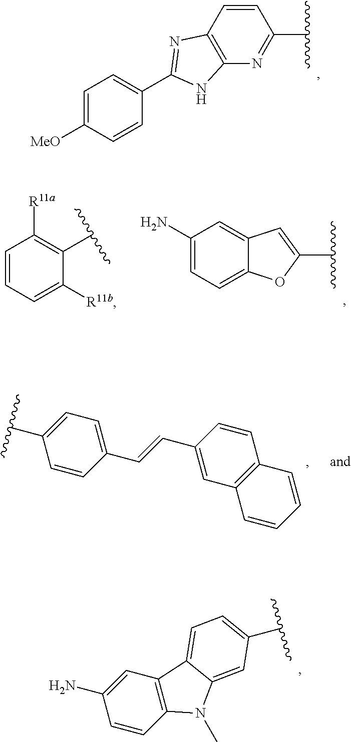
wherein R¹⁰ is selected from hydrogen, halogen, —OH, —NH₂, —NHC(O)CH₃, methyl, and methoxy; and
wherein each of R^{11a} and R^{11b} is independently selected from hydrogen, —OH, methyl, and methoxy; and
wherein each occurrence of \textcircled{A} is independently selected from:

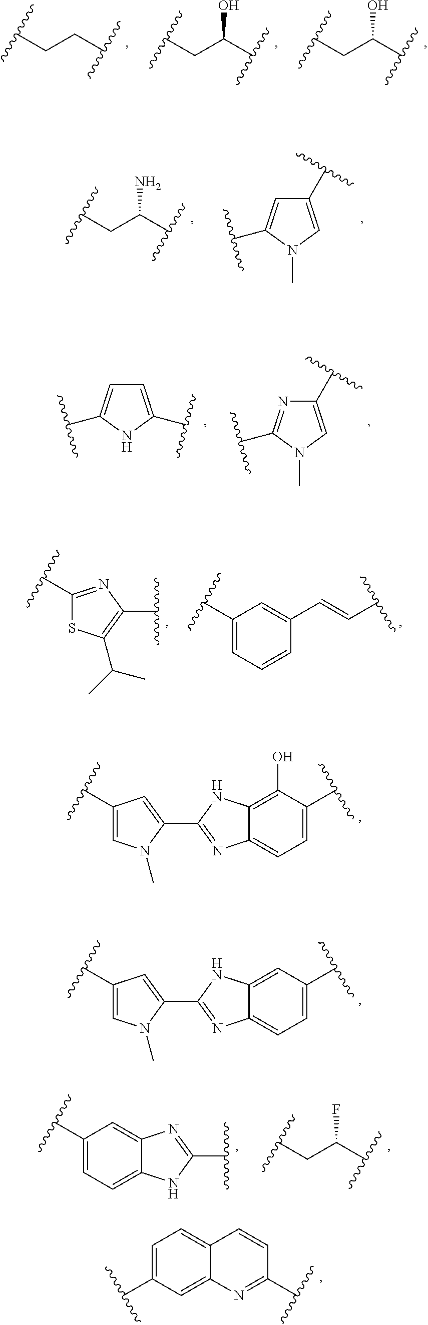

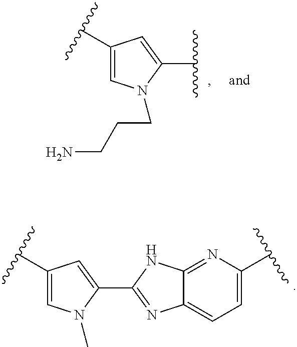 [SMILES: *c1cc(*)n(CCCN)c1 ; *c1cc(-c2nc3ccc(*)nc3[nH]2)n(C)c1]

152. The sequence selective DNA binding compound of claim 77, wherein the compound is selected from:

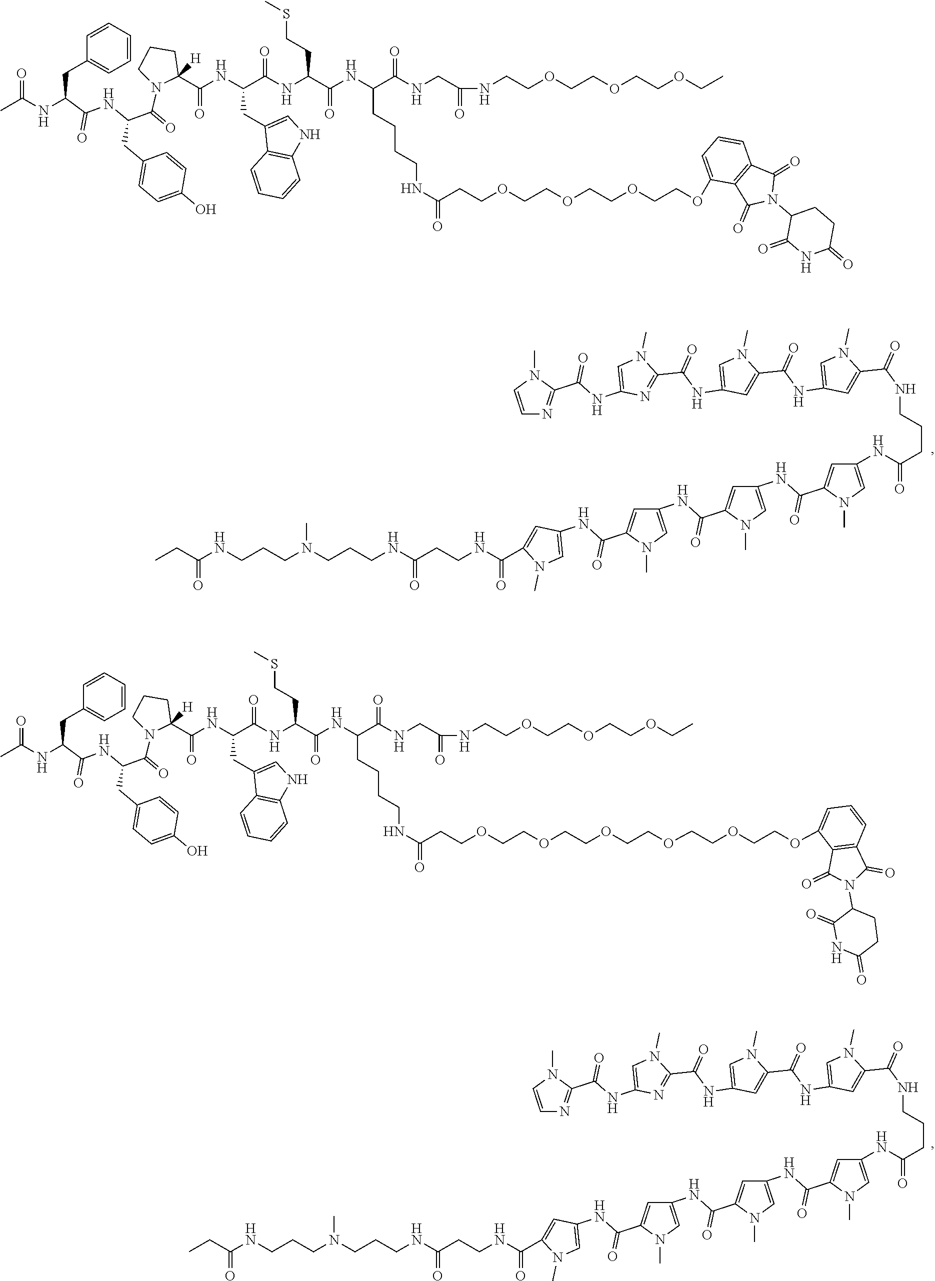

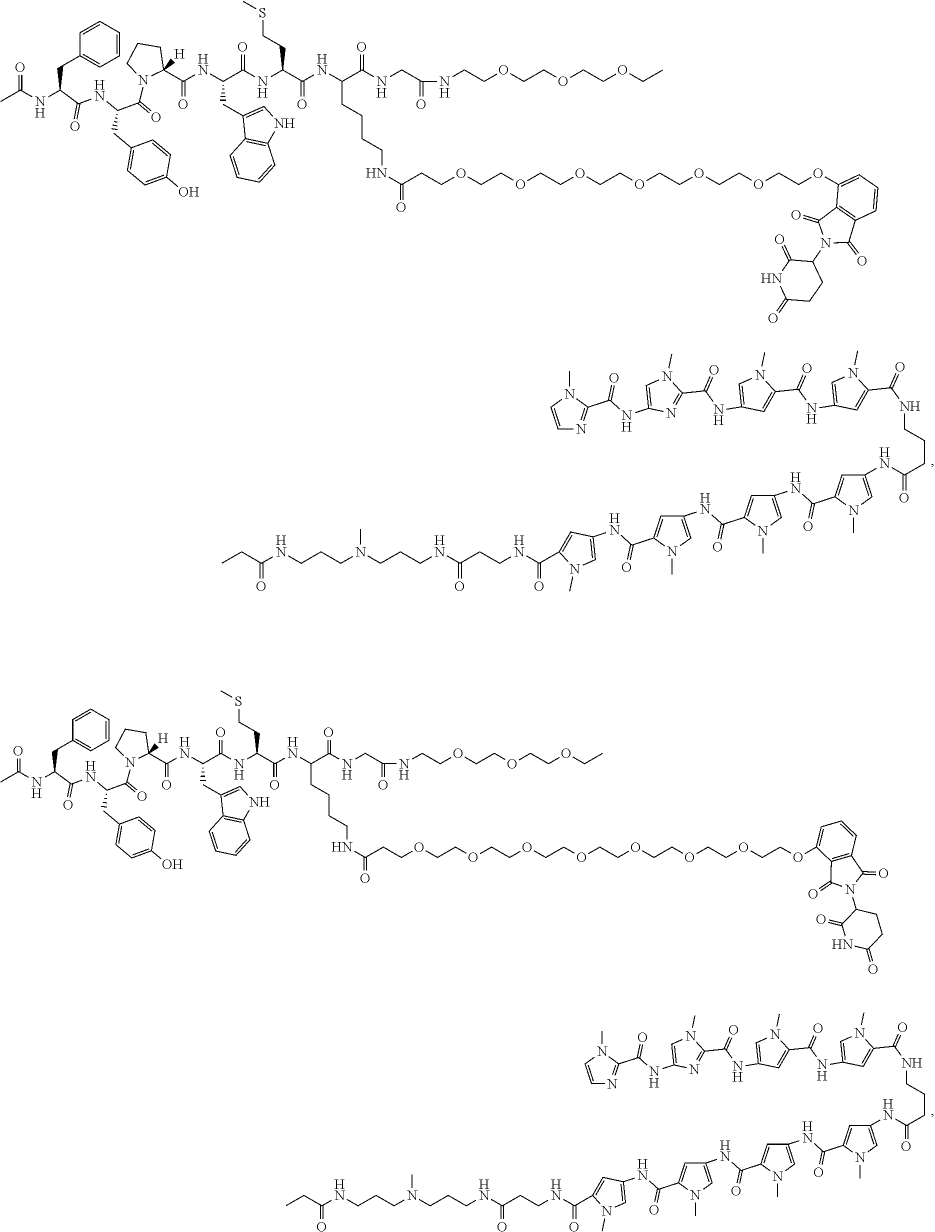

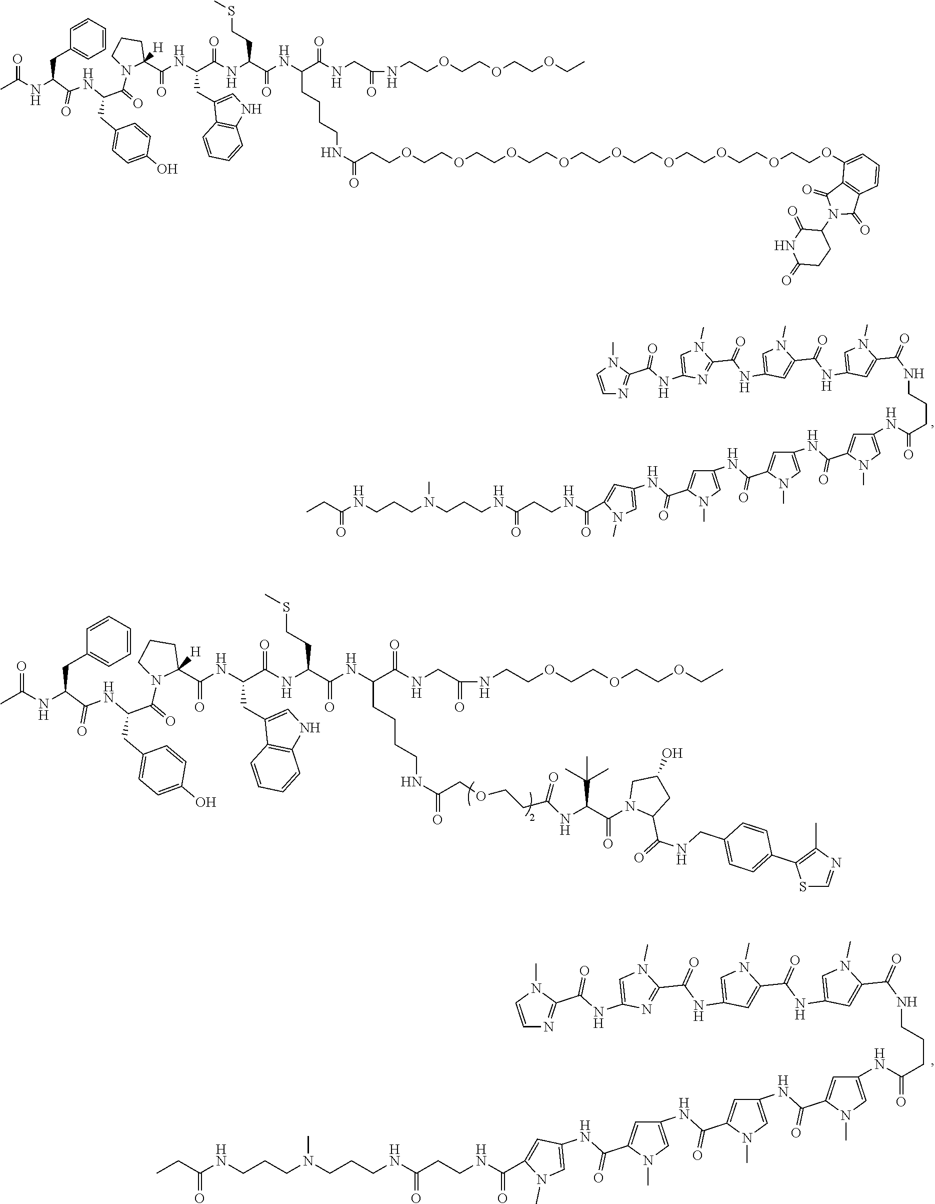

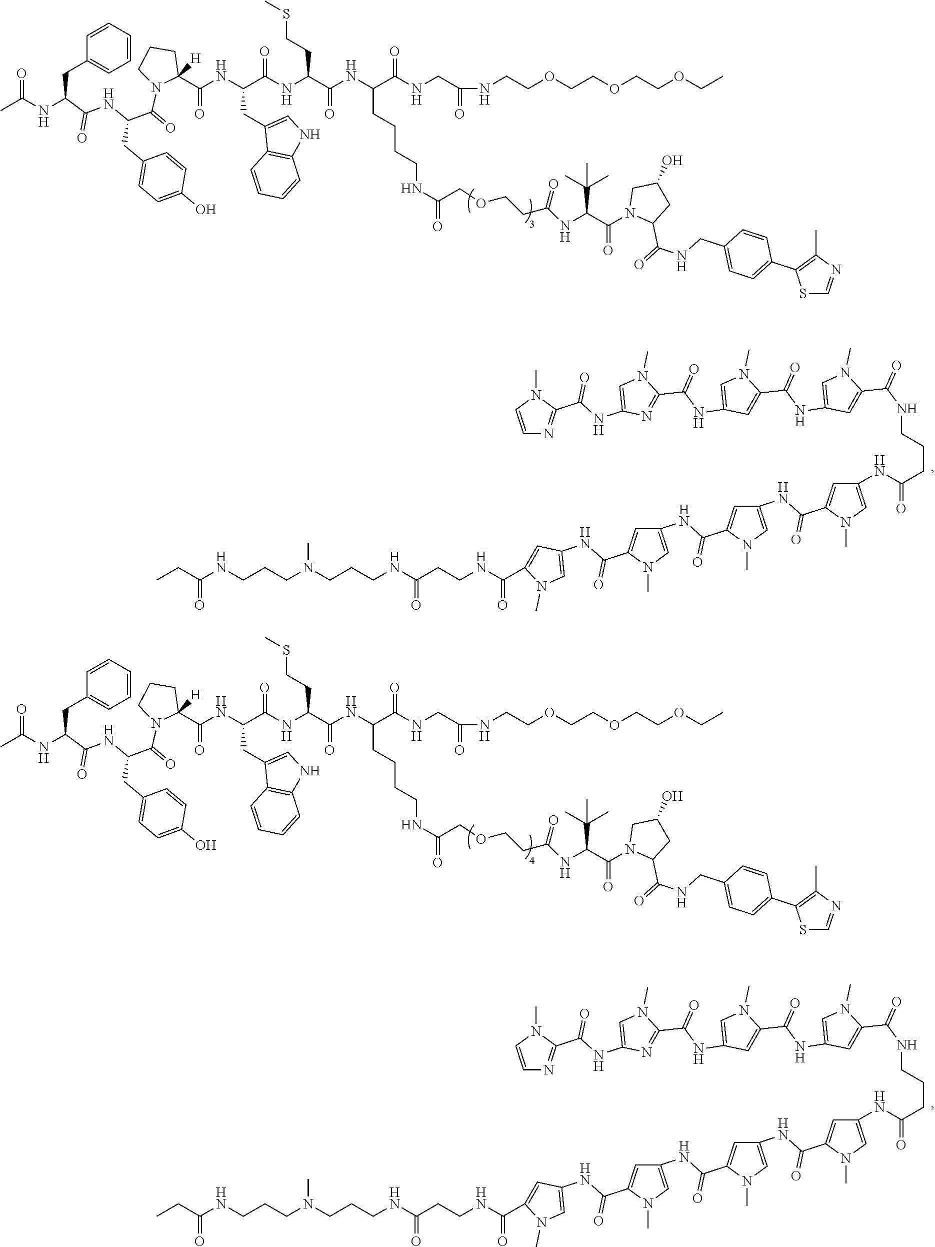

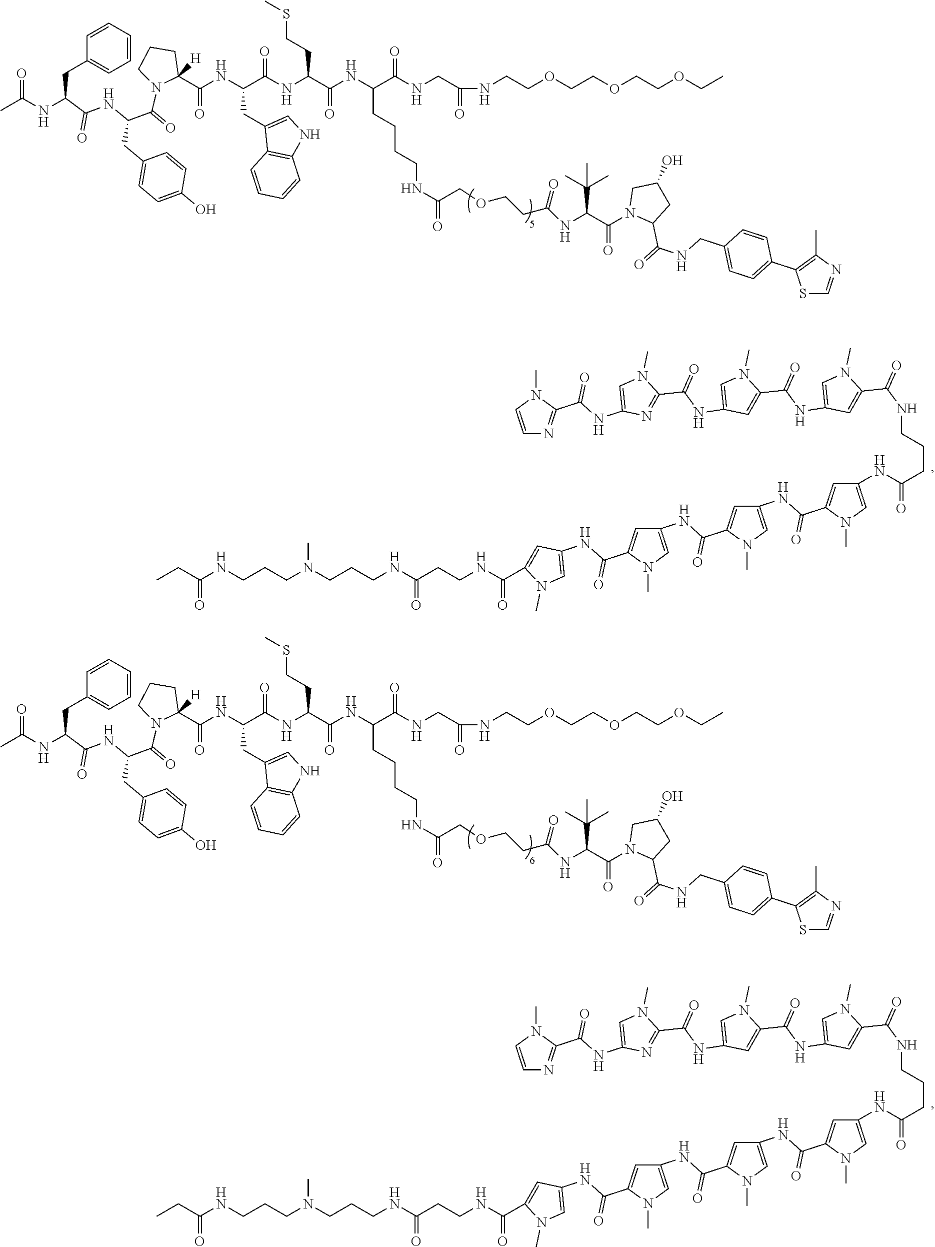

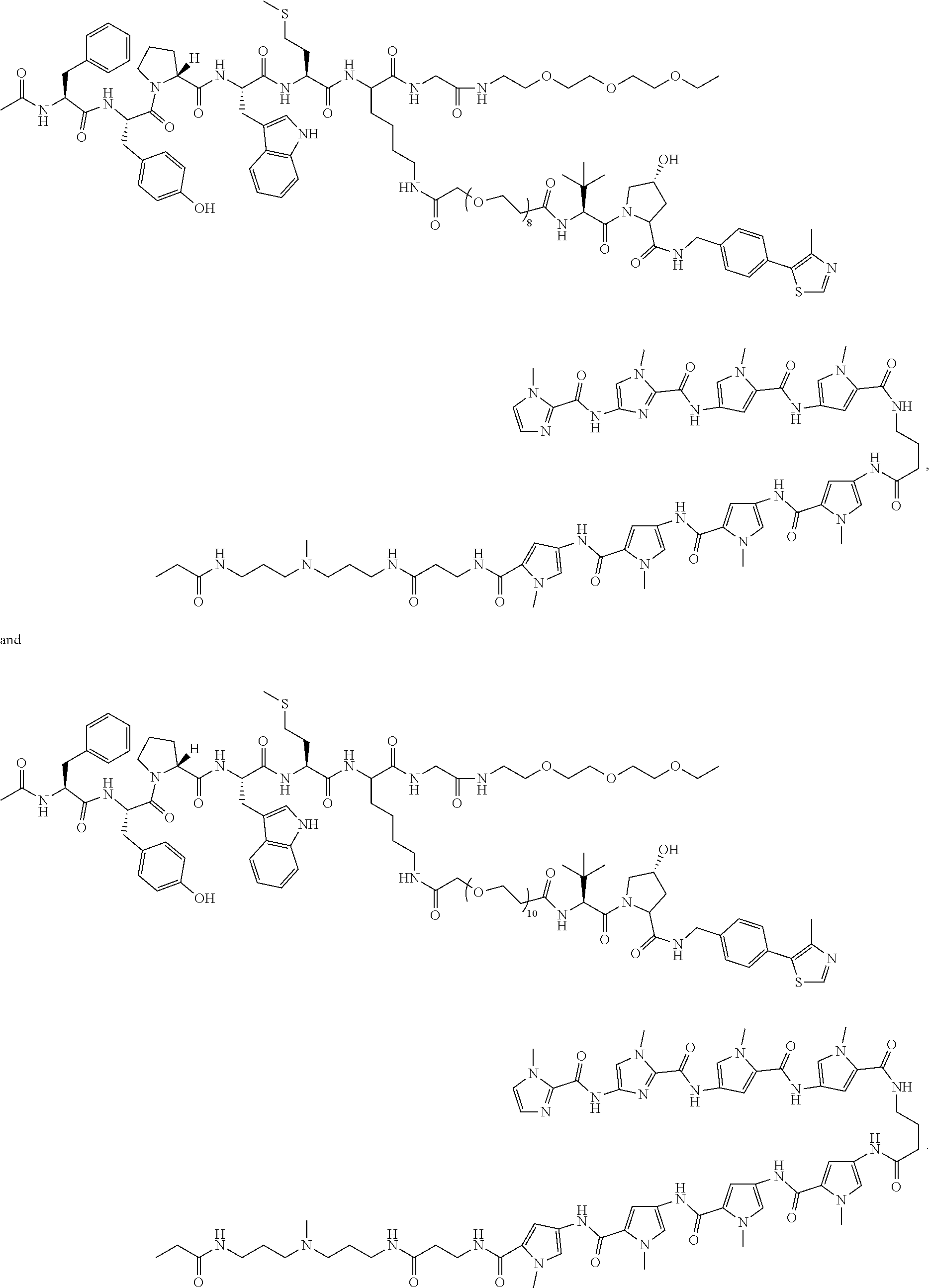

153. A pharmaceutical composition comprising the sequence selective DNA binding compound of claim 77, or a pharmaceutically acceptable salt thereof, and a pharmaceutically acceptable carrier.

154. A method of treating a disorder comprising administering to a subject having the disorder a therapeutically effective amount of the sequence selective DNA binding compound of claim 77, or a pharmaceutically acceptable salt thereof, and a pharmaceutically acceptable carrier.

155. A sequence selective DNA binding compound comprising a polyamide moiety configured to bind a deoxyribonucleic acid (DNA) sequence ^{5′}WGGWWW^{3′}, wherein the polyamide moiety is tethered via a first chemical linker to a PBX or TCF3-PBX transcription factor binding moiety selected from:

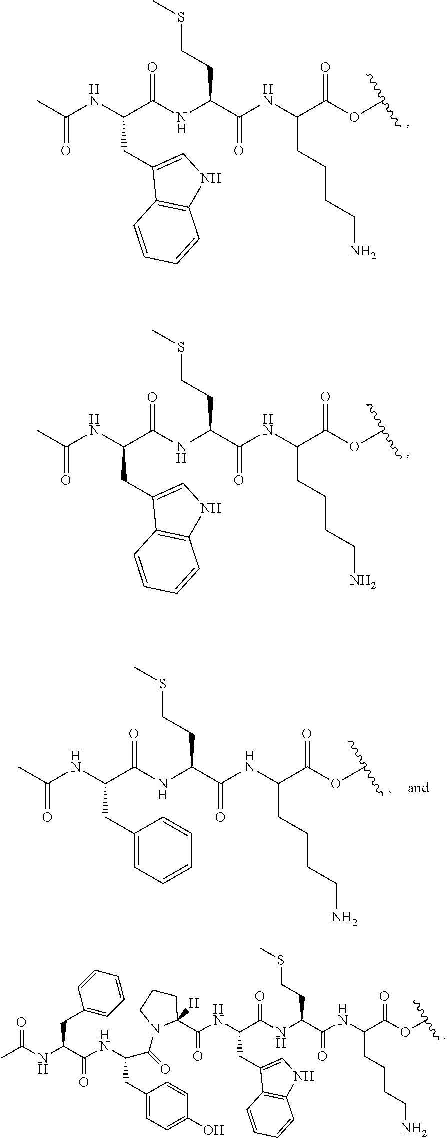 [SMILES: *OC(=O)C(CCCCN)NC(=O)[C@H](CCSC)NC(=O)[C@H](Cc1c[nH]c2ccccc12)NC(C)=O ; *OC(=O)C(CCCCN)NC(=O)[C@H](CCSC)NC(=O)[C@@H](Cc1c[nH]c2ccccc12)NC(C)=O ; *OC(=O)C(CCCCN)NC(=O)[C@H](CCSC)NC(=O)[C@H](Cc1ccccc1)NC(C)=O ; *OC(=O)C(CCCCN)NC(=O)[C@H](CCSC)NC(=O)[C@H](Cc1c[nH]c2ccccc12)NC(=O)[C@H]1CCCN1C(=O)[C@H](Cc1ccc(O)cc1)NC(=O)[C@H](Cc1ccccc1)NC(C)=O]

156. The sequence selective DNA binding compound of claim 155, wherein the compound is selected from: 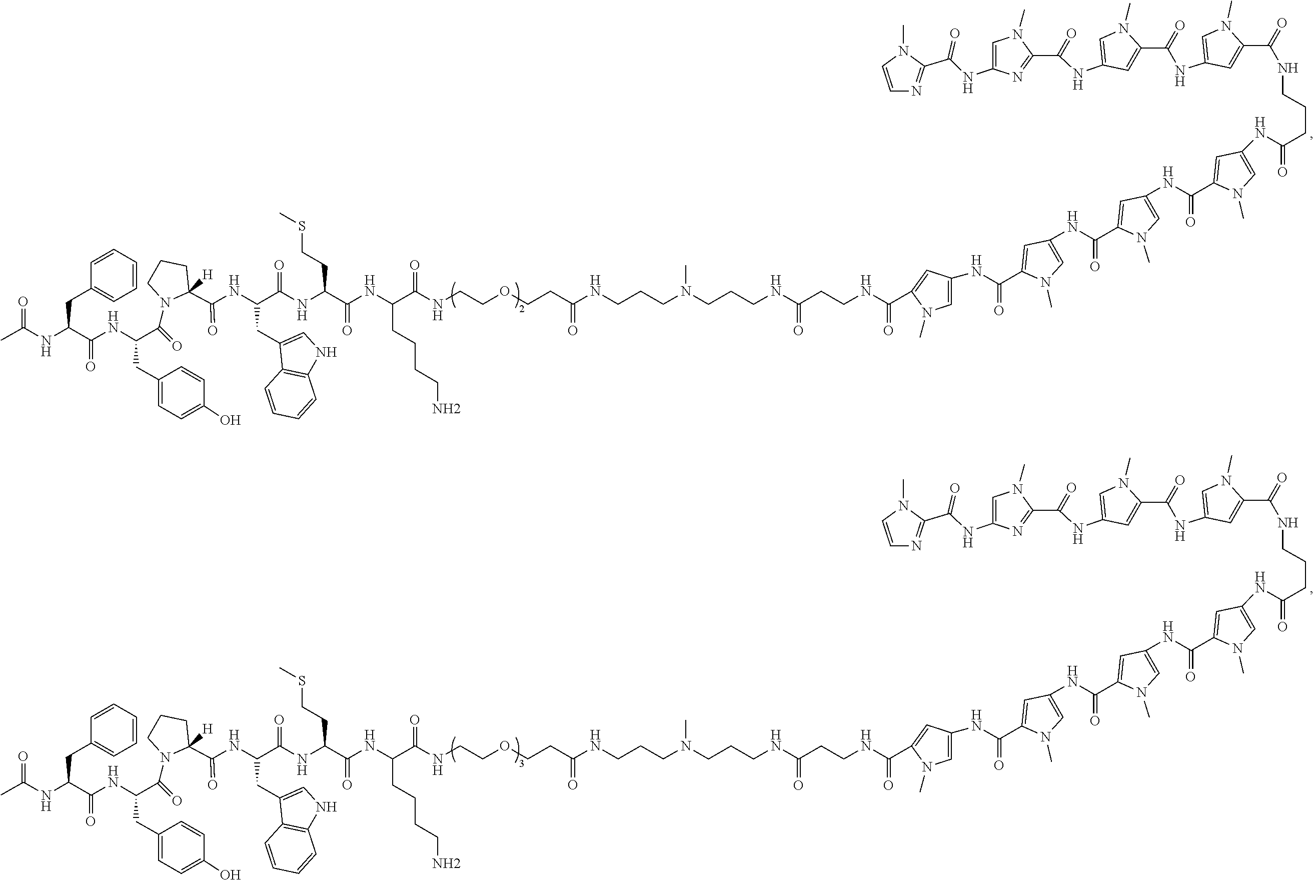 [chemical structure]
